(19) United States
(12) Patent Application Publication
(10) Pub. No.: US 2015/0037794 A1
(43) Pub. Date: Feb. 5, 2015

(54) MARKER FOR DIAGNOSING FORELIMB-GIRDLE MUSCULAR ANOMALY IN MAMMAL INDIVIDUAL, AND DETECTION METHOD USING SAME

(71) Applicant: National University Corporation Okayama University, Okayama (JP)

(72) Inventors: Tetsuo Kunieda, Okayama (JP); Takashi Hirano, Kanagawa (JP)

(21) Appl. No.: 14/358,487

(22) PCT Filed: Nov. 16, 2012
(86) PCT No.: PCT/JP2012/079843

§ 371 Date: Sep. 29, 2014

(30) Foreign Application Priority Data
Nov. 18, 2011 (JP) 2011-253314

(51) Int. Cl. C12Q 1/68

(52) U.S. Cl. CPC C12Q 1/6883; C12Q 2600/156
USPC 435/6.11; 536/23.5

(57) ABSTRACT

The present invention uses an isolated polynucleotide having a part or whole of GFRA1 gene, mutant GFRA1 protein, and mRNA encoding the mutant GFRA1 protein, each of which includes a loss-of-function mutation of GFRA1, as markers for diagnosing an animal as affected by forelimb-girdle muscular anomaly or as a carrier of forelimb-girdle muscular anomaly. In addition, an isolated polynucleotide comprising one or more selected from the group consisting of MOK2630, MOK2637, SNP B, and SNP D can be used as markers for diagnosing whether a bovine animal is affected by forelimb-girdle muscular anomaly or whether a bovine animal is a carrier of forelimb-girdle muscular anomaly.

### CROSS REFERENCE TO RELATED APPLICATIONS

This application claims the benefit of Japanese Patent Application No. 2011-253314 filed on Nov. 18, 2011, the entire disclosure of which is hereby incorporated by reference.

### TECHNICAL FIELD

The present invention relates to markers for diagnosing forelimb-girdle muscular anomaly in mammals, and methods for diagnosing forelimb-girdle muscular anomaly in mammals using the markers.

### BACKGROUND ART

Forelimb-girdle muscular anomaly, which is also referred to as “SANMAIGATA” in domestic animals, is known to be a hereditary disorder whose main symptoms are tremors and astasia caused by hypoplasia of the forelimb-girdle muscles (Masoudi et al., Animal Science Journal 78(6), 672-675, 2007). The occurrence of forelimb-girdle muscular anomaly in bovine causes a great economic loss to breeders because there is no way but disposing the animals with forelimb-girdle muscular anomaly in many cases. This hereditary disorder is known to be caused by a recessive mutation. But since the gene responsible for this disorder has not yet been known and carriers of the mutation cannot be identified, the occurrence of the disorder cannot be prevented.

### SUMMARY OF THE INVENTION

### Problems to be Solved by the Invention

An object of the present invention is to provide markers for diagnosing forelimb-girdle muscular anomaly in mammals and methods of diagnosing forelimb-girdle muscular anomaly in mammals using the markers.

### Means to Solve the Problems

The present inventors have found that a nucleotide in the bovine genome corresponding to a nucleotide at position 1060 of cDNA of the GFRA1 gene (GenBank Accession No.: NM_{—}001105411.1) is cytosine (C) in normal bovine animals, while bovine animals affected by forelimb-girdle muscular anomaly and carriers of forelimb-girdle muscular anomaly have a nonsense mutation in which this nucleotide is replaced by thymine (T) and have lost function of the GFRA1 gene. The present invention was thus completed.

As used herein, a mammal may be a human or a non-human animal, and may be a laboratory animal such as a mouse, a rat, a rabbit, and a monkey, a companion animal such as a dog or a cat, and a domestic animal such as a bovine, a horse, a sheep, and a pig.

The term “bovine” as used herein refers to animals of the genus Bos, which include domestic cattle of the species Bos Taurus, bantengs of the species of Bos javanicus (wild cattle), and zebus of the species of Bos indicus.

In the present specification, the position of a nucleotide is represented in a sense strand of the DNA double strand unless otherwise specified. In addition, the position of the nucleotide is indicated by nucleotide position numbered from the 5′ end to the 3′ end of a referenced nucleotide sequence, such as position 1060 of cDNA of the GDNF (glial cell line-derived neurotrophic factor) family receptor alpha 1 (GFRA1) gene (NM_{—}001105411.1; SEQ ID NO. 1). In a specific bovine animal, the nucleotide needs to be at the position corresponding to that specified in the referenced nucleotide sequence and the number representing the nucleotide position needs not to be necessarily identical to that of the referenced nucleotide sequence when the genome DNA containing the nucleotide has a deletion or an insertion of nucleotides.

A marker according to the present invention is for diagnosing whether a mammal is affected by forelimb-girdle muscular anomaly or whether a mammal is a carrier of forelimb-girdle muscular anomaly, the marker comprising an isolated polynucleotide having a part or the whole of the GFRA1 gene, the polynucleotide including a loss-of-function mutation in the gene. It is preferable that the mammal is a bovine animal. In addition, it is more preferable that the loss-of-function mutation is a mutation of the nucleotide at position 1060 of a cDNA of the GFRA1 gene of SEQ ID NO. 1.

A marker according to the present invention is for diagnosing whether a mammal is affected by forelimb-girdle muscular anomaly or whether a mammal is a carrier of forelimb-girdle muscular anomaly, the marker being an mRNA encoding a GFRA1 protein whose function is lost, or being a GFRA1 protein whose function is lost.

A kit according to the present invention is for diagnosing whether a mammal is affected by forelimb-girdle muscular anomaly or whether a mammal is a carrier of forelimb-girdle muscular anomaly, the kit comprising a pair of primers for amplification of a nucleotide with a loss-of-function mutation in the GFRA1 gene in an isolated polynucleotide having a part or the whole of that gene. It is preferable that the mammal is a bovine animal. In addition, it is more preferable that the loss-of-function mutation is a mutation at nucleotide position 1060 of a cDNA of the GFRA1 gene of SEQ ID NO. 1.

The kit according to the present invention further comprises a restriction enzyme. It is more preferable that this restriction enzyme cleaves a polypeptide having the nucleotide amplified by the pair of primers in different ways depending on whether or not the nucleotide amplified by the pair of primers includes the loss-of-function mutation of the aforementioned gene. It is more preferable that the restriction enzyme is MwoI.

A diagnostic method according to the present invention is a method of diagnosing whether a non-human mammal is affected by forelimb-girdle muscular anomaly or whether a non-human mammal is a carrier of forelimb-girdle muscular anomaly, comprising the step of determining whether the GFRA1 gene in a genomic DNA or an mRNA isolated from the mammal is wild-type or has a loss-of-function mutation. In this diagnostic method, the mammal is preferably diagnosed as affected by forelimb-girdle muscular anomaly when the genomic DNA has no wild-type GFRA1 gene or when an mRNA transcribed from a GFRA1 gene does not contain an mRNA transcribed from a wild-type GFRA1 gene, and the mammal is preferably diagnosed as a carrier of forelimb-girdle muscular anomaly when the genomic DNA has a wild-type GFRA1 gene and a GFRA1 gene with a loss-of-function mutation, or when an mRNA transcribed from a GFRA1 gene contains an mRNA transcribed from the wild-type GFRA1 gene and an mRNA transcribed from a GFRA1 gene with a loss-of-function mutation.

It is more preferable that the diagnostic method according to the present invention comprises the steps of diagnosing whether the mammal is an animal affected by forelimb-girdle muscular anomaly when both alleles of the GFRA1 gene have a loss-of-function mutation, and diagnosing the mammal as a carrier of forelimb-girdle muscular anomaly when one of the alleles of the GFRA1 gene has a loss-of-function mutation. It is more preferable that the mammal is a bovine animal.

In the diagnostic method according to the present invention, it is more preferable that the loss-of-function mutation is a nonsense mutation at nucleotide position 1060 of a cDNA of the GFRA1 gene of SEQ ID NO. 1. It is more preferable that the nonsense mutation is a mutation of C to T.

A diagnostic method according to the present invention is a method of diagnosing whether a non-human mammal is affected by forelimb-girdle muscular anomaly, comprising the step of measuring expression level of a mRNA encoding a GFRA1 wild-type protein in the mammal or expression level of the GFRA1 wild-type protein. It is preferable that the mammal is diagnosed as affected by forelimb-girdle muscular anomaly when the expression is not detected.

An identification method according to the present invention is a method of identifying a non-human mammal as a carrier of forelimb-girdle muscular anomaly, comprising the steps of: determining whether or not a genomic DNA or an mRNA isolated from a non-human mammal has a loss-of-function mutation in a GFRA1 gene, the non-human mammal having not yet developed a symptom of forelimb-girdle muscular anomaly; and identifying a non-human mammal having a wild-type GFRA1 gene and a GFRA1 gene with a loss-of-function mutation in the genomic DNA or a non-human mammal having an mRNA transcribed from the wild-type GFRA1 gene and an mRNA transcribed from the GFRA1 gene with the loss-of-function mutation. It is preferable that the mammal is a bovine animal. It is more preferable that the loss-of-function mutation is a nonsense mutation of C to T at nucleotide position 1060 of cDNA of the GFRA1 gene of SEQ ID NO. 1.

According to the present invention, a method of determining whether or not GFRA1 gene is responsible for forelimb-girdle muscular anomaly in a mammal affected by forelimb-girdle muscular anomaly, comprises the steps of: determining a part or the whole of a nucleotide sequence of a GFRA1 gene in a mammal affected by forelimb-girdle muscular anomaly or a mammal that is a carrier of forelimb-girdle muscular anomaly; comparing the determined nucleotide sequence with a nucleotide sequence of a wild-type GFRA1 gene; and determining whether or not the determined nucleotide sequence has a loss-of-function mutation.

A marker according to the present invention is for diagnosing whether a bovine animal is affected by forelimb-girdle muscular anomaly or whether a bovine animal is a carrier of forelimb-girdle muscular anomaly, the marker being an isolated polynucleotide having one or more nucleotide or nucleotide sequence selected from the group consisting of MOK2630, MOK2637, SNP B, and SNP D.

A kit according to the present invention is for diagnosing whether a bovine animal is affected by forelimb-girdle muscular anomaly or whether a bovine animal is a carrier of forelimb-girdle muscular anomaly, the kit comprising one or more pair(s) of primers for amplifying one or more selected from the group consisting of MOK2630, MOK2637, SNP B, and SNP D.

A diagnostic method according to the present invention is a method of diagnosing whether a bovine animal is affected by forelimb-girdle muscular anomaly or whether a bovine animal is a carrier of forelimb-girdle muscular anomaly, the method comprising the step of determining whether the genotype of one or more selected from the group consisting of MOK2630, MOK2637, SNP B, and SNP D is a normal type or a disease type in a genomic DNA isolated from the bovine animal.

It is preferable that the diagnostic method according to the present invention further comprises the step of determining whether the genotype of MOK2630 is a normal type or a disease type in the genomic DNA isolated from the bovine animal, in which the bovine animal is diagnosed as affected by forelimb-girdle muscular anomaly when the genotype of MOK2630 is homozygous for the disease type and the bovine animal is diagnosed as a carrier of forelimb-girdle muscular anomaly when the genotype of MOK2630 is heterozygous for the normal and disease types, and the bovine animal is diagnosed as normal when the genotype of MOK2630 is homozygous for the normal type.

It is preferable that the diagnostic method according to the present invention further comprises the step of determining whether the genotype of MOK2637 is a normal type or a disease type in the genomic DNA isolated from the bovine animal, in which the bovine animal is diagnosed as affected by forelimb-girdle muscular anomaly when the genotype of MOK2637 is homozygous for the disease type, the bovine animal is diagnosed as a carrier of forelimb-girdle muscular anomaly when the genotype of MOK2637 is heterozygous for the normal and disease types, and the bovine animal is diagnosed as normal when the genotype of MOK2637 is homozygous for the normal type.

It is preferable that the diagnostic method according to the present invention further comprises the step of determining whether the genotype of SNP B is a normal type or a disease type in a genomic DNA isolated from the bovine animal, in which the bovine animal is diagnosed as affected by forelimb-girdle muscular anomaly when the genotype of SNP B is homozygous for the disease type, the bovine animal is diagnosed as a carrier of forelimb-girdle muscular anomaly when the genotype of SNP B is heterozygous for the normal and disease types, and the bovine animal is diagnosed as normal when the genotype of SNP B is homozygous for the normal type.

It is preferable that the diagnostic method according to the present invention further comprises the step of determining whether the genotype of SNP D is a normal type or a disease type in a genomic DNA isolated from the bovine animal, in which the bovine animal is diagnosed as affected by forelimb-girdle muscular anomaly when the genotype of SNP D is homozygous for the disease type, the bovine animal is diagnosed as a carrier of forelimb-girdle muscular anomaly when the genotype of SNP D is heterozygous for the normal and disease types, and the bovine animal is diagnosed as normal when the genotype of SNP D is homozygous for the normal type.

An identification method according to the present invention is a method of identifying a bovine animal being a carrier of forelimb-girdle muscular anomaly, comprising the steps of determining whether the genotype of one or more selected from the group consisting of MOK2630, MOK2637, SNP B, and SNP D is a normal type or a disease type in a genomic DNA isolated from a bovine animal, wherein the bovine animal has not yet been developed a symptom of forelimb-girdle muscular anomaly; and identifying the bovine animal in which the determined genotype is heterozygous for the normal and disease types.

### BRIEF DESCRIPTION OF THE DRAWINGS

FIG. 1 represents an electrophoretogram showing normal (1 to 7) and disease (8) types of MOK2630 in an example of the present invention.

FIG. 2 represents an electrophoretogram showing normal (1, 2, and 4) and disease (3) types of MOK2637 in an example of the present invention.

FIG. 3 represents a figure indicating a nonsense mutation in exon 4 of the GFRA1 gene in an example of the present invention.

FIG. 4 represents a figure showing patterns of DNA fragments produced by digesting, with MwoI, PCR products amplified using, as template, genomic DNAs of Japanese black cattle: a normal animal (lane 1, Normal), a carrier of forelimb-girdle muscular anomaly (lane 2, Carrier), and an animal affected by forelimb-girdle muscular anomaly (lane 3, Affected), in an example of the present invention.

### EMBODIMENTS FOR CARRYING OUT THE INVENTION

Embodiments of the present invention that were completed based on the aforementioned findings are described in detail in reference to Examples.

Unless otherwise noted in embodiments and examples, all procedures used are according to standard protocols such as J. Sambrook, E. F. Fritsch & T. Maniatis (Ed.), Molecular cloning, a laboratory manual (3rd edition), Cold Spring Harbor Press, Cold Spring Harbor, N.Y. (2001); and F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, commercial reagent kits and measurement instruments are used as described in protocols attached thereto, unless otherwise noted.

The above and further objects, features, advantages, and ideas of the present invention are apparent to those skilled in the art from the detailed description of this specification. Furthermore, those skilled in the art can easily reproduce the present invention from these descriptions. The embodiments and specific examples described below represent preferable embodiments of the present invention, which are given for the purpose of illustration or explanation. The present invention is not limited thereto. It is obvious to those skilled in the art that various modifications may be made according to the descriptions of the present specification within the spirit and scope of the present invention disclosed herein.

### ==Markers Related to GFRA1 Gene and GFRA1 Protein==

Forelimb-girdle muscular anomaly is a disorder resulting from a recessive mutation, or a loss-of-function mutation, which causes the function of GFRA1 gene to be lost. More specifically, animals with no copy of the Wild-type GFRA1 gene in genomic DNA are affected by forelimb-girdle muscular anomaly. Such animal is referred herein to as an “affected animal”. In this case, all copies of the GFRA1 gene of the animal may be mutant GFRA1 genes with loss-of-function mutations. On the other hand, animals with at least one copy of the wild-type GFRA1 gene shows a normal phenotype and do not develop symptoms of forelimb-girdle muscular anomaly. However, if they have at least one copy of the mutant GFRA1 gene with a loss-of-function mutation, they become carriers for forelimb-girdle muscular anomaly. Such animal is referred herein to as a “carrier”. If they have no copy of the mutant GFRA1 gene with a loss-of-function mutation, they will neither develop symptoms of forelimb-girdle muscular anomaly nor become a carrier. Such animal is referred herein to as a normal animal. The normal animal means the one in which all copies of the GFRA1 gene are wild-type, and does not mean a carrier showing a normal phenotype and the affected animal in which symptoms of forelimb-girdle muscular anomaly has not been developed yet.

For example, assuming an animal having two alleles at each GFRA1 locus in the genome, i.e. having no additional copy of the GFRA1 gene other than the inherent gene, it is an animal affected by forelimb-girdle muscular anomaly when both of two alleles have loss-of-function mutations; it is a carrier of forelimb-girdle muscular anomaly when only one allele has a loss-of-function mutation; and it is a normal animal when both alleles are wild type.

As used herein, the term “forelimb-girdle muscular anomaly” refers to a disorder known as “forelimb-girdle muscular anomaly” or “SANMAIGATA” in bovine, its counterparts in mammals other than bovine which correspond to “forelimb-girdle muscular anomaly” in bovine, and disorders associated with hypoplasia of the forelimb-girdle muscles or the pectoral girdle muscles. This term may refer to any equivalent disorders that are not known with the name of “forelimb-girdle muscular anomaly” in certain mammals other than bovine.

As used herein, “to develop symptoms of forelimb-girdle muscular anomaly” in a mammal means that hypoplasia of the forelimb-girdle muscles or the pectoral girdle muscles is noticed in the mammal due to being affected by forelimb-girdle muscular anomaly. Particularly in mammals with four legs, recognized are morphological features including droop of the pinna and scapular projection due to hypoplasia of the forelimb-girdle muscles, as well as abnormalities not found in normal animals, including decrease in motor function such as astasia and tremors. These physical abnormalities or motor function abnormalities are not specifically limited by their level. An animal “affected by forelimb-girdle muscular anomaly” means that no copy of the wild-type GFRA1 gene is genetically present in the genome and symptoms of forelimb-girdle muscular anomaly may or may not be developed.

Forelimb-girdle muscular anomaly also related to mRNA transcribed from the GFRA1 gene. In the animals affected by forelimb-girdle muscular anomaly, mRNA transcribed from the GFRA1 gene does not contain mRNA transcribed from the wild-type GFRA1 gene. The carriers of forelimb-girdle muscular anomaly have mRNA transcribed from the Wild-type GFRA1 gene and mRNA transcribed from the GFRA1 gene with the loss-of-function mutation. The normal animals have mRNA transcribed only from the wild-type GFRA1 gene and do not have any mRNA transcribed from the GFRA1 gene with loss-of-function mutation.

Accordingly, an isolated polynucleotide having a part or the whole of the GFRA1 gene and having a mutation that causes the function of the GFRA1 gene to be lost can be used as a marker for diagnosing whether an animal is affected by forelimb-girdle muscular anomaly or whether an animal is a carrier of forelimb-girdle muscular anomaly. The polynucleotide may be DNA or RNA such as mRNA.

As used herein, the wild-type GFRA1 gene refers to the GFRA1 gene without any loss-of-function mutation as well as the GFRA1 gene without any loss-of-function mutation having one or more nucleotide substitutions, additions, or deletions, lost of whose expression in the body of an animal causes forelimb-girdle muscular anomaly. An animal from which the wild-type GFRA1 gene is derived may appropriately be selected from mammals depending on the subject of the diagnosis. For bovine as an example, the wild-type GFRA1 gene refers to the GFRA1 gene of SEQ ID NO. 2 (Gene ID: 534801) as well as the GFRA1 gene of SEQ ID NO. 2 having one or more nucleotide substitutions, additions, or deletions, lost of whose expression in the body of an animal cause forelimb-girdle muscular anomaly.

If the animal only with copies of the mutant GFRA1 gene develops symptoms of forelimb-girdle muscular anomaly, that mutation refers to the loss-of-function mutation herein. The GFRA1 gene in question is referred to as a GFRA1 mutant gene having the loss-of-function mutation. On the other hand, even when the GFRA1 gene has a mutation, it is not referred to as the loss-of-function mutation if the animal only with copies of the mutant GFRA1 gene does not develop symptoms of forelimb-girdle muscular anomaly.

The loss-of-function mutation in the GFRA1 gene is not limited by the position in the gene or the type of nucleotide as long as it is responsible for the development of symptoms of forelimb-girdle muscular anomaly. For example, it may be a point mutation, a deletion mutation or an insertion mutation. In addition, a mutant GFRA1 protein may or may be expressed as long as it is responsible for the development of symptoms of forelimb-girdle muscular anomaly.

For example, when the loss-of-function mutation in the GFRA1 gene is a nonsense mutation and results in truncation of a translated protein, the position of the mutated nucleotide in the gene is not limited as long as the mutation causes the function of the wild-type GFRA1 gene to be lost. The loss-of-function mutation in bovine may be, for example, a nonsense mutation in which C is replaced by T at the nucleotide position corresponding to nucleotide position 1060 of cDNA of the GFRA1 gene (NM_{—}001105411.1; SEQ ID NO. 1) or a nonsense mutation located upstream of the position. The loss-of-function mutation in a mammal other than bovine may be, for example, a nonsense mutation at the nucleotide position corresponding to the nucleotide position 1060 of cDNA of the aforementioned bovine GFRA1 gene or a nonsense mutation located upstream of the position.

When a mutant GFRA1 gene with a loss-of-function mutation is transcribed to produce mRNA of the mutant GFRA1 and a GFRA1 mutant protein is expressed in an animal affected by forelimb-girdle muscular anomaly or an animal that is a carrier of forelimb-girdle muscular anomaly, such GFRA1 mutant protein with the loss-of-function and mRNA encoding the GFRA1 mutant protein with the loss-of-function may also be used as a marker for diagnosing whether the animal is affected by forelimb-girdle muscular anomaly or is a carrier of the forelimb-girdle.

In animals affected by forelimb-girdle muscular anomaly, expression level of the wild-type GFRA1 protein decreases to an extent that symptoms of forelimb-girdle muscular anomaly have been developed, compared with that of normal animals, or is lost. Accordingly, expression level of the wild-type GFRA1 protein and that of mRNA encoding the GFRA1 wild-type protein can also be used as the marker for diagnosing whether the animal is affected by forelimb-girdle muscular anomaly.

The wild-type GFRA1 protein is a protein encoded by the wild-type GFRA1 gene as well as a protein encoded by the wild-type GFRA1 gene having one or more amino acid substitutions, additions, or deletions, lost of whose expression in the body of an animal causes forelimb-girdle muscular anomaly. An animal from which the wild-type GFRA1 protein is derived may appropriately be selected from mammals depending on the subject of the diagnosis. For bovine as an example, the wild-type GFRA1 protein is a GFRA1 protein of SEQ ID NO. 3 (GenBank Accession No.: NP_{—}001098881.1) and its homologues, as well as a GFRA1 protein of SEQ ID NO. 3 and its homologues having one or more amino acid substitutions, additions, or deletions, lost of whose expression causes forelimb-girdle muscular anomaly.

### ==Diagnostic Method Using Markers Related to GFRA1 Gene and GFRA1 Protein==

By using these markers, it is possible to diagnose whether a mammal is affected by forelimb-girdle muscular anomaly or whether a mammal is a carrier of forelimb-girdle muscular anomaly as follows.

Whether a given mammal is affected by forelimb-girdle muscular anomaly or whether a mammal is a carrier of forelimb-girdle muscular anomaly can be diagnosed by isolating genomic DNA from the mammal and determine whether the GFRA1 gene in the isolated genomic DNA has a loss-of-function mutation. More specifically, (1) the animal can be diagnosed as affected by forelimb-girdle muscular anomaly when no wild-type GFRA1 gene is present apart from the presence of the GFRA1 gene having a loss-of-function mutation, (2) the animal can be diagnosed as a carrier of forelimb-girdle muscular anomaly when both of the GFRA1 gene having a loss-of-function mutation and the wild-type GFRA1 gene are present, and (3) the animal can be diagnosed as normal when the wild-type GFRA1 gene is present and no GFRA1 gene having a loss-of-function mutation is present. Any method can be used without limitation to determine whether the GFRA1 gene has a loss-of-function mutation. For example, the nucleotide sequence of the GFRA1 gene may be determined. Alternatively, RFLP may be used to detect a certain known nucleotide substitution.

When the loss-of-function mutation is a mutation of C to T at nucleotide position 1060 of cDNA of the GFRA1 gene (SEQ ID NO. 1), genomic DNA fragments containing that nucleotide may be amplified by PCR and the resulting DNA fragments may be digested with MwoI to examine whether the PCR product is cleaved. It can easily be determined whether the subject nucleotide is wild-type or mutant, because the PCR product cannot be cleaved with MwoI if the nucleotide has a mutation of C to T while the PCR product is cleaved with MwoI when such a mutation is not present. For example, when only the PCR product cleaved with MwoI is detected by separation of the PCR product digested with the enzyme using electrophoresis, the subject animal can be diagnosed as normal and neither as a carrier of forelimb-girdle muscular anomaly nor as affected by it. On the other hand, when the PCR product cleaved with MwoI and the PCR product not cleaved with MwoI are both detected, the subject animal can be diagnosed as a carrier of forelimb-girdle muscular anomaly. When only the PCR product not cleaved with MwoI is detected, then the subject animal can be diagnosed as affected by forelimb-girdle muscular anomaly.

Alternatively, whether a mammal is affected by forelimb-girdle muscular anomaly may be diagnosed by determining the expression of the wild-type GFRA1 protein or the expression of mRNA encoding the wild-type GFRA1 protein in a tissue of that animal. The tissue may be, for example, blood, semen, muscles, nerves, a bone, a kidney, a liver, a thymus, a skin, and a fertilized ovum, but is not limited as long as it is a tissue in which the wild-type GFRA1 protein or mRNA encoding the wild-type GFRA1 protein is expressed in the normal animal. As a result, the animal can be diagnosed as affected by forelimb-girdle muscular anomaly when the expression of neither the wild-type GFRA1 protein nor mRNA encoding the wild-type GFRA1 protein is detected.

In addition, whether a mammal is a carrier of forelimb-girdle muscular anomaly may be diagnosed by examining the expression of the wild-type GFRA1 protein and the mutant GFRA1 protein or the expression of mRNA encoding the wild-type GFRA1 protein and mRNA encoding the mutant GFRA1 protein in a tissue of forelimb-girdle muscles of that animal. As a result, the animal can be diagnosed as a carrier of forelimb-girdle muscular anomaly when both of the wild-type GFRA1 protein and the mutant GFRA1 protein are detected or when both of mRNA encoding the mutant GFRA1 protein and mRNA encoding the wild-type GFRA1 protein are detected.

An animal can be determined as a carrier of forelimb-girdle muscular anomaly or as normal when the expression of the wild-type GFRA1 protein or mRNA encoding the wild-type GFRA1 protein is detected and the expression of the mutant GFRA1 protein or mRNA encoding the mutant GFRA1 protein is not detected.

Any technique may be used to detect the expression of the protein or mRNA encoding the protein as long as it can specifically detect the wild-type GFRA1 protein and the mutant GFRA1 protein, or detect mRNA encoding the GFRA1 wild-type protein and the GFRA1 mutant protein. It may detect the whole or a part of the protein or mRNA. Any detection methods known to those skilled in the art may appropriately be used. Examples include Northern blotting and RT-PCR for mRNA, and Western blotting and ELISA with a specific antibody for protein.

By diagnosing mammals in the manner described above, it is possible to identify the affected animal that appears normal but is expected to develop symptoms of forelimb-girdle muscular anomaly in the future. In addition, although carriers of forelimb-girdle muscular anomaly do not have phenotypically any symptom, the occurrence of the animal affected by forelimb-girdle muscular anomaly can be avoided by identifying carriers in the manner described above and isolating or removing them from a breeding population or avoiding mating between them.

### ==Gene Therapy==

The mammal affected by forelimb-girdle muscular anomaly has no wild-type GFRA1 gene. Accordingly, the disorder can be treated by creating a transgenic animal in which the wild-type GFRA1 gene is introduced.

For the mammal that is a carrier of forelimb-girdle muscular anomaly having the loss-of-function mutation in one of the alleles of the GFRA1 gene, the loss-of-function mutation in the GFRA1 gene can be repaired in the allele having the loss-of-function mutation to be a wild-type GFRA1 gene. Conventionally, with mammals, embryonic stem cells have been established (Biochem. Biophys. Res. Commun. vol. 309, p. 104-113, 2003) and knock-out animals have been produced (Nat. Ganet. vol. 36, p. 671-672, 2004). By using such gene recombination techniques based on developmental engineering, a certain nucleotide can be substituted by a desired nucleotide in mammals to repair the loss-of-function mutation.

### ==Method of Specifying Mutation Responsible for Forelimb-Girdle Muscular Anomaly==

In mammals affected by forelimb-girdle muscular anomaly or carriers of forelimb-girdle muscular anomaly, it is possible to determine whether forelimb-girdle muscular anomaly is caused by the GFRA1 gene or which mutation is responsible for the disorder when forelimb-girdle muscular anomaly is due to the GFRA1 gene, by determining a part or the whole of the nucleotide sequence of the GFRA1 gene, comparing the obtained nucleotide sequence with that of the wild-type GFRA1 gene, and determining whether the GFRA1 gene includes the loss-of-function mutation.

For example, when a mutation is present in the GFRA1 gene and the mutation is a loss-of-function mutation in an animal affected by forelimb-girdle muscular anomaly or an carrier of forelimb-girdle muscular anomaly, it can be determined that forelimb-girdle muscular anomaly is due to the mutation of the GFRA1 gene.

The isolated polynucleotide having a part or the whole of the GFRA1 gene, including the loss-of-function mutation determined in the manner described above can be used as a marker for diagnosing whether a mammal is affected by forelimb-girdle muscular anomaly or whether a mammal is a carrier of forelimb-girdle muscular anomaly.

### ==Markers Around the GFRA1 Gene==

As shown in Example 2, mammals having a loss-of-function mutation of the GFRA1 gene in homozygous state develop symptoms of the forelimb-girdle muscular anomaly, while those having that mutation in heterozygous state are carriers of forelimb-girdle muscular anomaly. Genotypes of MOK2630 which is a microsatellite marker on bovine chromosome 26, MOK2637 which is another microsatellite marker on bovine chromosome 26, SNP B, and SNP D (Table 1) are strongly correlated with the facts that the bovine animal is affected by forelimb-girdle muscular anomaly and that it is a carrier of forelimb-girdle muscular anomaly, and thus strongly correlates with a loss-of-function mutation in the bovine GFRA1 gene. Accordingly, one or more isolated polynucleotides including one or more selected from the group consisting of MOK2630, MOK2637, SNP B, and SNP D can be used as markers for diagnosing whether a bovine animal is affected by forelimb-girdle muscular anomaly or whether a bovine animal is a carrier of forelimb-girdle muscular anomaly.

TABLE 1

| | position on | | | | | | |
|---|---|---|---|---|---|---|---|
| | chromosome | normal | disease | | SEQ | | SEQ |
| | 26 | type | type | Primer 1 | ID NO. | Primer 2 | ID NO. |
| MOK2630 | 36222000 | 1-7 | 8 | gatcacctaacatcttgcctaatc | 4 | ctattgctatccagtcatgtgc | 5 |
| MOK2637 | 36977078 | 1, 2, 4 | 3 | tgtgtctctctatctatccctgtctc | 6 | cgttcctgcctctgtctctc | 7 |
| SNP B | 33733727 | T | G | aaatagcagaaatttagaagcagca | 8 | tgtgagcacgtgtgtgtatga | 9 |
| SNP D | 37013762 | A | G | caagacaacgcttatcccacag | 10 | cagtcctaagaaactaacatagcacc | 11 |

Because MOK2630, MOK2637, SNP B, and SNP D are in linkage disequilibrium and the genotypes of MOK2630, MOK2637, SNP B, and SNP D strongly correlate with each other, genotyping of only one of MOK2630, MOK2637, SNP B, and SNP D allows prediction of the genotypes of the remainders. For example, the genotype of one of MOK2630, MOK2637, SNP B, and SNP D is the normal type, then the genotypes of the other three can be predicted as being the normal type. When the genotype of one of MOK2630, MOK2637, SNP B, and SNP D is the disease type, then the genotypes of the other three can also be predicted as the disease type.

MOK2630 refers to a stretch of DNA with a repetitive sequence that extends from position 36222000 on bovine chromosome 26 in bovine genome assembly (Btau4.0). MOK2637 refers to a stretch of DNA with a repetitive sequence that extends from position 36977078 on bovine chromosome 26 in bovine genome assembly (Btau4.0). SNP B refers to the nucleotide at position 33733727 on bovine chromosome 26 in bovine genome assembly (Btau4.0). SNP D refers to the nucleotide at position 37013762 on bovine chromosome 26 in bovine genome assembly (Btau4.0) (see, Table 1).

SNP B has two types of the nucleotides, T and G, of which T corresponds to the normal type whereas G corresponds to the disease type. SNP D has two types of the nucleotides, A and G, of which A corresponds to the normal type whereas G corresponds to the disease type (Table 1).

Alleles of MOK2630 are defined by the number of GT repeats of their nucleotide sequences. Alleles of MOK2637 are defined by the number of AT repeats of their nucleotide sequences. In order to identify alleles of the disease type of MOK2630 and MOK2637, the alleles of MOK2630 and MOK2637 are examined in one or more normal animals and one or more affected animals, and an allele that is hardly detected in the normal animal but is detected in the affected animal at significantly high frequency is determined among the examined alleles for each of MOK2630 and MOK2637. In order to identify the alleles of the normal type of MOK2630 and MOK2637, an allele hardly detected in the affected animal but is detected in the normal animal at significantly high frequency is determined among the examined alleles for each of MOK2630 and MOK2637.

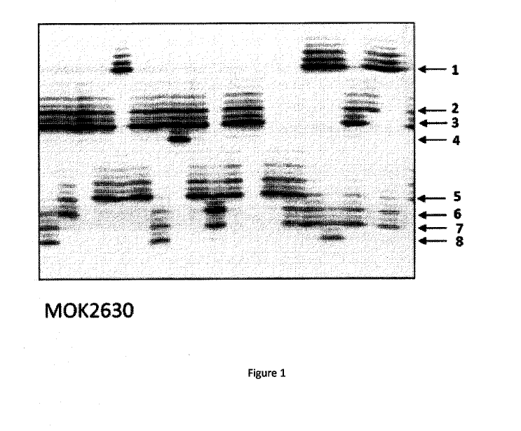

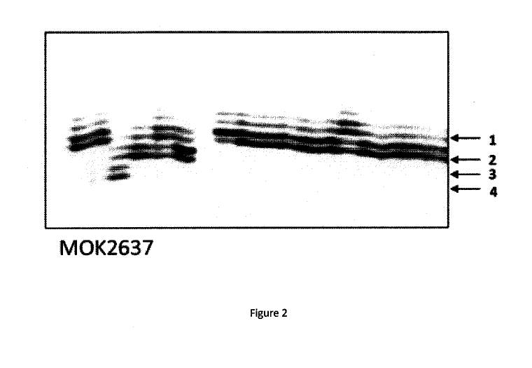

For example, alleles of MOK2630 and MOK2637 were analyzed for animals of Japanese black cattle using the pair of primers shown in Table 1 and compared between the normal animals and the affected animals. As a result, MOK2630 had 8 alleles with different number of GT repeats. The first through seventh alleles in ascending order of number of repeats are of the normal type and the eighth allele with the largest number of repeats is of the disease type (FIG. 1, 1 to 8 from above). MOK2637 had 4 alleles with different number of AC repeats. The first, second, and fourth alleles in ascending order of number of repeats are of the normal type and the third allele is of the disease type (FIG. 2, 1 to 4 from above).

The number of nucleotides that make up the polynucleotide to be used as the marker is not specifically limited. The polynucleotide to be used as the marker is only required to contain at least one of MOK2630, MOK2637, nucleotides in SNP B, and nucleotides in SNP D. If the marker contains two or more of them, any combination may be used.

### ==Diagnostic Method Using Markers Around GFRA1 Gene==

In order to diagnose whether a bovine animal is affected by forelimb-girdle muscular anomaly or whether a bovine animal is a carrier of forelimb-girdle muscular anomaly using MOK2630, MOK2637, SNP B, or SNP D, at least one of MOK2630, MOK2637, SNP B, and SNP D is genotyped in genomic DNA isolated from the animal.

The genotypes of MOK2630, MOK2637, SNP B, and SNP D may be determined by, for example, directly determining their nucleotide sequences or using PCR or RFLP. Any method of genotyping known to those skilled in the art may be used without limitation. To determine the nucleotide sequence using PCR, a pair of primers such as those shown in Table 1 may be used. Although direct sequencing of nucleotide may be performed for all nucleotides in the polynucleotide comprising the marker, it is enough to determine at least one selected from the group consisting of the nucleotide sequence of MOK2630, the nucleotide sequence of MOK2637, the nucleotides in SNP B and the nucleotides in SNP D in the polynucleotide comprising the marker.

Each of MOK2630 and MOK2637 has the unique number of GT and AC repeats for each of the alleles of the normal and disease types. Accordingly, to determine the genotype of MOK2630 or MOK2637 in a bovine animal to be diagnosed, its nucleotide sequences or its nucleotide lengthes of the alleles are examined using, for example, electrophoresis and then the alleles are determined to represent the normal type or the disease type. Alternatively, to determine either the genotype of MOK2630 or MOK2637 in the bovine animal to be diagnosed, the nucleotide lengthes of MOK2630 or MOK2637 obtained from the animal to be diagnosed may be compared with the nucleotide lengthes of known alleles of the disease type or the normal type of MOK2630 or MOK2637 to determine whether the genotype of MOK2630 or MOK2637 in the animal to be diagnosed is a normal type or a disease type.

The type and amount of the tissue used for isolating the genomic DNA are not specifically limited as long as a required amount of DNA can be obtained to determine the nucleotide sequence of the microsatellite or the nucleotide in SNP.

Since MOK2630, MOK2637, SNP B, and SNP D are in linkage disequilibrium as described above, genotyping of at least one of them can provide diagnosis of the mammal as affected by forelimb-girdle muscular anomaly or as a carrier of forelimb-girdle muscular anomaly. More precise diagnosis, however, can be performed by determining two or more, more preferably, three or more, and most preferably, four genotypes.

When two or more of MOK2630, MOK2637, SNP B, and SNP D are genotyped for the diagnosis, any combination thereof may be used without limitation. For example, the combination may be MOK2630 and MOK2637; MOK2630 and SNP B; MOK2630 and SNP D; MOK2630, MOK2637, and SNP B; MOK2630, MOK2637 and SNP D; MOK2630, MOK2637, SNP B, and SNP D; MOK2630, SNP B, and SNP D; MOK2637 and SNP B; MOK2637 and SNP D; MOK2637, SNP B, and SNP D; and SNP B and SNP D.

More specifically, in bovine animals affected by forelimb-girdle muscular anomaly, the genotypes of MOK2630, MOK2637, SNP B, and SNP D are homozygous for the haplotype consisting of the alleles of the disease type and the SNPs of the disease type. In carriers of forelimb-girdle muscular anomaly, the genotypes are heterozygous for the haplotype consisting of the alleles of the disease type and the SNPs of the disease type and the haplotype consisting of the alleles of the normal type and the SNPs of the normal type. In normal animals, the genotypes are homozygous for the haplotype consisting of the alleles of the normal type and the SNPs of the normal type. As shown in Example 3, the population of the normal animals contains no bovine animal having haplotype consisting of the alleles of the disease type and the SNPs of the disease type.

Accordingly, a bovine animal can be diagnosed as affected by forelimb-girdle muscular anomaly when at least one of MOK2630, MOK2637, SNP B, and SNP D is genotyped in the genomic DNA isolated from the animal and the genotype is homozygous for the disease type. Likewise, the animal can be diagnosed as a carrier of forelimb-girdle muscular anomaly when the genotypes are heterozygous for the normal and disease types. The animal can be diagnosed as normal when the genotype is homozygous for the normal type. If different results have been obtained from the genotypes of a plurality of markers, the number of markers to be examined may be increased and results obtained may be judged, although the result obtained from a marker with a higher linkage to the GFRA1 gene may preferentially be used.

By diagnosing bovine animals in the manner described above, it is possible to identify the affected animal that appears normal but is anticipated to develop symptoms of forelimb-girdle muscular anomaly in the future. In addition, for carriers of forelimb-girdle muscular anomaly, the incidence of the animal affected by forelimb-girdle muscular anomaly can be avoided in the manner described above by identifying the carrier that is phenotypically normal with no symptom, and isolating or removing the carrier from a breeding population or to avoid mating between the carriers.

### EXAMPLES

### Example 1

This Example shows that the presence or absence of a loss-of-function mutation in a bovine GFRA1 gene can be detected by RFLP to diagnose an animal as affected by forelimb-girdle muscular anomaly or as a carrier of the disorder.

DNAs were obtained, by phenol/chloroform extraction, from semen, blood, or muscle tissue of 26 animals of Japanese black cattle with symptoms of forelimb-girdle muscular anomaly, 37 normal animals of Japanese black cattle, 6 carriers of forelimb-girdle muscular anomaly of Japanese black cattle (a sire of affected and normal animals, 3 dams, a grand sire, and a great-grand sire). The carriers were determined based on the fact that their offspring had developed symptoms of the disorder. For these animals, a 345-bp region including exon 4 of the GFRA1 gene was amplified by PCR using the following pair of primers.

| (SEQ ID NO. 12) | |
|---|---|
| GFRA1-F2: | ATGCTCCTCACGGTACCTCTGTCCTAAA |
| (SEQ ID NO. 13) | |
| GFRA1-R3: | GTTCCCTTCCAGAGCTCAAGC |

The PCR products were digested with the restriction enzyme MwoI and DNA fragments were separated by agarose gel electrophoresis.

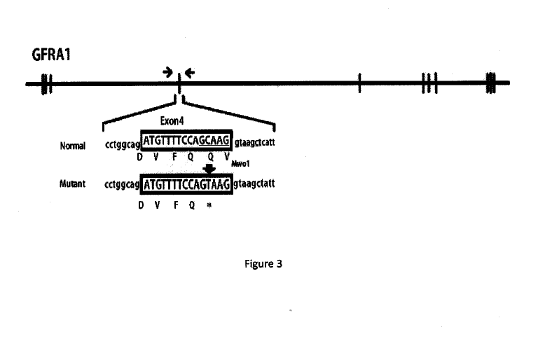

As shown in FIG. 3, a mutation has occurred in both alleles of the affected animal and in one allele of the carrier in exon 4 of the GFRA1 gene. This mutation is a nonsense mutation of C to T at nucleotide position 1060 of a coding region of the GFRA1 gene, which causes the function of the GFRA1 gene to be lost. The amplified PCR product is not cleaved with MwoI when it has this mutation and is cleaved with MwoI when it does not have the mutation.

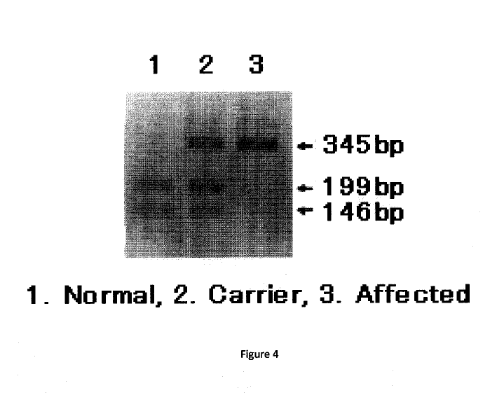

As shown in FIG. 4, bands of 199 bp and 146 bp were detected for a normal animal (lane 1); bands of 345 bp, 199 bp, and 146 by were detected for a carrier (lane 2); and a 345-bp band was detected for an affected animal (lane 3).

As described above, it is possible to diagnose that an animal is affected by forelimb-girdle muscular anomaly or is a carrier of forelimb-girdle muscular anomaly by determining the mutation of C to T at nucleotide position 1060 of the coding region of the GFRA1 gene.

### Example 2

This Example shows that it is possible to diagnose an animal as affected by forelimb-girdle muscular anomaly or as a carrier of the disorder according to the presence or absence of a loss-of-function mutation in a GFRA1 gene.

DNAs were isolated, by phenol/chloroform extraction, from semen, blood, or muscle tissue of 26 animals of Japanese black cattle with symptoms of forelimb-girdle muscular anomaly, 37 normal animals of Japanese black cattle, 6 carriers of forelimb-girdle muscular anomaly of Japanese black cattle (a sire of affected and normal calves, 3 dams, a grand sire, and a great-grand sire). For the isolated DNAs, a 345-bp region including exon 4 of the bovine GFRA1 gene was amplified by PCR using the pair of primers in Example 1 (SEQ ID NOs. 12 and 13). The carriers were determined based on the fact that their offspring had developed symptoms of the disorder.

All 26 animals with symptoms of forelimb-girdle muscular anomaly showed homozygosity for T at nucleotide position 1060 of cDNA of the bovine GFRA1 gene (SEQ ID NO. 1). All 6 carrier animals showed heterozygosity for C and T at nucleotide position 1060 of cDNA of the bovine GFRA1 gene. All 37 normal animals showed homozygosity for C at nucleotide position 1060 of cDNA of the bovine GFRA1 gene.

In addition, the nucleotide at position 1060 of cDNA of the GFRA1 gene was examined for 125 normal animals of a pedigree obtained from a population of Japanese black cattle that is different from the one described above. All animals showed homozygosity for C.

As described above, it is possible to diagnose with 100% probability that a animal is affected by forelimb-girdle muscular anomaly or is a carrier of forelimb-girdle muscular anomaly by determining the mutation of C to T at nucleotide position 1060 of cDNA of the bovine GFRA1 gene.

In addition, the mutation at position 1060 of cDNA of the bovine GFRA1 gene is a loss-of-function mutation of the GFRA1 gene, so that the results indicate that bovine can be diagnosed as affected by forelimb-girdle muscular anomaly or as a carrier of forelimb-girdle muscular anomaly according to whether the loss-of-function mutation is present in the GFRA1 gene. This can be applied to mammals other than bovine to diagnose affection by and carriage of the disease characterized by hypoplasia of the forelimb- or upper limb-girdle muscles.

### Example 3

This Example shows that affection by and carriage of forelimb-girdle muscular anomaly can be diagnosed by genotyping MOK2630, MOK2637, SNP B, and SNP D.

DNAs were obtained by phenol/chloroform extraction from semen, blood, or muscle tissue of 26 animals of Japanese black cattle with symptoms of forelimb-girdle muscular anomaly. The DNA fragments containing MOK2630, MOK2637, SNP B, and SNP D were amplified PCR using the pair of primers shown in Table 1. To determine the genotypes of SNP B and SNP D, the fragments were cleaved with restriction enzymes MseI (SNP B) and BamAI (SNP D).

For the genotypes of MOK2630, MOK2637, and SNP D, all 26 animals were homozygous for the disease type, i.e., 8, 3, and G (Table 1). For the genotype of SNP B, 25 animals were of the disease type, i.e., homozygotes for G (Table 1) but 1 animal was a heterozygote of the disease type (G) and the normal type (T) (Table 2).

TABLE 2

| | number of | | number of |
|---|---|---|---|
| | homo- | number of | homo- |
| | zygotes of | hetero- | zygotes of |
| | disease type | zygotes | normal type |
| MOK2630 | 26 | 0 | 0 |
| MOK2637 | 26 | 0 | 0 |
| SNP B | 25 | 1 | 0 |
| SNP D | 26 | 0 | 0 |

MOK2630 (61 animals), MOK2637 (119 animals), SNP B (118 animals), and SNP D (117 animals) were genotyped in a similar manner for normal animals of Japanese black cattle. No animal was homozygous for the disease type of MOK2630, SNP B, and SNP D. Only 1 animal was homozygous for the disease type of MOK2637. In addition, 11, 18, 8, and 28 animals were heterozygous for the disease type and the normal type of MOK2630, MOK2637, SNP B, and SNP D, respectively, whereas 50, 100, 110, and 89 animals were homozygous for the normal type of MOK2630, MOK2637, SNP B, and SNP D, respectively (Table 3).

TABLE 3

| | | | | | fre- |
|---|---|---|---|---|---|
| | | number of | | number of | quency |
| | | homo- | | homo- | of |
| | number of | zygotes of | number of | zygotes of | disease |
| | animals | disease | hetero- | normal | type |
| | examined | type | zygotes | type | (%) |
| MOK2630 | 61 | 0 | 11 | 50 | 9.0 |
| MOK2637 | 119 | 1 | 18 | 100 | 8.4 |
| SNP B | 118 | 0 | 8 | 110 | 3.4 |
| SNP D | 117 | 0 | 28 | 89 | 12.0 |

As described above, by examining MOK2630, MOK2637, SNP B, and SNP D, it is possible to diagnose with a high probability whether the bovine animal is affected by forelimb-girdle muscular anomaly.

### INDUSTRIAL APPLICABILITY

According to the present invention, it is possible to provide markers for diagnosing forelimb-girdle muscular anomaly in mammals and a method of diagnosing forelimb-girdle muscular anomaly in mammals using these markers.

1. A marker for diagnosing whether a mammal is affected by forelimb-girdle muscular anomaly or whether a mammal is a carrier of forelimb-girdle muscular anomaly,
the marker comprising an isolated polynucleotide being a part or the whole of a genomic DNA of the GFRA1 gene, a part or the whole of a cDNA of the gene, or a part or the whole of an mRNA of the gene,
the polynucleotide including a mutation of the nucleotide at position 1060 of cDNA of the GFRA1 gene of SEQ ID NO. 1.

2. The marker according to claim 1, wherein the mammal is a bovine animal.

3. The marker according to claim 1, wherein the nucleotide having the mutation is thymine.

4. A kit for diagnosing whether a mammal is affected by forelimb-girdle muscular anomaly or whether a mammal is a carrier of forelimb-girdle muscular anomaly, the kit comprising:
a pair of primers for the amplification of a mutation of a nucleotide at position 1060 in a cDNA of the GFRA1 gene of SEQ ID NO. 1 in an isolated DNA having a part or the whole of the GFRA1 gene.

5. The kit according to claim 4, wherein the mammal is a bovine animal.

6. The kit according to claim 4, wherein the nucleotide having the mutation is thymine.

7. The kit according to claim 4, further comprising
a restriction enzyme, the restriction enzyme cleaving a polypeptide having the nucleotide amplified by the pair of primers in different ways depending on whether or not the nucleotide amplified by the pair of primers includes the mutation.

8. The kit according to claim 7, wherein the restriction enzyme is MwoI.

9. A method of diagnosing whether a non-human mammal is affected by forelimb-girdle muscular anomaly or whether as a non-human mammal is a carrier of forelimb-girdle muscular anomaly, comprising the step of:
determining whether or not a genomic DNA or an mRNA isolated from the mammal has a mutation at nucleotide position 1060 of a cDNA of the GFRA1 gene of SEQ ID NO. 1.

10. The method according to claim 9, comprising the steps of:
diagnosing the mammal as affected by forelimb-girdle muscular anomaly when both alleles of the GFRA1 gene have the mutation, and
diagnosing the mammal as a carrier of forelimb-girdle muscular anomaly when one of the alleles of the GFRA1 gene has the mutation.

11. The method according to claim 9, wherein the mammal is a bovine animal.

12. The method according to claim 9, wherein the nucleotide having the mutation is thymine.

13. A method of identifying a non-human mammal being a carrier of forelimb-girdle muscular anomaly, comprising the steps of:
determining whether or not a genomic DNA or an mRNA isolated from a non-human mammal has a mutation at nucleotide position 1060 of a cDNA of the GFRA1 gene of SEQ ID NO. 1, wherein the mammal has not yet developed a symptom of forelimb-girdle muscular anomaly; and
identifying a non-human mammal having a wild-type GFRA1 gene and a GFRA1 gene with a mutation in the genomic DNA or a non-human mammal having an mRNA transcribed from the wild-type GFRA1 gene and an mRNA transcribed from the GFRA1 gene with the mutation.

14. The method according to claim 13, wherein the mammal is a bovine animal.

15. The method according to claim 13, the nucleotide having the mutation is thymine.

16. A method of determining whether or not a GFRA1 gene is responsible for forelimb-girdle muscular anomaly in a mammal affected by forelimb-girdle muscular anomaly, comprising the steps of:
determining a part or the whole of a nucleotide sequence of the GFRA1 gene in a mammal affected by forelimb-girdle muscular anomaly or a mammal being a carrier of forelimb-girdle muscular anomaly;
comparing the determined nucleotide sequence with a nucleotide sequence of a wild-type GFRA1 gene; and
determining whether or not the determined nucleotide sequence has a mutation at nucleotide position 1060 of a cDNA of the GFRA1 gene of SEQ ID NO. 1.

17. The method according to claim 16, wherein the mammal is a bovine animal.

18. The method according to claim 16, wherein the nucleotide having the mutation is thymine.


SEQUENCE LISTING (13 sequences)

SEQ ID NO: 1 - DNA, 3022 bp, Bos taurus
cgcgccagggttggcgagaaagctgccgccgccgagtgcttcgactccaggcagcggccggctcgctctcggaaagtaacgttatttatttatttgcttgcgttcaacctcttggattggatccagtagacacccccttggactttgggaacccgcgtctgacctctccagcctttaccctgcgcgtgcgggcggctcccgggaatacacgcacatgcacccccgggggccccctccccgtgcccagatccgggtaggggtgggcccttacccggggcgctaaaaagtcccccaaggcaggagccggggaggggagaggggcgggggaggctccagcgctctccgctctggtttcaaagcgcgctctcccttccaggttgggtcggacctgaacccctaaaagcggaaccgcctcccgccctcgcgccaaccgcccgcccggagctgagtcgccggcggcggtggcggcggacggaccggggagtttcctctcgccctggatggagctgaactttgggcggccagaacagcgcgaccgtccggggatcactgcatgctgagctcccttggcgagaccgagcggcaggccgggattttcgggggggcggggaccagctgcgcgccggcaccatgttcctggcgaccctgtacttcgtactgccgcttctggacgttctcctgtcggccgaggtgagcggcggggaccgcctggactgcgtgaaagccagcgatcagtgcctgaaggagcagagctgcagcaccaagtaccgcacgctccggcagtgcgtggcgggcaaggagaccaacttcagcttgacctcgggcctcgaggccaaggacgagtgccgcagcgccatggaggccctcaaacagaagtccctttacaactgccgctgcaagcggggcatgaagaaggagaagaactgtctgcgcatctactggagcatgtatcagagcctgcagggaaatgatctgcttgaagattctccgtatgagccagtaaacagcagattgtctgatatattccgggtggtcccattcataccagatgttttccagcaagtggagcacatctcgaaagggaacaactgcctggacgccgccaaggcctgcaacctggatgacacctgcaagaagtaccggtcggcctacatcaccccgtgcaccaccagcatgtccaatgacgtctgcaaccggcgcaagtgccacaaggccctccggcagttcttcgacaaggtcccggccaagcacagctatgggatgctcttctgttcctgccgggacatcgcctgcaccgagcggaggcggcagaccatcgtgcccgtgtgctcctatgaggagagggagaaacccaactgcttgaacttgcaggactcctgcaagaccaattacatctgcagatctcgcctggccgattttttcaccaactgccagccagagtcaaggtctgtcagcagctgtctaaaggaaaattacgctgactgcctcctcgcctactcggggcttattggtacagtcatgacccccaactacatagactccagtagcctcagcgtggccccctggtgtgactgcaacaacagcggaaacgacctggaggaatgcttgaaatttctgaatttcttcaaggacaacacatgtcttaaaaatgcaattcaagcctttggcaatggctcggatgtgactgtgtggcagccagccctcccagttcagaccaccactgccaccaccaccacagccttccgggtcaagaacaagcccctggggccagcaggctctgagaatgaaatccccactcatgttttaccaccttgtgcaaacttacaggcacagaagctgaaatccaacatatcaggcagtactcacctctgtctttctgatcgtgattatgaaaaggatggtctcgctggggcttccagccacataaccacaaaatcaatggctgctcctccaagctgcggtctgagcccactgctggccctgcttgttttcctctcataagtagcgttagcttcgatgttgacttcttgaagacaggtcttcaacagcagactctaaggagcctcatgctttagcagaaagctctgcatcacgttactgtggacaggcagcaggaaatagagcagccaagcattgtcttttatcatttctggaaacacaagcgtgcatacctgtgggaaaactggtggccactcagtgttctacaaacactacaaatggcaccgttgccagctgacacaaaccagggccttggttttcatgttacctgacaatggcagctttgactgggacaggagacaggacgtgctgccctgtcttctgaagcagatccatgcccaggatacacagctcttatcctaaggattgtcttgactttcacctgtgatggcttggccgctctgtggatcggtttggtctgaagtcatcagatatctgctcgcactgtgttctgtgtcctctgtgatagactgtgactgactctgcgaaaaagaaaaaagtttgggggatcaagagagtcacggtcaccattaggtccatggagataagttggcccagagacacagaacagggactctggatataaggatctgacttgttaactgcacgtcccttccatcattctcttccttcagtgaatagccccgaaagtgcacttctgaagtctggttgaaatgctgagatttgaaatctacaaggatcgagtgctagtagccatgtttctttgcaactgaacagaggcgaggaaatttcacccaatagagtatccgaaaagttggctttcttctgacatcagtagagtggatggaggagaaaacccacagtgcccatttcatcctgattccctttgcccacggatgtttgccttgtaagaattaatccacttactagggcgggaaatctgttaaacccaagatgaaggcatagtcttttagctctgttggtgtttgaagtacatttatatccaaatgttaataaacataaatgtataatactaaaaaaaaaaaaaaaaaaaaaaaaaaaa

SEQ ID NO: 2 - DNA, 233802 bp, Bos taurus
  misc_feature (94959)..(95008): n is a, c, g, or t
cgcgccagggttggcgagaaagctgccgccgccgagtgcttcgactccaggcagcggccggctcgctctcggaaagtaacgttatttatttatttgcttgcgttcaacctcttggattggatccagtagacacccccttggactttgggaacccgcgtctgacctctccagcctttaccctgcgcgtgcgggcggctcccgggaatacacgcacatgcacccccgggggccccctccccgtgcccagatccgggtaggggtgggcccttacccggggcgctaaaaagtcccccaaggcaggagccggggaggggagaggggcgggggaggctccagcgctctccgctctggtttcaaagcgcgctctcccttccaggttgggtcggacctgaacccctaaaagcggaaccgcctcccgccctcgcgccaaccgcccgcccggagctgagtcgccggcggcggtggcggcggacggaccggggagtttcctctcgccctggatggagctgaactttgggcggccagaacagcgcgaccgtccggggatcactgcatgctgagctcccttggcgagaccgagcggcaggccgggattttcgggggggcggggaccagctgcgcgccggcaccatgttcctggcgaccctgtacttcgtactgccgcttctgggtaagtcgaggccgccactggcttttttatctgcggagtctttccgggagcactagcggcagcaaacccccccccccccctcaattgtgttgctggtgcggtggggtgggagcgggaagtttcgttgctgaacccccagcaaccccggctggggagtggggcgcgggcggcgcggcacttgcccgcagtttctcttccttggtttttgtttcctacgttttgtattgacctagcctgcgggcagtgaaagggtctgagccctccagccccgagcccgacgctccttgcgaatccacgggagctcgcggaggcgcggcgggcaggttgggtgtggacgagtacccgaccaggagagcaaagttggaagggaaaaactttttccccaatagcatctcagggctccccaccgggcgggccggggaccaggactcgccctcaccgtggccagtgcccagcccgctgctctcgccttccgaaagcccctgggcttcagtcgggccgggcggagcgaatggctggaaccggggccggcggccctggaggtcgagcggactctgaggccggtgggtcccggggcggcagggccggcgctcgcgatgggcagcgctggctcgcgggcaaatcgcgagaaacccgcaagcttcgggatggagtagggggagaccggtggcaggaggtgccggggcgggaagatggactctggggcgtagcggggcgggcgtgccgggtttgccgggagccccagaagcagggtcctgcgcgcgtcctccctccccgcgggcgcgtcgcgcccctgccccgccgcactcacgcgccctcccctccgcccgcagacgttctcctgtcggccgaggtgagcggcggggaccgcctggactgcgtgaaagccagcgatcagtgcctgaaggagcagagctgcagcaccaagtaccgcacgctccggcagtgcgtggcgggcaaggagaccaacttcagcttgacctcgggcctcgaggccaaggacgagtgccgcagcgccatggaggccctcaaacagaagtccctttacaactgccgctgcaagcggggcatgaagaaggagaagaactgtctgcgcatctactggagcatgtatcagagcctgcagggtacgcgcgaccggctcctctcccctcccccagcccctgaaggcagggggcccctgggccccggggtctggctgacctcatcttccactcagttctccccagctgcagaatgaggacctttcatgggccctcagggacgtcggcagctggtgtttgctgcctccctagcagctctgtcggggcctggttgttggcaaacccgtgatggggtggagggacatcccagggacccctgctgacctctctgaaacttggtcagaacatcgtagttaggaatccattaaaggccgcctcgaggaacctgcgacttccctccaaggaggagcactggctccacaggataatcggaaagcttgcactgcgtcttcttgcactgcgtcttgcttggagtttctctcttctccccttttattctgacacaagataaagaattcagaataaaaagtctagtggaatctattctatctttaccatccccccacctccaaacctgatcaaaggcagatgtggggggaaaaaaaattgtcatcttagatgatctgagtcttgaaccagctagtgtggttcagccttggttcatacagaaatgacttagagagggacaggtctacttgttacagtcattcattacaatggcagttttttggtccttgtgtgcaggaggtggctcatgtttgtaaatctaacacaaacattcagtgccttcattctccactgagactcttgctgaaaatctgtgatatacagatttgacctctcagctgctacttcctccagaacctgttaatgaaaacctatttcagaggctgactgtttaatagccagagtacctgctttctgaccgcatgccatgttttgggagcaaaaaaaaaaaaaaaaaaacaaaatcgggggagtgggggctctttggggtcacctttcttccattggctcctggaggcaatctgggcttgtgacgtcactgattccttcctctttggtttccttcctttgcaaagagtgggagagttttctggcaagatcagcctgggttgttctccaatgttttcatggcactctggggagagcaaggacaacagaggcagagggaatgaaaagaaataaccagagccaaatagatgcatttgaaatctttgaaagtttcagcatctttttgttacacaatcataacacaattagaattcagattcagtaacaaaaactgcctgaaatatgcctgttttccaatctgaccccagtctttctgttgtttttaggaaatgatctgcttgaagattctccgtatgagccagtaaacagcagattgtctgatatattccgggtggtcccattcataccaggtaagcagattcatacttccactctgatgccttcgccggcttgaaatccgttggcttgcgttccctttggctctcaagcagtgtataaggagatagttgaatggcacagtgtttctgggaaattagaaacaggatgatgataatgaagaggcagaatgcctagagatgattgtacgacttgaaagtaattctaaatgaagtaaatgtggtgtggtggtttgaaagaggttggagggaagtgacttggttgaatttcttccccccgccccccgcaataaaatatgattggccaactcttgcaaagggagtgccacttctgtctgtaggtggcaatcgtattcccttcttgctgcatactgtacacagtgcctccctggaaatgggatctatttaaatgatgctgctttactttacaagatgcttgaagatcatcttagattgttgaagacttggacagactcacgtgtctgcctttgactgaaaagattaagctctactatttgttaacgtttttttgttttgttcttaatttttcccgagcaagtgtgtgcatatatgaaagaatatgttcctgccctttcaaaagtggtctcacttcccgtggtttgttttgtctttgcaaagctacatttggtttggaagtgaacttagttttagaattaagttttccataacatctgcattcagatagccccacaaataatgagtgaggcagtgatttcccttaatgacatacagattccagactgatgttgtctttatttgtttacaattaagactttctcaagtatacatggccatcacaaattgtatgtttcttggtgattaaacgtggtggttcacactgaatgtgattctatgagatggtggtttggtcctggtgagcgtcttctagtgttctaaatgttttatgctttcctgcagaaagtcatagaaccaagacagtatgtaaatactgaaattatgtgtgagtgtattatacacacattcactcaagacatccgttgagcacctgctgcattctcaccattgggagtttggcagtgagccagatagaaggcatatctgctcccgtgaatcttagaagcagccagcaactggcatttattaagctcttgctttgtgccaggcactgtttaagatcttttcatcactggcttatgtagtcatcatcaagtgtgtcttacatattgttatttccattttacagatgagatgattgaggcaaggggaagttaggaaaccagcccaagatcacagagctcctaactggtggaaccagaatttaaactcagatttatttgcagggttttgtaacatttgtctcttactgctcagactgtgctcctcagaccatcagcatgaactgatgtcacctggaagtttgttagaagtatggactctcaggctacttcccagagctgtgggatgagcatttacattttaggaagctccctgggctgagtgctttatatgctcaagaaacttgagaatcactcttctctattatttttttggttcagagaaaaagtgtgtgtgtatgtgtgtgtgtgtttgtgtggcctctttctgggataaaatataagaggacaagtttagtgtcagagacattcatacatgatcctaaataatgttgatagcttataccccaaatccctattaatgttctctgtcccttcggcccagccaggacagtggctcagacagtgtatcaggaatgtgcagacaagagtgtatgaccatcagagtttttgtgttagcagatcagtaggaactagggtggggtaccagaaacctggctgaccttcctggtaccgagaggggcagtgcctcctgctggctgcctgtgccctggaagcattactgctcagtctttgtaccaatgcctggaaggatatgaacccttctcccctgctgggaaagtgagagtaaggaaataggtaaggaagaaggttcatctgggttttaatttcaaaactgttatgaactatagggctgtaatagactcattttactctctctgggtctctctttttttggtataatgagaccattgaagtaaaaatccccagtcctaacagtccatgattctgagggtagattgaggtcatctccaaaatcccccccttttcaatccttgcattctttctccacttaaagtgacctggccattcttggataccactggcttgtctggtttttttttttttttttgatgggtatcaaatgcttctgcctggactaagtcccacccattcagttttttggatcgctctctcacacagatatgttttcataacaaatatgttgtaagacccagcctgctgaggacccagaagtcataactcagagccaggaagatcttgtctggagctacattttatatctcaagggcattggatttaggttccagctgtaatgagctttgacctctacagtgactgtgttccttaagtgggtataggagagccaaatggagcctcgagaggtttcgagccattgaataatgcatacgatccagtttggcatttgtaggatggagagggacagaggatcgggatgatgaatgctgctcttcatggtttgaacctgagtttctagaacatctcttctgttttaagaatgtctctgaggaaactgggacagcagggcctttgccttttacagatagggtagccatcagagtttccatatttgggtgtctctgcccaaggtgaagagaatgtttatccacatacgctaagggtgagctgaatcctttcactgaacaagacatgtttgacgttcttgcatttccattttccttggactcatcaaacccacagccatcattggcactgtttacttgatcactagttaaaaggccatatggagatattatcagtgatgggaacaaggaagggaccaaaaataaggtaatgttaaaactgtcatctgaagcctgaactgcagaagggaaatcaagcaggctagtgtggtgagaattgaggtaggaagttggagggattcttctcctggaagcacaatagcagaagtggaagcattggcctccaaggcactttgaagggctctgggaagcgccctttctgcgggtggatgctggtgtggtttttaggggtcccaccacacagtcaagaaagtgaatctgtgaaggaagaagtgagtcagtgcaagaggacgtcggaagagaggcccaaacccaggaaagtgtcttgggagccgagagggcagagctgtgtgggtgctcagagcgtcggaggttggagctgtcaagtgataatttcacactgtgactcttagccatgcctttactctttcacttccattctgctttgccttttcttcctcacaaaagttcaaatgcagcaagaaataaatagtaatttgaaaggaagatgagccttttagcgctgtttttggactctctggaaggggagcgcaaagcagtgagaaatttgaaaagtggaagtggagtgggtgctgagaaaagcctggagatggaggatcagagagtggatataagcccctcgaaggcagcaagagatattttttaagaaagggctccaaggttttaaagtattgattgggaggtcggattttgaagacaagagtcaagaaatgtcagaccttcttcaggggtaatcaaagtcaatggtatttgcaaacaagagtcaagtggagagattgtgggacaagactggtgccagtatttgaacattctttatctgctttggtgaaagacaaagttctagttctttggctttgcagccatttcccttccaaactcccagagagtggcttagattttcagaggaaaccttaggatcttgaggttggttcccatgtaattggaaatggcttgccgcagcatcagatgcccctaagctgtcctgagtctggctcctttttgaatgatgctgtaaatcatgagccaagtttaatatttctgttttaatgcaccatcatatttatgtaactattaaatgaaaagtttcttaaaatgtttacatgttgaagaagagccttataagaccatctgctgtcttgtcaaatcatgacaagagagaaacgcgtgtaagtactagctgctaggggtgtgtggtggtcctctccttttcattcagctctctaatcgtgtgtccctgtgttgagaacccttataagtcttttgttctctattctggtacgtattaaactccttcacggggcttccaggcggtgcagtggtaaagaatcaacttggtaatgcagaagaagcaagagatgcagattggatccctgggttgggaagatgcccaagagtagaaaatggcaaccgactccagtattcttacctggaaagttccatggacagaggagcctggcaggctgcagtccatggggttgcaaagagttggacacgactgagtgactgaacacacagcatacgaccatcttcctaggccaccttggctctgccctccctctagaagccactgcccctctccagtcaagctcaccgagtagaccattcccaggtattgcatgcatgtttgctttcccgtgctttgcctttggctttgcctctgtttggaaggtttctattagttcttcaagaccgagatcaggtttcatctctgagggccgtgctggaattcccccagacaggcacttccactcaggattctctcacgctgcagttcgcgcttgcctttatggtagctgttattcacagcctatcgagaggacttatttatgcgtctgtctttctcactggaaggtcggggatttgcttgagatgccgggtgtttgctcagcatcgagcatccttcccggctgtgagcaggcattagcacaaccatatgtgctaagtcacttcagtcctgtccgactcttgcaaccccatggactgtagcccgccaggctcctctgtccatgcgattctccaggcaagaatactggagtgggttgccatttcctactccagaggatcttcctgactcagggattggactggcgtttcttacacctcctgcattggcaggcaggttctttcccacaagtaccacctgggaagcctttatcaaccataggtggttgaatatcagagaacagatctagtccaatcctttgcttttacaaaattgaagttcagagatgggaagccatttggcctgggccacattacttgagggtctcctccagaaccttttcttggatctggtactgccttagagcatgtcggcattgacctctttctgttctgcctactgtctgaacagacatcatctatctctgggacctcagcttccatggacactatgactcagagccttccctcctactcctctccagcctctcctgagctctaaaccagcatttccagttgaaacccagctgaatccatcaggatatatatatctcagttacttcaaactcagtgcatccaaaatggatctcatcatctctccccttttctcctcctctccctgagtctcttgtcttacgtaaaggcattgcctgcctaactcgtaagctttggaatcatctccaccacctcccctcccctcctcaactcctcgcgctggacccatcactaagtcctcctgaaattacttgctgttttttgaattgcctttctctccattcccttagtctacctatcttacctttcactcgagctttgcgttggccttcttactggctccacgcctgtgtctagtttctccttgttcaacctggccacccattgccagatgttaccttagcaccttggtgacttccaggagtgatcgggtttcagtctcccttgttagattcttctattaatcgacttcccatctcaccccagtcgcccgtgtctaaagaaactaggttgaccagctgctgtcccaccagccctctgggcagctttgtgcctctttcatctttgtatgtgctggtgctggagaagggacacgtgctccagaattgcactcacctgggcggatctccagctctgccgcttgttttgggcaggtcatttgatctcatggtacctcagaatccttggatataagatgagatggtgatactacctcctttacaggttcttgggaattaaaaaggctatatgtaaaaagccttgcctcctgcctgaaatataattggtcctcagtaaaatctcagtcatttttaatactgtgccaaggccgtgttagctgtcagctcactcctgactccaaaagtggggctcgttttcgacacaccatattccctggtcagggcttcccaggtagctgagtggtaaagattctgcctgccaatgcaggagataccggagacgtaagttcgatccctggatcggcaggatccccttggggaggaatggcagcccactccagcattcttgcctggataatcccatggacagaggagcctggtgggcttgagtccatgggctcacacagactaggatgcgactgactgactgaacatgcacacgtgattccctgatcgtggaaccatgattctatgtctagaaaagcatagcaaccccaagctttcagaaaggctgctcatcaactggcttggacaagaaagaagaaatctatccactatcaaaagggccctggaggaaagccctaacttttaataacccaatatgagtcttaaatcccccctggggagggtcttccttttgcctgctctgatccccctgttcctctggggccggatccttcctttggctcttgctgcagctggctgtcaccccgggtgggagctcttcctgtacttaaggattcttgtgatcagcctacccttctcagcttgactgattcccattcttctaacctctccccataaatcttctctgcaacgcccctcatagcgggaagaattactgctgcctgagaagggcatgccttcaacctgggaaaggcttccgtctccacaggccctgctaagtacacttccatttcactttcacagaccgctacttttggcttttattctctcactctccggcccctcagcgatccatgtttatggtacaacagtaagaaattacaaacaggtaggaaaggaaagcgaatttaaaccattcatagtctcagctcccaaagataaccgctgtaaacatctgatatgagcctttgcagcttttttcctatatatatgaatatatataactgtgtgtgtacatatatatttatctttttttttcttttttacaaaaatgaagtcatagtctacacctgctttcttattacctgcctcatgacctgaggaatctgggtcactgaccccttcctaagggcaagctttattatttagatgaactgtcaaccttgagaagttccgtgagtaaggcctcaggtctttgcagtgtcttgatacttctggcaacggcaggaatgttccagtgcaggctgtggaagtagagacagtcagatattttgcatagaattactgtaggtcccgttacccaaagaggggagataaaacattggtgagtgacaggtaattgcatgaactgaagattgttacaaggtgcaaaattatttatagccaatatgtctgtcctcattccttccccttagtcttctgaagtatttctttttgccacctcttaatttttctgctgtcgaagcttattttttccaaaggatgtcctttttttgttgttgttgctattgtctggaatacagtggagaatcatcaagagacgtcatcaggatcgttttatccaaagagcgctggtccaggaatcaagagcctgtgttctgcctccagcagctagccagtgaccttggcaagtttttgcctttccagagagacaccttctccttttgtaaaatgagggcagccgctgaagatgacctgtaatgtcttccttctttctctaagggcctgactatggaatcttgtgttattctgtgacttactgtttttctatccggccttttcttcctaccagagaaaatcatcatctcgccatatacattttcatattaatgacatttgtaaagcatgatttattcaatatgtaccttactttgcaccagagtgtatttaaagcatctcatttgaatacatgcaagaagataaaataagaaagtggagaaaataaagaagagggcaaaacgagagtaggaaagagaaaaggaagccaggagtgaagacaaaatgcatgtcataagattatattttaacaaatcaaattattggaagtacagtgtgaaaacaaagggataaatataatccctgatggaagatgccataggacttttggaggtcattctagtccaatccttttattttactgataagaaaactgagagttccataaatttccaggacggtgaaacataaattgtcgaggacagtgatgacaagtaaaattaggtatgtgtttgtttttaaattgatatttgaaattctccatctcctggcatttcctcacctcctttgaattgtccttcactcgctcctcagccatgcacccttttagtttgccagactttgttctcacagttagagcgagaagcacagcctttgacttcagttaaagggttggctgtccattgtttcggaattatttcagaacaagggttaattaataggtctctgtttggcaacagaacccagtggccagaatgtacattttcttatcttttgacccccttcagaattgtctgcaatgaaaactaagaaattatttgttgcttgaagcctgcccagccttcacattcaaaaaaaaattaatcttttgtctggatttttttttgttaaagaaactaaccacttcccgtaatttagagactcaactagctgggtcagtgatgagaacgaatgggtaatttgcataaggggctaatttattatgtcccctgggcatttctttcatctctcttcccattctaattaatgttctaagagggtctatcaccatatcagaaattaagggtgaaagcagccattgttaacctttattcttatgattttttatcttctgtgatcttttaaccattttcttttcatatccagggatttacacagatagagctgggaagtgcagggagagattttaggggtgtgctcttttctgatctttcttcaaatagtgggttgttgtaaaaataaaaatgactcagggtagctttccaaccacagagggatttgtaaaacacacagaagggtacagtcatggtgcagaattgttactgaatttgagccccttagaaacatctatataaacatgtatatacacacgcacagatagagatatatttatatatctgcctgttcatgtgtatatgtgtgtatttatagcatatgtgagtgtggatgtctgtattattaaacatttcgcctctaaaagatcttccttgacacgtgtttcagtcggttctggctgtgacaaagacagctgtgtgtgagagagtgtgacacagctgaggaaaggcaagagaacaagcaatttcttcgaaaatcaattgcaatttactcccactaaattggcccacggtccgccccgtccgagcattagcatatgacagacttttaatcatttggtcaaatggcctatttctggtcaaaccacagttgcctctggggctctgcccttagtcctagagctgtattgtctctagagctgctgtctctttaaagaggatctaactgtggggagaggagttcctaggggaggccccgctgagtgccctcaaggccattatttccttttgattcccacctatgctaaaaggtgagccttcctggtggctcagacaataaagaatctgcctgtaatgtgggagacccaggttccgtccctgggtcaggaagatcccctggagaagggaatggcaacccacttcagtatccttgcctggagaattccttggacagaggagcctggcagcttaccagtccatggggtcccaacgagtcaaacttgactgagcagttagcacttaatagtatgcgaaaaggtaggtataatattctgattcctgaaagggaaacctgagatcagagaggatgattaactttctcagcaccacaccgccattaagtagaagagttaagtctcacatcttatttctgcatcttcaaagcctgtgctctttctactgttatgtgatgaaattttaagcacagacctgatcctttattaaggaggaacctacgtagttcctgctgtcttcccttaattcccagtttcctttcctctgtcttctaccttaaagcactatttgacaaaaagcctttaaagttgaaatataaaccaatatctattattttagtacttagccctgcattccgatgggttcttaattgacaagtaaaatccagaacacgcgtgctattaaaaccattcctgcggaactgcagaaatattaaccaaagaagcaatttctggtttgtatcttttttttttcatttcagtgtgagatggaaggattctaaagtagagcagaatgcctagagccattttttttgtcatgttgttgtataaatagcattacacagtggtaaagaaaggagtacatactttagtcccactttaaggtgacctaataggctattcatttgtcttccatcatattggtaccacttcccttgacgtccctctgatggtgtaagaagcaccatgccaactgttattcctgttgccctgtgtacattattgttttgtgtatagttaatagtgtatgaaaataagtattgattgcattctttgtagaaataggccagcaggtttcctaaagcccatgtttatgaaatgtttcattgattccgttagtatgagtttcaaataaactcttgattaagaagataccaggagatcagagaattatggagagttcttgatggaactattatattttcatatgtttaggaatggcaagtgggatttgaatgggaggagtgaccaggtgaactctaaacatactcctgatggaaatggttaccctccccaggagaccttaagaaaaataaaaataataattagcaacagctgtgagaggcagtgagctaggtgttttacacacaatagcttgtattaaccttgtttagtagaaggtgctcttagccccacttcctggctgagaagactgagacccaagtaaggaactggcccgagggcacatgcctaagaatgggtggagctgcattttcacccaggaggcctgctccagaacccatgctgtagtgtctgtctgcatccatgtaacaggagagatagtccctgaaagtcaagagcacaaactgcatgaagtcatagacattttgtgctgagttatatatatgtgtgtatgtgtatgatcggagaaggcagtggcaccccacgccagtactcttgcctggaaaatcccaccggcggaggagcctggtgggctgcagtccatggggtcgctaagagttggacacaactgagcaacttcacttgcacttttcactttcatgcattggagaaggaaatggcaacctacttcagtgttcttgcctggagaatcccagggatgggggagcctggtgggctgctgtctatagggttgcacagagtcggacacgactgaaacgacttagcagcagcagcagcatgtgtatgatatacacaggtaatatatattatatataaaataagtacactataacgaagagaagaattggttggtttctgtgatctctgttgactttcagtattttggtgtgatagagtggcatttctgtcaaattcattcatcacatacttattgagaatgtatgacgagcctactgagaggcctaggtgattcagaccctactgttgccttttaagatttcttttttcttgttttagggataggaataaaacaaaacaaaataggttataggtatataacaaaacaaaaaaaaataggttttatggataggtataaaaaaagcagattatgttgcaaaatggtaggcaaatgagtagaactccgtacaggctcagatgtagccctaaggagagaatatttgactagatggagaaaaggcttctgaaggggactttgagctgggttttgaggagtggatagaagtttcctacctagagatggtgtggggggagcattccaggcaggggcaacagcgtgggcaaagatgctgagtgtccaggttgtagttataagctgctggagacgtcagtatgagcagtgctggaagacaagctgggcagagatgtgggtttttcacgctcagaggcttggcctttgtctcagcacaaccagggaaggatgacggctcgtgtatcttagcacaatcgggatttttaggttgaatcgtatgaaattgccatttttgcaggtcagcatggtagaatagcacacatttcatatggctcaacctaatatagttttgtgtcataaaagcacgtagggttctggatgctcccagactggagaatgacatagggttagggtttgtaactcctcatctcagaaagtacctgtttatctcagctcctgatggacacagagcaagtgtaggtcaagtttaagcttctctctttcagaccttctccctggggggagaagatgccaaaggtacccattgcacacagctccccatttaggtgctcagggcgggcagaagagctggagtcagtgaggccgcctggacctcacagggtgggctcggaaagggagtggggcgcctggagagcaggtcacagcgggactgagtgcaggaagcagggtggtctggggccaggttatgaaaagtcttcatgctgttataggagacatattgacttgtagcaggagaaagggtgtcctggaaaattgttcagtggagccccttagccactgagtgatctggctggtgaaaagggttagcctggaggtcactgggattgtggacagttttggatcccccagaggaatggctccccacctgcctgctttttttttcccccaagattaaatgtatctatttttggctatgctggatcttcattgctgcacgtgggctgttgcagagagcgggggctactctctgcggtgcatgggcttctcattgtggtgtcttcccttgatgtggagcacaggctcagcacatgggctttgttgctctgaggcatgtggggtcttcctggagcagggatcgaacctgtgtcccctgaattggcagtggattctctacctctcaaccactccaagcttccctcttagctcagtcggtaaagaatctgcctgcaatgcaggagacctgggttcagttcctgggttgggaagatctcctggagaaggaaatggcaacccactccagtattcttgcctggagaaccccatggacagaggagcctggcaggctttgtagtccataggatcgcaagggttggacacgacttagtgaccaaaccaccaccatctcaaccattccaatgcttgccgtctaccttctgcagtgcctaaatccaaccccagtgctgtcctccagcctggaatcaggatgggcttctgctttttgacttggggacagtggtcagtcatgggtcaggtttgatctccaggccagagttgtcctcctggctgggagagatggccctgctcaccttgtctgtgtccacctgttgggaggacttgatgtatgggcttgtttctgcatttcttttctagatactctttaatccttctggctccctagtcctgggtctcacttggcagttttctgtgccacattcttgacttgcttcagaacgttttgcaagagtctgtctgaataaaaactcaaagattctaaatgcagggagttgtcttaattgaaagagatgcccgctaaccccagggcaagcccagtgagagaaggatgaggaaggagggcctttgggattgcagccagtaattgctgaagctcacggagtggttcctaagaagaaaccaaaccctctctgtcgttttttgagcccagagtgtgtttatgatggtgttttaatgataccgctggggtgggagcaggaagaggttgtactttgccctttatggctgctgctgatctcctgtaagaacgcacccagagccagggacctttttgctcaagagccactgggcagaccatctgtattcattccggagattggggtggggctggggaggggcaggggaatgaggtcccagtgatcctaggaaattatggaatgcagaatagcagtggtaaagagcaaagtcttggggttaaacacacccagatttgagttccacatgccagcacttactagctgtgggacccagggttaatagagactcggcctataagtggggataataatagtatccaattcataaagttattggcggtaacacttaggatgagcactgagaagtgcttgggaaatggtaggtgaatgactgccaggagcttccttttatatgtgccctaacttcctgccttctccctcctctcccaatttattggcatgaaacagcaatagtagattatctgagtggatgtagatttcttttctgctctgaaaggaaaaaaaagacaccaatagctatgcgggaaatgatgatcaccaattcagttgagtccaaaacaccaattatcttctctaaaaataatagtagttgcattctctgagaacctcctgtgtgcctggcactgggctagttgctggggggatggtggaaccaggaagaggatgcagggcacagggattggcacagggagccggaaggaagagccacaaagggaagtaggatgcattagagagaggaaagctctgggagacagaggtggagtccaggtgagccagggtgcgctggtagaaagggaaaggaacccagcagagaaattaacataagataggctagccctgtgttcagcagctgtgagcaagctgcaaacctagtgtgtctttttcttgattattctcaagaatgttttaaaaaatgtgatgtttgcaaacaaagcagaaacagtcgaagcattgttttctattagcagtataaggttcactggctatggctgttttttcatccttgtcttccaaattgtaattttaaaagatgaggcatttcatattcaagagtttcctttgtctgaaataaattattcatttctacccacttttagaaacaatgaacataatacttaatgcatcacaattaagcgggtgtagcttcgtataaaattatgttctctgcatcttggtggcaaacagagcagcgtatcctatatcttgtcgacatttgttcctcataagcaaaacataaagaaggagagactctgaaatgtgttttctttcccctcccgatggaggaggggtaactttagacccgaccagtcttgtttttgacattgcacagctgctgggggctgtgtcactgcagagaaagtgaagttactgcctctatattgtgcgcggggtcaggtgagggtcaggattcttgagcactgcccattcatctcagccccctttgtctgggtttgcgatccctctagaatcctctctgagaagagtattgcctggaaatgacttccttctggatttagtggcaactgcttcctgaatgctggccggttggtcacacaatgcactgtttctgagcctctggcattttgaagtacctttttctttgtgtatgtgacaagattctaccttagggaactaggcattgccagcaaggattgagatgtccatcgaaagcctgttcagtcttgaaggggatggtccccaggaactcaagtcccagctgagaggtcagagtcaaagccggctatgattgccagagagcaattgtgaagaacatcacttgtgaaatgcctgctggcttcccagcagtaggataccagatgcctccttagattatttcatttcatcctgagtgttggcctggggtggggtggaaacattatttccatcttatgtaagaagaaacccaaactcagaaaagtcaagtgactgccgaaggccacacagctagtttttggaagatcttgaattcatgccaaatttgtccagctcctaagatctgggttctgataagctcttctgcttgttgccttttttaattgttaggtcagaaacaaaagcaaccaaatgcaagatgggttctcagttaactctggtctcagcactattcgaatagaaacaatagtatctttctaattttaaagagggttgaaaggtagctagtagagcttcccaggtggctcagcggtaaagattctgcctgcaatgcaggagctacaggagatgtgggtttgatccctgggtggggaagatcccctggaggagggcagggcaacccactccaggattcttggctagagaatcccatggacagaggagcctggaggatcacagtccatgggattgcaaagagttggacacaactgaagtgacttagttcacacccatgcacaacttagtgactgagcaacagcaaccaggaagatgccctggacttggccaaaggttttcccagatacttccctgcccttgaattctggtatagtgtgttgagtacctgccttggctggactgactgtgtctggcctgtgggtgttctcagggtctctgcttccacctccgtgctgatgcagctcctctggacttgattctgagtcccttgctctgaccagtatctcccttggagtcttcgtactcagttcgaggagccctctttgagtctctttgagctaagtcaatccaacttcatgctgggtggtgccgccaaatattggagttattccggtcgtaactcagatgtgctccatgttaaattcgcctctcttaaaccagggggaaccaggaagaggcttatttagaacctttagggagatactttcataggttggggagagcagtgcccaagtggaaagaatgtggatttggagtcagacctgggtttgcatcctgacttcatcatctcataactttggcaagttatttacctgcattaagcctccatttctcactttttccagtgtggataatatctacactgcagggtcattgtgagaactgagttgtgagacattgtgtttaagtagaaatgatagagggcttccctggtggcgctagtggtaaagaacccacctgccaatgcaggagacacaggagatgcaggttcgatccctgggttgggaagatcccttggagaaggaaacggcaacccactccaatattcttgcctagaaaaccccatggacagaggagcctggcaagttacagtccatagggtcgcaaagggtcagacatgtctaaaacaacttagcacatgcacgcagaaatgatagaaattcttacttaaaaaaaaataatgattcttactaaaaaaaatgagaacttactcctcagcctcaagacattgcactgcacccagggagagggtgacagtcctgtgcccagccttctggatttccctcagaccaggtgactgctttctgcggacctcctggtatatctgcagttgccaccttgagctgcacggtgaccagtggcctggttcagctcatgaagccaagctgcactggctgtttctgttttcttgctgttatctgaaatcctgccttacagctccttgatttccccaaacattctgcagcctggtggagtagggtcggtcctggacttgttcctccatgcccttgcccttaattaagagaaagtgcagcacacactggtagaccttccagaaagtgctcttacgccgatcctgtggctgctactgtcggggtgcaccttggagactgggggaaggagagtgcttcgtgggttcacatgcctggtgaggatgctgagttgaatgtagaattttcctatcaggagcaaaaccaaagagctttttagataagcgctctgacgtgggccagaaacgccgggcaaatatttaccctacatgtaaaacactcactaagttaaatgttatttagaagccctaaaatagaaaaatttacactcagggttttatatctgatgattgaaaaaaaaaaaaaaaaaagtcgattgctgtttcccctttcctctgagctgctttttgtaacgtggaatgacagcagagcagtaccctcctggcctgcccattctggaactttccctctcagtccacctttatcccatgccccttttagacgtgtacccttccctcccactcccatccccagtgactctgctcttctgctctggatgccctgagtttggacctggcaccacctcatctgtccctccttgtggatctccacatgccaactgtgggatgctggctttggcagacttccctccccactggtttccagcttctgttgactgggttgtttaaactccaagaagaccggcactgggggccttgagaaggtctatgctgctgtcgcacacctgggtttgagactgagcatcccattaaaagagttactttcctttgctaggcccctctgttcctctctggcaaagcagtgagagggctgtgctggtggttctgacctggcatcatagagcattacgtaccatcatcatcctatgggggaggtttatgatatcctgatggctctccccaccccaccccaggtgtgctgtcttggctgaaaattgttgggctacaactccatgatttccatgggaacttgtcggttttgacctgccttgtggtctttgatgctttgtcaggtatcttaggatgacttcttccttccattcatcaaccagtttttaccgagtcaattgtctgtgggctggacacagagctaggtcctgaggacacagcatgggtgagacagatggggctctgaactcaggaagcttatggtctagtggaggaggcataatgagttcaaagaagttagtggtttttgataacagtaagtaatgtgaagacgatagaccaatatgacagcaaatagaggggagagggtaaccccaccgcaccaggacaacaggagggcctccctgagcaagtgactttgctcggagccctaaatgacaaagaactacccatcctcagcactgatcggagtgcatttagcagagtgcagaggcccccgggtgccctctccgtgaacagggaggccagtgtggtgggagacttgtaccgttttgccggtgatggtctcttgctgctgccctgggcagggggccagtgatgggggcaaggctgacactgggggtcagaagggagccacggtggaggtgggggtgcagggagcactaggaggctttggagccacggttcctgctttggggaggcacattcatgcaccttacacgagaaaggttccatgtctccgttaactgctctagaattctgaggtatgagcagagcagctgctggggttggacctggaggctttgtgtttgagtgtttctgactttgctctgttgccctcctgatacatgttttgaaaatataattctcgaatgagagttctctttcgagtctggatttatggtttccattgattagcctcttttttaaagacaatgaaatggaagttaattgtagaccattgaggatgaaagagtcttttctgatccccaagtccaggcctttggctgatgaatggatgactggactggagactagaatggttgaggggcttgctaagggggcagactgatcctggggcccagtcccacattcatgcctctcccagctgatatatgcagtgtgtccgagtaagtcatgcctgaggatggggaagttgaaggcatgatgactctgttccggcgggcatctgactttccttggaagaagaatttttaaataactgagtccctgtctgctttgtcttgtccctcatcttgccttgttctcttccagggagggtgatagattcttaagccactctgcttttttgcctgtccctcaaacacattgagtttgttctgtctcagagcctttgctctggcttcccctgtttgtctggagtgttcttctgcgttatcttcctttggctgattcctcgtcattttcaactcagagggcttccataaccacctagataagtaattcgcttccccctccttccttattccatctccttatttcattatcttcacaggttttaccactgtccaaaactttgtggctgattttcttgtttgcctatttctctcacttgaatgttaagttgcataaaagcaaagaccccgtctggcttgtccttttctctctcttcaatccaaggaagccacatttaggcacctagcaaaaatttgtcgaatgaataaatgggtgaatggatgtacagcgagctagggccaggtaggtgaagacaaattattcagtacttcttccatgcctagcacttgggtggaacagaagtgtacaaaaactaggctgggaactttaacagttttgttaaagaagtacatctattggattctttgtcttataaatggtggaaaactcacccctaagaggcttgaatgctgaattactaccttttagattatgcaaaatatccagagattaagtgggtttatgttatgtatggatatgagagttggactgtaaagaaagctgagcaccaaagaattgatgcttttgaactgtggtgttggagaagactattgagagtcccttggactgtgaggagatccaaccagtccatcctaaaggagatcagtcctgaatattctttggaaggactgatgctgaagctgaaacgccaatactttggccacctgatgtgaatgaagaactgactcatgtgaaaagaccctgatgctgggaaagattgaaggtaggaggagaaggggacgacagaggatgagatggttgggtggtatcaccgactcgatggacatgagtttgagtaagctccaggaattggtgatgaacagggaggcctggtctgctgcagtccatggggtcacaaagagtcagacatgactgagtgactgaaccgaactgaagtgaatggcgaacactgtcaccaagaacatggcttcttgacctctggctcttggatctgctcttggcattgaacagtcccctccaggggctgcagggtgacatcttcctgccaccgcatactcctccctcatggtcacagaaggtcctcagcatttccagaccgcaaagcctcacttttgccatccccggctagatacttggtataatgaggaacacagacagattggctctgtcctcatggagtttatagtctagcaggggagagagacacaatcacatgcatagaaagttccaggtataaagaatgctcataaggagagacaggtggggctatataagagccagtaatgggggtcagacttggtctgggtgggaagaacattctaggcagggaagggagaggttgaggccgtgtgatgtgagagagcaggtgatgaaggagaccagtgggagctgagactgaggagccacctctggctggttagactgggtgggggaggactggcctgcacacgcagcctgtgtgcctaaacgtcggaatgcaccatggcaggaactgtggggctgcatggttttgtcagcccactgtcgtcattgtcacatttgggaaggactgactacagtcatctgggaagaagaggccctgagcccaagctctggtttccagggctttcactcatttgcgtttcagaaagaacccccttttctttgatgccctctgccctcccaggctcggtctcctcagttgtgaactggggtcacactccgcctgccctgggtacttcctcggcccatcacagacaggcgtgagatgacacgtggcctgcgatgctttgtgaactgcatgggtcaaacccaggttgttttgattttccttcaactctggggagagactggattcaagacagtgtgaaatgatgagctccagaactctgggacccgaaggagcccgagggagctttttggttgacggctcctcttttctcctcctgcctcttggtccccagcccacccacattctacgaggagtttgagactacaagtctgtctcattcaacctgtggaacacacaaatggctcctcagtttgccttctatatttttcttttttctaactttttattttacattggagtgtggctccacggtaaagaatctgcctaacaattcaggagatgatggagacgaaagttcaatccctgggtctggaagatcccctggaggagggcatggcaacccactctattatccttgcctggggaatcccatggacagaggagcctggcgggctacagtccatagaattgcaatgagccggacacaactgaagcaacatccacatagttgattaacaatattgtgttagtttcaggtgtacaacaaagtgattgaattgcacatatacatgtaactattccttttcaaattcttttcccagttgggttggtacagaatatggagcagagttccctgtgctgtgcagtagttctttgttggttatccgtttgaagctattacatgtaaagctgatgtgtacaaaagcatcactaaataactgatgaattctccaggtgttttctgattctttctctgggctaggcgctgttctgtatgtcgaggatacagtgaggaacaagccaggaaagggccttgccctcaaggagccagggggagacagatgataaagggatgtagaatgaaagcaaagagaggagagagagatgagaggagcagagggtgagcactcgcttagacacagaaagccttctctggtgagatgtttgacctgaagtaaccgtggaatctgatacagtgtttgaggaaccagccaatgcccaggcccagggctggggaactgttggatcctgaacatggcgactgatgggaggacaatggaatgagctggggtagaagagccagctggtgctggtgacatggttggaggacttgctgggctgtgataaggactttgggtttgtaccacctgggacgagaagctcttggaaggactgtgaagggttcattgtagttgttgttcagtagctaagtcgtgccccactgtttgtgactccatggactgcagcaaaccaggcttctcttttcttcactgtctctcagagtttgctgaaattcatatccattgagtcggtgatgctatctaaccagctcatcctctgtcgtccccttcttcttttgccttcaatctttcccagcatcacggtcttttccaatgaaggatttgtggcaagataaagtcatgatcttatttgtcttagaagtagcactcttgattgcagatcttgcctctggtaggacaagaggagaagccgaaaacccagttagaagactgtgtagcatcaacccttgagccaatggcagctcacactgggttagtgatttggagcagttagattcgagagatgcacaggacttactgatgggttgagttttaagggtgggagacacagagaaggcagggatgacttcttggcaatgtgggtgctgtcatttagctgggtgaaaaatggtgtcattttctgagatgggaaagaccaggggtgagtgctgttatttgctctgggacgtgaggatttaggattccattttggacatgttaaactttaaaatgcttttagtcttccgaacagaggtcttcctctctggaattcagtgttcacagggctgttcatcttgtccatgttttgtgctttgctgtatatgttttttcatgaatgatagtccactgtgggtgggcatttaggttatttctgtgtttttgaggttacaaataatgccacaataattttttttttctggcctttctgtatgtatatggcagaatttatacttggaaacagagctactgggctatgggccatgtacattttatttttggtaacagttttattgagatataattcacttaccatataaggggatgtatattttcatgtgtttttaaatcttccaaaaccactttcattgtttttaagtagtcagtacatttttcaatttactaacatatttaactgatttatttgccaacccttctgggtgttctttttgctgatgcatatcttttaatagttatttcataaagtatccatgcctagtaattgctgcccttttttttctttctaaaatcatagacaatatttttaaattttagatctatggaaaagctaagcagatggtgtagaggttcctatatagcctcctcttccacagttctctcattattaatatcttgcattaatgtgggacatttgttacaattaatgagccaatattgacatgttattattaacaaatgtactgttccctttttctgttatgtagttctgtgggtttctgacaaatgtagaatatcttgtatccacaattacagtatcatggcttccctggtggcttagcagtaaagaatctccctgccagtgaggagatataagagacatgggtttgatccttgggtcaggaatgtcccctggagaaggaaatggcaacccattctggtattcttgcctggaaaataccatggacagaggagcctggcgggctatagtctatggggttaaaaatgagttggagaggacttagcaactaaacaataacagtatcaaatagaatggtttcatcactctaaaaatcccctctgctacatctggttatttttgccctttctttgagcctgtggcaaccactgatctttttactgcctccctagttttgtcttttccaaaatgccacaggttggaattatacaactttcctccatgtctttttgtgagttggtaattcatttctttttattactgaatgatagccattgtctgcatgagccacaggtgtttgtttgtttgtttttttaatccattcacttatggaaggatgtcttccttctagctttggttgctttcagtttttggagattacaaataaagctgttataaacatttgtgttgagatttttttgtggtggctatagcttttcacacctgacctgcctcttgagaaacctatatgcaggtcaggaagcaacagttagaactggacatggaacaacagactggttccaaataggaaaaggagtacatcaaggctgtatattgtcaccctgcttatttaacttatatgcagagtacatcatgagaaacgctgggctggaagaagcacaagctggaatcaagattgccgggagaaatatcaataacctcagatatgcagatgacaccacccttatgacagaaagtgaagaagaactaaagagcctcttgatgaaagtgaaagaggagagtgaaaaagtcggcttaaagctcaacattcagaaaacgaagatcatggcatctggtcccatcacttcatggcaaatgggaaacagtggaaacagtgtcagactttatattttggggctccaaaatcactgcagatggtgactgcagccatgaaattaaaagacgcttactccttggaaggaaagttatgaccaacctagacagcatattcaaaagcagagacattactttgccaacaaaggtccatctagccaaggctgtggtttttccagtggtcatgtatggctgtgagagttggactgtgaagaaagctgagtactgaagaattgatgcttttgaagtgtggtgttggagaagactcttgagagtcccttggactgcaaggagatccaaccagtccattctgaaggagatcagtcctgggtgttcattggaaggactgatgctaaagctgaaactccagtagtttggccacctcatgcgaagagttgactcatttgaaaagaccctgatgctgggagggattgggggcagaaggagaaggggaccacagaggatgagatggctggatggcatcaccaactcgatggacatgagtttgagtgaactccaggagttggtgatggacagggaggcctggcgtgctgtgattcatggggtcgcaaagagtcggacacaactgagcgactgatctgaactgaactgaacagcttttcaactcatttgtggacatagcttttcaactcctaaatacctaggagtgcaattgttagattatttttttcgtgtatagttagcattgtgaaaactgccaaactgtcttccaaatggccatgcattttgtgttctcaccagtgatgcatgagagttctgtagctctgtattctcatcagcttttgacatcgtcaggtttttggattttagccactttaattgatgtggagtgatagctcatggttgttttaaactgtaattccctagtagcatatgctgtcgaacactttttcatacgcttatttgccatctgtgtatcttctttggtgagaagtctgttcagatttttacttattttgaaaactgtgttgtttgctttcttattattgatttttaagagttctctatatgttttatatgtaatttttaaaatcagttatgtgtttcgttgcatctttttacacttcaaaatgtcttaatttagtcttcatttttggataattttcctgagtataatactttagtttggcagttagtttcttttagcacttttacaatattattccactgctagttggcttctgttgttgctgatgtgaagtctgttgccagtctactgattgtttcttggagcagccttttaatttttatttcaaatttttctttctttctactgaatctgaatagtttacggtttccctgttgttttctattcactgatttcactgatatttgggtaggatttgtttttaatttgtcctgtttggtaataggtattattctgtcttttatctgaacacctgtacctttgtgttgttctggaaaattcagtgatgatctcttccagtatgtctctgttctttcaattctcttccttttaaacttctgtaagacagaggtcttactctttcctcaatttctcagaactgtggatccatatttttatttctgtatttctctgagttcttcagccctgtctccctagtcattctctcttctatgtttcactgaactcatctatggaagtttttatttaaacagtatattttcattttagaatctctgtgtttttaaaaaatcttattttttgagctgtgttctggtggctcccgtttctctttctgtctcactgaatgtatatttttaaaaactttcacactcatctcctctctcattcttccagttgctgctcctatctactttggtgtaggttttaatatttcaactataagtttatctgtagtgacaatggctttccctcgaagtactgcagaattcttgaaggcaatgtaacttggcataaacagagtcaagtctgaagttttggaagcgtgttttaaaaatttgactaaaaacagaaatcatttcttttaaactccactatctataaaaagtcacttaagcagagaatgacaatttgtcccctccccagacaaatgcatattaaacaaaacatgtccttaacggctcttctttgcttctgtcattattttaggagagaaggtatccatgcaaataggttttttttttaattgcaatatagttgatttacaatgttgtgttagaaaacagtttttaataacagagagccccttcaaaaattgctgccttcttattccagaaaccaaattcaagaccataggctgttgaggtaatctctattacttggctgggctttatggattttatgggtttggaactcacacatcatatgaagagcattcttgggactccgcactcttaaaagattcaagcttctttctgtgtcttgccccaggctagtgaacagagatactctgggtctcttttcaagttctagaacttctgggtctctggtttcatgcagggagggctttcagagttttctggtcaaaggtctgtgaccatggtggctgttccctcacatccctccaacagtgtacattgcaatcaccagtgtacattgggttgtacgctggaatcattggaattccatgtaggttgtacatggaatgtacaattcaaaatacattggaatcacctgggagctttagtatcctgaaacctgtacctcaccacagagattttttttaaagcttctagttgattctaaatgtgcaacccacatttattaagttgaaaattattgccttataaatatataccaaggcttcccaggtgactcagtgggtaaagaatcctcctgccattgcaggagatataagagatctggatttgatccctgagttggaaagaccccctgaagtaggaaatggcaacctgctccagtaatcttgcttggaaaatcccatggacagaggaggctagcgggttatagcctatggtgttgtaaagagttggacacaattgagcatatacacataagcatgtgcccagttccccagatttttttctagctttcatgagtctttgttgttgtttttgcatttagtattttatcttttgggggagggtcggggaggagtttatatatctgcatctgttcgggttatcgtattgaccggaaatgagaagcaccacatttaaaccagtgtgtttagataatcggacttaaatagaaagtttcaagaggcctctctggcttgttgaattcttcgtgcaatttttaatttctctgaaaaagataagtctttttctggagccctcccctcttcattcctttcagcatgtgtgcttatcaaatatctttaattaaaatgaatttatagtttggggctgcatttgagttagtggcattctctgcttctcctttgtgatatgaatatttgaatattttctgtagttggagatatattccaaaataagttagatttggaggggggacacatgggaattatggacttttgggagggggaagaaagctgatagttcaaaaatcaaggtgaaaagaagtgatatttagaaaggaggtcatttttctagctgatttaagaaatatatttttgaagtgaaaaaggaagcaatttttgaagggattctttgttactaaagcatttgctttcatagataatttctctcctgaaataatcaaagaatcaaattagggcagttttaaggatttgttttgttttatatggattaggttttggggataaattgtcattatctgcgaaagtgactttttataggaggtggagtttaaaaggaacaatctctgcttttagtcagaatttcaaaacaggtttccaaatcttcagacttgactggtctggtctgtgttgagttacatctgcatataattgtaaccatagacacttgtgtccctaagtatgtacatctgcaaatgattgtattttcaattgccaatgcacaggttgggttttaggctatgtctaaataagaatagtggagagatgaagcaattatctggataattcaagctaaaaaagaagagtcccccatattatatcttcacttttctggatatttcacttgagaggatgttttgattgaaatcatctgggtaattgcctcagttttccacttcactttgtatgtgtaaacacagctgttgtatgattgacttttttcttccaatttccattttggaaagcaccatttttcaatgtcgaaactcgtgcttatggatcgtatacttgaacctacttttcatatacttgggtactgtctgccttctagggaaaatggctgggtagcaggttgattttcctgcacctaaggttgaaggagcttctttttgaacctcgtgatatttctagctaacctctagagaaatcagatgtctagccagtgctatcaagtcctgcacattaaaaaggcttcggtcttcacctgtatctaacaatcacaaatagcatggcaaatactcctgtgtttagaaaaatatccaatttgaccatcctatcagttccagatggtacctgcttctgatgagcctctgtttactgccaaagtggaggctcagccactgcattttaattccgttcccaaactcatctgcattgtgcagcaatacagattaatacccatggagctgctgcttggatcagctgaggatgctaacgcggtcagggggacaatgatgagattctcagtgaggaggtttcggagaccttcagagactctgcatcgttgcatcagggtgagagcacgataagagctgagaaattgtgtggctgagatctgttttgacagatttggttctccagggcccctctgctttctcagcctcttccaggcaatttgaaatgctcacaaccagagagttggaccaaagcatggggaccatgaacctcctggtgacaggttcttggaaatgtgtgaattcattttttccccctgttgttgatttttaaagttctttaagttctggcaaaagacaccctgatactacaatgtctgagagtattgtttttcaaaaatgcacatgttttagaaatccagtgtatcgttaaattataaattatccattatgtgaaaatagttgcatcttaagtaaggacttgcttttgaggattgagagccattttattaatcacttaagtactttagcacaatgattgagagcataagttctggaacatatgcttttgtgcatgcctgctaagttgcttcagttgtgtctaactctttgaaactctgtggaccatagcctgccgggttcctctgtccgtaggattctccaggcaagaatactgaagtgggttgccacgcgctcctccaggggatcttcccaacccagggatggaacccgtgtctcttacatctccggcactggcagacaggttctttacctccaccaccacctgggaagccctatatggtttgataaccaggtcccaattccttaagtcttagctggataatttaaacctttctgtttcatcatctgcaaaacaggatgatcttgacatacctttacgtaccttatgagattgtcttgaagattaaaagcttagtgtcttccctgacatagagtgagcactcagatggtttcattcatttattccttttagtctacaaatatttgtgtggttacagagaaacataagatttagtctctatcctctggcaagccgctcatggctaataggtttgaaaggcaggtgaaagctacctgcgataccctatgatgaatggtagacagacataccctttctagttgtttcctgcctatatgaagaccttcttttttcccactcagatgtcatttatgtgtccataaacggaaaatgttcaaagtgagtcattcactctttgggttctttggtttttgagagaagctaggactcaaccccttgaagagaaatccatctttaggaaacacctctttgtaaatttagttttgtttctgtttatggagaagaaaatagccatgggtacaaagattttagtcttaatttttgctgcctggggagatggctgaggcattgtctgagcccagaaagaataatgcgaggataaagcggagtccgtgagcccagggttgggaggtctcaccatccagcaagcacagagctgtggagcagttaagtcacaaggatgcgagtaaaaagccaagtgtgttaggttagcctggcaaaacagtaggagctttgttctaggatttaggattatggcagctgcttcctaagactcaagtcacttggattttcttggtggcttgatgaaccagatacagaatttttaaatgcagtggcttcccatttttgataatataccatagagaaatcaaacacctgctgtagacgcctctttttctctcgctgaaggtgaagtgctctgatttcccagatccagcaagtcctaagagatacattgtggccttttagtctgatgaccctcaactcatatgatgaggatccctgctaatagggctccactgtgcaacattttcaagattgcatttatggttttgctcagacttaactcttcaaaagttttgataaatcctgggagacttgattgtccgctttgctattgttgttagattaaaaaaaaaacaaacccaaatataaattatcttttaatgaagaccagtaaccttcttttgttgatgatgatacttgtctataacaagataaataatcattttaattattaccatggcatgggaaactactttttcaaaagaatttcattctaagtacatgacagactaaattagcatttaggaagtatgaaaaggaattcattgttaagttacgtttttatcttatgtcctatctggctttctttctttaattgaatgttattattttggaccacataatatttgctctgggtaaaataacaataactcattagattcactgcctctgcatgagatcaataacaaaatttccattatttattttcccagaagacctttagacgctgcacttgcagcatggtataaaggaaagggatttttataatgatttactttaaaaagattttcacctctgatatatatctggcaaagacccactagaattgcactgtgataataataattataaaacaaaaatgtcaagaaattgttctgtcgtgatctgcattttgactatgaatgaaacctgataaacacaggcgtgcaaagcataacagtgtttagacacaggctttgttcacacaacatttattaacatgatgtgttagaaagaactatgtctataaacatttttaatggaaacccacaaatgaagatgaacgtgtctgttgattgattggctggatgtcgtctgtctctttctctctggacatcttggtaaaatgcatttttctatttgtttctaacatagctgaatgtggctcatgatattcctgctttgagatttctgttctccgtgtttcagggtgagcccagttgaggaaatgaaaggcaaacacataatcttcagaaatttctcactttaggggaattgcaaatgttaatcctatagctagcatcctagccttcaagatgggcaaaaatgatagattcttttttggtattgcaacctttaaaaggaatggccatttgttttgcatgtcaatagtttggaattaaatggggaggaagagattgaactattctgtctcaggaaaatctgtaatgtcgcatggattttagattaaatggattagattagaataattggagaggacattatttcttcattgagcagatgacaaccaattaagttgtaattctgagcccttgttgaataaataactcttattatcaaacacgacagcaaaggatcagaggcaggatgggtgtgtgtttctccaggcagtcactcactttccagaaatttcacatgtatacagaatacacagagagaatgtagacacagcatggtatatgtcatgttctgagtcattttaagtctggttcatctgaaggcaggcatcccatgtctttggaggggctggaaggctgagtgcagatggtgatgtttgtaatgttctcctaacacaagtcaggagaattaaacccttctgataccttgtgaggtcacagggaggttaccttcctggccagttatccctggagcctgtagtgttgaaagacacagctttcctgttgtcagatactgtaccttcccagcagctgctcacgaaggcctgtgaagaaatcagctgtgtttgaacgcacttaatgttgtatataattctggtgttaaatttattggacggctgagccgaacgtaaaacagtgtgtaaaaacaaggctacatgtgtcagctttttggcacatttggaaggagctgaaggtttgggggatgtctgcactgtagaggaaaatcggctgtgaagtccgggagtgatctggaagttggctgtctaatcctcagtgacgctcagatgtgtcgttgtggcccccggggtctaagcgcaggtgttgtttggccccaggaactctcttttcccacctccttgtgaccttcttgagcagccctttggggcttttggccaacagaggtgatgttgcatccattctaggatgtccttcatccagaatctgtataggattaaacaatatacagggatcaggcgggacccgggaatggctgaaagtctttgttctgctgttacgatacagtatacagaagtagatgtgggtatctaagggagtggagaagaatagcggttaacagtgaagaatgcaggaaagaaagagttgctcctcttgctaattttttttttctttttatattttttgcagttttgttggctcattaatctactcattaaatatttattgagtatctgctacatgccaggcattgtgccaggcaacaaacacagcaaattgcttaagagtttaggcttgaatcgaatcaacttgagtcctctctctgccacataccaactgtgtgactttggacaagttccttaacctctttgagccataaggtttctcatctgtaaaatatcatagtgtaagcatccatgtgtctatcagtagaagtgtggttaaatatattctggttcaatcacagagtggaattccatatagctataagaaaagtatgagatagttttctatgtaaagatctccaatataagtaaaaatttccaagtgcagagcaatttgtataatatgctaccttttatataaaacatgagacatatatacaactgtggttggatacataagacacgaataataatagttatatgtgaaaggccttcccttctggctcagtggtaaagaatctgcttgcaatgcaggagatataggagatgcaagtttattcctgggtggggaagatcccctgaaggatggaaatgcaggaaaggaggaaagggcaacccactccattattcttgccaggaaaatcccatggacagaggagcctggcaggctacagtccatggggtcatgaagagacagacatgactgagtgactaagcatagcacatgtgaaaggggtgtggcaacttggcctctgggggactgcagtgggagggagactgtactatatacctttatgttggtgtttttaaaaatatttttataccatgtaaatatattgcctcttataaaataatgatagtagagccttacaagttagttctgaggattaaatgagatcatgcacctcaagttcttatccaaagcctggctcaaaataagcacgggttttgttgctgtggtggcaatgttgaaacaaagttacatgtaagaaggaagcctttcctatgaagtggagaaaggatactcacaggatattaccaatgtggtgcccgggaaggaataaaacattctggaatcctagttgagattgtttcctcagtgtcttggggaagagatagggagccttgcagaggtgatgacaacgctgctgctgctaagtcgcttcagtcatgtccgactctgtgcgaccccagagacgggagcccaccaggctccccaagagacggcagcccaccaggctcccccgtccctgggattctccaggcaagaacactggagtgggttgccatttccttctccactgcatgaaagtgaaaagtgaaagtgaagttgctcagtcatgtccaactctgagcgaccccgtagactgcagcctaccaggctcctccgtccatgggattttccaggcaagagtactggagtggggtgccattgccttctccaggtgacaacgctatataccttcaaatcctttcttgattaaataaatgtgtcatttagttctaacagctgcttcttccctcatatgggatacccacagtggatatttaatttgtatatatatggaggatttattcagtagttttcactagttgcttggttttaatcatattttcccccacctctgggtctgtttttcctcagatcacctgtctcctggaaggcaggaaccatattaatttagcttgttttcttcctagtgatttgttcttacttggtaatttttataaatatgaaacagtatgtacttttctccttttgtatctggtcctggcaaccttctttttggaatggaaggaaaattttccacccggggccctgttccgtgctgaaagggaattgttgttctccgttctgtggttcctggcaaaggcattgccttcaggatccttccggggagttatttggtgaggcaggggagggagtggcagccccattgttttggggcccctcccctcgggggacattgggtaggggtggttattctcagcagacactagggggcgcccacgttcatctcctgctggttatgtgtctccaacaaatgccaaagcaatggtaaataggttcccgtcagaatagacgtgacctttatggctccctttactggattcctcctctgtcactgtcctgaagggcaatggcaaagctcacaggggtgatgtctttctcagtttaccctatggattgaagcccgccaggctgctctatccatgggattctccaggcaagaatactggagtgggttgccatgcctcccctccaggggatcttcctgactcttatatctcttacattggcaggcaagtactttacctctagcaccacgtgggaagcccttgggctgccataacaaacaccatagactgggtgccttgaccaatagaaatatgtttctcactggaggctgggaaggccaagatcaaggtaccaacaaggtgtaggtttcatcctgagtcctctcctcctggcttgtaggtggctgccttctctctgtgtgctcaaatcatctcttcgtgtctgttggtagttggggggtggttggaatgtggggagcaagctctttccggtgtctcttcttcgaagagctctaatctcagcatgagggttctcatctctacctaactgcctcccaaaggcctcatctccaaataccatcacactgggaagtgggggtaccagcatgatgccatttgaggggagggtggtcatagacattcagtctggtagaaatggacatctgtcagctttggggagggaaagttgctgaaactgattctggtggactctgccatcttactcagtgtggggcagccactggctggatcgacttctttggtttggattggggttccatgtactgggggtgggttatgagttagcaaaatgttcggatattcaatcagatgtggtcagcaccatttcactcgttgttagggagagtacatgcacttggagatttatttcttaaaaaaagaaatctgtggatcagaaaatgaaactttgattaacagcctggagctccttaggaaaggatgtactttcagacaagctacaatggtcctttcagctacacgtgtcttccctttgactggccttggcaaaccagctccaagggtttggtgtggtttggttatctgatctctgagaggggtacagatgatcctgactcagtggatctggagcttgaagactgattttcctagtgctactctgggttagtggtgtaggtccttgtgtaacttcagctccatgcagaggccctgagtaatacagtttttgtatgtctgtagtataatttacattacagagtgtttgtgtatataagggggacagtaagagatcaaatgcataatctaatggagggaagagtttacaggcccatgtctatgaagaattagctaatctggaccccaatgagtggacctcccttgagtgaagtctccattgctatgtatcctgagtttaagctctactctgaggattcagaaatatccaccccaggtgaaaaaggctgtctaaatgttaggattctagccctgatttccccacaccagttggagctggtttcttctttatgtttgtccttttatatcgctagatcccaagatgctctccagtcaaaataagtggtgtgacttagttaaatgagaattcaatgagtttctgtagctggaagttcttcccagctctttaaaagttacactggacagggaacaaaacaactcagagtgggcattttcttgaaaggtaaccaaaaatcagtgatcgtggggtcatcagagcacttttctggattagcacaaaaactctgcatgcagcagagttgtaaatgtaagtgcagttctacctattcccaaagagtaaagagatttaaaatgcttatttaacccaggggctgaggagcgaaacaaaaggaaccatatccaaaagtgaatcgattagcagctgcccattccaggcatcagttatagtaaataaaagacttttccccaaagccatgttagtgaggtttagggaaaataaattctttttcccccttttatgtgagatggacttttatacataatttgtgttggggggaaaaaaatgaaaaagtgtgttgtgttaaaagaagccaggcaaagtatttagattgttgagttttcaaagaggaagccatttgggctgaataggagaaagatgctgatagattgggagtgtctcatgggttgcggctctggctgggaaggaaggaggagccaggtgagcaagggagcaggcaggctgtgcctctgtggagccctaggtgtaacagggcttggtgggagagggtcaccagagaggagagtgagccctgggtccttaaaccagggagactcagcatcctagctgggccaggccaggttgcagcagtatgcgccctgggcatctgtgcagggaaatcattagctggattgctctgagatactggggattaatcctgggaactccctgagacaaataatccttataatttaatatgctcacacactgccaggtgtactcagaaatctgactcactcaattcaatgtaactatactttttgaattatcacaacatagaacagaagggagtgtcttagaattctggtttaggccagaacttaacgttttctttgacctgaacaagcaagctgtactgaaaggtcagagatctcctaagcaggaaaatacctgagaagtgggctttccaggagttccctctgaagtgacttatcaagatcaagatggcacttcacactgatgatgacccttagttgtagatcctgggaacatctgagcctttggggcccttgaattggagatggtcccataaccttcctgattacttttttttaaaaacacctttgaggagtttaatgattaaaatgtgttccacttcagaagacttcagaacaagctgtaaaaagtaatttgcaattcttcctttctccatatctatgccgaatgtggtgtttaattactccataatagctttttgttctggtgatttgttacaaaaatagacaagttctttatcatcattaaacaccaggattcgtggttttttttttttttttaatttggaatcatttaactattttaaaaggtttaaagaaaaatcccgagaactttttttgagatgtggaattttaaacacaatgccttgatatttgcaataaatgaattcattaaaatggtgtatatttaaagaaaagtcaaggcactaaaatactcatctctttaaatcacccaatagctacttaagacaagattctggagggtggccaaatgatactctgtgatgtatattccctgggaggatgaattctctggtgtttttattcatggtatttctgaatagtatggaatatgtttaatctttaacctgtctattctaacagtttccagagttcaacagctgttctggatagtttgacataaagaggcagccctcttatctgaaactcttgacctttgagtcctcagatatttgttgaaaagaagagaatgtgaaccattgaggaagagagaggtttgcttcccctctctcttctctttggccatcctttccataccagtaataacatcatcattcacatagccatattttggggggggggaaaatccctctgaaatggtcctctttgcagatggaagtgcagttgattcatatcttggctgaattcattttaaataagcttgtgtgcccaggtcttgatttaggtgctgtggaaatgtgaagtaatgcctgccacagagcattagcttttatattgtagttagaatttaaatttatgggtatattccaacagattatttttcttcactaagcaggtgagttgggaaaaacttggtagctggttttagttaagacaaagatcttgatttggaaagggaaaagggaatactactttttaaggctgggaaaggaagacattatttaatcaaggtagccttcaaaataagaaactttcacctggatttacttaacatatatcatacattagaaaaaatactttacataaattatgtcttttgactctgcaatgagatccaagtgatttttggactagttatctccatgcttttcatgatgcaaaggcacatttgctgtgatgtgtacagatttgtaactttgtatcatagcagtttagagctagatggatttttagcaattacttagttcaacctcatcagtttaggtatgtgaggactgaggttcaggaagttaatcataagttgactcatgtcagagagtggtgaacaagctggaactagaaatctaggctcctaggctccagacctcccagactcaggactgtacagggacccactgccattgccaatccccatacctgtctctggaaatctcttaaagcacagctgaaaacagttccctgttcagaaggcttcatggttccccttggaatgaattttaaatatatatatataacaacatttttataatggaaaataatgtccaaacttcccttaacatcatattcagtgccttccccaacctggaatctagccccctcttacctttctagcgtcctctctgttttcttctgccctaagaacactgagacgctctgtgtttccacatccccatgttttccttcggcttctgcttttggagtgtatcttctctctgaagcatactcagttaatttcagcgcgtgcctggttgctcaatcttttctgatctttgtgaccgccttaaacactagtctgccaggctcctctgtccatgagattttacgggcaagaatactaaagtgggttaccatttccttctccaggggatcttcctaacccaggggttgaagctgtgtctcgtgcatctcctgtactgtcaggtggattctttaccactgagccacctgaagcccagtaatttcagtggctgctgcttatttagaacctttctagctgacagctttgatacaattcttattttctgtcttataattgtgtgtatgtgtgcacatgtgtgtgtgcaggcctgatttctttagtagaatgtggggctctggcctgtttatatgcatggcttctgtctaatagttgctcaataaatatttctagaattgaaaccagtcttgtttatgtcaccaagaacccgtccctgtactagttagagaagctagatttcaactgagaatctcatacacacacacacacacacacacacacactgtagcactttttaagaaatttctggggaacaagtattttccatctggaaggctgtccaacatagtgagaagaacctggatgctggcatgagaaaggcctgattctgaattcaagcgcctttatttaatcgcactgtgaccttgaacatgttatcagtctcagtttcctcatctggggataatgaactcgtcttttagggatgctattgggattaaatcagaaaacacgtatgcagtacgttgaatagggtaggcactcaggcagtggtgctgagaatccctttggttaggaaccatacttttgacaagtttgtctgttctcagttaccctatttgctattcagtgatttgctctttgggttgatgaaattaaacactgagacctccagaaagttgtcatataggtggatgactacttccttggatggatgatgtagtcaagtacaaagcggctcaaagaagctccaacacaattatttttatcagtgataactgcttaagatcaactatcatctcttccttaaggagaaaaggaagagggagaagggtgcccaccagtccctccatcatgaactaagccccagcagttagttccagaaactaacatatttccttcaaacatgtgtgtgttggggggaagggcaatcctacaactaactttctgtatagaagcatcgtgctgctgctgctgctaagtcgcttcagtcatgtccgactctgtgcgaccccacagacggcagcccaccaggctcccccgtccctgggattctccaggcaagaacactggagtgggttgccgtgtccttctccaatgcatgaaagtgaaaagtgaaagtgaagttgcccagttgtgtccgactcttagtgaccccatggactgcagcctaccaggctctgctgtccatgggattgtccaggcaagagtactgtagtggggtgccataggtttatggtataagccaggagaggagcaacctcctcatctccatttgtcactccaagtgtctgcattgaacacatcaggctacacttaaggaggttgtgtgttagtcgctcagtcgtgtccgactctttgtgaccccatggactgtacagcctgccaggctcctctgtccatgggattttccaggcaagaatactggagtgggttgccatttccttctccaggggatcttcctgacccagggattgaacccgggtctcctgcatttcagtcagatgctttaccgactgagctactagggaaaccccttaaggaggttggggaggatcaaagtcttaggagcaagactctgggaatactactcttgctttctctaacctccttccacacaagaagaggtctgtctgatactatgatgctgagcaggtcctagatgaataatgacgcgacagttctgtagggtcgacaagaacatacagaatcaacccaaagtgttggctccttctctgcggcccagtgctttggtgcagtttacactttcagtctcatctttgatgggagttgtatggggtcattccatagaaatgcttgagcttctttgggacagaatggacgagttggttaagccagttgttttgattgtgcagtcactgccttggtcacttgtcctcattgcctgatatcccaccgtttggttgggtgtaaccagagtgttggttttcttttgctgccccgacagattattccaaactcagtggtttaaccaatgtaaagttgttatcttgcagttctataggtcagaagtctgttttgggtcttactaggctaacgtcaacttgttaataagactgtgttctttctgcatacgctaagggagaatctatttccttgccttttccagcttctcaaggacgcttgcattccttggctcatagctctcttcctccataatcaaaatcagcatcttagcatctctctggcccttttttctgtctgagcatctctctagagaaaggctctctgcttttagagattcatgtgattgtattgtttccacccagataatctgagataacctccccattttaagacccatcatttaatcacatctgcaaagtcctttttgccaattaaggtaaaatagacacagattgctgggattagaacatggacatctttaagggaccattattctgcccagcgtactcagagataaaggagagggtgttggccctggctatcctttgaagccatctggcttcctaggagtctctagctggagataattgagatatctttgaacagagggcttcagacttaggatggtctacaatgtcatttccatcataatttctttacttttccaaatcacaccatccagggtttttctaaggcaggcaattgcttactgtctgccaggcctgggagaaaatggaagtggtgggaggagaatggttaatgagagctttgattacctgggccctgcaacaggctgtctagaaatagccatgttacagcgggttcttctgaaacacaaaatcaggtacatttggtaacgactgctataatgtgagacaacgtctcaagccacaaaccagctccagaaatgagctttgttctttacagcccactttgccccaaacataggccctttaaaataacttggtaggcacttactgaggaacagccgagagggcagaggccctgttgtatgcgtctctgtatgtccagcgctgagcactgtgcacggcctgccttgggtggtagaacgagcgagcgtggtacacggcgtgccctccacttgtgtggaagaagtgggtcttttatgacaggatgctgaatgaagccctgtgttccctacttctaatcactgcacccccagaagaggtgaaggatcctgagagattttgtctctgcttccattatgccgagattggaggtaattaagccaatgaaacttgccttccatttcagtttactggagccactgaacagaagtaggtgtaggaactgaagagcatctcccttgaggaggcaggattctggtgcatattatcttttcaaaacatgtcaccctaagcttctattcacaccgcacacaccccagagtggtgtttgtggaatgaaaggatgacagtgtagttcagcgcggaagaaggttgctcgctttggacttcagccgtcccaagttggaagcgtagctgtgccacttaccaggactgcgactctggataccttgcatcacctccaaaccttctctctctctctttttaaaaaatattcgtttatttatttggctgccttgggtctgagttacggcatatgggaaactcactgtggttcacaggctcaacagttgtggggtgtgggctctctagtttcagtgcgtggatttagttccccagtggcatgtgggatcttagttccccggccaacaatcgaactcacgtcccttgtgttgcaaggcagattcttaaccactagaccaccagggaagtccccaaaccttctttcttaactccagagagccaaggaataaaaaggtcagttggcacacttctttgggcatttgctgagtaaagaagaaacaataagatgttgttggtagggggtctcagctactttgtgatctcaattcatctccatagcctgccacatctggggtccatttctgtgtcctctccaggggcacagacatacctctggatttcttataaagtttcaacaaggtcagaagtgtagagcttgcctctgccccataggcccagttcttattaccctggacatgtggatcaaaaaggaagtcaaagaaggagaagcgaaaatctctctaaacagctcagggtttataagtgagggctgaagggaaccctcgcagatgacatgcaaagccccttgtagatggaatttctttcagcagaaattttccttctgtgaataggcgcctgtcatttccgtgtgatctttaatcattttcaaaaggcaacccttttgatcgtgagtcttaaagagttcagatgaaattttctgggcctgccttatttgggaaacatttgtgctttgtgaagactcgggcacgccaatatttaaaaggccaagccgagcatcttcatctctagtccaagggaccaattggcagagggcagcaaacaagagaccttttgtgcctccgtaattgcagagcagtgggcattttataaccactgagtggacttcagagttaattaccatttagaagtctgtagaagataatcttccaaaacactgggggtcaggaaagtttcagtgctttatttatatttaaaaacataaaccggcactgccccatagaaatagaatatgagctatagatgtaattttaaatgtccctagcttcacattcaaaaaataaaaaaagcacaggtgaaattaattttaatgtttactctcacccagtctgtccaaagtatcatttcagcatgttattcagtataaacaattattaatgagcgattttaccttttttgctcagtccttgaaattcagtctattttacgctttcaattcagactcaccacgtttaagagctcggtagccacctgtggctaggttacactgaagcactaggtggggatcccctgagaaatgaggatgttgtttttcatttatgcacagctttctcgctgcccagtatggtacccagtacctaagcatgattcttcagatgtgtccataggatgaagtgctgcatgcagttatgagattggcttgcccggctgtggcccaaagaagtagtgaccctgccagtcattagaagtgtagccaacagcccaagactcactcttttttccctcagtctgtttattgagatataattcacataccatataattcactcacttaaagtgtacaatatagtgggttttagtatatttgcagagttgtccatccatcagtcaattttaaaatgttttcattatcccaaaagaaatcctgcgcccctgaactgtcactcccttcatcctctcctgccatctaccctaggttactatttattttctgtctctagatttacctgttctggacgtttcatacaaatgaaaccatacttagcatgctgtttttcaagattcatctgtagcgtgtattagtacttcttttcattgccaaataatatcccattgtatggatataccacattttatttaaatatttcccagatgatggtcatttagggtgtttttagtttttggctattatgaataatactattgtgaacattcatgtatcggtggatgtgtgtgttcctttttgggggtaaacagctaggagtggaatagctagatcatatcataaactctgtattaacgacctgaggacctgccaggttttccagtgggtacaccattttacatttccaccagcagtgttcccatctccccagatctttaccaagttatgtttctgactgtagccattctattaccctagcggtgtgatgtggtatctcactgtggtttgatttgcatctccctggtgcctagggatgtggaacatctttccgtgtgctaactggccatttgtatatcttctttggagaactgtctgttcagattctttgcccatttttaaactgagttatttgtcttttatttttgagttgcaattcatctttatatatagtttaggtacaaatgtcttatcagatacgtgatttgcaattattttctataattctgtgggttgtctttgcactttcttgatgatgcccgttgagacactgacgtttttacttttgaagttgctcagtttatctatattctttttttgttgttattgttcgtgtttttgatgtcgtacttaagaaactctggcctaatccaaagtcaaaagatttatccctgtgtttccttttaagagttttatagtttttcctttttgaaccattttgaggtgatttttgtatatggtatgaaatgaggttatttattaaataaattaaataatctaattattttgcatgtagatagcaagttgttccagcactgttagttgaaaatattattttcccccattgaattgtcttatcagaattcagttgatcataaacgtgagggttctttttatggattcttaattctatttcatagatatgtgtctaaaatacataaccatgccacagatcactgtcagatcagatcagatcagatcagttgctcagtcgtgtctgactctttgcgaccccatgaatcgcagcacgccaggcctccctgtccatcaccaactctcggagttcaccgagactcgcgtccatcgagtcagtgatgccatccagccgtctcatcctctgtcgtccccttctcctcctgcccccaatccctcccagcatcagagtcttttccagtgagtcaactctttgcatcaggtggtcaaagtactggagtttcagctttagcatcattccttccaaagaagtcccagggctgatctccttcagaatggactggttggatctccttgcagtccaagggactctcaggagtcttctccaacaccacagttcaaaagcatcaattctttggtgctcagccttcttcacagtccaactctcacatccatacatgaccacaggaaaaaccatagccttgactagacgaacctttgttggcaaagtaatgtctctgcttttgaatatgctgtctaggttggtcataactttccttccaaggagtaagcgtcttttaatttcgtggctgcagtcaccatctgtagtgattttggagcccagaaaaataaagtctgacactgtttccactgtttccccatctatttcccataaagtggtgggaccggatgccatgatcttcgttttctgaatgttgagctttaagccaaccttttcactctccactttcactttcatcaagaggcttttgagttcctcttcactttctgccataagggtggtgtcatctgcatatctgaggttattgatatttctcccggcaatcttgagtccagcttgtgtttcttccactccagcgtttctcatgatgtactatgcatataagttaaataaacagggtgacaatatacagccttaacgtactccttttcctatttggaaccagtctgttgttccatgtccagttctaactcttgcttcctgacctgcatacaaatttctcaagaggcagatcaggtgttctggtattcccatctctttcataattttccagtttattgtgatccacacagtcaaaggctttggcatagtcaataaagcagaaatagatgtttttctggaactctcttgctttttccatgatccagcagatgttggcattttgatctctggttcctctgccttttctaaaaccagattgaacatcaggaagttcatggttcacatattgctgaagcctggcttggagaattttgagcattactttactagcgtgtgagatgaatgcaattgtgcggtagtttgagcattctttagtattgccttctttgggattggaatgaaaactgaccttttccagtcctgtggaatgggtgaatttaactcagatgaccattatatctactactgcggcaggaatccctcagaagaaatggagtggccatcatggtcaacaaaagagtccgaaatgcagtacttggatgcaatctcaaaaatgacagaatgatctctgtttgtttccaaggcaaaccattcaatatcacagtaatccaagtctatgccccaaccagtaatgctgaagaggctgaagttgaacggttctatgaagacctataagaccttttagaactaacacccaaaaaagatgttcttttcattataggggactggaatgcaaaagtaggaagttaagaaacacctggagtaacaggcaaatttggccttggaatacggaatgaagcagggcaaagactgatagagttttgccaagaaaatgcactggtcatagcaaacaccctcttccaacaacacaaaagaagactctacacatggacatcaccagatggtcaacacggaaatcagattgattatattctttgcagccaaagatggagaagctctatacagtcagcaaaaacaagaccaggagctgactgtggctcagaccatgaattccttattgccaaattcagacttaaattgagaaagtagggaaaaccactagaccattcaggtatgacctaaatcaaatcccttatgattatacagtggaagtgagaaatagatttaagggcctagatctgatagacagagtgcctgatgaactatggactgaggttcatcacattgtacaggagacagggatcaagaccatccccatggaaaagaaatgcaaaaaagcaaaatggctgtctggggaggccttacaaatagctgtgaaaagaagagaagcgaaaagcaaaggagaaaaggaaagatataagcatctgaatgcagagttctaaagaatagcaagaagagataagaaagccttcttcagcgatcaatgcaaagaaatagaggaaaacaacagaatgggaaagactagggatctcttcaagaaaatcagagataccaaagggacatttcatgcaaagatgagctcgataaaggacagaaatggtatggacctaacagaagcagaagatgttaagaagagatggcaagaatacacagaagaactgtacaaaaaagatcttcacgacccagataatcacgatggtgtgatcactgacctagagccagacatcctggaatgtgaagtcaagtgggccttagaaagcatcactacgaacaaagctagtggaggtgatggaattccagttgaactgttccaaatcctgaaagatgatgctgtgaaagtgctgcactcaatatgccagcaaatttggaaaactcagcagtagctttgtagtaagttttgaagtcaggaaatacagtcttccaattttgttcttctctttcaagattttttggcagttcaagatcccttacaattccatttgaattttaggatcgactcactgatttctgcaaagaaataatggaattttgatgggattgtgttggatctgtggatgagtctggggagtattgccatttttttccaccatagattaattttccttttctagaatttcatattaatgaagctgtacagtatatactctttttgtgtaaggcctcttgcactcagcctagtattaatgagattcattcatgttctgttattactatttatgttcctttatattgttgagtagtgttctatttgatgaatatattgtacttatggttatttatctgttcttctgttcatggacacctggactgtttctaactattatgaagatttacatacaagttgtttttgaggaatttgttttcatttgtcttaagtaagtacctagaagcagaactacaggatcatggtgtaggtgtatttttagttttataagaaattactagacctattcccaaagtagtgcagttttacatcccccctaacaatgaatgagagttttagctgttatttatcctcactggcatatggttttgtgaatttctttaactttagccatcctggtaagtgtatagagatatgttattgtggttttcatttgcattttctttatgactaataatgttggacaatttttgtatgcttattggtcattggactgcaaggaggtccaaccagtccatcctaaaggagatcagtcctgggtgttcattggaaggactgatgttgaagctgaaactctgatattttggccacctgatgcgaagagctgactcatttgaaaacaccctgatgctgggaaagattgagggcaggaggagaaggggacaacaaggatgagatggttggatggcatcaccgactcaatggacatgggtttgggtgaactccaggagttggtgttggacagggaggcctgtcgtgctgcggttcatggggtcgcaaagagtcggacacgactgagcaactgaactgaactgaactgattggtcatttgtatatcttcctttgcaaactgctgttcaaatattttgcccattttagtaacagattgtttgtctttctgttagtgagttgtagatgaatttacctgctcctaatcactggagtattttgctgttttgtttttttttttctagagatgttacaattttagcatttatttgcaggtctatgacttatcatgaatttttctgtgtatggagtgaagtaagggtctaaatttgttattctctgtatggatgttctggagaaggcaatggcaccccactccagtactcttgcttggaaaatcccatggacggaggagcctggtaggctgcagtccatggggtcgctaagagtcggacatgactgagcaacttcactttcacgtttcactttcatgcattggagaaggaaatggcaacccactccagtgttcttgcctggagaatcccagggacgggggagcctggtgagctgccgtctatggggtcgcacagagtcggacacgactgaagtggcttagcatagcatagcacatggatgttcaggtaccagtggctgaagagacttgcctttcctcactggattgcattggctcctttgtcagaaatcacatgattgtaaagctgcgggtctgtttctggacttaactattttgctccattgatctattcgtctatccaaatgttagcaacactctgtcttgattattatagctttattgtatgttttgaggtcaagtagtgtcagtcctctgactttactctttttcaggattattttgattactctagattcttatgtttcctatacattttacaactggcttttcatttcccactagaagtctggtggaattgctgttaggattgtgttccatatgtagctcaatttggggagaattagtatcttaaaaatattgagtcttccagctggtaaatactgaaatgttttctgattcacttatcgtgctgttcccttttccctcgttttcatgttttgagtcccaagaggtgcggtggtgaggaagggccttacaatcagcttgcctgtgggatctttgacagattaacctgattggttaaatgggaataagaatgattgttacttcttcgagttttggggagcattatgtggaatatttaagaaaagttctcagaattgagggcttcataagtgttggatttgtttaggactgcccttcaggatctggcttagattgtacctggtccatgaagactgctcagagtcccaaccttgaagtatctcttttcttcactgtgctgttttcctctctgtgtagaatttagcaccttttccttgagtgtgtatcacttgccttatcgttataagctctttgactataaggtctgggttttagcttctttggggtccattaagtgctcattatagtgtgttagctctagtagacactaaaaaactatgaagaattttaagttttttttttttctgaatgtgatcacatctactatcttatattttcctcaaagaaatagttaaagcagagtttattctcaattacagatgaggaaactgaggtggagcccaagagaggctaaggtactaaccataaccatttagggacagagctaggaccagaatctagccctctggacatgaagagtaatactgtgatgttttattctattatataatttttatatattctgtattatattatacttgtgtatatatcatatttattatattgtatatatatatatttttattatgttatcatgcttgtgctcagtgctcagttgtgtccaactctttgcgaccccatggtagcctgctaggctcctctgtccatgagatctctcaggcaagaatcctagagtgggttgtcatttcctaccttaggacatcttccagacccagggatggaacccatatctcttacgtctcctgcatttacaggtagtttctttaccactgagccacctagaaagccctattatgttatatatgcatattatatacaatatgtatattacatatgcaatgtatgttacagtatttatatatatatatatatatatacgtatacagagaaggcaatggcacccgactccaatactcttgcctggaaaatcccatggatggaggagcctggtaggctgtagttcatggggttgctaagagttggacacgactgagcgacttcactttcacttttcactttcatgcactggagaaggaaatggcaacccactccagggctcttgcctggagaatcccagggatgggggatcctggtgggctgctgtctatgtggtcgcacagagtcggacacgactgaagtgacttagcagcagcacatatatatataaatttcttaaggtctgtgagatgctggaatccctttattcagggcaagccaacctcacaaatccttcatgaaaatgacatttacatttcatgactttgtgactaggtgaataatataatacaggtttaaatgaacttccccttttcgttttgaaactaggtgaaatataccatacgggtttaaatgagcaccttttgcaaaggtgctcagtgggataccgtctgtggtaagcacatttactgcctgctgactcttcagcaggatggacatttacaactacagaggttgtttgtaaggtgtcctcagctcttctctctgccccatgatctgcctggcttcacggtagaagagcccagcacagtgtgaggtcctgctgtccagccgtgactgacgtggatgatggagggacaagagttgcgtttgctcctgagcctgtgagtgggtgactttaaaaacatgtacttccttccacgagaaggacttgtgctctgaccgattcatgatgcatctagagttcacctttgattacttgctctgagctacccatgaaaaatactagcacttacgtgatgtagagtgtggggtaatacttctaggctttaatgtttgttcatgtgataagcattgatcctgcttccagagcacacctgtgactaggagggattgcttgctcccctgctagagatgttttctatatctctccctgcccctccggcatttttccatgctggagggtggtggacctggagagcaggctccgtgatattttactagagtctgattgtttgattgtagaatccatgcaagtacattttattcatcccagtataggctggggtgtcaaagaacttcgctgatcttagtgatactctgaaactcaatttctccaaagtctctgtccagattacctgcttccagtaagtgccttctgtgtatcaggccctgtgttgagtctttaggagcttaacatcctttaatcctgacaacagcctctgtcactcccaccgtatcaggtggagatgctgagacagagagagggcttctaactttgccagcggcagagccgggaccaaaacgcaactccggagcccgcatgcgtattacctcccctgacttaccaattacttccattctttccctcccaccacctctctccacctgtcacaccctcctttccatgaatcctagtaagatgacagctggatcaggagcagctgcacacatctttgtaactcagccatgtgtccttgtgttttcttcttttcccgatcctcttcttggaattctcctcttttcattcagcgctgcatccttcccccttgccttagctgttgccataaacctactgagatagttgctttttttttttttttaaagagaatttgtaaaattttaaaaaggtaaaggcgcatctaggtggtttctgcaattggaccccaaatctgcagataataaaggttagaactctagacatacagggattatggtattaaaaatatgggcatggctcagattaactcctgggggtgggaggcttggaaagaatgcatgcgaagtcgcttcactgatgtcctgctctgtgcgaccctatggactgtagcctgccaggctcctctgtccatggaattctccaggcaaggatactagagtgggttgccatgctctcctccagggcatcttcccaacccagtgactgaacccatatctcttacttcttatggattggcaagcaggttctttaccactagggccgcttgggaagccccaagaatgtgatatgtttgctcaatcatctatccatacatctaccaactgggctatcattgagacagtgtaacagtggtgtagagttagtctctgagaccagactgcttaggttgaatcccagatcacttcttccctgtgacttttgacaactttaattattaccttctctgagtctctgcttccttatctgcttatagttctaactacctcttatggtgattgataggattaaagcaaagtctgtcatacacttaaaatagcacctgatatatgagaagctttgaaaacacgttagttattgttactgttcagtcaccaagttgtgtccatctctttgtgaccccataatctgcagcatgccaaaaagggccagctatgctctgatattcttccctggatttggacacatcagtggacaaaacatccctggaatcgtagtcctttcagcttagtgagaaggagagagagagaattacaagtgtgagagaattgcaagtgtgtacctgtgcactaagaagacacgcaggtggggtttgagtgtatcacagagggtcctactgaaagatcctgattgtgttcactatacaactttagagcaggaaagaattgcggagactctctagtcaaaccttctttggaatctagaaagcttagatgacttgccccatttattcagagatttatggaagagccaggacagaacctgtatctcctgaaactgatgtcagactagtgaacatcacgtggccaaacctcctccttgaagacttgagccaatttgctgtaacatttactccaaaatcattctcatcaacaatgcttgatgctggggcagcttctctgaactcagactttaattgtttctggctcccgggagctcttatgacagtaatttgtgccaagtcatcagaataatgatgatgccattgtcttacatttacttaaaagttgtccatcctggtaatgatcacctggcaaagaaagattgtctagattattctactagacatttgtaaatatctagtttggatttaggagactgggtaaccaatcaaattaaaacccagatctcacagacctgcctgaggaattcacatatgataaaactaatcagttggctttgaaatggagatgaaagcatctttagggaagtatatgttggatgggggtggaagtggataaaaactgattcaaagaagcataacccttaaaagtttcagatgatttataatttatctgtaatgggaaaaatactttaccatcaagataagatcttggaatacatttgaaatcactagccatattatggatatgtttgtggatgtatgtgaattgtagggaatgaaattttttctgtgaatgtaatttagcgtgaatgagatgacaaataagctatatgccataaagcccatttgatataatgtgtgtaattttaaacttaaaagcatcatgtttaagaaccataagggacgttagacatcatctaatacaagtcactttgcagatggaggaacagaaccccagagaaggcaagtggcttggcccatagtgtgaactggaatccagatatttagattattgagtttggagatttgttttagaacgtaaggagtgatacccacacgcttacttttagtattttttttttaacccatagtggttttatggcattgttaatcatagctataattgatttcttttgcaagatgaaccaattcttcagccctttatacatgttggtgcttaataaaagtattttctgtgactcctaattaatgctgaaataaatttagagaagttagaacaatattgtttgtcagcatcttccttggtttgagctattaatgtggtttcttatgatttcccagctagctgatttaaaagaattcagagaaagtcatatatgctgtgcttttcaaagcaaagccctggttgctagagggatggagtttaatatagtttcatatcacagaggttttaataatgcagtctaaagtatttgtacatgaattcattaggcccagattagtcttattataattactcaaaaagtaattcaatttaagagaaaacattttaaatggatataaaataccctgttagctgggaagccatactgttctactttatcagctccataaagtactagcagatttgaacataactttcacagtatacagaatgcaatcataaaaagtattattcctaaatctatttttttatgttctttcagtagttaagagctttatccttctgtgctaggaatgagatatactcgggatagagtgtcataaaaatgttagggacttaactaactcacagcctgtgcaccgcctccccaactcagtgtcagctgatgggccacaagtccagagcctcagattgatgctggtcgcacattccagaggaagtggagcaggaagagactttggggcttttgcaagttctttcttactcaacaacagtgacaggcgaaggaggggtcttcatttctaacatggtaaaaggtgaggggagccaggagcaaggaaaacaatagaactttccctggcccacatccaaaatgggacagagaagaaaaaggagagcatgatgatatagatcatacaagttaaagaacaagttagtgtttggcacagagggcaggagatggggagtgaagccttgcagttgttccttaactaccaaggctcaccgggctccgggcggtgagaagctgcctgggtacccctggggaagcaggaggggcagggagatgaggtctgtggccacgtggctgcctgagctgtctgtgctgtctggcttcggagacacagaaaactaatcacagcccccgtgtggtgccatcgaggccaggcaggggctctggctggactccaagagagcacagacctgcactcggacaggaaggggttccacttaagtggtttgttgtgttcaaacttttactaatttatttgatgagtagcagatccctttccaggcaaattcttcattgaaccccactgtcctgagcagagtggttctcagttcagctgccacatgcagtcttaccttttttaaaccctggacctccactcatcacaccctagggcagggatgggtgagccccttccctggtggccttgagacataatggcacaggcactgccagaggggtgaagtggtgccaaaaatgaaggccaggctctggatttctggaacatctggtgctcattacaaccccgtaaaactggagtttggagataggatggcaatagatctctgatcttaaagattaaaaataattcattcataaaataatatttaataattccttagagtttcttaatggtatgtagctacggatgacaagaaggaattaaaacgatcatttaattttaatggattttcctcttatgatattttacagtcctgtacaagtcacaactggtgattattgtcaaataagagcccctggagacgggaatggctacccattccagtatttgtgcctagagaatcccatggacagaggagcctggtgggtatacagtccatagggtcgcaaggagtcagacatgactgagtacctaagcacattccccacatacaagtcacaaatgatgattattttcaaatagtaggcaatcaataaatgtctgttggctagtaatgctataagttatagaagtctatctaaatattagtcagtgtgcactttatcagagaaggcaatggcaccccactccagtactattgcctggaaaatcccatggatggaggagcctggtggactgcagtccatgaggtcgctaagagtcggacacgactaagtgactccactttcacttttgactttcatgtattggagaaggaaatggcaacctactccagtgttcttgcttggagaatcccagggatgggggagcctggtgggctgccttctatggggtcgcacagagtcggacacgactgaagtgacttagcagcagcagcagcagtgcactttatatgctttacctcatttaatctccaaagcaatttatgagccagcacaaaaattagctatttttattatctgcatgttgctgatgagaaaaacaaggcttcggaaggttaaataatttgcctgtgatcacacagcgagaaatggaactagttctgacttcaggacctgagttctttgctaccttgctaccctattctgttgcttattgttacaccaacattaaattcagttattgaatgtctaccataaaccagctccatgtcctaaaagtgctttacatatatgatctgggttatccttatgaattttgaaagaaattttgttatcgtcattttttacatatagaaactgagactcaaagaggttgaatagtggcccaagatggctaggatagtagactagcacgtatttgaacctggtattatgtgactttgaagtctatgagctgcatgctgattaaatgatctaacttgaccgtcatgatactcctatggtaggacagaagcttatccctatcagctgattttacaagtcagaacataggctacaggaagcttcactttgggtttccacctggtgaggggtgggacccagtcttaggtcatgggttcaggtggccagaatcatgtggagactgagccctggactgtatttgggggagaaggtgttaaccctgctccctgagaaagtcacctgtcatttttggacctagacttcttcagtttaaagctgaactattctggagtgcaggggaggctttggtgagccccacttgtcaaccttgaaaataaatttacagagatctgcagaggtatacagactgaattctagaaactcttcaatatctgtgcctagagcttgctggtttttgaaacttcgtgagggggcagctccgctgaccttgaaattgactttggttagtggtgaacttgaccccatccaaggaagtgatgttcattccaactgctccctacgtcccttcttttgcccctggttagttaggtcccagtagcatttatggggctttactgtaatgacatttgaaagagaatggtacttgaaagagaatgttctttctgtagttcaaaccacaggaggactcacgagacagctctttccccaggtgagacttcctaaatgggagttaagtacaggcattttcaagaaactaaactcccttcatacataatttaacctagaaaagaaagcaaaattgagagagtggtatgatcacattgttcgagggccagtaactacttcccccagtcttagagaagtcaggcacactttcactatttggcatttctttaatgctgtgggatgtgggctaaatgtgattctataataatagtgggggtttctctaaccatagttatgagctgagctatggattgaaatagtaaattattcctagcagactgaacgggcaataagactgccatctagttcccctcataaaacaaagcaatagataaataaaggagatccaactcacttaacctgcgtggctctgtgcaccgctaattattaacccaggcacaagagaagggttcacccagagtgtggttaaatcatttttaatagaggtaaacacgagccatgtggatgagtagcactcaagcaagtttttaatcagcaacgtttaggaatcacccagcctaattgccataacttattaataataataaataatctggtctttttatggctcctgtctctggaggactcagtgcattaaagatctctggggtgtgaatctctcagtagctatgggggtggtgggcaaatatccttgatgctcattcctaacagaagaaagtggacattgaggccaagaaaggcaaatctgagttcgttctttcttacttgcttcttcctacatctgaatcgccaatttatagatgccctttcatctgtctataggaccagaatggctctggtgggggtggggtcggagaacaggcttttctttgttgacgtgaatggttagggttgaggttttttcccttgggaagtgagtatgagcagagctttgtctgaaggagttcatttcctctgcacaagtatccctggtgtcctttgggttcaacctgactgtggctaggctgggtactggaaactcctaactccttaattgcatgatttccagttcctggcggagcagagaagatggcaccgagtcttattctcagtcattctctgctgcagcggatgggggatgaggaaaggtttctgtaaatcatcagacttaacctccccaacgcagtggcatgagcgttaaaatctctccaggtcggcttggttcaaaggttgtaagtgattgactcccaaataattcttagtggttggttagactggacggagatgactgccctcgaagtttgagaccatttaattcctataagtgactagttcttgtgggttggaggtggtggacacatgcaccagaatagaagatgacaaaaggtatttctgtggctgatgtcctctgaggtctgcagttcggcaggtgtaaaatggcacagaatctaccttcgtctcacttattttccattcttctgtccatgaatcatttgaacatgagcctggatggctgcttctgtctggaaaagtgaaattttaaagaggatacccaagcagttattattcagagcaaagagctaagtaatgctaagaatggaatgatttatgtcatagagtacaaaagacataaatttagagtgtaaattacaagataactcatgtgcaaaattgttgtaatatttatactagtgtttattaaaatttacatttgaataaaaaggtgaaaaatgtaaatgaagctgaaaagtggatttgaaaagaatctgaaatagcttatatgaaaatcaggagagttttctgggatagaagataggaatagtactctatgtaggtttgtctgttaaaccatgacatatacagtgagattggaatgaataaggtctctgattcctgaatttctgcctgaatttcctccctccctgcccactgatactttccagtaactcagatcagtactgaaagccatggagaggaggttgaagtttctggacggatgtcgaggctggatccgtatttgaaaggctaatctcatttcactgatgttttcaatgttggaaatttttttctgtattcattaaaggataatgataaatctctaatttgtactttgctactaatagggatgattttgtgcttcattaaggttaaatgaatctcatgtcgtcctggctcagcctttcacagacaccatgtgtgatcttgggagagatctcttccttctctgggactcagtttcttcatctataaaacaagggggttgtagcagatgagtgttgagagagaccagccccctgcccttccctcctgtgtgggaacaccctatgccaggcagttaggatccacaaaggccatcaagacgcatagctcttgttctcaggagcacatggtcttcataaataactaattatagtatgaactctaattgcaacatgcacaagttactctgggagcacagaggcggaaagagcgacttccttctctgcagggataggctctgagaaacttcacagtggccgtgacatttgaagggggaggaggagggcttctccaggtgtgcaggctggtcagagggtggatggttcagactgtgcattgcaggcggcagatgcacgtgactgtggtaatttagggagctgcacatggtccttctcaccgtctctggaccgtcagggcatgggggccgagtagctttgtatacactgctgtgtcagaacttgggccagtgatgaattactgaggttagagagtaatgtatagcatgtataaattaaagcagggtttaagttttcctgtaatggcaccagtctggcccatttacattccagttggctttcctgagtgtgagagaaggggtggtgatttagccaactccctgggagttgtaagcctatgttggtctcatctgtcctatagccactggcctaggtgtgccctggggagcatgtgtattgcatgtgagtgacaccccttatgagggcacatgtgccccagtgggggctttcctggtggctcagatggtaaagaatctacctgcaatgtaggagacccagatttgatccttgggtcaggaagatcccttggagaagggactggtaatccactccagtatccttgcctggagatttccatgggcggaggaacctggaatgaaaactgttctcactggttttctctccttgcagtctgtttaacccattaagtacatggttatccacaccatttgtggccccttggctctttgtgaccttgtgggagctgatgttttagctgaagactgacagcaaatacagatgttgatgatataattctcatttagtttttaagatgcaggtaaattttaaattcagcttgtaattgtagggcatgttcattgttaaatttaaaataatacagaaattcttatataatatttaaaatggtcccagatgcaattacctgaaggttaacatttctgtgaccatcatcacaggtctctagacatacacacccatacatgcacatacatgggctcaggatacatgttgcatgttactttctgatttctttgctcaaaaataataaaaatcttccccctttttaaatggctgaaccatagaatggatgtattgtttttgatttgattctctattgacaggcaaatagacagtctctgttgcttgtcctttatacacaatgctgtaatgaacatctgtgtagactttaggtatttcattagatgacatccttagcagtggtggtgttaggttaaggatgtatatttaacttttccatacatactgtcaaactatcttgtagaagttttgtacctatttataaccccatgttggctcagatggtaaagaatatacctgcagtgtgggagaccctggtttgaaccctgggttgggaagatgccctggaggaggacatgacaacccacttcagcgttcttgcctggagactctccatggagagaggagcctggcaggctacagtccctagggttgcaaagagtcggacatgactgagcatggcacaaccccatgatcagggcaggaactggcttacatctgcatgggggtgtctgtattgccaatgctatcctccgtctttgaattactggatgagaaagtaggaccagacaggaatattttgggggtgattgggagtctgtagaagcagggtgagcctgatgtcaaaaagtaccttgtagggacttccctggcagcccagtgttaagactgcaccttccaatgcaaggggtaaggtttcgatccctggtcaggaaactaagatcccacatgcctcgtgaccaaaaacccaaagcataacagaagcaatattgtaacagattaagtaagactttaaaaattgtccacatcaaaaaaaaaattcttgaaaaatgtacctgacttatgcattcatgttcaccaaaagatatggacaaaacatcacagtttgtaattgcccaacaattgatcttcagaaacaatccaaatatccatccacattaggatggagaaataaattatgataaaatcataaaatcacccagtagaatactacctggcagtgagaatgaatgacctactacatgccacagcatggatgacccccataatcattgtagagtaaagaaggcagacagaacaaattcgttttggattaatccatttatacaaagcctgttggctcaacaggtaaagaacctgcctgcaatgcaggagatatgggttcaatccctgggtcaggaagatcccctggaggaggaggccatggcaacccactctaatattcttgcctggaaaatctcatggagagaggagtctggtgggctgcggtcgcaaagagctgaccataactgaagcaaatgagcacagagcccagaagtatacctagtggttactactggagggcgttagccagtgcttgttaatttatgctctttgctaatatgtgtgttttcagatgccagacctagtgcttggcatatagtagcctctcaaatatttgttgaaaatgatgaataagtatttttttttcaacttgtcacagattttgacttcagattttatatgaagacctggtgttattagttgtgaattgtatgtggagaattcatacagattttgtgcttattatgatttaaatatggaactgtagtagattcacaggtctttcctctgtttatcctgtggtgtgcagttgtcacttgggttgtttataaaccagcccggcagcttagtagcaggcaagaagatggtttgtggaatcaggagattgggttcaatcctgagctcattgcttattcctctgtggtcttggatcagtttctcaaccatccagaagctcagtttccctatctatacagtaaggataatattgcaacttgcctcatagggttgtagtgaaaataataatattaaaatgaatagtaactggcaacttttgtatgtttaccgtgttctggacaccagtctaagactttaaaacacaggacacagtttgtttaatctttccaatgcaaatgtatttgtggtttaggaatctgtaagcgacagggttagtaagcatcgagccaggatttaaacccacgcagttcccttccagagctcaagcttttaaccgctgcctctgagtagaggaaatattgtactaagagtgtttaggacagtcagtgtgtgttagctgtgatgctcatcactgtcatttgcaacataattactaatttttttctcctttctctctgcctttttcctcctgcttttccttcctcctggcagatgttttccagcaaggtaagctcattgcctcctctctgcttttctattgcttgtgctcttttgggaaagagtatcacctgtgtgcagagggttatggggctttccatccttctgtggacgtgccacggggactttaggacagaggtaccgtgaggagcatggcccggggtggcctgaagtgccatgagccttccagagcctctgctatccagcctctgccccttcctctggaccttgcttgggggtagaacgaggactggacagttgggacagggacattgggcacgggttcatgccaggcacctgtccgagggcagggctgctgtgggcttgtgagacaggtggggagcagatgtctctggagggcatcaggtggaggcccctgtttctcagagctccttcatccaggccagcttctgcttcaggtctccacacgccacctccacctgggggcttctctgtcaagcaaaggtgtgggcaagaccatggctggctggtcactttgtgccctttagcccaggggaacctcacaacccctccaattgggccagtgattgagaggaacccggcaagggtatgcagtgtcttacacattttcccagtcaagtttatatgttctggggagctggagagggacagcgtaggctacgtgtccttctgcctcatttttaagtccccattcaggatacacagcttctgcactctgggagtctcgcgggcatggccttactgaacccactagatacacattcagggcaactgcctctttccgtttttcatcaccatctcaaactactggaagaaagtggacagaccggattgtttatatattcaagatgggaagcatgtgtattccatgcctgccacctcattctcgaatcccaggctgccttaggataaaagagaccagtgtaaaaaaaaaaaaaaaatccaggcatcccataagtagcttccaaatgattttatgggctggatattttctcgacgattattttaccccaaacatgcagcgtgtgatggagaaggtctgtctgctttctctgatcgaagtgcagttgtgggtaacaacgaagggacttgtgttgtcttttaagagtgcggagtcatagacctacttttataaataattcataaatctaaaccagcctgaagtaataagttccatttggctggagacgaagcgatgggcaaaattgagaaggtgccatgactgtgggtgtaaggcatgtgctgccgtcagggtcctggctacagtgcgttttcctccttgacacattgagggttcaatgacatgctctgggaggcagtttttatttttttctataaactagagagccaagctctccccaatgccaaactacatgtcttcttttgagacataaaatgcgtgagtttggaaaagtctcgctggagccggtgtgccgaggcccctggagaatggagagagaaaaggttggtatcagatctggcttttctacctaaaaccatgagcgttcagttattgtattcagtataaaaaaatggcttatcaggctgctcatgtcaagtttgcctcctttttaatcagtcatgccattccattgatgggatttgactgttggtaacaatgatatgcaaaacttataatcacctgcaaatctcggactattgagacttaatagcatcttccacagacagaagctgctagctacccctggccctgtgagatagtactaggattacagatggggagaactgaacttgcttttgtgaattatttacatggctctggccttgcaggactgtttcatacagagggacattctctgaaatgatggccaagaactcaccagctggtaccccgccttacctctgcttctggccattctgcctgtgaaatgaatgctcagtaaaatgtgacagtaaaaatgtgtcagtgacagtaacctcaggaaaaagacaaagcttctgtgtcttgcagacaatgtctgaaacgtgactgcacagcttcttcttggggcgggggatggggggtgggtgcacggagactctgctaccagattgtgccattaacttgaaacgcaggggccatgaagtgtgctagaaataaacagcagaaaactatgtggtttcaggtgaacaggagccacagggccatttctgccgcccgtctacacgaggccactgtgagtggctctgggctggccatgccatgggcatcaggcctggcttgctttggtttacccagggactgaagggcagctgggttgcggggtgctggtctgcaaaaatgtactcttggccgtgagtgagtgaggtctctttgaccagaggaatccttgcatgttggggctgctgtgcactagggctggcagaagagggaaacagacttgcaagttgacaacatcagatgttctcagaggaaacaagttttttcctctggatagcattatcttttctttcactacagatcaatcttgttatagcgggtgacagcgcagcagctactgccggcctttctctgctggtactgtggtgtgtgcacatgggagccgatttctaccctcagagccgtttctgagcttgtctcggttctttcatctgtaaatgccacttgctgtgacagttagaccacagggcccgaggacagtcagggaccagtgggtccgcagctgggttttccgtgtgtaggagttctggggaccatgggcccacatgttcaaggagccttaattgacccacattctctggcatccacagagctcatgagccattggatggctctcaggaattctgaagagccacagaattgctcttcataacatcaaatgcaattgaaaatgctgtacgtgcgcatgtgtctagaatcttgagttttagggcacttttatactgatcgtttaataatgaagtcaagactgcacacacacaccgcacagctcatttgaagttactgaagcacctttgaatgctcatccatgggctcaaccttgattctgaaatcgaacagaaactgagtggcttggaaatgagaataccaccactcagctgggcatttgaaagcgtcagcaggcaccatataaagatttcaccattgagttaattctctgctcagtgaactgaagtcctttatagactctgtgcctgaagaggcatagccttccctcctcctcacttcttacctggagctgggatcttgctattagagagggttttcttccagctcaagtaatttttgatacctttcaaacatagcgcacttgtttatcttgtaaccggtaactgtgtgtgaggtactttctatgaggataaaggagtgactgagacggcgaagtcccctgttctcatggaactcatggtcgagcttggggaacatttggtgccaggcatgcaaattaataaacaaggtaaatttcaagagggatacaagaagtaagggcttctctagtagctcagctggtaaagaatctgcttgcaacacaggatacccctttcgattcctgggttgggaagatcccctggagaagggataggctacccactccagtattcttgggcttgccctgatggctcacatagtaaagaatccgcctgcaatgcgggagacctggattcaatttctggattgggaagatcccctggaggaaggcatggcaacccactccagtattcttagctggagaatccccatggattctccatggtctgtagtccatgtgattgcaaagagttgaacatgactgagcgattaagcacagcacagcataagcagtgaagaaaatatcatggatggtacgctaggtgctcagagggccagggatgttacatggatgtggccagagagtgacaagggagaccagctattggaggatctggggaccagcaagaaaacttgcaagggaggggcctggtgggcaggaatgcccccatattccagaatcagaaagaggctggtgtgagagaggaagttgtatgaaatgagcagagagaggagggcaggcatggtctagggatcagaggaaggaagtccatggtggtagtggtgagttttaagtagaagagtcacatggtcttagtaacatttaaaagcaccacactgactgctgtgaaaatgcacgagaacagcatgtaagaagttaaaaccaggaccgaggctgagtgctgagcatgctcgagccgggctgagatggggccctgccctcaggccctagttgctcccttgaggaagtgtggtgggtagtgggaacttccacccagccagctcacctctgctgagcaccatattttgtgcctgtgtgaagtcttgtgccatgacttaaggctcgcaaattatactcctcaaagacaccaataaaatattcttctatgtgtcttcagattgtaaaaagccactttgctccagtgcatatgtatgtgttcatcatctgtattacatatacattattaatgtatgcgcttccctggtggctcagatggtagggaatccgcctgcaatgcaggagacctgggttcaatccctgggttgggaagatcccctggaggagggcatggcagtctactccagtattcttgcctggagaatccccctggatagaggaacctggcaggttaaagtcgatgaggttgcaaagagctgaacacgactgagcaactaagcacagcacattactgtgtgtgtataaatatgcctagtagacatcagtttttcccccagtcgggctgttgctttgttgaggaggttgggccaatagctttgcccagcagcctcggttttcttatctgtaaaatgagagttggggagatccttgacccaatgtggcctcctgctgacgctctgttttaagaagttttaagtgaacttgagttaataagagtacaaaaatgtcccatgctgcagattttgagtattgcccagagaacatgggtggaaacaggttgagaatgaggtggctgagaattaaggtgaaaagaagcacggttgcagaagtgtaacgtactctcaaggctttttggaaacatcagttcttgcctatgtgtgttaataatggaattcacactgggtggcattgcttttactaatctgaaaagaacaataaatcatagtttgctttctggcagcccagcccagctcgttaattgttgaggtgggtttgtgatgacagtgaccactgccatttataatgtgcttgacttaagctcttttgcatttttcaaatgtgttggtctaactgctggagttggatcttaggtttttcccaaccacttcagaaaacccatctgttataaaactctgaccctttagccatgaattatggtccccctggtaacattatacattatgaaatattgttttctttatgaatgtgggatttgggtgaataaattaaatctattgttaccacctccagggaaaaactccaagttgtcatatgatgctaactagtaaatgagaaaaatgcgtaaacattcttatggtggaaagtggctggttccctggtcacacttttctatgctaatatttatgttgtatatatatcatccccctttctgttcagactcagtagaatattccgagtcatgtaggaaatgcattcatctttccagcacaaactgaacattaccatttaatttcacgtagattcactttgtgtgagtgtgttgtgtaggaaaacgagaggcatatttaaataatcacattctctgagatgcttcataacattctgcgtgggctctgcaaggtgatctgttccctgccagctggcctcgagtaatgcttttccttgtagcagattctgtgtcctcactccaatcctgaagtatgcatttcatttgaggtctcagagtctccctcccagttacttgagtttcacaattgccagatcaatgaaagggggtcagttttattgttgagatggttgctaataacaagaggaatttttattttgcaaattttattttattgggatgataataatgatgatgatgggaccatcagtatttgtccacgtcctgtattccaggcattgggacaattgctttctacacattttctcgttgaatctgtccccgtattttttgaagtaggttttataaccaatagtcccacttcacaggggagaaaactgaagcacagagtagaattcatctagtgatgtttatataagtaatgagtagtagaggcaggattttattttatattttgtgggttcgaatcccaccagggtcaattttattttttcctttcctctttttctgactttttcttaaaggctcccacccttctccccctacactatctcagtgttttccagctggtttcagttccaacctcagcctctgctctgctctcttctttacttacctgtctgccctggggggcttgggccgaggagagcaagactgggagtgggagacaaaaggggagggaaagtgggtgtgaatgctgagataaaggcagggccctggctctgggaaggagaagacaatggcaacccactccagtactcttgcctggaaaatcccatggacggaggagcctggtgggctgcggtctatggggttgcacagagtcagacacgactgagcgacttcactttcccttttcactttcatgcattggagaaggaaatggcgacccactccagtgttcttgcctggagagtcccagggacgggggagcctggtgggctgccatctatggggttgcatagagtcagacatgactgaagtgacttagcagcagcagttggctccgggatacatggggatttaataaccactttatactatgcactagggactcaggaaaggactgggactggactgggggcagagggtacaatcttagacacaagagactgggcacagggagaagggacagggaccagggaaatatatatttatttattttggttgaaagaccttattctacagtaaagttttagattttctgaaaaattgtgaagctagagagtcaccatataccctgcatcccatttccccattactaacatcttacattaacgtggttcatttgttacaactaatgacccaatattgatccattattattagctaaactccatggcttattcagattcccttgcatgagtgcatgtgtgcgtgatcattcgctcagttgtgtctgattctttgcaatcccatagactgtagccttccaggctcctctttacatataattttttcaggcaagaatactggagtgggttgcatttcctcctccaggggatcttaccaagccaggggttgaacctgcgtctccctcgtctcctgcagtggcaggcggattctttaccactgcgccacctgggaagcccattcttattcccgtagtttttacctaagttccatctcttttcggggatcctatccaagacatcacattacttttagtatccatgtcttcttagagtcctcttggcagtgacggtttctaagacttcccttgttttcgatgaccttgacagtgttgaggagtcctggtgttttgagaagtctctgttgtagatgtccctctctgggaatttgaatgatgtttttctcatgattagacagaggttatgggttttggggaagatcacagagataaagctccatttgcctgacttcatatcaaaggcatttcctgtcatcatgatctatgactcttgatgttgatcttgaacacctggctgacgtagtgtttgtcaagtttttccactgcaaaattactttctgactcctttccacactgtactctttggaaggaaatctatgcatagcctagtcttagcgaaagtgcggagctatgttccattgttccatcttattatacagaggctagctactcaagttatttttctacaagggagatttggttcctatgcttcatttattactttattcaatcatatatttagatcaggatggacttatgattatttattctattttgttgtggtcatttagtcgctaagttgttcctgacttttcgcaagtccatgggctgtagcctgccagactcctctgtccgtggggtttcccaggcaaaaataccagagtgggttgccatttccttctccaggggatcttcttgacccagggatctgactcacaactcatgctctggcaaaggattctctatcttacgtgatagttgctcagttgtgtctgattctttgcgaccccatggactgtagctcaccaggctcctcacctcatagaattttactctaaaccttgggttacaatgtaatacacatttaatttattttcttggcttgtccattggaagctctttcagcagactccttgtgtccctttgacatatcactgtcaattattttttgaggccttctgcaatactttctgatactataagatgctccatgctcatcttgtgtatttcccatctctgtccttgactcaggcatttcctaaagaagccctgcttccttttattggagaatgatgttagaaaccaagatctgggtgctgggtatgcttattgttattggggtgtcatttcttttaggccgtctcagttaacagagcaaaaaacacatgtgtgtatattctaacccatgtatatatgcctgtctgtttctatgtacaaccagttgtatctatattaaatgcgagttcttactggtgtgtctagctctcatcattaccacatggatcattctaggcttctccccttgcttatctgcacattcctactcccaaggttaagtcagggttttcaactcacgaagtctgacttaggttttacaacacactgtcatgattcccactgaaggactttcttctactcctggtgaattgcagtcgcagatccttgaccttccctaggctcaacaggaccacccagatgcagaggggtgagtttattagatacctatctatgtcagttccggctgttgtaacaaaatgccataaactggggagtggggtggggtgtcgaatagcaagcatttatttctcatggttctggagcctgaaagtatgagatcaagttgctggtagattccatgtctgctgcgggctagtttcctgatctggagaaagctgtattttctctgtgtccccaggaagcagaagggattgagctctttggtccatgctgatgtctccaatgagtgtatgtgtgtgtgagaagcaagcttgcattttcacagtctcctctaatgtctctattaggatcatgcttacaatccctgcaatgacattttatcttgaaatggttgtgagttcagctcccctcccagttcctctcctagatatgtccctgctcctgctcactttgtaaaactcaggataactctctgctgggttaccctgactcactcgtcccaacaggtggggaatttgccttctactctgtggagtttcgtatccccccctcctgcctccttcagcacttctctcaactcatgttgttgtaatgatctgtttgagcaagtcttcccaccccagattaagcccctggacagaaggaactgtgtcttttaatttgaattcccagcaccaagtgcagtgcttagcacatagtgggtgctcaatagatgtttgatgaataacagtggactaaaacgacaaagctcagaggaactgcattttgtgtgtatggtttatgtgtgtgcacatgcatgaacgtgcacacacatgtacacacacttccttttatattcgacaatttaagtgcttagtaaggttgaagcagatgctgtcactgcatcagttggatgttgacagcaaagccaaatcttttcttatccatgagtccaaaagtcttttttccattctgtttccttcaccagtgttttgtttttgttttttttttgaggggacaggttttggacccagaatgaatcagtaggtattacgtactgtggtctgtgtgggtatgaacagaaacatgaggtatatagcagacacatttctaccttccaagagttgacactttattggaaactcaaagccagtctgtctaggggccaatgggcactaccctttctcagtgctggaggaagtcatagagggtgggggttcttccgctgaaagagcggggctggagatggtttaaagaaggcggaagacctgagtggacaggaccagggcagacctgtaggtgggcagatggaagacagtctgaatggaggtggaggcaaagacaagtttggtgttgtgcttgtgggatgttgagagtgcagtaaactcaccaggctctaggttaggactgggtgggaccagtgatggaggcttcacagactgtcaagggccaaaagcctctctgctttcttgtgccccaaaatctaccagtgtgattgaaaagattttgtctaccaagtgcatagtattgttgttgagttactgcacttccttattcattttattatgccttttgctgtttaaggttgaccaatcggtgtttctaatctttacagtgaattagttatgcccttgagttgatggaagtttaatccataaaaaaactagcttgatattttagatagcttgaacaatagttttcttggttatttttaaatctaggcagcaaaatttagatttgttctaggaaccacaagaaagccattgtagctttttgagcagagaaatagcatgtactgtgtacacgatgaaagtgctatgtgttgggtggcagaggataaagacggcctctgcttacttgtgtacaatgtgccaggcacatactttaacgcctttcttccccagaatgaccctaagagtttcacgtactcatttatcttcattttttatccccatttttcatatgctgaaatagattcagaaagctcacgtagctttactttgtagagggtctcccatctagtaaagaacagggcctgaattcagaaccaggcaattggacttagttcaaattcttaccaccgtatcacaaagtcattttggagggaccagattaatggttaatgtaggtgttagctatatgggtgttcattgcaaattatctcacctctgctatatgtttgaagacttcataataaaatatggagagaacaaagaaaccttagttgggttcttcataggaatagagatcacatggcctccacagtcatggataacgagaaggtcagtcagaggttgggcctccctagaggagtgggccggtcctggggtccagctgtgggactccaggtgaggtggggacccaggtgagagacaggtggagagcaggctgagtgctctgctagtttcctgggtgctcttgtaatgaataaccaaaaacttaagccctcaaggcttaaaacaacagaattttattctctcacagtttcagaagtcaagggtctgaagtcaagatgttggcaggaccctgctctctctgaaggttctaggggagggtccttccctgcctcttaaccaggctgcggaggctcccagaaactcttggttttcctcagcttgtagctgcatctttccaatctcttctttcaccatcatacagcctcttctcataagtacaacagtcactggatttaggggccactctaatccaatgtgaccatgtcataacttgatgacatctacagagaccttatttccaagcaagatcacgtattgaggttcagagtggacatgaacttttgagttggggacaccactcaaccatgacaagtgaccagccaatctgggaggtccagtataaggagaggggattatgggaggacaaggtggctgaaaagaaagtaaagggttaaaagatagggagggaaccctgagcagagtggagaagaaaattgaattaaatttggtaagagaaagaaacaagggctccagagagaggaaaacaaggcaagtaggttttcgaggaaagggctgtgtctcaaccatgggcctctctgtttatttatctcctgaatatcttaaagaattaaaatgtaaatctcggaaattaaatctacgggcacaatgtcaacagagagggcttttgcagagcagacaaagctgttctttttccttgtgaaacagccatggtgggaaaccacgtctgtatgcacaagatttatttgagatggattaaggttcgtgatgagcgaatctctgaaggattggaggtgcagttctcataagcctgcaaaagttcagctccttatctgaggcttatccaaacctgacgaaaccttttaggccagcatggaggttagggacctccagcagctgaaagctaatatcctgcgtcaggaaaccacctgcattgtgaggggcagtggggagagaggcgtataaattaatgtgctcttggaagggaggccctctgtggcttggggcttcctgagcgtggaccacccactctctttggtggggggattacaagctcagtcctttgagccctttggaggagggaggtaatacctcctggttcttgagatctttcaccacattcagttctcatgtcagattcagttctcggtcacctccttaggcatccttttgggctcttggaccccagaacattcttcctgtagcaggctgctctttgttaggcaatcacctcttcagagacgccttccctggccatctttgttgaagatatttccccagacacgttcctccattgcctcatctatttccttagagcactgaacaagatgggtattggggtaatagtgtgccccagggaacttcctggtggtccagtgattaagacatcgcacttccaagacaggtggtgcctgttcaattcctggttagggaactaagattccgcatgccacaaaggacagcaaaatctttttcttaaaatttaagatataaagggtacaattaaatggtttaaaaataactagtatgccccccaaattcatgctccctgaaacctcacaatgtgaccttatttggaaatagtgtgagtgaaggatcttgagatgaaatcgtcctggatttagagggggtcctataatgactagctattcttgtaagaagaagagaggccccagagaactacagagagaaggtcacatgaagatgcaggcagcaattggggtgatgctgccacaagccgtggaccacctggagccaccagaacccggaagagacaaagaaggattctctgctagaaactttagagggcatgcggctctactgatgccttcattttaaccttctgtcctccagaactgcaagaaaatacatttttgttatcttcattcaccaagtttgtggtcatttgttgtaacagtcctaagaaactaacatagcacccaatactgtctgaaatcatttgttcatttttttgtttgcttatggttttcctgtctctccagctggaatataaactactcaggtaccagggccttgctgtgttgttcatccctacatcccagtgtttagaatagcgctgagcagagtgtaggtgctcaataagtatttgtcacattaatgaaccctgtgggataagcgttgtcttgatctctgggtgataaaaatgtcacccagagaggtttattcactcacccaaagtcacacatttggaagagtcgggattcaaactcaagccttctggtcctcaggttgaggtttagtctactacatcacaaaagctttattgaaattaaaaatgttatccttagaatctttatgacttatttgcaatgcatctcccccaaatagtatagttttataccttaccaagccagagctggaacgttccctgggaggtttggggtcttagcctgtcctggagcagaaggaagtcctcacaggcacagggacaggcagcctccatgcacaccccaccggagcccaaggctggactcccctggggcctgaccttccatgagtgagctggatgctgttcacacctgggccagatgagtatgccgctatagacagaccgggaaggcacagcactccggtggattgtaactctgatggtcacccctcctctcctctccccgcaaaaagaaaacagaaaacttggatttgacttttttttctccttttcactcaaacacatctctttcacccagtagggcctgctgtttgtcttagaattgatcatcctgtggatttgactcctgatgaatctcttggtgcctgatcttaaccctcacctctagttctcaaggccagcaggctcacgtcactcgtcattttagttcctgagggtctgcttgtgcctgcatcttttctagcccaccctagtgccatttacctgcttctctctgtctctctctttctctcacacatacacatgattttaactttttttttggtaaaaatgtacatactttgatttttttattgaaggagatatgctttgatgcacccgcagaccttctctagcctctctgtactgtaggactatgtgacttcatatttagtatctacaaacaaattttccagtttttacagtttttagccaaagggagttacattgtaggacttttgtataaagcttgtctttccccttattataactttctctctctctcttttttttttttttttttggtaaataaaacgtgattcttaagaaaaagtagtatagaatataggttttctaatatcctttcctagtgcatgtctggttatatgaggatgactggattttggaaataagttcaaggtcactcaaagttcatatcgctaactgaagtgagccagtcatcagctgtggtattgccagtctgggtctgaaatagaatgtagtgttaagggtagcagaatggctttcctgtctcctccactagactacaggcttcttgttggggggacccatcttagtcatccttttatttctaggcctgctacacagtgggtatttaatgggagtttgaatgaatggatgggtggatggatgactaatagctttatggtgagccttattctgaaaaataaccttgtatagaatttagaaaaatgattatatgtgtggccactctaaaggtacatattagcattctgttctgttcaaggaggcctctggcggtatggtcatggcttatatgtttgagtggataagtgttcgtcgcacagtcgttcccgactctttgcgaccccatggactgcagcccaccaggctcctctgtcaatgagattttccaggcaaggatactggagtgggttgccatttccttaaattcttgtcttatgttttctttggggaagggggtgatgaagtataaaaggatagctttattgctttgctaggtaaagggagacacaccaggcttctgcttcaaaaaactatgtgtctcaacctcagagaacttgatgagggtttttataatgatggatcaaaggtgtggttcttatgttttcttatagtcactaaagtgtaattgctctaaaatcttaaaaatgtatagagtgaaaagtagaaaattcatgtcatagtacactggcattgaggaggacttggggacttttggggggatcgtacctaactactgtgttatcagggaagttagcttcaatgcttgaattttctcatctatgagatgaagggattagacaacatcttgaagactaaccctgtgtagtctattattctatgaaatgatgacttccaaccacttctgctttgatactgtaagctgtttataccttgtctctctccccaaccccacccccagtttgaatctttccttcttaactagcttttaggcctctcctccatcatggcatccccaggggcatggtgagctgtgtatgtgtatttcatctcactttgaactaagtgttaatcaccctgagactgccttggttttttcaacagctcatcaggactttatctcttctgtgaacctgcttcttgtttgaaatagtggtattcaggtttccaaaaagtcaatccgaattttgggggcagcttttgaagccttactcaaaaggtttctctgtttctggtggggattcccagtgtggagcagcccggctggcagcacaatgctccttcagaaactaatgcctgtctctttctccattcatcccctattcttttagattttaaagatagtatggcagacagaatattggcccaccaaggatctgtgcctcgaaatccttgaaacgtgtgaactacgttatgttgcatggaaaaaaagactttgcttagggcacccatgtatgcagggatgaggggtgctcaaacattatcaaatatggtttagaaaagcctataagacgcttcttaaaaaagaatacaagatatatatggaaaaggttgtttgatgcatagtttgggataactagaagaaccctaaaggaaccctcttccttggtgaggcttagggttcactgaacagagtttgcaagtttgagtctgagctgaattgctggaaactaagtagggaatctgagagccccactgatggtcccggttaccctcccctgaaacagcacttgcctgcctcctacaagaatgctgagatactgtttaaaggctgtcaatttgaagggcacaaaattagcagtccatttggaaggctgtgacactcagtctgacagtgaaagtataacaaaaaaggtagaactaaatcagtaggtcaacttaaagtagggaagaagttctccccaaagacactaaggaaggctcagcgggggatttttttttctaaacttggtcctgaaggaaatgaaagtattattttagtgtaggggggctcataaactgtggcctgctagctggttatttttatgaataaaactatgttggaacacagccacaccccctcatttgaatgttgtttatggctgctttcgtactgtagtggtggttgaacggtttccacaaagcctatgtgatctacagactgaaaacatttagtatttgccaacccctgtcctcctttcctccgcagcaggtgtttggtcagcagacaaaatgaatcagcggtttcagagatgattctcagtctgttgagggaattaacatgaagatgttgatggactgatgaaatgagcttctcaatgtctcagaagggctcctaaaagttggagtatcttcttaattcagtcatgtagcaaaaaattattgatgctcctctatgtgtgaatcagtaggctaagggttggttataaaaaagtttctgagtgtttttgttttggttctaattgttaagcattggtaatccatggatatagggtaattctctgtcaaccagaatacttcattattcagaaaactggtccttgatccagctgaatattgtgcacttagttagatatcgacaagagctatactagaaattttaattaacctttcaaactccctttcgatttcttcaagataatttagagagcatggagagtctactgggttcacccaaagtaagaagtagtttaaaggtgacccagaaaggtgagctgggtcaggaatgattcattggagatggctttgttgtcttgactttctggtgggaaagaatttctgtggttatttttagattttctgttctcatttatcagtcaactttggcaacattgatttacctaacagacccaatttgtaaatagtgaaataagtccacaggaatatttagaaactaatgtctgcatatacagcaggttgagagacggcaaattattagtaactcgttcaccatcctaaagcttctcatttaagagtttataatgagcttcctttttatagactgttgactggttgactttggcacagggctggcacaggtaaatggagggagggagtgagtgctacctggaagcaagttcaaatgtaagaggacccccactggacttggctttgagactgtctgcagcctctttgcctgtcatctcaggtagcagaatacagaatttttccaacaaagacaggatttgtgtctcttaggagaccagtccttctctgctggctacatcagaaaatacaagacagatgtgattgatggtgagggattggtgacatttccatgaacttgggacttaatgaagccctttatctcttatccccaacgtagttttttttttttcacttcatctccccagaatgtatttggttttaatttcctttgaatggtaaagaataagttaatagatttttaagaatcaactcaaaaaaccccaacgtcttaaaaatcttttgtgtttcctgcccctccccagactttaatagcccctggagtgggtgtggaggtggcaggagtcttttgtactttctaaacctgctgtttgcagaagtggctgttatgactgccacaacataattgctcttaaaatgcctcaataatgagaaaaaaaaatctatttttatttcataatcttgatgctccttggcttccattttttcaggccacttttgtaacttccgaaaattagcctgacaaatatggtttcccagcttgatcagagagcagctctttgtcatgggggttggctgtgtgagtggaggggagctggggagagatgagggtggaggggtgagctgtgagaagggcggagggacttgattcctaggtggcagctgtttctgccttcacttgaccactttaaaacataaaggcagaaagtgtaccttctcacactcccctgggactgcctctgtttctccaggcttggttgagaaagaaaggaaacatttcaaattctctgactgttcttggtgcccagaactggttgttgggacggccagtttggatcttgagcactgtggagggcccagccaccttcatattgagttttccgtgtttttataagccctgcttctaagaggatgtgcttgggctgaggctggatggacagaaacttatcagctttgagagagaggttttctcctggggtgaaggtgcgctaggcaggttcttctgattctcagattcttcagtttgactcttcgtggattcagcctccagacaaagccagctctgtatttgccttggactgttcactcacccctcactagacccttggtgttgatctgacttgggatgccctggaagcagggactccatctactaacttccctggtggctcagctggtaaagaacccgcctgccagtgcaggaaactcaagagacctgagttggatccctgggtcatgaagatcttctggaaagggaaatgcaacccattccaggattcttgcctggaaagttctatggacagaggagcatggcaagctacagttcatggggtcccaaagagtcggacacaactgagcgcatatgcacaatccaccatcaaagatggaggtgctggggacaggtaattgaatggtggttcctgggcataacagcccctaacaactaggtgcaaacagagacctacctcagctcttttccttgatgagaggcctcaaggcacccagcctgcaaaccccaactcccagtctctggtcggaatgcggatgtatttgtttgctttaagatagacattcttcacccagagggacaaagaattgcttgggggtggggtggggcattcaaaacaagttcattttatctcagtacaggcaagaagccccaaaccaccaagtaatgaaattattcctttactcacagggacgaacaaaagaaaggattcatttgtgctctaccagacaattcctaccctcctgttggctgcaaactaacttggatttgttcatgttatcctgatcaccgaaatatcatcaggcctttagttttcttttaaatgctttagctcatttcaaaatgtttttaggtacagtccacctcccaaccctgtcccggaattgtagaggatcaattccaggacctcccatggataccaaaatttgaggatgctcaagcctcttatctaacatgatatagaatttgcatattacctacacccatccttctatatattttaaattatctctagaaatctctagattatttataatacctaatacagggtaaggcttccttggaggctcagtggtaaagaatttgcctgccgttgcagaagacacaggttctatccctgggtcaggaagatcccctggaacaggaaatggcaacccactccagtattcttgtctgggaaatcccatggacagaggagcctggtggtctatagtccatggggtcacaaattgctggacaagacttggcaactcaacaacaacaacaatactaggtaagtagttgtaagtacatgtaaatgctatgtctcactttatggaacttttgtagaattttttttcttttttctgaatacttttgatccgtgattggttgattccacaatgtggaacctgtggctccagagggccaactgtatttggaggagacagcctagtaaaaaccttgtcatctttccctggcaatgtgtgagttgtcccttcttgcagggtgaaaggtgtcattaaaattaagtcaacttgcaggatgctgtgtggtttagaatgtcagtttttcatggtgtggctgctgaaaagtttattgccttgtgccaaaaaaagtcattgtgatttcatttttgaaagttgaatttaaaactcagaatacactttctttataattatgcttcttatttggcaacatatggtgaaaatttatacggcctatcaatgtaaaagaacagagccgaggctctaggcttttatggtgattacatctacctgtatgggggctttatgtgcttacaatttgaatcatttatagatgcacactcaacagagttctagcataactagtactgtgagttttttttgctgactaaatgataggcaaagcaattttccacaaatgtctcccagtaggctgtcatcatagaagccttgtaatattgtttttaaccttcgcactggcagctatccagaaattattgcattgataccaaactgccttgcaccaggcagttgcccttttctccatgggaacccctgaacgctcaagtaggtgcctgctccccctctcgagtccccttgttttttatttcagagagatcttttacctgttcttgttcagctgtaggtagccgggcacacggaagtttaaaaatatggcaaaacaattctctggattgtgttattatgcctttaatgcattcaattaacattcagggctttgtcaccaaataaatgacgcacatcggtggcacaaagggaatgaagggaaaggctttgtgggttttttttttccccccttagacagagtcagatcagacttccttttttcttccttctctcccagctctttgctttccgctttctttcagaacctctcgtaaatggcttttgggaattggcagatcctgattctgtgtttgggggaaggatttttgagacttctttggaaggactgaatgagacatacattctgcagttttcctgcataggaggtggagcagagggtgggatccaggtggcagtcttcaggtctctgcaggaaaggagcccccagtgggaggaaatgatagctccccaccccaacttcctgtctgggagccctggctccagtggacctgacctcctgggagcccactgctcttgaacctcagtaagtggggtgcagtgctcagctcttctgtctaggttgagagaagtacaatctggggatgattcggttctagaagtttgatttcaaacacccgtgaagttttgggtacacccactgtgtgtcgtggtgggtgctgggaacctcacagagaataaacgattctgatttattcgatgggagccagctgttgtgtttgcatgctgtatcagctctgggcctgacttttgactgctcccctcagctgcatcccaattataaaatgtgagccctccccagctctggtttctctgcctgccttgcctgggggctctcatcaggtttgaaatgcctccatggtgggtatttgtctccttctagaatggcaccccactccagtactcttgcctggaaaatcccatggatggaggagcctggtgggctgcagtccatgggatcgctaagagttggacaagactcagcgacttcactttcacttttcactttcctgcattggagaaggcaatggcaacccactccagtgttcttgcctggagaatcccagggacgggggagcctcatggtctgccgtctatggggtcgcacagagttggacacaactgaggcgacttagcagcagcagcagaaagcctatgcccaccttcaccttcttcaggaaaagagttctttcaaaaaaaaccaaccccccccaaaaaaaacccctctagcctaatacatttgccttgaccgtctaaaatgatgagtgctataagtggagaaatggataactattatatatccctggttttcaaagaaaattctgtctttggcttggaaagaaagcattgtactggataaaattggaactgtaaactgtaatagaaaatcaaactcaaagcgttgaatataattggggaagtcaatggttttcaatctactttctttatttctcttgcctaagagatgtttcactcaagaagtatccatgaacagctgtatgactgaatttcagaacctgtttatgtactaattctgctgcacctgatacttgaagggaataaaacttaagatggttaatgtatctagttgcagatgtaaatgttgccccaaagagtaaaacagactcggggtatatccggagtaggggtgggagtgtgtggtggcaggacagagggacgatgcattccgaggcagagcggaaggtgaatgtgtagatgaacacagcagagagctcagtggagctgagggggtgctagtccttcggtaacaagatatgttggtcaagaatggtccttggaggaaatgtttcttgagctgggacttggaggaacagaagtattgtgtgccggttgccccccgagaccctccgcctcaagcacttgccctctttggggggaaggacgtttattccacctttaaatcagggaagaggcagaaccacatctctccctgcaggtggattcattaactggcccagggagcatgctttggtgcattaaaaatgcccaattgcctgcccatctatcttttccttataattctagtaatatacatgttacttgttcaatcatgtccaattctgtgtgaccccttggactgtagcccatcaggctcctctgtccatggggattctccagggaagaatactggagtgggtggccatgccctccttcaggggatctccccgacccagggatggaacccacgtctctcgcattgcaggtggattctttgctgtgtgagccaccagggaagccgtatttaatgcctctgaactgtaccacataagtggttacaatggtcaattttatggtaggtatgcctaacgttttaaccatttttgtgtgtacaattcagaggagttaaatacattcacactgctgtgtagccatcgccactgggcatctctagcactttttcacattcccagattgaaaccctgtaccccttatacaatagctcctctttctcccctaccctcgcccctgagagcctgccttctgctttctgtctccgtgaatttgacaattctagacacctcatataagagaagtcataccacatttgtcatttcgtgcctggcttacttcattgttgtctacttttttggagactggtttatcataaccaagagtctatgtaacaggtatttgttcttctcaacctcggctcctatgggcatattttcatgtgactagatccctccctctgcagattttttggcttaagtggtgttgcctcaaaaggggccccacaccccaaccgtcctacgaggttctgtttccccatcttggtctctgaattctccttgttgccctgtgctgtgtgctagggcttcccaggtggtgctagtggtaaagaacccgcccaccaatgcaggagacataagaaatgcgggttcgatccctgggtcaggaagatcccctgaaggagggcatggcaacccactccagtattcttgcctggagaatctcatggacagagaagcctggtgggctacagtccatagggttgcaaagagcatcctatctttgttttttttaattaatttatttattttaattggaggctaattactttacaatattgtggtggtctttgccatacattgacatgaatgagccataggtgtacatgtgtcccccatcctgaaccccctcccacctctcgccttatcccatccctctgggttgtcccagtgcactgaagagcatcctatcttaagatcccttccccctcttggcccctgtattctgcttattgctctaatttttcctgtctgcctgaattatgtataggtttatggtttcctcctcattgtctgatggctccaccagcttacaagatctgagagggctggggcgtctaggcatctagatgtctatctggagcatgacctagtgctcacttatcacatgataagtgctcagtaaacattgtagtgtagtgtagtggacggagggtgaaggaaggcgtctgggagggtgaaggaaggcgtctggctctagccttggctgtgaaccctcatgaggtcatacatgtgtctgtgttttgagcacttcagagataaatgctaactcttctcattagagtctgcccaacctttacaaaggagtttctagcacaagcacttggatttcagactgctccttttcctagtgcaataagattgtgaacggctttgctctagcccttccatgggtatgtactcggagaaggttaatacccacctaaataaaacacaattaaaagctaaaacatttcttctacagaaaagtcaatccccaccttcctgtgcctgataaaatgataaaatgccttatggctgagctgcttcactggattattcaagccgctaactcttagattattatgctaattagactttattaatattaaaaataagatagcagttttcaggcctggagaattcgaatataactctttgaagaaggctaggaagtgtccgaggggcctcactgagcacctagtccaactgagtcactgtgtgaaggagatactgcaggaggtgacttccggggacacccagagttctccagaaatgctggccgagcaaacactggaacagggttctgagaaaacagtgtgtaaatactaaacactcacagagtatttgcattcaggaagagtgaagtcttcttctagaattcttggtactttttaaaacaaatataagtattgccctcattgttttttacacctcactgccctgaaaacctcatcttcactgacatgcaatgctttaaatagcagtcatctgaaggcacctgtgaaaatcctcaaggtgcctgtacattgagttctcacgttcaagttcccaggaatattactggttcttgagaaaagtgcagagatgtgaccccgtgtgcacaggtcatctctagtttgggggagtaacagcttgaagggacattagcttgagcctggctggtgagtggaaaaagcatttgatgtttgcttaaactcacatggacttgagacatgaactgcggttaatattgttctgtttctttgaggaaaaaagtcatattccttataaataccatactttcatttgctgggaaaatctcattttttttatgttctgttcacctgaaatgcaaataggagtttgcttgcgattaattaagcggatgatagatggcacttaaaatgaacattcagcattaccccccatctgtatacagagtacagagataattggatgggaaacaatatggcatttaagtgtaagtttttttaaaagagcctttcccctttgacttatgcagataaagagtctggcctttcagaattgtgtagtgtcactggcatnnnnnnnnnnnnnnnnnnnnnnnnnnnnnnnnnnnnnnnnnnnnnnnnnnaacaatatttaagactatgatcccatttatgttaaattattagatgtatgtgtatatgtgcaacatatttaggaaaaggcttgaaggaaaaatccatcaagtggatgatagtggtacctctgagaatgggaggcacaaaggttgatttttcacactacccttaattatgtctgtattagttgaaactttcaatgaaatagtaatcaccttatcactcatgtgataaattttttaaaggcagagaccatcagaaaaatagtgcatattggagattttgaatcctcatagtactttatagtatgttgaaaagtaagcatctacagcttttttttttttttttaactttttatattcttaaaactgcttggatccaatttgttctagagaggaggggaaggtaccagttacttcctggacttctctgtgaggaactgttccagtcaccactttcccagacactctcatgtgagtataatcagtgtctgcaaagtgctaggtaacattcagaaaccacttactatgtgccaggcatccttctaactgctttacatatgttaccttattaattcactgctgccctaccaagtagatactatttttattcctgatttacagatggggaaactaaggcacagagcagttaagtgaatcacgagagtatcacatatattataaaattatacatgttatttacctacacttagcactggatttttttttttttttacccattaggattaaactaaaacaaataactaggtggtgttaagaactgacacccaagggtgctgccactcatgtcacctcatttacacctcacagtgactccatttgacaaagaggtaactgaggttcagcaaagggtcctgcctgatgttcacaaccgagctgaggttagaatccagtcttcctgatgctcaagcaaagggcttgttcccctagttacttaccctttgatactcaggtatctgcatgaagatctccttattttctggtgtatcttaccctcctcaggacatgacttaatgactaaacaacaaccctgatttttcgaaagcctcttttcctttcccacatgtctgggagttcacaacaaagctggataccttcctaccacctgcacagacacattcaaaaacacacaccctaacaggtttatcgcttctttacagatctttggattctgcagagcagatttgttctatgatgtagaagcctctgacttggaaaccccaaaagacattgtcacaaactgtgaagtcttcaaccttaaggtgggttgcttaagtagcctttgtcatgatcagtgccaaaataggtgatattttcacacacacagcatatattgttcttatggttgttggtgtgtattgtgtgtgtgtgtgtgtgtgtgtaagagggacagacagagagagacagaagaaggagaaagagagatggtggatgagggagtagggatgacaaggaggagaaatacagagagaacagggaaagtacagttgggacagtacaaattgctgatatgacacaaaaaatatggattttttccccccaagaattgagaagggtaagatttgctggaaaagtaaaatcatgattaattcaaccaacctctaatgaattgaaagtggctctgtagtcaggactccagtgctgacacttaatttcaatattaggcatagaactaaaaaaaatgctaaaatctactatataaaggtgtatgaaataagactcaaaacagtattttacatatgtcccagaaagtttttattcgataccaagatattcttcacagtgttttttatatcagaaaaaaacaaaaaacaaaaaaacctgagcataatccagtgcccatgtaccagcaccaccccaagtagagggggcagccttgtcaccatgaacatggctccacagatttcttaaaaattaaaatctgaacaaggagaagtacacagagataagtgatcatcctttaggagtaggagatgtttgtaccaaaaacctcgactctcgttgagaatataaattataaggcactaattttccaaaggttgatctatagataaggatcattttatagtgtaatttcatcagtttatattgagatgggaagaataataatgagtgttttcacaatataatgtttatttcatctaaaggactaccctttactctgagattaagtttctgactctgacaagagtcagaaaataacaaaaatattaaaataatcaaatgttatttgattattaaaataaaatatttctattgtgaaataatgatggtaataaatgatagttttgtttgtattttgattttgtttctttgtattttgaatgtgagttggctagataagaatttgagaaatccttttttgatatcctacttttaaaaaagctgtggaatctaaaatctagatgccattgacataaattaaattaaacatctagcttctttttgtgttacatgatgatatccactcttctgtttccaccatcaggaacatagaaacttcagtgctgtgcaacatcatttaggccgtcccttccactctggcgagcatgagcattctcctgctggaaggcctccatttaagcccagtggatctaaagatcatggtcatccccatgagtcatataactttagatgcccacctcctctagaacacaaaaatcactcagacagtccaccatttcaagcaagggctcctctaccatttccccctccagggttaagatgtcctcaccctccctttggcaccaaggggaaccataggccccctcatgaccatagttctgatgagcaccacccccatggacatcatccccatggacaccacccccatggacaccacccccatggacatcatccccatggacaccaaccccatgnnnnnnnnnnnnnnnnnnnnnnnnnnnnnnnnnnnnnnnnnnnnnnnnnntctgagggatctttctgacccagagattgaatccgagtctcctgcatctcctgcattggcaggaagattctttaccactaagccacctgggaagtcatccccaaaataatgaagttcatgaaaaaaaaaaaaaaagaggccctaaactgcagctttcgtgacggtacagagttcgcctgcctagatatcaactaggggaatttagtcctataggacattgtgaaaggtacaataatacctatagtttaaaaagtaacgataacaatggttcccagcctttattgtccagctagcattcagcagactctgagaagagaatgtgtgtgcaaatgattccttaaggaagaactgcctgaaaaactgggaagggagcaggggaggcaggacagagaagcgggagaagccaggacagatttggatgaaggccttttggcatgtgggctcctggggaaaggggtgtcccgccaaatacgctctccacatgggtccccgctccaaagcgaagaagcttctagttcatcggccagggctctggttaaacagccaagaccccagggtgtaactgatgtggccagttttccctgagcctcctgctttgagaactcaatcctgccgcctgccatggcagagaagaggcaccgagggggcctcgtatctctgctgtgacccctgagaagacgtttcaattagtcctggggcagccagccagggagcccggctacggtgccacctcctgttggaggtcagggccttgtgagagagcagccttctctggtggccaaaagccaccccagcctgtctggcgattggtttggggctgtacccagaagctgcaggtccctgccaggcccagtaaaggaacacgttgttcctctggggaaatgctgtcacttatcattagcttggtgagttgagggcatatttgaaaattagggaagctgatggatgctggcatgtggacctagagagaaggagcccgcctgccctgtcttcctctaagctgggttatttctcattcttctagttgctcagttgtgtccgactctttgtgaccaggtggactatatagtccatagaattctccaggccagaatactggagtgggtagcctttccaaaacctctcctctaacagccagtcaggaggtcctggcagcctgtgcttttgaggggctcccctggttgtctgggggctttggagggcatctcattgcttgcctggcaatatgacaacctggttaatagaaagtcaggaaagcttcccctctgggcctgtgtttacaggatacggaagtcatagaatctgttacaggtgtgcatttcatgagtagttgggcttaggcaaaagcaggtgactttcttggaaggcttcctggtgtggtgggcaccacaccttgtgaaaatgatccatgcgacctcaggaagcactttctgcagccggtgccctgggatgctcccgagcactggcccaggcagtgacgctcacaccagtgaacttcccaggggaggcagtggtgtgacgtgaagaacattagtctggtgatctggaaatctgtcccggatcctgatcttgaagtcagagcatgtccctggggatgttgagatggagaaaacactctcccagccttcagggatctcgagcagaaactggatctataggtgggcagtgatgctcagatcagccctgatgggtcaccaaacacagagcagcctggcaccaaaatgcctagtcaaggacatcagcccttgttacctttatctgaatgagggacccagtgaccatcgagaaggagtaggtgaatgagcaaaggaagaaacggggacagggtgaaaagaagagggaggaccatgaactggacgtctcttcccagcctgaggagccctcggcctttcccagagtggagaaggatgggaaaaggaagaactgctagtgtcttcctcttcctgcttctcatgcttttaccagtttatggcatataagcaccttgccctccatccactgggttcagcccacttggtgctgaaagtgggggtgaaacagcatgacccaccagtattcaaatccagcctcttgatcttgagtaagttaacctctctaagcctgagatcctccatggaatgggtataataaggggcaccttgtgggctgttgagacaatttcataacataatgcatttaaaacccttagtagacagtggcaagggatacacactgcaggcagtgagtatgagctgttagaatgaggatggaccagttggggtgctctctgaatggtgggtaacctcccagtgtgaggtgctgttcttgcccacttggctttatctgatccgaggggatcaatcagcattgaactccagtcccagggagcaaagctgcttccttgtctttccagaatgtaccgatgagatggtattgaaatagtgaaagaaaagaaaattgatcaacaaatgccatgcttgttctcttagaggaaaagaatacatttttttctccctttaatactcaagacttgggaaagtcaggaagtaaagaacaatgaagagggaaaagtgtgtccagacagatatttggggaggcccttggacttccctggtggctcaaatggtaaagcctctgcctacaacacgggagacccgggtttgatccctgggttgggaagatcccctggagaaggaaatggcaacccactccagtgttcttgcctggagaatcccatggatggaggagcctggtgggctacagtccatggggtcacaaagagtcggacatgacttcactttcactttctttcttttgggtgaccttgattctgatggataaaaaattgccctgtgctgtcatcacaaactcctgggctactttaggctggaagtgtgggggaaccagggtctcaagattgttgtgaagtgtattcctggtgggctcagcccttggtgagacttgaaggtggaggtcattgcccaagtgactacagccatcagtcctagacttggcttgctgcctgctgcgctccaagcccaagcctggacagctctcacaaccggcaaagtggtagcgactagtcctttgcatgtctttccatctgtggtgcgtgagaatggtatgggtggggttttctagtggcccagaagctggacaggagacaaatggtggtttgttggctcctgagtcactgagtcacggagctgcttctggatcatctccagttcagaagatggggaacaacttggtctgctgtgatactggccttagcatctagggtgttcctgaatgtcgggcagtgactgctgctggccacagagtcgagagcgctgcagatccgcttactcagcaactttgcgaagtgctttttaaattaggctttgatttctggggcttgggggcaggggagtgattacagacagaatgatgttcagtccaaactctgccacttaaaacacgtttgctttttaagtgaacatataaaacatgttctcttcctgtggttcctaaatgaaattcagagccagaaagaaaattcttgtgttattattaatatcattttgtcagttagtcatcctccctgatttatagaagacagttgtcactacattgtcaaaggggcagcatcaggccgtttggtttatctaggtttatctagctttggtatatctaggtttccccccttcctttctcatctgtgccctccttcccacccctgacccccactcacatgtgtttagtctgtgttcttgaatatgtgtgttcatgaatatatgtgtatccatgaaaaagatgcttttatgtgcccattttttaaaaagcacaaatgatattgcacttcagagctggttcagtttcttatatttccacacaaaactttttaaaagatcaactcgtgtttttagaggtagaaagagttcatagcttcttctgcaggagcattttaataaattcttggagggacttccttggtggtccagtggttaagacctcgccttccaacgcagggggcttacaagttcgatccctggtcagggagctaagctctcaccatgcctcgcgaccgaaaaactaaagcatagaagcaatattgtaaaaattcaataaatgcactttaaaatggtccatatcaaaatagtctttaagaaaatagtaaattcttggaaaaagttgtgctattatggctatttcgcaaataactaagctgagacttcgagcctaagaacatatatgaaggtgtacagcagagcttcccaggtcgttctagtggtgaagaacctgcttgccagtgcaggagatgaaagagatgtgggttcagtccctgagttgggaagatcccctggaggagggcatggcaacccatcccggtattcttgcgtggaaaattctatggacagaggagtctggtgggctacggtccataaggtcacaaagagtcaaatacgactagaacgacttggcacacatacacggcagagccaggcctcacctctaactcccgttctgtgctctttccaccactgtgtccgtgacctcagttttttaaaacctcttggtgacttgagcctgttaaagggtctgatcatggtcatcttgttgattacatccttatcaagcccttggacagagcctgagcacccaggcttttggcctctcccctcctgttttattagtctcacaaaggctgtgctcacagacacagccatttctacccatgttttctgctctttgctactcaacaggagctagcagtgcctcccatactctagtgcaaaggtagtcttcccagcatgtgattattcttatgggcataagaataatagtgtctttgtttctaggttctcacaaatggagtccagtgggcttgtttcagttcattattagcatccacctcaagagcttcatcaaaatcggcgctctaccaggaaacacattacttatggatgacaatagttgcgtgcgatgatttagaagggaggagaagattgggcttctggacccaaggcttttccttattagaattgtggctctgtgaaaggcttataaacactgttcagaaatggaacaaagatctcagaagagggatgctttttctaaagagactacagacacaatttacagacaagtctagaaagaatattatttgaagagggtgatgatggagctatggaagctgagaagggatggagcttttaattggaacaaaaaagaagcttcaatcccagaattcaaaaccgaaacagagcttagaatccttaaaagaagataagaaggaggggaaaagcaggtagaaagaggccaatgtctgtctgttgttggggatagaagctagaaagtaaggatgagggacaggcacacctcggaaatactgcaggtttggttccggaccaccacaataaagcaggtattgtaataaagtgagtcacacagtttttggttttccagtgcatataaaggttatgtttacactataaacatatatttactacacctagcctgctaggtgtacagtagcattatattgctaaaaaaagtacataccttaattcaaaatactttgttgctaaaaaccatcttctaaccatcatctgggcttccctggtggctcagtggtaaagaatctccctactaagcaggagactcgggttctctcccttggtcgggaagatctcctagagaaggaaacggcaacccactccagtattcttgtctgggaaatccgatggacagacgagcttggcagacaaaagagttgaacacgacttactaactaagcagcaacaataaccatcatctgagccttcagtgagtcttagtggtcacatcaaagatcagtgatcacagatcatggtaataaaaataataataatggaaaagtttgaaatacggcgaaaattaccgaactatgacccaaactgagcaaacgctgttggaaaaaatggcaccagtagtcttggtagacatgaactgtcaaatttgcaaaaaatgcaatatctgtgaagcacgataaagcaaagtgcaatcaaaccaaatgaggtctgcttgtaattagttttgttgggaggaaaagttaagaagttagtgacaaaatttgtagtgactgaatattggctgaaagttgatacataactttaattatatctttctgttatttgttactatattgttacaacccttattatttagcaactgaataatgagagagagcattgatattgatgaagaccttcaagccgaaatatttatcacgggccccaactttcttggaatatgtgtcagggagcatagattcacaaaagaaaatttcatatttgtgaggacagtataagtatttggcttttatttatgttcccacacgctacacaaataggttttctgtccagtgtgctggaatttatgctgccaagtcttattacagtattgtaaattgaactacgtatgatttttcctttggaaataccattatttttcctgcatgttcacctcagccaagcactttaaaaacctttgagtcaaaaaggtgttgtctgaattaattctgtatttgttcaggaagaaatatttgctgagtggcgtggatgggaataatgattattagggaattgaattcttttgacaaattgtggattaacagcattacaggagtcaggaaataacatgtctcctttgcaaggtcatcttggtgggtaacttcaagtctgggggacagtgcatagggccagcctctctggaccaatgactcctgaattctagactctgccccttgctcttctgttcccataaaacatgtgtctctctagagtacctgctcacttccaaacccctgtgactcttcagagccttcaggactggtgagttgtctccagctgcctcgtgcaggcagcctttgccctatgatgagcacaggacacagtccagccttctctcccactgactctcccgctgtccccaacactccagccgagcgagtttattcaggatccctcagcactttctgggctctctttctgagcctttttctgcctggagtagcctgtttccagccctctctttctgtctaaatcccactcctcattccaggcccacaccatgcctctccatctcagaccattctctcagaccactggccccaaagggccgactgtgtcttttgtctctggaggccagttctggtgtctgcatgagtgaaggaccatgtattgtgattgccaaggggatggttcaaatgccagctcagccacctaccagccacgtaaccacagctggccacttatcctccatgcctccgtttcctcatcagcaagaaggtgatgaaagtatttacctcataggttttgttttttaaaattttaaaatgtatttatttttgactgaaggataatcactttacagaattgtgttggtttctaccaaacctcagtgtgaatcagccataggtgtacatatgtcccctccctcttgaacctcctttttacctccctccccattccatccctctaggttgttatagaaccctggtttgagttccctgagtcatactgcaaattctcattggctatctgttttacatgtgggaatgtcctcatagggttttaatgaagagttagttactctttatgaacttagatcagtacctgacatatcctgggcttaatatgttaaatagggtcctgagagtatagccatttgatactgagggcctagttggttaggttggggttgcagttggtcttctcatctggaccatcatcttcttgggggccagccaaccccatgcttctgtgtagctccctctccatctaacactgtataacacatcagcatggttgaggaaggaaacagatgagagctgggcctggctggggagagctttggctcttgcaaacagaggtggtccccctatatacagagcactccagtgcttatgagggcttcaaaggatggacttgaaactcctcttagtgaccccgaggatgaccccttgggggaagggccctagaagagcacaggggagaagggggaaggtacagggcgatgcttctctatttgtgcgcctcttttccttttggaacagtgtttctttggttccactcaaggctcccttggtttgagtatatttactgactagccaacaaaggtccatatagttaaagccatggtttttctggtagtcatgtacagatgtgagagttggaccataaagaaggctgaacaccaaagagatgatgctttcaaactgtggtgctagagatgactcttgagtcccttgggctgcaaggaaatcaaaccagtcaatcctaacggaaatcagtcctaaatattcattggaaggactgatgctaaagctgaagctccaatactttggccgcctgatgcaaagagctgactcactagaaaagaccctgatgctgggaaagattgagggcaggaagagaagggggcaaaagaagatgagatgattggatagcatcatcaacacaatgaacatgaatttgagcagattctgggacatagtgaaggacagggaagcctggcatgctacagtccatggggttgcagagttggttactgcttagcaactgaacaccatcaactgacttcagcaaagacgtataccatgaaggaccttgatcagatcacagatggtaattaaccccagccctcttctgggtgtccagtgcctatggtttctccctcttttagaacttggtgcagttaactcatcggttatggatgtgtctcctgcacccaattttgaaaccttttggattgcttctccaaactgctaaatgacattcatgaagcggtgaagttgttgagtgcattcctgcatgggaactcacagaacagtgtaaatctgcccatcttaagaagcctgacatctgactaaatgacaattaaataataataaatcaagaagtagttaattaacaatacattctgtgtgagttttggtgggggagagagagcccctatcccaccacacacacaccttgtgggctggagtgctccaggggagggctcctggtgctgtgaagatctgtgtgtttataattcagctttatttgaaaacttcgcaatttaccagttgggaataagccaagccctgacaaaatctatatgcagttcaaggaacactgcaggcagaactgtccctgcttttgaaagtgtgcattttataagtctaatgtgcatactttattgtaagaagtgccaaagtgcagattttgagaatgtagttgtagcagtaggacctgttgttagtgattatggcggaatgggcttagactggggtctcatggctgctctgatcctaggctccttggtggagactgtttcgtggtagctcaagaattggaaggactgatgctgaaactagagctcaagctccaatactttggctacctaatgcgaaaagccagactcactggaaaagacccagatactgggaaagattgagggcgggaggaaaaggggaagacagaggatgagatggttagatagcattatcgactcaatggaaatgagtttgagcaaactccaggagatagtgaaggacaggcaagcctggtgtgctgcagtctatggggtcacaaagagttggccatgacttagtgactgaacaacaaaaacaggagtcgggacatttggctttgtcctcaatgctcccaaatacacaggtgtgtgtacttacttgtcaagaggaataaataggaggaggtgagtagaggtgtttaatatctatagtgatatgatgtagatgttcaggaagcagttttattgttccttgtccgtgtgatacattttccagggtaaagtaccatctcggtggtgcctcatgttcctgtggagattgggagtgaatgcttgctccctctttttttccccccgctttcctgcatagtctcatctgccctcctggttgcaatcacccacctgtattctgatacctgccaactggcccaggccttcctcccaagctccagatcctcatttctgatgcttatccctcagccttctctgcctcatccgtgccccaagcccaggcctgtgcgcttcctgtgtttcctatcagaaacactgtgtcagccctgagcctggactcagcctatttcctcctgttcctcccctctccattcaccacgtggatattccttaggatccgggattcttccttctgggtcaattttgagtcctcctcgttggtcccattcctgtatctcctctcttagcccaggccctagttatgttgcctggactatcacacatgcctcttgatggccatgctctctctgtcctccgcactgccgctgtggtcaccgtcatcacacacagatatgcccctataattgtctagttcaagaaacctgggtgactccttattgctggaggaggagggttacgtttcttataagcctttgatattcttcacagtcagggctccatgttcctttacaccttagcttatctagcatcctctatttttagtgactttttgcctttcccaaacaagccagatttggtacaacctctgtgcttttgatcatgctgtctgttcgtctctgtctatcttgtttccttcaagaatgactgctcagccttcaagcctcagctcagaagtctcttctctgtaaagctctgtacacagaccccagctctacatcatcacctgccatactgcctggcagtctcctgcatgtctgttggtgcattagtagaaggtaacttcttgaatcttatctttgtatctccagggtcagtccatggtaggcttgatatcactggtgcagggatggtttgaaagatagaaggagggatagctggatagatggaaggatggaaggaaggagagttagggaagacaggaaggaagggaaaagggggaaaggtggaagaaaggaatgaatgaattccgttcggtattctgttctccaagcagacgcttctgtctcgtctccctgatgcctgaggatgtcatggactttttgtttgctgctccatgtctaaccaggagcactggcacattgtaggctcttagtatttatcgatggatgtgtttatgaaaaatgtgctttaaaactcttgggctatggaaaagaaaagaagaaaagctggttggtagtttttttctgcttataaattgaagattttgtttgtcatctttttaaaaaatagtgcatttgctggcttacttgtggtggaaacagtctgcctgcatcagacattagtagcccctatattttttaaagcctccacttaggaaaaagctaaaggaagggcatttggtaaccagctgggaactccatctcccatgcaggcctgctttttaacttaatgcttaggccctgcaaagttgaaatggccaggtccctttggaaaataaggcaatgaacttaacaaaaagagaaaatcaagatttccattttctcctgaacatggaaagcagcataactaaattctttgtttccttttattaccgtattgatggttacattttgtaactatggttaaatggagcagtcccgtggtgagggatgcagggtaagcagtgccccgtaaacatagggaccctgaggatgggaaatgtctgtgcagttacctttactcatttttctgaagctcataatttagcccacaaagtcaagctaagcaagggaatcctaagatgaggttccccggcaaggattgtaatgagcgtaaaatcaacccgcattcatctcgccctttaatgatctatgcatggtaatctaactttgcattttttcccttctctgtccttggtggaaggcagcctgctgggctctcacttgcccacgctttctgataaatgtcaagcctatcttgtggagctggcacgtagttagaagtcatatgtttcctccaagtcatcctttctcccaaggataaggctccatgttgaggataaaacccagtgcacattctgcactcagctcagaacaaacgctattaacctggaggaaagctgacatttctgcaggctgctgcagcgttggtattttatttgcttcccctggggcctgactgtatcctgcctttcgccgtgatgtgctcaggtgaagcttgaatgccgtgtgcaggttttctaacaggttgcggaagaaagagatttcatttggagtcctaggaccaagggaaccaggtgcacctgccacccaaaattgattaggatagtgcatttgcaggcaggacccgtggttggggctttctcatcagttacgggaaatggtcgtgcaaatcttttggaaaaaaaaatacgataaatccatagctctctcagagccatttatatattatattttgctttcaatatcgagagaaaaatcatggtttaaagcttattaaaatgttacccatatagatagttttaaaagataatttatattagcactggtgcatagctttttaaattaataattcagagctgtgactagaggtttcagtaatctccagtagtatattatctgactgttccactgagaaattagcacttatggactaaggaataaaattgaaaaatctaacccatttggtgagtctgttctcagttacgagagccttcttaaatgaaagtctattagtgtgagaattctgatttctagaatcaaatctgttttaggtcattatggagttttataaagaaaagcctcctctgctcagcctaacaacacgttgaagtcttaaaagtaggcagactattgatttttagcaagagttttactgccccagacactgaagaatgattctcatgactaaaatttataggttattaaggagaagaatgcagagaaaagactagttgaaagattcagaaaattagctcattttgttcgttagaattcactaagcaccctttcctgaaatatagatatatcagagtctggaaagggcctcttggttacatggtaaatttatatggatcagaaatatagtggaatcgtattttagcccacaaggacacgatcactctttggaggtttgcgaggaaaaagcaaagtgctctttttaaagaagcttaattcgaaaagatcagggttggcagatcggaccactggtggactttttttttcacctgccaaaaaacctgttcagtccaaactaaacagacataacagaaagcttcttctagattaaggaggtagtcatgcaatgttttgtttttaaaatatcaaattctgtgcttcagtttcgaataggggaatcgttctgaatccttgggggtactaatctgtctggtacctttcaaagggaatgtgtagagttactggaaagccaatcgtgtgaaagcagtaggggtactcttcattgtacttggattaaaatataaaatgtgccttatgccagaggcagggcagtataggagaaatcactgcacggggaaggttgaaagggcatctggtctcagctccatcattgagcttctgtgacactggacactctgaacctcagtttctgtatatgtgtgatggggttcgtagcagccatcatgcctccatcatacccaccctcagaactgtgatccagcctttcctataaaggaatcgagaccacgtggctcagcaaccggaaaagcaccggtcccatagcacctgctccaggaagtgaggtagaagttctcactacccaggactgcaggggtgaagtgtgaagtgttcgtcaatgagtcatgtctgactctttgctactgcatggactgtagcccgccaggtgcctctgtccatggagttctccaagcaagaatactggagtgggtagccattcccttctccaaggtatcttcccgacccagggatcaaacccaagtcttctgcattgcaggcagattctttaccatctgagccaccagagaagccccaaagaggtgatggtggtggtggtttagttgcttagctgtgtccaactctttgcaaccccatggactacagcccgccagacccctctgtgcctgtggaatcctccaggcaagaatactggagtgggttgccaacccagggctcaaacctggtctccggcattgcaggtagattctttaccctctgaaccaccaggacggtgttctcagtatccagggctgcaggggtgaggatgagaccaaatcatctatacgaaagtactctttaaagtgtattgatgtacagtatactaacgcatatatatggaatttagaaagatggttaacgatgaccctatatgcgagacagcagaagagacacaaatgtatagaatagtcttttggactctgtgggagaaggcaagggtgggatgatttgagagaatagcatttaaacttgtatattattatcatgtatgaaacagatcaccagtccaggttcgatgcatgagacagggtgctcagggctggtgcattgggatgactctgactctgagggatggaatggggaggggggttcagaatggggaacacatgtacatccatggctaattcatgtcaatgtatggcaaaaaccactacaatattgtaaagtaattagcctccaattaaaataattaaaaaaagagagacacatgtaacccagtgttcattgcagcaatatttacaatagccaggacatgaaagcaacctagatgtccattgacagatgaatggttaaagaagatgtagtacctatatgtgatggaatattactcagccataaaaaggaacggatttgaatcagttgtattgaggtagatgaacctagaacctgttatacagagtgaagtaagtcagaaagagaaaaacaagtattggaatatatatatggaatttagaaaaatggtactgatgagcctagcagagaacagacttgtggccaaggtgagggtgagacgaattgagaaagcagcgttgactatcatgtgtaatgtgtaaaatagatagctagtaggaagttgccatatacacagggagcccagcctggtgctgtgtgatgacctagaggagagggatggggggaggggagggaggttcaagagggagaggagatatatatatatatgattgatttacgacagaaaccaacacaacataggaaagcaatgttctaccaataaaaaaaaaagtgtgttgatataagaaactgagcctgtaacaacttatgattgtaaagagttatgtatccaataccagagacacagaatatattccgcagatgtgtgtatatgtgtatatatatgtgtatatatatatttatatagcatgagtgaatgaatttacgtatcaaaattgatactgactccttcaaagtaacccctgaaaggtgcagctcacggcccactgaggatgtttttgtatcaggtatcttgtcctcacggctctagatgtcgacaacagctaaccaataggctctgggtctctgagcgcgccctttcttaagcccgccttctcacagagatgccttcctccagcacagagctgagcaagtcggtctgtccagcaagcaagagggtggggattgaaaccctcaggtggtgactgcgcagccctgttggaatagggtccccacaccttgtcctacacgcaggccatgcgctttgtgactcctgccagctctccagcctcctcctacacagactgcgctgcaggccggctggaactactcaagtgccccctaagttccagtccctgtgtggcctcctgccttcacagctgcccccctttctgcttggccctctctctctggacctgtccctttcgagaagggacacaagtactactttttcttgaaaacctctgcatcttctcttccaccgatgtctgggcttcctcctgtttcccactgacaaccttaccatgttggactgtgatttttaggatctgtgtctgccttccttactagattctgaacttagcaagaggaggattcctgggtctttgtttctcctccctaccacctcagcctcataggctctcactaaacatctgttgggagggtggatggatggatggatggatggagatatgaaagcttccacgtcttacctggtaacttctagtatatcaattcccaggatcaaacagcctggcaaagagattggggactgcatattctattaagatagtgagtcatcccggattctttctgctcttgttccagcaaaaccccaaaggctttataaggcttttggcaaaatctggctcagaaacatttgacctggtctcagtttctaaggggttctcagagctgaggatagtcaaggataatgtcttgaaatgcagcttccggacaaggaaggcattcagttggtggggggcagggggcggggggaagttgctgttggagatgcaggagaagggaagaggtgaaaccaccaagctggaacgtgagacatttgcgtggggactaatctgagtgtttttttccttgacacaccttcccctctgcttctttccacaccaacacctgcagggtgacccttcctggggactcttctttaattgcaagtgggagggagtcagtaggcagaaacattcgaaacctcagagtccatgggagagggcatgtgaaactgatgaactcttggtctgagaagcagggaggttcacgggaagccactgccaagatgtgattgtggttcagcatctgccagcagtcaccatcgttccagctgtgtgtgtcctgcttcaggaagcctaataaatctggtgatttaattacactaaattacttcatcagagaacttttttatgctgcaaattcttactttaataaaccagagagaataaagaaaaatttcttgaatatctttgccaaatcagtctaggcatccattttcaatataataactaatagcggaggagattgaaatagatgttattacagatttttacaaggcaaaatatagttaaacaggatcagaaggttaattaaaattatagttgggtcttgtcattacccagccaccctttttcttctgcacagttgctcccagtttagcatctgtttgataaaaatgcttaaaatggaatcctcatttaaaagagaattaggctcggtattatttctgagcctgatctcatgtacctacatcggaaataaatgcataattaaccctttttcatgataaattaacattcattttcctaggattagtaggaaatagcataatacagggcagggtttgccttcattccgacagtctcccgggacctctaacactcttaacggattagtgaagcctatgattcacagtgtgtctacgtacatgaaaatacaaggcagttcctgactgtcagagagagagcgtgtttcctaattgacagcttttacaactaaacatggaaagattagcctgcaaatattgtgaggatgactttggagtcctggccagggtgtgttagccaaaggaagtgtcctgtgaatatttaacattgaaaatgaattcttctagaacttttcgacatcaggttaacagcagccatgcaaatcacgttccctggagaacagataagctttcaggagaagctctttgtctggtgccttctcgtctaaaactcttgggggttttctttgctatctgagcccagctttatcaactgataggtttaaatttacataaacagtctagcttgtttcatttgatttattatgatcaggttggaaaaagcaggggcctgttggagaatagaatcccgagcctcgttgagtctttaactcaaaatttccaaagccttgaccaacatgaataaattgccttcagatatttattgaacattccaaagtcacagcgtcagatctgctttctatgtgtttaagggaagaggtggggaaggagagagtggaagagacttctgctattgcagtcagttacaaagaggtacaggttttctttgatttggcccaaaatatgggagtttagcagggcgagtggagcagcatctgttgagtaactatgatttccaggtcatattcctagcattttgcatgaattatctgttgagaacttgaaagtagagtgagatgcttgtgagccttgtagcccagagagagcacaaggttaacctctccctcttcctttgcaatgtcacatacccagtggatgctaacaaaatggttaaggattcttctcaatgtgtcctattcacttgttgtcgttcagcgactcagtcgtgtccgactctttgcaaccccatggtctgcagcacaccaggcttctctgtccttcactatctcctggagtttgctcaaactcatgttcattgagtcagtgatgcaatccaaccatctcatcctctgccacccccttcttttcctgccctcagtctttcctagcatcagtgagtaaaagtcgctcagtcgtgtctgactctttgcgaccgcatggactatacagtccatggaattctctaggccagagtactggagtgggttgcctttcccttctccagggggtcttcccagcccagggattgagcccaggtctcccacattgcgggcggattctttaccagctgaaccacaagggaagcccttcccagcatcagggtgtttttcactgagtcagctcttcatgtcaggtggacaaagcattggagcttcagcatcagtccttccagtgaatattcagcgttgatttcctttaggattgactggtttgatctccttgctgtccaagggactctcaagagtcttctccaacaccacagttctaaagcagctcgatacttcagcgctcggccttctttatgatccaactcttcacttagtagactccgtaacaagacaggctcagtgtcaagttgtggataacttggtcattttcctcctagtctatccttatagtgagttcattgaggcctttgatgagtgggtagttagggtcagctttgggggaaggagtattttatttcacttttaaagtttaggatacagatgagcctttgaaaggcaaagaatgaactataatactcaagtcaaggagaatcttaatgcccacatggggcactgggtgaatctgacaaaggccctggagttcatcctgtgttttgaatttgcaggcagaacttcttagtggagagaagcaccagtgcagggaatgggtacaaagtgagtaaaggtcagggaatgagacccagcccacggcctgcctcgagaaatggctgctcatttcccaggggcaaaggctgccccatctggataccccctgaaataccacttggggttttgttttccaagggaagctactgatgtgatcagaagtactaacagattgtttacatagcagaggcattgtgggttttatctctgaaagaaaatctagtatgtatgtaatctgcacgctgaatgtggttggcgtcaagaaacagttggatcaaataaaagtaacaatgaataaaacagctctaaaacagcagccgatacacatgaaagatgcagcacccagccccacagcctctgcctgccagacagtggaagagaggctctctggggaccttaggaaaacccctagctcagacccgtcagggctaccctgtcctctccctccactgcccccctgccgtgtcacctgaagggggaccgagcatttacttccacaggtcagcccctctgcccttccttcaaaggccttctgagcaggtgggaccagaccaggaccaaggatgacacagcagcagacagacatttggggaaggaagtagctgatgcaggtggatctcgggccagggctgcccagtgtggaaactggtttcacaggctctggatacacttgtgctttgaccctatgagaaactttggaaaccagtttttccctgccttgtcctctcttgagacctggtaacaggtttggggagagcactgatgcccaggctcctccaaaagtcaagcctctcaggtgggcaggttttttgctttgtctaactcattgccatattccaagtacctagaacagtgccttgtgcctagtaggctctcaaggaataattatcagtggacaatcacgcttacctgtttgacctaaaaaggttatgaccattgactcttaataagaatttcatgaatcaggactgaactttgcacctgatccagaggttcactctcatgggtatttcataagcagggttatattgggattctgagtagaaagcaggcttcctcaaagtgtgtcatggtatcacagaatgcttgatgaagatacaggttttggagatcgggcctagacccactgcttcagctgatctgaggggcagagcctggtaatctccatcttaaactccaggttctctgaattattctgcacagtacagtctgagagcttttggtcttccggggccaccaaatgagattctaacaaagaaactgggaaaggaaaaaaacgacttgggtcacagctaattttgaaaatgaactccttcagcatgcagcaggggttccataccaggcactgggccaaatgcacagaggttttaagaggatggagacagtctccatcagcaacaaggttaggatctggccagggagggaatgatgtgaatagagatgaccttgaggtagagaggaagagggtggcatcagataaagtgttaacagttgttcaggggcgagaaagacagaggggagcaaagacaaaggcatcatggagctggacctgcagtggtgggttggagtcagacagtgggtgacagcaggaagacaagttcaagtctgaggaattttttagcctaagtactgatatgggaaagacttgcagagcagatggcaaattaggaagaccttgagagtctggtgtcagggggggatgcccatgaagggaacagttgacagtctcctgctatttcaaggttaaagaaaatatgtctgagaacacgctgttggatttgagattaaggagggcattggatctttcaggggaggaacttccatggccggttttggctgaaatcagagttaaaggaatgggttggtctgttaaggaaaggataagaaaagggattgagtgttaactctactctttccaacttcaggacggatctgggattgtccatagtaccatggcaatttgaggagatgaggggaggtgttttttggagaggggatctgaatatatttatgcacagaaaaagactttgtggagagattgagattaactgaagatgcaagagataagggaagcggccccacaaaacaaggatagatatgggaatgggtttaattagccatgaggtcacgttcctgcctctgtctctctttcatgtgtatatgaggagggtgtgtgtgtgtgtgtgtgtgtgtgtatgttagaaagagacagggatagatagagagacacaaacagacagaccaagaccaaggcaggaactggaaacttcatagctgcacagtggcttccagaattgttctgtgggctgggagagtagcaataattccccagtcttatcctggttagaagtgatttcagggaccaaagaaaatactattaataactgtgggtgggtagttgatctgaatcaccactgagagagcagaaagaagggggaagcagaggatgagatggtgttagatagcatcactgattcaatggacatgaatctgagcaaactccgggagataatgaaggacaggggagcctggcgtgctgcagtccgtgggatcgcaaagagtcggccaggactgagcgactgaacaacaacaatccacgataggagatgcatatttgagtgataaaaacaaagtatcacaatgcagagtctttatatgtctgctcttttgagcattcttaagatgtttttgaccactgggatgtgttgaagacaggccaaggtacttaagagagagccaccaaagaaatttaagactagttgattcaaaacttatcgcaggtgcagagattttaggactaaaagctctgagaagcctggtgtttccagagaagagagtctgaagaccagttataaaatccagtaactcgttattaagagctgtctgtcctagcaaggtagaatgcgaaaacacaatattgggaagtagtgtcagtaacatatgtgaaaagaacaataggtttttttttttatataagaaatttttcgaagatttcttataaaagaaatcttccaaaaatttcgtataagtgatttcataagtgaggaacaagatgtcagccatacttttaaatgatcttttttgacactggtcgttgaaaaaaaaaacaagggatggtttttggagggaggacaggtctggtcaggcttccacttgctaaggatgacactgagttgctgctgtttggaagtggagtaaagatcttatttttcaagtgcaaatcgtatctgactctaatagtacgaattctgtcaagtagtccatagaataaatttgcacacagtttaacataacacaagtacattaaataggatttggcattaaaagacccaacaccaaatgggaatccgaaaataaatgcccacattctccccagaagaccattcaccctgaggccttgggcattaatttaactgaatgaggaagccactggggcagctgtcaggagcttatttgatttgtgaataaacaatgccttcattttaaatgctgttcactctagcccaaacccaagcaggacctacagcacaaaagagactaattggttacacaaaaactaccaaaaaaaccaagcagtctgagggttttagggggccagtgttgaatttttattcaaagggaaatacagaataggagaggtgctggagggtttgatgctctgtgagtgctgcctcgcccccaggttgtgccttgctgttcgtggatacggtgcattcagacatccagtaatgaggagctggggtgtgacgggccatccaggagagtgtgatctttgggcttgtcagtctgaattagccagcattctgtctggggcacacagaacccgtgtgacaagaggggctaagaagcacggctgtcctttgtcgatcccctcaggggatctagggctgtgcagacctcttcagggacaggaatgatgtcagctgaacagaggttcagtgggagcatctatggagcatccgcagcaaggacggttgacactgtgtagggaccactggcgtgcaacgtccggatcaccctggtgctggggagagggactaaattgaagacacagaccctcaagggctcacctgagtcaatccctaaatgtgaagacctgtgaccagcgttgaaggagagtatgaggctgtggctcacccagggtccccctgcctctatgacccccacagtcaccatgtgccttccagtctgcaaagggaaggtggggggtccaggacccctaagggagggccatgtgctttgcgttctgtggacctgccttgggctatctcatacgggggcgagggacgcagcccagcccagcggggagggaggcctcctgggaaaggccaaggcctgtttccaaggagcaaattgtatttattcatgaattcacatgaatttcaagatgctgccgggcatttgcacttagcaactgacaaccagcaatgaaatgggccttgaaggattgtgatgctagaaattttggactgtggataagttttttttcaacaacgtcttccatgagttcagaacagatagctcatctggggtgtgggtctctgctctggcccctggtgtttctccagcaagtactggcttgtaggcacgtagtgaatgctgaaccccagcaagggtggtgggcatgtacagaaacgagactgaaagtgttatttagagtgctgatgagtcctttttctccccaagtcacatgtgttaatggagcgtggggttatgccctattcacagatttttatggtattctctttcctgaagagccagctaataaccagaggtgttcagtgaccttctttatgagtgggcttctcttatgggtcattacattgatttccccgtccaggcccagggaggttaagagtggggcacttggtctttcccacactgtggaccaggggtcgcctgccagcagcagcctgtggcatgagagtggtcagttgccttgcgaccattacaatgaggaggtggctgcttcccattctattccacatgaaggtatatcttgccctttgtaagacagagcctccagatccaaaatctgtctttgtgaatctagtcaccttgtccttccactcaaggcaggcgtagccgtggcaaagaagtcttttctaagtcggtgtctttttttaagcaagtgggatataatttgcataccattgacgtcaccctttggggttcccccgtggctcagcagtgaagaattcgtctacagtgtaggagtcgcaggagtcgcaggtttgatccctggatggaagatctcctggaggaggcagtggcgccccactccagtactcttgcctggagaatcccttggacagaagagcctggcaggctacagccatgaggtcccaaagagtcagacacgactaaagtgacttagcacacaggcatgatatcaccctttagagtgcctgttagtatattcacagaactgtacaaccgtcacagcacagggtcctttgaaggatggaatagttgtcttttacaaaatctattttgatggttgaaagttttaaacatgggatttttgcactcctgtgagctttttgcgttgtgaagtgtccgtgtgtgtgtgtgtacgttgtgtgtgtgtgtgtacgttgtgtgtgtgtgtgtacgttgtgtgtgtgtgtgtgtgtgtgtgtgtgtgtgttctccagaggctctgtagatgaactttgggatggactgtgaggcctgaccacgtagcaagaagggactgtgagccagctgagctttggaagtcctacccaggaaggcagcagtttggggaggccatcaagattgcctcagcccccagccacttcctcactgattaatttggtgttcaggtattcaccaaagctgatggagaagtaatgctctgagctctgagactagagttaatgctactctgtaggctctctgatctctgcctctctctacctagacctaggtccctggccttctctggcttgactttctcccactctgatccatgacatgggccaggccaggcctggctgcagagactccaggtccagttactgctggcttaacccagtcttggcacctttggattctgccagagctgataagacatgatgtggtcagaacttaggagagtcccaggtaacaaattcaggaagataatcctacgaacctgagggggaaagtggtagcaggaagcaacatggaaaacaaaccccttcttagctgagtgttactatctgaggttgtggcctaagggaattctgagtgtcttaaatggccctctgctgctgctgctgctaagtcgcttcagtcgtgtccgactctgtgcgaccccatagatggcagcccaccaggcttcgccgtccccaggattctccaggcaagagcactggagtgggttgccgttgccttctccaacgcataaaagtgaaaccaacgcataaaagtgaaaagtgaaagtgaagccgctcagtcctgtccgactctgagcgaccccatggactgtagcctaccagctcagtactttgttaacaagggccatttggaaatgagcttatatgtgagccatttgcagatgagcttacaatttttgtgcacaatagtagtggattttgcaaaggatgcccttttttcatggtgtagttgtaatcggcttccttcttgacataggcggtttccaggctctgttacagaaacgccactatttccaaatttgttcttgtacaacatggtggggtttatcctacatgagaatctgcaggtttgggagttggaaggtgagagtcttggattccgaaccagcaggagaaacaacacatcagatgctgttagtcatgggagaagcatcagattggagaattgatctctcagtgacctcactgaagaaaacttggagtttcagaagaccatgcagagtatgttttctagagggaaatttccccaactgtgcttaaagatgcaccctcccaccctaattttgttagtcttatgaatgaagcccgaggcactggggctcacctctcttctcccagttggcagcttagctctcatgcctgtactgagttctgcctccccagcccatcctgcaggctgaaaaccagcctctgtctctggttgcctgctgagaaggagacacagcccctggtatgtcccggtttctactcttgcatggatttcctatgagaaaatgagcagtaaattaagatcctgtagccatagaattaataattcattgttgttattgacaggtgtcctcctttccgagtagttcaggaacccatggtggcttgcgtagaattgaatgtggcatgtgagtggagattcagatctattcatggctgtgtgcctggtggtccctgccacttgggggttgtctaaaggcatgatttgttaaaggcaatacatccttttgtgaaacttatattgcccattaattggtcattgatctccagcccatttaggacagttattcttacagataattgtatgcctttgtcatgttgtataacactggatcttagtttattgcttgtggattatggtttcatcccaggcacttgaatgatgtctaatgatttctcactgacattcatccagcaaatattccttgagcatctagtgtgagtgagatgaaacaccatgcctgtgctactcagatgcataagtgatgcttgctgcccgctaggcattttctagactggttgtgaaaataagacatacacccaaataactattacaggaaagtgcttagcacccttctatagtgttggcaggaatgtaaaatggttacatcggtttgggagagtaatttgacaatgtcttttaaaattaaaaatagcctttcctatgactctgctatcccgtctctaagaattgagggtaaaaacgtgatgcctaaagacataaagtctggaatacttattgccatattatttgtaacaacagaaaataaaagccaaaaggaaaaaaaaaatcttttatgttcagcaacagaggactggggaataagtgacagttaattacaagggggtagagtgaagatggcatggatgaggaggttgtgtgatgaccaggaaggatgtccttaatatactggttggtgggggaaaaaaaatcacttaattgtttttgtaaagaaaatcagtgttgcgtatttgaatctttagggttcagtcatcaatgggaggtgggattaattagtagcttacatgatttgtaagggcatcccaggtggcactagtgataaagacccacctgccacaggcaggagacctaagagacgtgactttgatctctggattggaagatcccctggaggagggcacgacaacccgctccagtattcttgcctggagaatcccacggacagaggagcctggcgcactacagtccacgggtcgcaaagagtcagaccagaccaaagcgacttagcagagcccagcgtgctgtgtaaattgcaaattggtgttctctttgttttccaaatctatctaaatgcagctctggggctgaggggtggggaagcatgtgctaccctctgaccctctgtacatggccacctatggcccgcacgccgcaggagggcacctagggtcagcagagcgggctggcggctccttcgcagccacagtctctcacctcacctgtctgcggacagtgtgaaccaaagccttgccgggtacagagagccttctgagttctcacttccggccagccagacccaagggcccgggcaccatggagctttagaccctgaaaggagccccgtcctgtggggaggtttcctgtgacagaaaactctggcttcctctattctggggcccattctgggaaggacggtgggggacacgtgtcacccgcttgggcatcgtccacacggtagcctctaaaggctctgaggaggtctgcagtgacaacgtgtgctcacgggagagggactgggcccaggaggcgggaagacggggtagggggaggaaggggaacaggaaagaaatgtgctccgcttcttttgaaccaggatggcccgggtcttttggactgaggaaaccttcttcacgtgacccccttcctccagttctgaggggcacgtgagggaagcgctgggttgggagatccacgggtgtagagggagttggggacagtggggttccgggttaccctgcccccttcagagaggctgggccagaaggtaacctggggccctgatctttgtgactttgaggtcaggacagagactggtttgcttttcctccaccattttggcctgcgctcgctcattttggcccatggctatgtcatccgtatttgaataccgtcaggacacacgctggcatctgttcttaacgcagctgtttctctcagggtgtgagagtgcactcttgccttccagcgcaacagtctgctgtggacgatgggccgcccctgtgtttggtggcgccgaggggcccagggtgtgtgggtgcatcatttgtggggtggaggttgtgtcagtgtggggccttcagcactgctaccgagcttggagcaccctgcacccccgtgagctcgcccctgggcacccgctgcccttgcaccagggtagcccccaggactctgggttctagctttctgcaggaaccctctttgttttgctctctctgagtgttccctgggggcagagggaacaggtctcctctcccccttcctcatctctggtggatccagcatccgctgagcattggttagaccccagtgtcttcaggctgactctgtcctcccagcactgctggcttctcctctggactgatggtctcctgtctgttggcctggccccgctccaggaccttatgcctcatgcctgccttgcattagatgcctgatgttcaactgcaacttgcatagagccatctctttgggcctgcagactttcctcatgagtaaaatgcacagcaaaccacctgttttgcccaccatccaagtgtttgcctgaatcattccttcgacaaatatcttttgagcctgccaccctgcccatcattgatcccagccgtcagggagttttcattccagaaatctggtcatggcatttctgaatgcgcttgatcactggcacaagggagagcttcagtcatggggtcactctgactacattgctctctgggcagcatgctgaccaggagtagccctctcaccttccagcccatggatgttgactcttggcagagtgtacttactgcctccactggtgggtaggagaggggatggatctggtagggattctgcatcaacataactcagggacaactagtgaaagcgggaatgacaggtcacacaccaccaggattagttagtagaagtaaatccaggatggatgatgatcttcctagttggaggttttcagacctgccgagcttactctgaggaacagaaggttccggtcagttcaggtttgccgtgtttatgcaaagctaggggcttgcctggtggctctgatggtcaagaatccgcctgcaatgtgggagacctgggttcattccctgggttgggaagaacccctggaggcgggcatggcaacccactccagtattctttcttgaagagtcccatggacagaggagcctggcaggctacagtccatggggttgcaaagaattggacatgactgagtgactaaacacagcacaggggaacctcagagcacatcagagatgcccctcatcaccaccccgagattcaagacgagaactggactctgtggccatctgtctatccattcactcagtccttcatcaggaatcactgctgtatacggcactaggaatgcaaagcttatgaaggtagttctgtagtaaagaactctcagtttagcaggacagacagacatgcccacaagtgcgggacaagggtcaccggttctgtgagagtgtggcctggcccagaagaagatcagcctcatggaggacaaaggcagcatggtaaagctggcagggaaggctttttctttagggacaaggaggaaattggcagaagagtgaggaggtgctaagggcataggggcggcgtgttcagaagcatgaaggcctggtcaagcagaaccctagattctatggaaagtggtagaacagatggtgtctccttttgccattagtgacctaagagcaggtgggcagagtattccccaggcggctagttcagaaatgtgcaagaaacagcatatctgcctctcctgggcatttgacaccctgatcttgcagcttttgcgaatcagaccctcagaagaaatcactagaaccctactttggaatccacctggagaggaatttgcattcaggtcagccagcaagattttctcaataaactctttgggaatactctccctccagattttactgtccactcacgtaacgcatccagagagctgaggccagaggaagtgtttggattcgcctttccttttaatgtgtgatgtttgaaagcaccttttcacttagttctcacctgagtcattagacgtgggagaccaacgcgggttgagatcgagtccattgtctcatggtttgaagacagtttctgctgctaaactgcaaaggagttattcgttagttgagagtagttggttataattagccagcattggcaatgttaaatttataaatgagggcactgtagtgccacacattgaaggaggagataatgaaccctcccctagtttggtaaaacaatgggtgtaactcaaattaaagaacaaaagagaaacagtataaagcctacaatgaaatatcgtctctaattgggttttccttccacaatagcacagttgaaatgcagaagagtatcaacgtaacctttccatttgctgcctttgtcagccaaaaacagatggttaacatgatttagatttttgttcttattgtagtacttagccgtttaattggaatccgattatcaaaccactttgtaaacaacctagaattgaggtgtttgttttatcactgaaaaaaataaataaagaatcagttgggatggaacgtgtgatcgatggaactggaaaaaacactgtctggcagacgtgcggggcaagacagagttttgaaacagttttagttgtatttcagcattccgactaccacacggttctatacacggacaaggtttgtaaaatcatgtaaatattacttgtcttctgaatgaaacacgcaaaataaaattctcggtaaattctgtggaaaactgaagaaaatcattgaaacccataacttaatttgaaattactcctaatgacctgtaatttcctccttttgaaatagtcccattacattaatccgattgatgtggtggtgatagattattctcattaaaaggaatcttcttttgttgtccttgccaatctaccatctaagttctggaagcaggaactgttcagaaagctgtggttctatcccacccttcagtttggttgaacatggagccctttatcgctttcttccttgactcaagagacccatttgtagatgcaaataatctcgttaggtaccagcttcacacacagccagtttttcggggccacccaggtctgtgtggaatggtggccttctttgttttctagcattttctttcagcgggtgtgctggaaagcctgttccaacaaagggcttctccctttccaacgggctagattcatcacccagggttggtctttataaccatctatttttttttttaccaaatctgtaaagcgttatggctctggcacagcccgtggctccaccgtgcatctagcgtgatggctttgtggcacggaagcagggctgtttacaccctgacagccaaaccttcaatttttcatgaggtactagagaactaattgtgccttctcgcattttccccagcacactgctttcagagcttgcctttgttcaccgggtttccttgaatctgactgttgataaaaattgcccagcaaatcagtgaatctgggcggtgaaatataaaatcatattttatcatcttgtattccattaatgtttgatcagtttctgggctctggaagaaaggtctttgtttccatcagggccttctctagatgttgacggatgacttggagcaaatcgttcttaccttggggctgcctgtccgcaccctgtgagtgacaggcaggtaacagctggcctcctttgtttacctcttccctacctcgactagtgggtggaggatttagagggagagtgtgtcgctgtgttgctttaagagccaccagttcttgaaaggtatgcaagcacaggaacctgcattgaatttgttttgttctgccagtcctcagcttcgtgaccgggggcaagttgcttcacctgctttaaatccattttcgtattttaagtatgggaataataagatatatctcctcggattgctgtgaggattaaatgacgtcaaattcctgacatataagagttctgagaaactcttatgatagtattactttaagaggagactgaaagtagcaagtattttcagctttactctatctagtaaataacccccccactccagaacacaggtaaggttctggactgttgattctgttactcagggtgattcccttttctgaagaactggtagagttgctttcagttggatgtctcctggttagtgtctggttaattgtctattgatttaatagtctccactgattaggcggcagcagccaaggcggtgggactcagggacttcaaagctggaagtgaggggacctggtcttatgcaggagcagatctcctttcctctctgctgcaggccaggactatgtatattgtaggtgctcaataagtgcttgtgatgaaggtgttaggggtcggtatttttaaagttaacttttcactgaagttaactatacagctagatgcagctttgcagagtgaacccagccatggaagcaccacctgatggagaaatagggcgttgccagcatccaggagcccactctataccctcctggtcactgttcccccagaggtggccagcctattctgccctctttcagcatggattgagtttgcccatttgcacatttcatacatggaaattatataatatgtactttttgaggcttcttcctttttacacaactttattatgttcatgagatatatccatgttgccacatggagtgacagtctattcattttcattgctatatagtattccatcttgtgactacatcacaccttgcctgctctatagttgttagacatttggtcatattcagtttggggctatggataatgttccagtaaaactctgctcaatgttatgtggtagcctgggtgggagggcagtttgcaggagaatagatacatgtatatttttgactgagtccctttgctgtttaccctaaagtataacaaccttgttagttagctatacctcaatacaaaataaaaagttttaaaaattcttgtatgtttcttttggtgaatatgtatatacttttatgctaaataaaattacaatgcttatatgcagttttatgctaccagtttatttttccaaagtggttgtaccaattgatgctcccccagtaggctataagagaactacaactccaaaatctcccttacatattttttaaaaatttgtcccttcattttagttatcttggttggtttgtagtgggatctcattgcagttttattttgcatttctctgatgactaatgatattgagcactttatattgtttttttcggccacttgaaagttgtctttagtgaatgcatgtttgagtgttttgcccatttttcaactggttcatctgtctttctgtgattcatttgtagagtgcttataaagccccgacacaagccctttgtcagataaatgtattgtaaatatctcctcccacaccatagattgcctttcatttttgaaatgataccatttgatgaacaaaagtttgtttatttagtgtattccaactcatcagttttttctatgataactgctctttgtgtttagttttagaaatttttgcctatctcaagtcattaatatattttcctattttctagaaagtttgttatttactttcagagatagtagaattactactttttaaagaactctcaaacagggagttatccaaggggacatcgtaaggtgatgactctttaatttccaaagcagcaggtaacacccaacttgtcaggaactctagcatggcttaggtgggatgggttttataaagccctgttgctccacaaaggacctgaccctgaccaggtagccataggagattgctgacttttcacaactgtcctagtcttcctctgtagtaacccacttctgctctaggaattccagatagatttataaagggagagggcatggtgaaatggtagccgtgggtgctctgagaaagtcttaattttttgttcagaactccttttacttaccttgtctctgcatcctttgaaaatcctggcgtccatcttctaaccagttcccctggctcaggaactccatagattgctggctttgtactgttaaacagattacttaactgtcctgagtctgtttcttgatctttaaaattgggacaataaagaggacctcttagggtatttatttgttggatggatggatggatggttggttgattgattgttctgtccattacataagactaggtttataaagtactcagtccagtgttgacatatggttttgtgttcagtaaaagaacttacagggcttcccaggtggctcagtggtaaagaatctgcctgccaattcaggagacacaggttagatccctgggtctcctgccaattcaggagacacaggttagatccctccctccctggaggaggaaatggcagctcacttcagtattcttgcctggaaaatcccatggacagaggagactggtgggctatagtccatggcgttgcaaagagttggataccactgagcgactgagcacctggaaaaggacttatttttctactccctgcctaactccagcaatagttatttgttcaataataatgtcatcagcttagcttatctagtgcagcaggtactaatataagtttaaattatgtaagacgtccactttgtccatgtacagctttgtcctgtgatcatggtagcaaaggtgtgcgattttgagataacatccaaataaatgcataaaagacactgcatatttaaattggagtataagtgactgcagaaagttcttcttcacttctgcactttctgcagaaagcgaagaagaactaaagagcctcttgatgaagtgaaagaggagagtggaaaagttggcttaaaactcaacattcaaaaactaaaatcatatcttctggtcccatcacttcatggaaaagagatgggaaaacaatagaaacaatgagagactttatttttgggggctccaaaatcactgcagatggtgactgcagccatgaaattaaaagacgctgactccttggaagaaaagctatgaccaacctaaacagcatattaaaaagcagagacattactttaccaacaaaggtccatctagtcaaagctatggtttttccagtggtcgtgtatggatgtgagagttggactatagagaaagctgagtgccaaagaattgatgcttttaaactgtggtgttggagaagactcttgagaatcccttggactgcaaggagatccaacgtgtcagtcctaaaggagatcagtcctgagtgttcgttgtaaggactgatgttgaagctgaaactccaatactctggccacctgatgcaaaggaccaactcattagaagagaccctgatgctgggaaagattgaaggtgggaggagaaggggacaacagaggatgagatagttggatggcatcaccagcgtgatggacttgagtttgagtaggctctgggagttgatgatggacagggaggcctggcgtgctgcagtccatgggattgcaaaaaaccgatacgactgagtgactgaactgaactgatacctgactgctaccaagtgggtgaatatgctgttagagaggggcgtttggatgaacattgttaaatggtggtcatgaagatgatattgtgatattacatagcgtggagggtacaaaataattgcagtagcctcccagctgggattcctgcttcctctctggtccacctaacaccaccatgcccatccactgtccacccagcagacgtgagttgggtcttgtcacttccttgatgagaactcttcagtgatttcccgttgcacttaggatattatccagatgctttcctgggcttctgaatctgcatacctgacccttgccagcttttccatcttatttcctacctcttctccctctcacattgtgttctagacacaatagtctccttcctggagtctccaagtcaccaagcttggcttaatccttgagatctgagaaagctcttccttgacttctccccctatagttgggtggtagaattgattgtttctcttagcatgatattatatatctcttagcacgatattatatatcttgtttgtgtatttgtttgtttttcttgcttctctccctcactaaatctaagctgtggttcactcctggaacacagcagatgctcaataactgaataaataaatgaatgaccacctgtcatcagcctctgcacatcctggattctagggcccccagcctgccaggtggtcatcatggatctgttctcccacatcctaccctcaccaaccaccaggccggctctcaccctcaccaaccaccaggccggctctcttcccctttcactgacagtcacagattccgggcaggctctcttccttccattttgccataggagcttgaacctctcatcttcctggcagattcatcatttccattcaagtatcatctccagttccattttatccagtgataagttactgctccagtcccccattaatgatcataggctacctgcccttacaaagagccggtggttgtgacttaatacgtgcccttcccagtttggcctaatgcgtgcccctcccactttggcctcgtggtgggtgtgtgtttgtagcctccctgtctgaggggcttccttcatcagtagcataaagtatcaagcttcccctttgaaatgtttcttcctccatggtttcagtggttggggaaaacagaaaccaactccacctgcttaattcatagaggggactaggaacctgggtgtgtcttttggaaacctaggcactgaaggcccatttggggcttataaaagggtcttgcctcaatacccttttttggttttagcagtcagcggagagcactggaatcctgttcatcagaatagagaaaggaagaggaatatgctggactgggaatatgctggcctggggtgggggtgggagcaagaaggaccttcaggaatgcagtggactgagaggtgaggcctctgcctgaattcagatcgcagccccagcacctcgcctttctgggcctccatttcctcatctttaaaatgggaagaacagagtaggatgatgcgggtagagtgcttagttgacaccaggctagacatatagtcaaccctccataaatgttaactgatataactagtaatcttcagaaagctgtaaatattaattgatgtaaccaataatctttagaaggctgtaattgacctgcccccagtgggtcattcttggagcacttggaacatgttaagaaggaagattctatatgagtctctcttggcccctgggaccctgattccaaactttagaagaatttcccaggcaagaatactggagtgggttgccgtttccttctccaggggatcttcctgacccagggatcgaacctacagctcctgcactggcaggtgggttctttaccactgagccaccagggaagccctttaaaatggtagtggctttatttttataaatactaaaccactctcatatgtgacccacccggaggatttaagggacttatttttatctttacataactgggaatgtgcataagagggtcctagatcgggtcctttttttgaatgccctcaatgaacatgtcagaaggtacaacattaaggatgggctgagtccttaagtagattagtaatgaatcgacctggactgatatgaaatgcttggaaattataaagcagtggagatcacgtaccagaaaaagaatacattttctacttctcaaattatcggtgccttcctgcagggggacacgtgttttctcccttcagtgtgtagtaggcagtttgtgtgcacacagcttaataccccttcagctatgcattttctttgatcctgagcttattaaaaaatgcaaatctaaattgtatcttattaatgtgatcctttgtgttaaagctgtgggggttaaaaatcccagtttctctggcatatttggatttttaatgtgcaaagtgagacaagagcctgactatctgcaggccatattttctaatcagctgttatatgaaagatcttttattgtgctgctatttcttgctttcttggaattcagggagataaagagtaatgacttcattgttataccccaagtattctcaggcatattgagacccttattaatttgctataacttctccaacatgacagtggctcttgtcttccaggtgcacactggaagcatattttaaagatgcgtgtaacatgggagatggttaatccatataatttgctagtgactcccctaggcctgggcttcccaggtggtggtcgtggtaaagaacccacctgccactgcaagagatgcaagagacatgggtttgagccctggaggacagcatggcaacctattccagtattcttgcctggaaaatcctatggacagaggagcctggtggactgcagtccatggggtcacaaagaatcagacacaactaaagcgacttagcatgcacacatgctcctaggcttctggggcacgcgcgtggtgactgtgcccagatagacctttgtccaggtatgtgcagtcacccttagacccagtgggttcttattgttagagcgatttctttcctttaaaaaataccgtgttggagattttatttgaatcgagtcaaaaattgattccatgagaatagagatgcatttttctcacttgtcttatttctagtttaccagcttctcatcttctcatccttcctgtcttttctccatgtcttgagaaaaagctgcatcctcgtgtttaatcatttttttacttaagatcaaagccaattattttatagaagtcatcatatggaaatgctcattttctgtgtattagtctgtcagtaggtacccacagtaaagctctttctggaacaaggcaggtgatagtggagtcatggcaatgtggaagaagtaagcgaactagttaatgataaaactagaacaggcaacactattggcgggtctgtgtgtgaggcagacattatctcatttaatcctcacaacacctccaagaggttcctactctgatagcatgtccccatttaacagatgaagagatggcagaatagaggttaagtaacttgtccagaatcacgtgactgtaggggtctgggttgaaagtctgtgcccagagccactcaggttcctgctgtgggaagactgagtgcttgtcctgcttctgctcttagctttgctgtgtggctttttctcacctttaaatgagaatattggactagatgatctccacacagagcctttaagttttaaaattcaccataattattacaataaagtgctccaagcttaagccagaagacaaaaggcacaattctgaaaacatcctccccgcctcctcccctttacctcccaccaaataatttgcatcagtatcacttttgatgacagcacagtggaaagaattctagcagtcccatgctatgatatccatcgtttcatgaagaaactgttacacaactcatattccaaaaaaggccccgcgtggctttctgacacctccacggtggtccccaggaaacaagtatctggggaaggaacgttcgtcggcggggagagtggagggtctatttttaacttgagatctttttggagggttgcttcctcctctgtcacctccagttggaggaggcgagatttcacctcactgtcattcactgaggtggccatcgtggttcataaaggaaattttctgttgcttgctgttgaagtgttttacaaaaggaggtttgatgtgctctgtgagagcgtggcaggtggcagcttcttgtgtcaccctttgacatgtgctagctgatgtgaagtgacatgtggtgagagtcacgcttggcgcgggcgctttcttttgtgttgataaaaacaggtagcctttctgtggggagtcaggagcagggacaagagccggcctctcctggaagaaggatgtgtcaccgtgtgcatttttagaggcatctcagacactcaggcatctggataaggggaagctgatgttgcagatacgtttagtttgagtttagcaggatatatttaggtgtgaacccccagctgtagccagtagtgcccatgagcttgacctttggcgatggtcagctgtcgcaagagctgtcgggctttcagtgcctctggagccgtcaactctcatagctgtttctgcagggctctctgccacccttccccaggagacttgggaatgcaggcacattcagtgggctccatgttccccgccagtatctcttaatggaacatttcgcttatacaaacataaacagtaacataaatgtaatatacaagtacccaccaacccggcttcctcaaatctcaacattttgctgtatttgcatctgatctgtgtgtgtgtgtgtctctctctctgtctctctcacacacagactcagacagacacgcacatgcagataaataaatcattgcagatcccttcatcatccccagctccccagggacggatttgctgtgaagctaatgaaacttaaggtgacggtccctttcttccacaatctctaagagaggccctagcaaagtgtctcaataattatatatttttataaagctttgcaaaattcacacaagtgagagattttagcgacaactggtgaagattgcagtctctttccaccctgacctttcttccatcacatattccctcctgtgtccagaggatcagacgcgtctcagacactcaagcatctggataaggggaagctgatgttgcaaatacctttagtttgagtttagcaggatatgtttaggaggctcccagttacttcattgtatttttaagttacagcaagacatcccggtggaggcatgatggcacagcttccaggaataccccactgcccactgttcccacccactagcaacacgacatgagggcacaggaccagaagtcatagagtaacatggatgtgtcctgtggtgcccacacctgatatttgtgggtagaagaggagaaacagtttgagatgtatagagtcagaaactagcctctgaaaaaaatgtcacatcattcaacatgttaagttataagcaaactgtcctactttgatcaatcctatcctcacattggaaatactctattagaaatatatttgataatgtagcatacacagtgataaacacatcgtgctttctgctcaaaactctgtgttcatgtttcaaagagctcctcctgttgtgtatgctgtggcctccaagcctgtggtgtgcttagactctacattctcttcccctcttgatttggtgtttctcattctcttgcatgtttttcttttttaaaaacatatgtgtttgtgtgtgtacactttttaaaaaattatttctttatataaattgttattgaaagaaagtgaaagtccctcagttgtgtctgactctttgcaaccccatggagttctccaggccagaatactggagtgggtagcctttcccttctccaggggatcttcccaacccaggaatcaaaccagggtctcctgcattgcaggcggactctttaccaactgagctctcaggggcgaccccatggactgcagcccaccaggctcctccgtccatgggattttccaggcaagagtactggagtagggtgccattgccttctccaaaattgttattaattcagtacaaatacttctacaacttattttcagacattgttttgagagttatacattttgatacatattgctcttgtttgtttattttcactggatttatagtttttatatggggttccctggtggttcagatagtgaagaatcctcctgtagtatacagtacagtgattgaatctgctcaggttcagtccctgggtcaggaaaatcccctggaggaggaaatggcaacccactccagtattcttgcacaggaagtcctatggacccaggagcctagcaggctgtagtccatggggtcgcaaagtcagacacaactgagcgactaacgctttcatagttttcaaggtgtgaatatactataatttatttgtcctatgttctgtctatgggcacttgggttataatgatgacgacatggaagtaaacagcacacaatgaaaatcttattcatctgtgtgagagatgctctatagctgtgacctgtaaactctttttgctcggatatttcctaaaaaaattttgaaaaactgtgtaactcttcatacatttttaactggcatctaaaaagtatttcaatctaactttatgtagttggaaaggattaatttgcataacattgtaatatagatatttaaaaataaaactgaagattgaaaaagaattcaatgaaatgaaaatagtgtaatgatttgactctcactagcatccatttaaacaatatactattaagatatcctttaatagttagaaagtttatattatttcctttgccctttgaatttgaaattctatttttccattctcagaattttatcttgatgcagtatatttttatgtttgacagtctattattgataatcccatcatatttctctctcatattgattcagagaagttctaaatgatttttgggtatcattctgttgtcattttttgggtttaagtacctttttctagtcgtggcatgactttgttctgtgtgtttggatattttttgttctactaggttttaaattttaatgcagttagtttattaacatttcctttatggtctgaggatttatgtgtcttctttaaaaatttcttatcttaaagtgataagtataatttatgttttcttctaaaggtttaaagttttgtttttcatatttaattctttcatttatttagaatttatttttgtaaatggattccttagatatctaatttttagttttcttcaagggaatatccagttgtcataggactgtttttaagtaggtcaacttttgcctagtgacagttattgctaaattcaaatatatatatatatatatatacacacacacatacatacaaacacatatatgtgtgtgtgttttattccttgggtatgcttgttccttgagatttttaacatgaaagggctaacttgcttagctggcattattttgtagtcttcacaggtaaccagcactacagttaagcccatgcaatttgtttagctaatattcactaacttgacctaaatgtaagtgaatagtcacgtgcatttcctgtcttagaataacaggggcaaaatttaggcaacttagcctcatagtaaacacaataaatgttaatgttaaaatatgagtgaatgattgaatgagtgaattgatgaaggagttatatgatagaattcttaaaggatacttgttatagcttagattattattggtttatcttatactttaggatgcaggaaattgatctcattaatggacttgtctttctaggaagccgttttctcttaggagagcagtacttctcaaaatgtggccttgcagaatcacatggaagtgtgttaaaatgcacattacaggatcccaccccagatccactgaatcagactctgtagagtgggatccaaggtctccaggagattcttaatcgctcatcagaattttaagtcacctgtttagtgaaatgtcattcagaatccctgttcagatattctacctgacagagcagtgtcatttaggccagctcacctcttttagcaaatccatcacactcgcaggtcaaaacacattagagtggagaaaacattagcttaagattggatcctggagtctcatattcctggctttttctgaggtcatgtgacctcctcatgcatgtttcttatttccctgtgtgttaatttccccatctgtggctcttcctgctcgttggagatacagttaaattggccatgttcattgtaggggagagagagttaaactaggaccagaagccctgccagctgctatcttgtcctcggcccgtaaatctaaataacattcgggtagcagttgcaatgtacagatattttgttatgagttcagagcaaaatgaccctttctgaaatctggcttgggggtccaacctggaaaccaccatcactggcatttgtatgttgttagccactcaaacagttgtttacacagagtgcctgagggtcagtggacccatcgtgctatgctaaagacaaccaaagaattttattcaatccatccttcttaggagagtcattaggacttctaggtattaaaaaaaaaataaaagggaactgacagttcctgtttgtctgagaagtgctttgcccagtgtcctaaaacccagcctcaggaactagttcatcacaagtctccaaaatactcccagccttttctcttcacagtaattcaatttgaatgaacatacactcagagtatatgaaccgaacaactggttttaagcagggataaagctgggatcatcctcacctccatcctgaatggtacaatcatcttttatttcattctgctcggcagtggtggcaattaaagctcccaaagtatgaaatgttttcatcaagtcaggcattaaaaataccggcctgtcagagagggcagtggacctgtgcttaactgccgctattcttcagctgcaccagggcatggagtggtgagaagcattcatttcatgcagtggggaaataaggccaacttcggctttaatctttattatgaggaggcgagtctctgttggagggtaatgagtggatgcaccccatttagcttaaagtagcagatctatatagagttgttaatttgtagataaatacaggtttttttttttttgtaccatcgatgtctcttgatccacacaaagtggttatattaatactgaaacattattaatcaatagtttgctctttaaaaatacatcagtaggactacccaattacctttaagtatatatcaaagagcacaaagcaatctgatgtgtactcaaagtccattagaggccaccagacatattaattaaaatttctagttacaatgaactttacaatgtgcataaaaattttctgctttgagtaacaaataggtatgggctagctgaaaagctggagcttgtagatctatttgagggcaggggagggccttcgtggaacctgggaatgattttattttacttgaatccaacatataaagggctgttactcaggattgtgaatggaaagggacagggtcctctcctgtgtgtgtgttgaagggaagggaggaattctcataatttgaaattaagcattaccaaaatggtgcgtaggccttccctcatagctcagttggtaaagaatctgcctgcagtgcaggagatcggggttcaattcctgggtcgggaagatcctctggagaaagaaatggcaacccactccagtattctcgcctggagaatcccatagatagaggagcctgacaggctacagtccatgggatcacaagagtcggacatgacagcaactaaaccaccacccaaaatagtgcctgtgtgagtgctgagttgcttcattcgtatccaactctttccgaacctatggactgtagcccgccagtctcctctgtccataggattctccaggcaagaatactagagtgggttgccatgccctcctccaggggatgttcccgacccagagactgaacccacgtctcctgtattggcaggcaggttctttcactactgccacctgggaagcctgcaaaactggtgggtgcagtgaaacttcactggagattaaagacgaaactaaacactaagatgtgtgtcatccagcctagccgatcctgctgtgtggccaggcagtgttgcatttgatcaatctggttgtggcaaaggcccacaatgaatgagccccacagcagggtctctgccctgcagtagctatttgtactaatggacacctctgaatttgtactgagaggtagataatctaccattttgtcatctaacaacagtagtgagctttcctttcagacttcggttcttactgccaacagcccagctgtatctcctgtccaaggagaagactgattgagaagactcaatcagctaaactatttaccagactggcccccaaatcacagacaatctaggagcttaaagaaaaggaggagagaaagccaagacatgagaatgctgtttcccccgcctcccctgccttgcccacaccacccccaccctcccccacacacatctgggttctgtgcatctggttattgcaaggagagagagcataagctcttaaggcccaggaatgggatttacagtaatatgctcactaaacaaaactcaacaactgagacctggatgccctgaggaatgtgctgtaatggttctggactctatagcctcgtacatttctaatgactttgtcagaaagtggcagcctcatcagatctaaggaggtggcatttagatatgagatccatatttaagcatcacatacccgagcatcgctgatttagggatgtactctccccctaccccttggcgtctgtccctgggccgttatccagcacaagtcatccaccgagactagtgtcaggtgtaactgattgtcattcaacgcaagtgaaatctttaggtggatattagtcactgctttagacatgttagcttcaagtggaagcagtcaattacagaccagaggttttatggctacattattaccatcatacgattaggttttcagcagtagcattctgtctgatcgatctgttcaaggacattgatggatcactttgttcttcaaatgtcaaatatgtgggacttctgtgctttatgtatttactgacacgctcatctcctgtggataccttttcccctctgtggtgttcagttttagcagatgattgccatccctttccttcaaagacagtgtggattatatgtaatcacgtttataattttcttcctattctctctccagtgagaagcacttctgagttaacagtactataaagaagtctttaccacactaggaggatctctcattggtgtcaagaaatagatttagacaattttatttagctttctaatatgcagagattaattcccaattgcagggtataggttagacttccaaaagtttgattctctgttattatgctatcatgctatgtttaatttttgctttttttttaacaaatgtattcctaagtcaaatctccaaactgcataagccaccactatcaccttcaaacaccagtaaatatgttcacacttttaatattgaacaggatttcttatgtgaactccttgacgggctcatagaacagagagggagctactttcagtaaaactgttaggtcttaaatcttaccctgaagatcatcagtcctatgcaatagataaaacaaaaactgggaaagctttattccccaaaaaatagccaactgggcttccattttgttttaagaacttctcagagtgcacacgatggtcctgggaatgccagctttcttactgagtcagagcaatcccatttctactttaggacttatagttctaccatatttctctttatgtttattttgtttatttggctgctcctggtcttagttgctgcatggcacatgggatcttttagtagcagcacacgtgatatactttgctgaccggggattgaacctgggcctctggcatgggagcgcagagtcttagccactgggcctctacggaagtcccctctaccaaattttctactttaggaactttcccaactctcttgggtcaattttattagctagtaccacagggagaaaaccgtccagtttttcccagtaagggaggcaactctagacacatcaagggaaattctcaggagttgttcattattcatttttttaaggccaaccgtgtgtttggcaatattgtggtgtgtgcttagttgctcagtcgtgtctgactcttgcaacctcatggattgtagcctgccaggctcctctgtccatgggactctccaggcaagaatactggagtgggtagctgttcgcttctccaggcaatcttcccaatccaggaatcaaaccctggtctccctcactgcgggcagattctttaccatctgaactaccagaaggctaaatcacagacctaaaaggtctggctttgtgtggttggctcttcttgtgtcttgaaacaagattttctgcaacacagagcttcttccatgaatgcaggggtgtttggggcccaggccttcctcccataaagcagaaattcacagctgcttttcttctctctatccctttcctaccagagagcagcgtggctggccactctgcagctggcctttctcacttctgttgtccctgaatgcccttgcaaaagggtgaccctgtacctctggcgtgtccacgggtgctgaggacagcattagcagctggcctgcaccctggcacttacgctgaatgtcattctgatctctccaggcataaagttgatggaatcatcttgaccccagcactcacatccagcttcagcccagggttaaatgtaaccatttgagtggccatttcttgaaaaaaaaaaaaaactagctaatacacaaaactggatctcttatcaaaaaaggcacattttgatgcttgaagaagttttagggtagaacacatgtattttgcctaaactgtatctttttttttcaggaagggggcttccctggtggcctatatggtaaagagtctgcctgcaatgtgggagacctgggctcaatccctgggtcaggaagataccctgaagaagggaaagggcacccactcccaggtggcgctagtggtagagaacctgcctgccaatgcaggagacattagagatgtgggttggacccctgggttgggaagatcccctggaagagggcatggcaacccactccagtattcttacctagagaatcccgtggacacaggagcctggtgggctacagtccacagggtcacaaagagttggacatgacttgaagtgacttagcacacacagtacctgttttgatgaagtgcatgttgtttagttgctaagtcggnnnnnnnnnnnnnnnnnnnnnnnnnnnnnnnnnnnnnnnnnnnnnnnnnngttgtttagttgctaagtcgggtccaacttgcactagagttggacaccacttggcaacctcatggactgtagcctgccaggctcctctgtccatggcatttcccaggcaagaatactggagtgggttgccatttccttctccaggggatcttcctgacccaggaatcgaacccacaactcctgcattggcaggtggattttttctttttttaccactgagccacccgggaagctctctgaagcacaaaactaagtggaaatatcaggttctaggagctgggattgctttcattgagcccagtgttcttcaggaacacactgtgtgtgtacatcagagacacagccgctctgtagctgatacgctcctgtgagacagtgaggaaggacgcgttctagaagcagcctgagacttctgggttaccccctgcctcaacctgcctcagcagccttgcagcatcccatggatgggaaccaggtgggagagcacctccagggaggagatgtggctaggtgcccactaaccaccatgcggagatagctgtatttactctgccattcctgcaggagacagagctgcagattaagttgcacagacacaatggaggtcctggctaaggaattttaatgtaaacattcccgggttttaaacttaatccttgagctcactgatacagtcattcaaatggggatctaaggaaagttatgctggggctcagaagggtaaacgtgtgaatgcatttggaaccaatttaaagctagaaatgaaatgaacaaggacgcttgctaatacaccattaaaataaaacccgtaggttcactgaggcacgtacatagacggacatacacgcccattctggtcctgctgtctgctttctctgcagctcatgagaggttgtgggggacatggggtgggagaggccttgtcttagcccaccttagaagtatccgggctgggactccctgataaatctattctgggcttcattatgaagttacactggaagcacagtcatggaggatcctctcctcaacaaggtcttcatggaatgaggcctgaatgagccacccactggaaggtacctcttggggactgcactcctgaaaatgtgaccagaaccaaccagaaagtctagatgtcctaggaaaataaatggaggtgcaggtggatagtaaaaagaaatgcttaactgactgaagaccaaggaattccaagtctgaccattggtagtacctgctcctgctgctgctaagtcgcttcagtcgtgtctgactctgggagctttataaaaatacagattttcaaaaacaaaaaaacaaacaaaaaaaaaccagattcttgggaggatagcacaggaaaaacttaggaatctgtgtgtttggggctttttttcccccttatttcttcaagtttgttctccagaggattctgaggtccctgaggtttgtcactgcttgaagcaaccaacttttcaaacacttaaaacctgttggcaaaaaagagagaaggtgtgctaattttcattgaaataggtcacctaagaatctttaaccaaacaaaattttattaacccaacttgatatgaaacactttcttgaataaagcaaaaacatcatttaaaaatatgtatataaataatgatgagcttccctggtggattagcattaaagaatctgccttcaatgtaggagccacaggagacatgggttcgattcctgggtcagaaagattcccttcagaaggaaatagccacccattccagtggggaattccatggacagaggaacctggcaggctacaatacagtcgcaaagagttgggcacgaatgaagcaacttagtatgcacgcatgcatatagataaaatccagattcacaaatccagttaagatggacgtcccttctccccatcattttctggaatgtggctacactaagtctagacatttctgcccagctggtggctttctctccctgaaaccagcttcagcgtctggctactatcttttgagaaagcacaccctgaaaatacaaaagcctgccattattattcatgcggcttttgtttatcttccccaaagaaacaattgagctgtagcagatgagtgatggttgccgggctgtggcagaggtaaaagccactgcaggtggagtggcttaatgaggtattaattgggaagcaggtgaaggcaaataggtagcacagcaggtatgtaaataggctcgtgggaagaaaggcattggattttatgctctttaactaaaaataaagacctgatatttagcttatctttgctgaaatcctgcaccgtgagggccctgacaaacgccaacatcatcaatgattcattattatctcagcatcgtccaggggacttaataggatcttcagcctcttcatttttcattgtttcagctgattctttggctttagaatgcatacttttcttctctgtttatatatcagaggtcagaaaaactgttatagattgtatagtgatgacgacatttaagcctccagcctgccttctggtagcatccttttgctgtgtctcattggagaaatgcagcacatcggtatgagtatggatagtaaaactaccaagagcacatacatccaaattcatcttgtccttccaatagtcacctcgaggaaaaacacttactccagggctctcattggatcaaaacctgtgcaggaaaaccacttcaatggtcattttcatgaccaatttgtaagtgcccccatatgaaaaaattcttacatatatttatcacttattttcttcccctaaacattacccaatggcaccccactccagtactcttgctggaaaatcccatggacggaggagcctggcaggctgcagtccatggggtcgctaagagtcagacacgactgagtgacttcactttcacttttcactttcatgcattggagaaggaaatggcagtccactccagtgttcttgcctggacagtcccagggacgggagagcctggtgggctgccatctatggggtcgcacagagtcagacacaactgaagcaacttagcagcagcagcaaacattacccagcatcatcacctatttcatgaccacacttagtgctatatgacatttggcttttaaaatccatccctgatgtagaggttcgctgctgctcagaatattccagagaacacgcctcctcaggcttctagggaagagttccatgaaacgtccagagtgttggcagcctctttaggataagtgtccagcttctcagagcaaagactttccatggcacacacttagaaatttctaaatttgggcatgctagtttgaaagttcaggcccattattttaaggctttcttattttatagtaactcagaaaactgttaaacaaaccactccatgtttcctttccagttagctctctttggcatgttggtggaaaagtgatgggaaatttaggttcttggttgcttggttggcccacgtttattgttttctcagggaagaagcagcagtagttttgaagaagtgtttagcacttcactgttgaaacgactagatatcagtgaagccttcacaggctaaatattagtggaggcacagtgtataccccgtaaaatgaattaggtttacattatcatacagatcttttcctgtagttgaagtactcattaattttactgctaaagagatgttagtaattaaatacctctaattaatgaaaatcagacaaaagcaagggctgctctttgtatcttcaaagatagagatgtttgtagatccatctcgttgtgacttttgtctgtagcaggaccacagatctgagctagttgggatcaagtgtttgggtccagagaaagtcatttaaagactttgcttttggaaaattaaaggagtaggcatttgaggatgttataaagatgatatgagattcgggggaaaaaaatactcataagtgagagtcagtctacatgtaatagcaaatttcatggttgaagatacaacagcgttgaagacaattaatagttctgaagcagtggctgagttctggggtaggccctcgaacacataattagattattaaaaatggcctgtcttaggctgactattactaaattgaaatccaattacggctttatgattttgctgaaaaatatttagctgttttatttgaatccttgtaagagatcatttcctctttgcatcagcgtttgcacatgtcttaatatattttccaaacaaatctgacctcaattatcttggaatctatcgtacattagataaaatatattttaatgagtgtttttatgtctaatatcatacttgtgcctctccacacttgaggaattgaaagatggcagtagaagactttgcgagtatctttttaatgagaggcatgcatattttagaaataactaaattgtgccatcttaatacacagaaaggcattatgttcaagtatgtgcctgagtgtgctgcaacgcagacttggttcaattctcagtaattggcattggaactgaaataaagcagttgtttgcaaagagatgggcttggaattttcatgcactcctgcagtgtggatttaaatgctaagttaaatatttatgaacccgagatttttataattgatcattgtatgatttatacacagtaattataattaggttgaaagagattttaaaaagccagtttcctggtagccaaggccattaaataacgctaagctgccagaacaaaaattagatgggaacccagccatcaagttccgagtgcctgtccccaggtcccgtgtacaatgaggcactgcccctcaccagccacctcacctgggcaccagctcctgcatcacctgctatcgtttttcatgcctttatttcaaagctagttgaacataaacaagtagaaaacttcagagatccaatgtgtaacaaaaggaaaaggtggaggaggttggaaaggttaattaaaaatgtaatgatcccatttgctgagacagcactattaatcattttgcagcagcaagcatgtgacttacttacacaccacttataggaggcatctaattttttaagatatggatggtgtggagggtgtgggccacagtgtgttctttatggaaacagaagctctaatgggtgaacttggagtgagcaagcatggggcgagtgcctgactcgtggttaatacagggcactgtcttcaccaggcagaagaattatatccaaattcacagatattcagaataattcagtcactgtgaaccctagagagaaatcttggatatgacttgaggcttaatgtagttgtggaaggtgtgaaaaaacagggcaaggaggagattcatcattgtgtaatacaatatgtctcaaacggtgtatgaaggacaaaatgggctccaaagagccaacattgccttttaaatgtaatttttagcaccccagctgaaaaaggccaagctcttgtaggatcgattacaagggtcacagcctgtacgatcagatttgccatctgatccacgtggacagcagaagaccagactgtccatgtgagcttccagaaaagcctgctctacccctttgaagagcttggagctttttgagtattttttatttcctgggataacacgttagagggagcctattaggagaagaacagtagttatgtcaacatggtaattaatcaagccagaaagtaagaggaaactggttgtgaattcaattctgcagccttaaaaagaaaggcagaaagatacccggaaattggcaagcatttgtaaacaacggtctttcactgtgtcttatgaactttctgcttaataaaaacgcatcttcatgcgagccatgcaaattttaatatagttactgttctattccacctcataacctatagaaatgatttctagatccttgcatctgtgtttcatcctgaatcattttgactggtttgctggtctgagcatttgccaataacggtggcgttttctaactgtaaataatgaagggctgccagacacagcgatagcctctttgatttgggacagtgtgaggattgtacttgagataaaggcagatccttgcgccagaggaatttagaatgtgaaaacgtctcccagtgcgtaattcatcctcgacacctccacatctccctgtattgcagggactctttacatttgctccgtctgaaggtcggggtggaaatcgttggttcgtatactcttcggagcagcaaacagctgagaggtcaagagctggcttggttttggttttcgttttttatcttccatccctgatcaagctgtatcctaaatactcgatattataatcagaaaaggcaatttactttataagcatcaaataaaaaaccctgaggcttgaagggaccttagatatatcatcttacaagtggggaaactgaggcccacccaggaaaagcactcaggtaactggaccatgtttcaaccccctgactctgaattcattgctcctttagcaacatctttttattctatggtctgggagctggccctccagttgctgtgaaggttatacctcccaagaaaacaggaaacaagtcacatcagatgtgatcacagccagaaaagcttagaatgatgacagagaaagccatatgtaacccgcaaagcaacagcggggtgatgtgtaactttgaaaggaaaagctgggtaatgggcttctccatcacacagtgcccggcgggtgctggagggagttctttagcccgccaaacccacatgcacacgccctcccccagaagaaagaatttctagtaaacagccttcattctgagaatgtttcccattcagacagcatcccagacattctaccgagttaaagaatgagtaacagcagatggagggggagattagagttggaggaggaggggggtagcaacagtttaatgctgagctctaaaatgagctgtggagaggatggtgctcagagttatggtaaagctgttccgaagccctcccacacaggcgagaaggtgcgcgtgcccagcccgtaaaccaggcgcgcgcctgtgcctccgccctcccgtgtgtgagtggatggggtgttgggtgtgactcagtgccctaaatccatgtcttctggccacttctccctctgccctcaaatctgtgaggtcgcagtgttgtgcactgggaaatgcatttaatacatatggggcatcaatcttcccaccaagtaaaagctcacttctgactgctatgtcctgtgatttcacctctgcttattgtgaacttcactgcagcagggtatctgagagagagctggcagtgaatttgtgaggactcttaagagggaggtgaaagttttcggtgctaagggttgttgttttccatattctgtgcatgtgagacttttctttaaaaaaaaagaataaggtatatacatgaatcaacttttttcagtgttttgtttgcacattttcaaacatacagtcgccttctagaagctaccatgacatttgaatgtagttgctctgtaagtatccatccacccatccttctgtgcatctagcagttcatcaaagttttaaaaatgcagatatcaatatgcttcccctaaatgcttcaggatatttaccattagccagtttttaatatttatttttctttgaggtaaaatttctgtatgtacagacgttgtgtcccattctgagttttgaccaaaatttctactgagatagagaatattactgtcactccagaaagttcctcttgcctttttccaatcagcccccacctccatcccctagaagcaaccactcttcagattatttttcaatcataaattaattttgccttttccagaacttgcaagtatatgttgagttggataaaaagatgttatggaaaaaaaaaatccaaacgaactttttggccaacccaaaattattcagtgtgtacttttttcgtaaagcttcctccctcccccttagaacagtgtttttgagatttatccatgccactgtgtgtattaatagttcatctgttcttatttttgagggttattccattatgtggatagaccatggattgctaggcattttcttactgatggatacataagctgtttctagtctttggttgttatcaataaagtgctgtcatactcttgtcctaatatttttatggacatatatcctcatttttgggggtgaatacctagaagtggaaatgctgggctatagggtaggtatatgtgtaattttataaaaatctgcccccagctttcccaagtggatggacgattttactttccttttggaatgtatgagatttcaggagctccacatctttgtaaacatttgaagttgtcagatttaaatttaaccaccaatatagtggttgtaatttgcatttccctgataactagtgatgtttgacactttttatgtgcttactgaccattcatacaacttcttttatgaagaaaaacccatttttcaattgggttgttttttatgttgggcttcccatgtagctcagtggtaaagaatccccctgccaatcacccctgcagaagacacggactggatccctgtgttgcagagattcctggagaaggaaatgtcagcccactccagtattctttcctaggaaatcccatggacagaggagtctggcaggctacagtccgtggggttacaaagagttggacatgacttagcgactgagcatgaatgcaaatatgttttagatatttgtcctttgtcagatacatgttttgcaaatattttcttctagtgtgtgatttgtcttatttgttttcttcatttcttaagaaattaagtgtcttttgatgagtttttaacttagtgaaatctaacttatcagtttatttaatgttgttgttttttgtactcttttctaagaaatatttgcctgtaattttcttcttgttttgcattagattctactatttgggtaatgctggcttcatgtatcaatttgggaagggttatgagaatgttttgtgtgaatgagaattagaagcaaaaagtgactcatttacctgtaagctggaaatcagttagtggcctcttgcctatcgtaatgtgccaggttgcacactctggaatctggctgaaatgatgataaggagatggtgctctagcagtgttatttatcagtgaaaacagaatcagctcaaagtgaggctgaagcatgccaggtgctgaatggagtgaactaggagccattggtttggacataaaatgattcatcctattcccacttcatgcaaaacttttttttggatttcacatataaatgtgatcagacgatatttggctctgtctgacgtattgtactcagcatagtgctctccaggttcacctgtgttgttgcaaatagcagtatttccttccttttatggctggatagggttccattgaaaatctacaccacatttttgttatctgtttgttccttgatgaacacttgactattgtaaatgatgctttagtgaacatgggagtgcagatatttctttgacaaagtgattttgtctcctttggatatatacccaggagtagaattgctgaatcatatgatagctcgcagtcgtgtgaaaatgttagctctatttttaattttttgagcaatctttttactgttttctctgttttctgtagtgactgtaccaatttacagtcccaccaactgtacacaaggcttctcttttctccaccatctcaccagcacttattatctcttgtctttctgacaagttctcttgatgacagttattctaacaggcctgaggtgatatctcattgtggtttgattttcatttccctaccaatcagcatcttttcatggacctgttggcctttcatatatcttctttggaaaaatgtctcttcaggtcctttgcccatttttaaacttgagttattattattattgttgttactttggtgattgaatcatataagttctttatatattttggaaattgactccatatcagaacatgatttgcaagtattttctcccattccataagtcaccttttcattctgttgatggttttctttgctgtgcaaaagctttctagtttggtgtaattcatcttgtttattttttgttgttatgggttatgtcatacccaaaaaatcactgccaaaacccatgtcaaagagcttttttccaggccaatttttaaaaactgtattatatggataactgccagagagaagtaaattttctggaccttgggtgttaagtttaattattttcattataattttatttgaagtttataaacataaaaccatcagctcttattctttgcttttatacaaactgtaaagacaacacacacaaattaagtgttaacaaaacattaaaaactatgaaatgtgagttgataacatacatgtaatatgagaaataatactgtctgctgaaacttacagtttgtatgtaattctctgtttcagcatgtactgttttgagtttgacagtttgagtttgacaggcctgcacttaatttttctgtcatttttctccatttgaacttacatgaactgtttgttcctatgtccctggctacatcatgtaagtattaggtcaacctctgcgcctgtattcctttcattttagccagagatcataaagtatatccacctgatgcgaagagctagctcattggaaaagaccctgatgctgggaaagattgagggcagggggagaaggcggcgacagaggatgaaatggttggatggcatcgctgattcaatggatgttagtttgagcaaactctggaagatagtgaaggacagggaagcctggcatgctgcagtccatggggtcacagagcgggacacgacttatcaactagccttaagggctttatccggatgatccagagtttggtcatattcctgtattttagatcatcattccaaaatgcataacttagaaatctccaagacttcctttggtagtccaatggttaagaacctgcctgccaatgcaggggacctggggggttagatccctggtctaggaagattccagatgccgtggggcagctaggcccatttgccacagctactgagctcacacactctggagcgcacgtgacgtacctagagcatatccccactggctgcaaccagagaaaacccaggcgcagcaatgaagacccagacagctttaaatgaaaataaataaaagaaatctacaattgtagtccttcaggattatccatcactgctgccagcgggcccaacagcaccacctctcaaaagaggaaaaaaccagaagtgtcaccagggcctggtggaggccctggggatctgttggggcatgtggtgaatatttcactccttgggtcctgaaatgcctgagtgtgaaaacagaggcgcaggagcgaagccactttgggaagatcatgatggagaaagtgcttcaggtgtgcttgttaaaccagcaggatgtgcgtgcagctgctggcgtcctgcactgctcacctaagaagggcggaaggtcttgtttatagccttgcacgttaaattgttgaattcgcatgtcatagaaacatgaacccggggcatcataagatactgctggtagtgcccgagtatatttttgtgatttcagatatttacacatagttctttcttaaagtgaggcacaccatcagtagaccaaagagaatttttcattccctgaaatgttggacttattgtgtctgttcttgtataaaattttgctttataacagtagaatatttaacatgagatcccggtggctcacatggtaaaaagaatctgcctgcaatgcaggacacctaggtttggtctctggattgtgaagatcccctagagaaggaaatggcaacacattccagtattcttgcctggaaaatcccaggcccagaggatcctggtaggctacagtccatggggtcacagagtcagacttggctgagtgactaacactatattcttgacatatttcttttttaagttttattgatttacagtgttgatttatgatgttgtgtcagtttccatacacatatatacattctctcccatttttctccaccatggtttgccacaggatactgaatatcgttccctgtgctatacagtaaggacctcgttgtttatccgttctatatataatagtttgcctttgctaatcccaaactcctgtatcttctctcccctaaccccactcccccttggcaaccacaagcctgttgtctttatctgtgagtctgttattgtttcatagatatgttgatttgtattgtattttagattccacatataagtgatatcatatggcatttgtctttctctttctgacttagttcacttagtatgataatttttaggtaccagtggtcagaagacctaaatagacctttctccaaagaagatatacagatggccaatagatggatgaaaagatgctcaacatcacattaagagaaatgcaaatcataactacagtgaggtatcacctcacatcagacagaatggccatcattaaaaagtgtacaatatgcaaatgctggagaggatgtagaggaaagggaaccctcttccactgttgatggaaatgtaaattggtgcagtcactgtggaaaacagtctggaggttcctcaaaaaacctaaagatagagctgccttttgattcagcagtcccattcctgggtgtatactcagatgaaactatcatttgaaaagatatatgcacccctatgtttattgcagcactatttacattagccaagacatggaaacaaccaaagtgtacatggacagatcaatgggtaaagaagacgtggtttgtatacacagtggaatattactcagccatcaaaaagagtgaaatcatgccacttagattttaagtgcacaatatggtattgttaactgtaactgtgatgctgtacagcagatctctagaacttactcatcttgcatcagtgaaactgtatacttcttgaataacaactccccatttcctcccttgctggaacaagggaggaaatggggagtaggaccacttatactattgagtagcagcaactatcatttgtactgttgaacagcagtaactaccattcaactctctgcttctatgagtctgagagttttagatgccttgcacatgtagagtcatagaggatactgtcctcctgtgactggtttatctcacttagtatcatgtccttaaggctcattcatgtttttggacatggcatgtttccttctttctttaaggtggaaaaacatcccaggtgccctttatatggacactaaacgcactgtcctgtctttcggtcccatccagtccggccgaactctctttcctctcgtgctaattctcaaatgccttcatggtccccatcaatgggggacataaagagaatgaaagggggtttcaggacccaagaagggactgaggcctgaatggaaacttcctgggctttgaatcccacagttgtagggtgcctgcctgttgcctcctagctgtctccagagggtcactgctgcacctaattgttaattttccatgggccatattatatataacttggctcttggaggtttttccatcactttcccctctctttagtaagacctatattgtgaaaaataggcttcgtttcccttgtccattcatttttaactttgcccattggacttggccctgccaacccacccagctaccccagtgggccccacctcctctgtgttctgtctccagcctcacagaaggcctcctggccgtggatggccctggtttctctgaaagcctgagcacagagggcttgggcagaaggcaacctatgttttgccagcccacccagccaccccagtgggccccacctcctctgtgttctgtctccagcctcacagaaggcctcctggccgtggatggccctggtttctctgaaagcctgagcacagagggcttgggcagaaggcaacctatgttttgtgagatttctaaggcctatggtttgtaagccccctgaaattctatccaggatactaactgaatttttctaatctgcaacctcagtactcatagatcccacattttctatgtggtaagttaaatggtaattgaggtttgcaaatttttacataatttatgctggtttccttccagtggcctgtgagctcaaacttaaatacagcttcgatgtagtggcttcttcctgtttagagagctggcataatattttgtcttctgggctcaaataaccatgcaattactctgtaatcatatagtaattagcatgtgctatgtgcatttattacataattatattcctggaggatcgcatggcaattatacttacattcagcatatcattttatatactatataataaacaagtatataatttgctgcttttttcttttatcctaatttcagtttaccagccaggagctggaaacttgttctcctgttgccttgtttagacccaggcaggcaggtgtctgagctcctttggcctcactcagtacctcctaattatacctctgagacaatgctaggagcttgtgtgcagcctcgggctcctagcctctgtgcccctgatgcttcctcattgtcacaaactcattaagttattagcttcaaaacacagagctgaggagagctgccaagcctgccaggtggtgagaggccaggtctccgtgcaagcagaggacctgtgcaagctgccccggcacccagccctgtggtcacctgcctctctctcctttccgcagtggagcacatctcgaaagggaacaactgcctggacgccgccaaggcctgcaacctggatgacacctgcaagaagtaccggtcggcctacatcaccccgtgcaccaccagcatgtccaatgacgtctgcaaccggcgcaagtgccacaaggccctccggcagttcttcgacaaggtcccggccaagcacagctatgggatgctcttctgttcctgccgggacatcgcctgcaccgagcggaggcggcagaccatcgtgcccgtgtgctcctatgaggagagggagaaacccaactgcttgaacttgcaggactcctgcaagaccaattacatctgcaggtaagtgagctggcagctgcgtgatgaatgaggtggcggagagaaccttttcgcggcttcagtggcccggtttctgttttctcaatttctgcgtggggtgggagaggggagtggttgggatggatggggccaaaactgacctctgcctccagtacaaggtccttcagaaactggtcaggcatcaggctcttgcactcaggaaggtttccatctttaatgtgagaatgagggaatgagggaaataggactgtgctcagtaagtttggagagtcacctgaggtcaggtgagcttgaaggcatgtgccctcactactagaactgagatctgccaggggtccaaaagtgttaagtaagggggatgagggctgatagaaatataggggctctttgcatcaagatcacaaagctggaagacagcagcaagagggtggagctgtgtccagccctgaagagaacttccagaatgcctaggtctgttctgggccatttaatcatcaacctgtctgagaatagatactttccatgcttctggatcttagccccgatcagagacctggcagagtctgggctgtgttgccgaaaacaggcacttgcttttcctttttggccatgtattaatttattttaatgtttcggggactccatgaatatattctatatatatatatatatttttttttttttttcagagcaaaaaagctgtagagaattaagtagaaggaaaggaaaggtttcttttctttctgttctcacttgctccnnnnnnnnnnnnnnnnnnnnnnnnnnnnnnnnnnnnnnnnnnnnnnnnnncattgctgatggaagccagtagattttgattagcagaggctcgagtcttcgcagcctgaggcaatgccggtgctgcattcagagcacagggtggatgtcaggttagagatgcagacgggggcatcgtcggtcagcgcctgtactctgcccccgggacagagtcaggagtcttcaaatacggtcgctgacaaaatcccacgcacttgatgaatttcacgggcggacaagtaggtgaaggtgtgactctatgcttgttttcttgtaagtttcccctttctcaggagcagaaggcagagaagaatctgtctgattagttgggaggattctggtgagggaaggttctggtcggtttttcttctgatagactggagtcccattctttccttagactagctctaatagtgaccaaaggaaaatagaagcgctcgttaatcgtagaacccccctcgtgctgaagactttctggaagtagtgtctccattttttttccaagacggaaattatatgaggaagaactcaaatgactgagctcgttgtgggagaagtcaggtgttcaggcagtgaggcccttcctcacctgtcacccctgtttcagtgagaccctcctagctgagcgctgcgagcagggatggctatattctaagggtcctgaggaagaggcagggcctccttgcagccataacctggggaaaccagtcatcccgggactctgagaactctctagtgctaagtcacttcagtcctgtctgactctttgtgacccccatggactgcagcccgccaggcccctctgtccatgggattctccaggcaagaatactggaatgggttgccaggccctccttcaggggatcttccccacccagggatcgaacacacgtctcttatgtcccctgcattggcaggcaagttctttaccactgagtgctatgaggggccagaccaagggagaaactgcggagcttcctctcggattagaatgccagggatctgaaaatctaaaaattatcacctgaagaccctgcacccgaatcccatgttcctggtccccttcggtctttggtttgagcagaaggaatccttttgctaagagcaggactttccagggttttccttggctggaagccagctacagccatccgtgtctctgcaggaaacattaccttttcactgatggttcaagccaggaggcctggagatgggaggctgctataagtggggaatggagcagtgagtcagccctctgtccactgggccacggctgggaggagggagagagcatggtgatggagccttccctcctcttctagcaaggtgtgtcttgcacgagaggtccagagggggtgcctgataccccctctgcagcctgaaagcctattctgagggttcgtttgcatcacttggcctgccctatgaaaagagagactctttggagggttggtgagaactagtcaaatcagattgcttccctgcttagctgcagtgagaaacagcaagaagtttgggaacttgcgagtgttccgaggaggaggaaggaagaatctgtaggtaaggggatggtttgggtactgtttagggaccaggttggctccagggaccatgtgtgttgcggcagccccggtggcatggagtgacttcagctgatggagtgacaaggaacaggggacagctggacagccctcacctaaggactgactgtgcccggtcactccgaaggtcgggctgtcgtgacagcagccaccactggactggaagtgacaaccagaagccgcttttgtgcgtctgccgtgtttctggctctgggctgtttgggagtctggatcctagactgttgagggccctaccaggggaaattggcctcagtgtgattgatgggttttataccctgtcttgagtcctctctaggaaggtgattcagtgacttcccctggtgttacctgtcgttatttaggctggaatttctttccctagctgagcctggacttattactcatgattaccccctccctgtcgcatgtggtaaatgtaatttaagctgctaggtaggctggcagtgttttctttcatcaggttttattaatcttgtctttaattttatgccttttattgcatccctgctcgatccatctcggtgaagccattgtagttactcctacccacagcagcagctggagctttggcggagggggattcctgttaaagtagctacaatgttaaagtagctctgacctagatccactggaggaagggaggtacccccagtgggatggcgaaaggcccaatgtgaggatccccaaagggaagggatgacaagaggggaaatcacttcctaaaaagcatattctttgaaagcttgcctttcaaatgctccattgccatctcaacatcagccattatcaaagagctgctgaagctcattaaaacttccctgcacttaatgttttccaactcgaagcacacaggtttccttccccagggatcagcagtgcttctgcacaaaacctgtctctccggccttacgccttcaaacagagcagatgcaacttcagcgttgccttttgactttatttgcctttactgccttccccttttagagaagccgacaggtttttttcctcttggagcggcaagggagaggcgagagcttgagtgtttagtcctcctaagcttgtctgcagaaaagcaaactgggaggcagtagcaggaagggggtgatgaaatgtaacagcctccaggtcttaccctgaagcgcagtatctcccttgccagttttctctggttgtcttccttagtgcctggaagaatgtcaggactgggtttgatcctaaagggagccgattcctttatccttgatgcacctggttatatgtacaaaatttaaactggcaactctcctccttccccccaaactctgacatactaagtaagctcagcacttggaccctggctcagttcagttcagtcgctcagtcgagtccgactctgcgaccccatggactacagcacgccaggcctccctgtccatcactaactcccagaatttactcaaattcatgtccattgagtcggtgatgccatccaaccatctcatcctctggcgtccccttctcctcatgccttcagtctttcccagcatcagggtcttttcaaatgagtcagtccttcacatcaggtggccagagtattggagcttcagcttcagcttcagcatcaatccttccagtgaatattcaggactgatttcctttaagatggactggttggatatccttgcagtccaagggactctcaagagtcttctccaacaccacagttcaaaagcatcaattctttggcactcagctttctttagagtccaactctcacatccatacatgactactggaaaaaccatagctttggctagatggacctttgttaataaagtaatgtctctgctttttaatatgctgtctaggttggtcataacttttcttccaaggagcaagcgtcttttaatttcatggctgcagccaccatctgcagtgatttgagagccccccaaaataaagtctgtcactgtttccagtgtttccctgtctacttgccatgaagtgatgggaccagatgccatgatcttagttttctgaatgttgagttttaagccaacttttccactcttctctttcactttcatcaagaggctctttagttcttcttcgctttctcccataaggatggtgtcatctgcatgtctgaggttattgggtgctacaaacaaatatgttcatagctctttaatttttctaattaatcactcaggcaccttcgctgttcttcattatcaaaaggactagcatcttcctcttgagcaagccccctcctgttttcatctacgaattatagacttttgtgccagaacttgttctgcttccaaagagggaagccggaggcaaagagggagccagaggctactgaccagcgctcctgacttgtgactcttgcagccacctcatggtgtcgctgttgtgagcagcctggactatgcttggtggtttggtggctgtgcccttcttggttattctgcacctgcagctcaggtgcgacatccttggatgcagactaacaactcttagttcctactcagtctgcacccactatttcccaaaacgtgacattttgccctgggggctgggtcctagggaattcccatcagattcagccctgcagcttgaacacggcatctcagactctcccttgactggcagtgcatcctaggtgggattgagtgttgtagttaacattttccttcttttcttgaagttttattgtgttcagttcagttcaattgctcagtcgtgtccacctctttacgaccccatgaactgcagcacgctaggcctccctgtccatcaccaactcccagagtttacccaaacccgtgtccattgagtcggtgatgccatccagccatgtcatcctctgtcatccccttctcctcctgccctcaatctttcccaggatcagggtcttttcaaatgagtcagctcttcgcatcaggtggccaaagtactggagtttcagcttcagcatcagtccttccaatgaacacccaggactgatctcctttaggatggactggttgaatctccctgcagtccaagggactctcaagagtcttctccaacaccacagttcaaaagcatcaattctttagcactcagctttctttatagtccaactctcacatccatacatgaccactggaaaaaccatagccttgactagacggaccttggttgccaaagtaatgtctctgcttttgaatatgctgtctaggttggtcataactttccttccaaggagtaagcgtcttttaatttcatggctgcaatcaccatctgcagtggttttggagcccagaaaaataaagtcagccactgtttctactgtttccctatctatttgccattaagtgatgggaccagatgccgtgatcttagttttctgaatgttgagctttaagccaactttttcactctcctgttttactttcatcaagaggctctttagttcttcttcactttctaccataagaacacttaatatgaaatctactctcttaacaggttgttaagtgtgcaatatggaattgtctacaggcaaaatattgtacagtagaactctagaacttactcatcttgcataattgaacttttgttcccactgatcagaaactatctgcttctgctccccctagttcctggcagcaaacagttctgttctctgcttctgtgagtttgatgattttaggtacctcatgtaaagtgaaatgatgcagtatttgtccttctgtgactgttttatttcacttaacataatgcccttcaggttcatccatattgtttcatattgcttttttaaggcttaaacagtgcttggtcaagggtatcgcataccttttcaacagattctgataatattgaattttaaaaggaaagggatcggggccctatgtctccaaggctttcttgtccacacctactccaggattgacttgcttctctgacaatgcaggggctttctagggggcgctagtggtaaagaaagcatcgaccaatgcaagagacataaaagacgtaggttcaatccctgtgttggggaagtccccaggaggagcgcatggcagtccactccagtattcttgcctggagagtcccatggacagaggagcctggtggggtcacaaagagtcattgggtcacaaacagttggacatgactgaagcgacttaacatatacacacactgccagtgcaggagactggttttcgatccctgggttgtaaagatctcctggagaaggaaatggcaacccaccccagtattcttgcctaggaaatcccatggacagaggagcctggcaggctatacagtccatggagttgcaaaagtgtcaggcacaacttagcaactaaacaaaaccaactcagtacccaggctctgcctccccatagttgctaagagtggtagctttgaccaagtgaccaagcatatacggatttacctttccccggaactaaggatcctataattgtatcctggaggcctggttgtgttgtgttttgttttgcttttttagcagaaccatgaaggtctgctttggtacagctccagaggaacagcatatgtaactttcaaaattctgccctagccaacaccgcattttccacactgaggctgaccctgttatacgtagaggaaatatgttgttgttaatgtgttaactggacaactggggaagcatgttacaagaccatgcagcgaatttcttctccagtgacttacttggtctgacagtgttaatatatctttaaaagaggagaatgtatttcctttgcttggaatcaaagctcttttgcatagtaaactgcgacttgtaaagagcaaggacatcctccgctctttgggctaccttcctgagttgaaaactagattcgacatcacgcgatctccgggcttgtgttgacatgtggcaagcgcacgagaaaatggctcagaggataattaatgcctaaaactagaaggggagattaatttggcacttaattttattttacgctgattcattcacaaatggaaatcagcaattagataatgcaaacttttgaaccatggaaagaaaggttcaggaatatgagaaaacacaagtaaaacattttccagttcatttgagcagttttcatttcaaatgattacatgggtgtctttagcgatgcagggtgggatggctcattttttaaggtgccccacctgcctgaactgcctgccacccagtctgggctggagccccccgggactcccatggggtcggggaaagctcctgtatgtgtagagattcatcaagcagaaagtagggctgtctccccccagcatctgctgaatgctggggaggagcgcacttcctggtggatcttccagatctccacccccgagccagcgcatccacttggcaccagctaagcagggtctgagcctgccatgtccctgtctcacaggggtaacctgagctgtgaatcaagcggaaacccactacaactttctccgtcttgtcgcctcctatctctctccttatctcaactttggcagaggcgggtgtgaagccatttgtctgttcctgctggagcctgttgttgaaaaatgcctgtccagatatactcagctttctggctctccccagccaggccctccctgcgcctgcgcagcgcaggggctccgcttccttcccccacccaagataagaggtgcttatcttgtcatctcaccatcaaagagtgggcttcccccactgaaacctgttgggaaaatcccagtcacagactttagccttgtttggctcgtgtgcccatccctggaccagccagagggcccatgtcctctggcccagtggaagccactgttggcagtgtctacagcaggagggaggggagagtaccagcctcagggaaatgaaatggggttcacagtgcacgcttccacccacctggaagtgcccatacagccaggcactcagagatgaaggcgcaggccttctaatgatctcagcgtccagcatgaactgtccccttgcccttgcctgtctggcctcttctctggctcccctgtctctgcttcatctgtttcagacactctggtccctttgctggtcctcgaatgcactatgcgtgtccacctctagcctttggtccagctgttgtctctgctggcccactctttccccagatgctcccagacaaactccatcacctccaagtctccttaaatctccttttaatgtgttcacactggtcaccttatttaacatgtaagtccttgcctactcccctaatcagcttctaacatactatataatttatttatttgttttgattatcatttgcctcttttgtccactataatgtaacttccccagttagggttcttgaactgtgatatatcccaagtgcctaaaactgtgcctgaacagtattctacacttagtatttgctgaatgaataaatgagtgaatcagtgtaaaacttgagggaaggtccgagaagggggttggatatagggatagatttgaggattatcaacatagaagtcatgagggtgtaaatatgatagaatttattccccagagaaagtgggtgagaggagaaataggccaaagactgaacctgtggaacaccaggtttatggtaaaaagtatctgtaaaggcaatggcaccccactccagtacttttgcctggaaaatcccatggatggaggagcctggtgggccgcagtccgtggggtcacaaagagtcggacacgactgatcgacttcactttcacttttgactttcatgcattggagaaggagatggcaacccactccagtgttcttgcctggagaatcccagggatgggggagcctcgtgggctgccgtctatggggtcgcacagagtcggacactactgaagcgacttagcagcagcagcagcaaaggccttacggaggcaggaagttggagagttggagaatccacaaaagacctcagcagagctgagggagatcaggttcttaggaataaaagtggggtgagccttgttgtgtcattcaaaggtatagagggtggaagaggagaattcagaatggatttagcatacaggaaatctacctgcccatctgtccatccatacctatatctctcccttcctccagttacccatccatccatccctcatctatccatccatcctcttatccatctgtctgtccatccgtccatccatccagctggtctgcagttactgtgccaggagtaaagacctggtttctcaccagccaaacaaaaaagaagacagctgcaaatacagttatttcagcctggtgtgggagtctgtggtcaggaagacgcaaagcgctacgggagggcagagataagaatagctgactgggccttggcctgggtgggggtcagcaaaacctcctggagaagctgccctctgaatagggctgaaaggatgagcggcatggaggagaccagcggtggtagaaacgggatagaggcccagggctgtggatttgaggaagacgtgggagcaacaacccatttgctgggttttagtgatgagaggcactggtctctgtgatcccagcccacatggaggagctggatatcagcctggtctcgctcactccgaaacctgttttctgtcattctttccaaacgattgtctttcacgacaagaatgtaatgtttttacatcgttcagccttaccacctttcgttccagacaggcagaatgatcatcccggccagcctctggatctcgacgctctttatggctcagtaactaagacagcatcggttttaaagacagttttcacagagcttaggacataatgttcatcacagcctcttagcatcattcgaaacattaaaaaaaaaaaaaaaagcgctcctcaggtaatacatgttcattaagttcttactttatctaatttccttttgagcactcattgcttattgtagagtggtttctctgtgacagcttactgatggcaaaagttgtttttacaaataggaaaaaatctttccagaccacactgattttcttagtattaactcaaccgcttactcgggcgcattcactgagcgcagtctgtctgacccgagggtcccagctgtgtaagtcacgtgactgttacccaggagttcccactcacctggttgcctgggctccctctataactccttgctgaactgtcgctggagtttagagtccttgaccctcaccttgccctgggcagcagataccaaggacttggctgtgtatctcaggggagatgaactctctgctgtcacagagtgacagctggctctaaacagtccatggttttgtccccaagttggggtcatccaagtgctggtcttcctacgtatgttctccttgccctctggccagttcatgaatcataggaagacctccatgctgtggcttgcgtcttgaatgaagtgcggataaaacaggcggtgggtgaaaagtgatgaaacggaggtgggtgggggacttgcagggagctctgggaccctagatttcagcccattttgttgaagatcccagttggtcccctctgctgctttgtgcagattgttgctcttccaaggggactcttctgctctgcccagtgaccttttcaggtactagggcccatgaagttcccagacagcatcacctagctcccctcttaagtctagacctatcacttctcggccttttggctaagatcaagtgtaagtctagacctaaatccaacatacagattttggagtctctctctctctttttttttttttttctctgttgtttcagaaggggatgctttcatttcatacccttctttttttcacatgtgttttatccacggagcaaacaattggggggaagagaggtcctagtagtcccagagggctttccccttatccagaccaacagcctttggggtccacgggtacccattctggcgggcatactgggcccttaagctgaggctgctccctcccatcccagcctccacagacactcaacttcttttgatcacgcgcagtttgttgctgacagtggttcccttagcctgtgacccagagccatctcattttgtgcaggaaagtccgccccccaccccgccctgcaccaggtggctgtcacgttgactagtgcctggctctcacacccagacttggagtcccactccacccaagaacaccccagatccccactcaggcagtgccccagcttagacatcctgcagtcacgttggcatttgcatttctacgaggcatttccggctcagtgaatgggaaattgctctgtgatttccgaaagcaattaattatgcagaaaatgttgcatcaaaaagaaaaaacaagcatttaaaacaaccatcaatgtgaaaagtgccaccaagctggagttgggcctttcttcccccttcagtaagctgtcagtgtgtttgtgagtgaggcggccctgggctctcatctgggttctgctggttcaaggacattaacagctcatggcctcagttttgcatctataaatggaggtaatgactccatcttcacagggcagccataagggttaaatgcaatatctaggcaaaggcgttagcacagagcctgccacctaacagagcaaacattcaataaatcttttttcctttctccctccctcacgctttccacacattttgatcatagaggacaaaggaagtgtaagattccaaatgcaacatttcccaccccatccctgctccatcagcagacggagacctttaaagtaggatttgtgggtggaaagggtagatttttactgctttacacttttatgcacccacagcattaagcagcacgcggaaattctaacactggtacatgaagctgtgctgctaagcttcgtcttgatacatcaccatcttttcagttggtaccattagggaaaagtgggaggcggaggtgggggttgacagagtccagaggtgtttggagcatgcgtcatctcaagaacgcttctgtctataagagagaattatcaccatacaaagatacccactgagatgcggatggagctggccacgagtttgttgggcctggagtcctagattcactattgtttaaactgaatgggcagtgggggtgcccctggcagcctctccgggcacgtgctgcttcagtgacccagagtaaccatgaataaagtgtccttagaggtgcttgtgtctgagtcagggatgtgaggcacccagcaaagggcagtcactgactcagggccgctgaatccgacccttcgtcttgtctgcgtggttctgtgccatgattcatctgatcctcctgtcaggaagcaagctcctgccaggcgcaggatggctcgtgcaagactcaggggctcagaatccacagagcaggggcattactgcaggaacgtgtgggtgacaacgatgatgatggaggtgatgacaaagagggtgacatttatgtgacatttgcagtgtgccaggcactgtgctgggcactccaccaccctggaagtgggggctgacatcctcctccctggtccacagaggagggggcccagtcttgagcagcttctgcagttttccaagtggtgtcactggaaccagagcccagggggtccaaggggctgcaaagccagagttctgtccgctgtctgcccgtttctggtgcgatgcgtgtgtctcctgaaaaccacccgctcggggtatgtggagtcggatattacaggtgattttgtttctaactctttattcattttaaagcaaaaattttaaataagagaccttgggatcttgataaggcataaatgccccctcattaaacattttggattggccaaaatgttcgttcagttttgcatacaatgttacggggggtgggggaggtggaagtggaagaatccctgaaagaactttttggccaacccaatgattcacttctcattaggacagtggttttcaaacttttggtttcagaactcctttatattcttaaaatcactgaggatcccaaagggcttttgtatatgtgggctgtctatctatgcttacagtattgggagtcaaagctggaaaagtgtttatgtatttattacttcatttaaaaataggaccaagatacccattacaggttaccagaaataacagaacatagttttattttttaaaaatccttacatttttccaaaacaaaacaaaacaaaaattcagggagaagcgtgacatcgttttacactttcagaaatctagtgtctgaccagaggactgctcagtcacttcagttgtgtccgactctgtgtgaccccatagacggcagcccaccaagctcccccatccctgggattctccaggcaagaacactggagtgggttgccatttgcttctccaatgcatgaaagtgaaaagtgaaagtgaagtcgctcagtcgtatccgagtcctagcgaccccatggactgcagcctaccaagctcctccatccatgggattttccaggcaagagtactggaatggggtgccattgccttctcctcttgccttactagatcaggggacatgtggattcttacatctgctatggcattcaggcggtttcagtactgcatgtcacctctggcctctggaaaaactccacagtactctcattaggacatgaaaatgagaaggataagccaagacttagcatggctgggaaaatagttctgactcaagaacacctaaaagtctctctgagggttcccagggttctgactcaagaacacctaaaagtctctctgagattgccaggttcccagacttcactttgagaactgctgcactaagatcagaatcgtcttgggctggaagacgtgtgcccacaagcgtccatgtggtctcctggaactgagacagtctgagtgtgtgtgtggctgcctcgtgtccagcctgttcactccagcaggtcacgatggtgaggctggaggaagtgtctgggagccgctgacacagcccttccaggtggaagtgggtaccagtaaccaggaagctgctgaacttcccatcggctgccactccggggatgtgtggcagtgagggagtctggcccagcttggtttcctgccctgggagatgccttccctacagacagcacctcccttctggtttctagtgcaacaaagctttttagtgtcatattcagcgcagtcaggcagaactctggactgacgtgtaaaaatacattgtgtccaagactcagtggtgcttttttctccgggaagggcttggtcagagtgttcccagtaaccgtctggtccttgcaatatgtggactgtggaccatgtcagaggcaacccttctctgctttctgcttcatcatcatttgctgtgggggaaactaaaggattgtaatggaatcaggcccgagataacccttagaataagagatgctgggaaactggcagtttgagagtcctgtgagcacctagacattcctattagatggttagctggctccatataacagctttaaacttcttagaatgggggtctggggagatgttcactccagcaaaggatcctgacattgtcttgtgggaaaacctttctgtggaaatagaccaaagcaaatgggaaatgcctcttccatgttccaagttcaaaaattcgagatgaagcatttttaactacaaagatggtttgagaaagctgactttctgctcaagccttccagcaggtggttggttccttcagtggctcagcagggctggggtgcccggtgctttgtggggcccagaacgttcccagtgatgctgatgctgatctggggcccacacttggaaagacactgtctcaaggaacgttgctacaggaacctggtaggctacagtccatggggtcacaaagagtcagacgcgactcaacaactgaacaaaaataaggaaggttgaccgggtgatagccaaggctaatgggcctcaccctgctttcggggtctgctagctgaagacgagaaggagtgggcagccacatgggccctgtggggcactgagacttaggaggggccctagaagaggccagcacactgtggctgaagtgtggactgttagcccagatttgggggctggtgcataggaggagacaacagctgggcagtttctgtgctgccctaagttgggctctatctggatggctgtgggacccactgtggaggactctaaggtcttctcaggccataactgagccatgggtggttcagaggatgctgaaccatggccaaggtgaatctatttagttcccgcctgaaccagtccaacagcagggcagaaccctgccgcattgattgtgcagaggaagtagacagtctggtcatccataggctgagcttggcctgagactggaattcatttggctgagttatgccagaaaaaaaggaaagcaaagccgagcgttttacgacaattatctgaatagaaatcttccttctaaaatgcccttcggagcactcttcggcatgaatgattcacatgaggaaagctgacatgcgtattctgctcttcttgttgctttttccaagtggaagtgtctctggttgtcaggagggaactcggatgggctgtgcctttggaggagctgtgacacaggttactcaaccacacatggatgttcaagattcaaaagtgctacgattccatgagcaagatcaccgtgaaccttgacttgttcaaataaataagcaggctgtctccctgccgctagctcattgctggtcatagggcaggtgcggctggtgggggaccagatgcagtgtgccagcggggctgcatctccatctctgtccctgttccacccagggccacacctcacatgcagcatctcgcatcataggtgcttagtgactgtggtccttgggaaatgggtgtctttgttgaacccagtgcagcctgtgccagccactgtgccaggcactaggtgataagcacccttggtcttgggcacctagatgccgaggtacctttgttagtctgttagtcagatgctggggttctgtctaggctgttccccagcatccctaggctcggagaaagcaggaagggctgttttctggatttataagaggtgccactaaacactgcctgtccgtgggactctgtctcaggccccttgggagagcatccctcttgagggagccaggcttagtgtcctcgtccactgggagtggagggtgctggcctcaggtgcctccagaacccagctgatgttgtgactctcctaggctgggggtcctggagaatgcgcttgcttcctctctacctgtcccttcacttaactcacctgccctgttacctgcccctcaggattgagactggagacacagagccggccttgaaagccctttggtagtagtaatggctgcactcatatccggtccttcgtgaagtgctgaactcggtgccaggcactggattctgggccttaatcctcccacagccctgctgtcaagtgcttgtatttcacactcagggagctgcagggacaaaacgcagcaggggctcttgggattggaaccatggtctgacttcaggactcacgcacacccttttacactatacccaacacccaccagtgccagggagggcccacatgtgtgtctgctctgctgggcgaggcacccaaagaagtggtcccggccagatcagtccctgtctcctgtctccagactcaggagtggcctctgtagccagaaaagggtgtttagggagctgctctcttctccctgtggatctggggagccccacgtattgcacccctcccttgatgaggatgaccgtctttacagcctggggaggtgagatgctcagggagggcccacctgggccaggatgacttctccacatggctgtgtcctcagaatgcatttggaccttactgagcagcagcaacagatattattttttattttatgtttttaaactgcaagcactattaaaccaaaagaatgcccactaattctgtcattacattttattgggcctagtaaagatcacttatatccctttaaatcagatttcggcttcttatatgcacagttcacagcaaaatggcggcaaatgtccaagagctagaattaaaagatggaacattaattcttctttcattgctacaggagtaagtgtaagctggaggatccctgatcttctttgcaaaatctggctctatcccttaccagctggatgttggcctttctggaccccagggtcctccactgtaaaataagactaagctggctggtcaggttatggggtgttatggggcatggtgacaggctgtgaataaacctagctgtttgtgcagcgcctggagcagctggacgtggactgctgtatcccctcatctgaacccaactcttaacaccaagatgtccatgaagaatattgaccagcaggtgactggagccgtacgtagcgtgtggcacttggattcatttgttcgctcactcattcagtgaatgttttctgaacattgccttattgttgttcagtcgctcagtcctgtccgactctttgaaattccatggactgcagcacgccaggcctccctgtccttcactatctcccagagttctctcaaactcatgtccattgagtcggtgatgccatccaaccgtctcatcctctgtcacctccttctcctcctaccctcaatctttcccagcatcagggtcttttccaatgagtcagctcttcacatcaggtggccaaagtattggagcttcagcatcagtccttccagtgcatattcgcagttgatttcctttatgatggactggtttaatctccttgttatccaagggactctcaagcatcttctccagcaccacaatttgcaagcaatttgctcagccttctttattgtccaactctcatttgagtacatgactactgaaaaaagcatagctttgactatatggatctttgtcagcaaagcaatgtctctgctttttaatacactgtctaggtttgtcatagcttttcttccaaggagcaagtgtctttcaatttcatggttgcagtcacagtccgactgtgccaggcctaatcccagttgcttgccttctcatggcggaagcagtgtatcattaaagtgaagaggtgaaaaagaaactgatggaagggagatgtggatgcaaaggccaaagccaacacgctccgtggggctcagagcggggaggagagttggtgggagggctcagaggagacccttgatgtcactgtaaatgagtcaaaataactcttcaagatcacccatttgggtggagtggttgagggaacagtttattagaaaatggtttatgagaagcatggaaatcagttgctcgctccgcaggggggaaaaggtggaatgtagttttaaattcaactgagagcattaggcaacatgtagaaactgggattgggagtgtgggtgcagtggggtatggagtgaagtggaagtgtgctgttgaaaggaggatgttaaacatgagaataggtccctaaaaagaggatgtattttctgaaatctgggggagtcatcatctgatggtagtggaataagtgatttattcttttgacgtgtaacaccgcaatcccgtctcctgaacagtgccccatctaggaaggaagatattagggactagttgtttgctgagctcaaagtctttgttgaccttaaaacaacttgcctggaagaaaaatgacctcgtgtgtatttactattgaacataattttccttaggaagtctgcaaccctattaaagattaataggagatcttcttttcatatgtttctgcttaaagtctgtttgtgcaagtgcaggctgtaaccattcagacatccgtgtgcctttccccctctccaccccctcgaagggcacagaaactaggctcctttaatatgtggcagattaacattcaactgagcggagcttggaccttggggctcaggtctcacaggacagccgtttgggggtagacagggacaaagagggagctttgcaaattggttaagtcaagaaaggctgtgtaattttaatttttcattttttgggaacccttgaagcagnnnnnnnnnnnnnnnnnnnnnnnnnnnnnnnnnnnnnnnnnnnnnnnnnnttttcattttttgggaacccttgaagcagcaaggtttgactgaagaaagctaggcagcatttgggagaatttctagaatttacctgcgtttcatcaaagaagtatccataattattgttcagtttctctggtcataaacattgtcgtgcaacattatgtccttctgggggtgcagtcagcacctccttaaatttcactggcttgatgccagccaggccagtagaggatatcgtatggggatcatgggtggtgacctatagaattgccttgacctgtttctgtgggtgtgattccactgccctataaaactcatttctctgcttgaagatgtagaggactcaccagaaaaatctctctttggtcctaatggaaggaaaaagcctgatgttaatcgtgagtttatcgagcacatacagaccctgaacaggtggattagtgacctcaggacaaagcatttcatctgccaacttattagcagatggattctaagggaagtatctgtgtggtcatctctaccctcgggcttcacagaggtctcaaggtcagagcagaacaggaaacagcagcgcagatgagtgctcccctcatctaggaatctgcttcggttgtaaacaggtgataggtgccaggtacacataatccttcaggaacacttggaaggacttcctgtcattcagccagagtaaattaagcttcaggacagttaaggtacttgccccaaatgtcacggtttagtaagcgattgaattgaaatttaagtcctggcccattttcaaattctatatgcaaaacctttactgagtcctgctttttcatagtgttttcattttctgttgctgctggaacaaattactataaatgtggtggctttaaataacacagatgtattctcttccagtttctggaggtcagaagcactacctggatcccactgagctgaaatcaaggcatctgcagggctgaattcctgctggaggcttaaagggaaaatatactctattgtcttgtctcatttcttgattcacggcccctccctccgtcttccaaatcggcagcagccagtccggtgtttctcacatggtatcaccctgacctcctcatctgactccctcttccacttttaaagacccttgtaattacattgagcccatctggacaatccagattaatctccccacctcaaggtccacaactttaaccgtatctgcaaggaggcactattactgtggcagctgacatagtcttaggtctcagattccagcgtctatctagcacgcatcttcatggaccactgttccgcctcccatagtagtttctctcctgagcgtctatcttgtttttaatttttggtgtaccatggattgtggaactagattcccaccccccaccccttcccagtggtttctagaaatcacagtgccaggtacctagctttaatctagccaggacaatgtgtgcattttgtactaaccacaccactgcctctctattatctttcttcggccccaaatacccacattagtttttgcatttattctgttgctttaaagctacatctgaattcatctcaaaattctttctgaagcaagagtgggcataaataaatgaaaccatgggggtgacctttgctgccctgggtacctaaggcagttgacttagagtttcttagattccaggatgaatgtcaggcaacttctgtaattcactcctaaagatggaggtgcctgcagagttcttgttgattttttttagtttgctactttgtgtccaactctttgtgaccccatggactgtggcccaccaggctcctctgtccatggcgtttcccaggcaagaatactggagtggttgccatttcctactcccgggcatcttcttgacccagggatcaaatgttcatctcctgcattagccagcagattctttacccctgagccacctgggaagccccacctgcagaattccacatgaagtaaattcacagcttgttggtttgaagtttttaggatgtggttctactggcgatagattgggtgaacatctagatttctatttctcctagtggccaaaagtcacaaaactgattactgaagttttcctgcagaaatattaaaggaaaaaaaagtattggataagattcacttcaagaggtttagcatctttgtcacagacagctgttggatacttgagagcataaatgatactttcattatccagaaaaaaaactcccaatttcttttctacctccatttcttcctgtaaaatcaagtaccaccaaagttaggttttctgcaataatgggcgttctatagcatggataatacaaaacagatctatttgatttgactatgcagcttagcgttgtttgtgatgtagctatactcccatcatgactgaggtctgcttactgcatgactagcggtgtgtgcattggagaaatttgctgagggagctgaagggaggagtctcagctgaaggacagctctggggcatcgagtcagggactgcaaggagcctcttctgggttgggatggatccccgtaccgactaccctagcttgcttttcagatttgccacgtagagatgagctgtcaagccctgtcagccctctgcagatgtggatggatggctgttattgttgatgctgggtgacttggtgaggggtgtggagattgagctgatgctgacctggacttcctctgggggctgctgcagttcaatgtgaatagaaaggctaaggaggcatcgcatctctgccctgcagcctgcttttaccaagcccaccacccacagatagaccccaagccagatgcgtggactaaaatgatgcatgcctccacctacattcgctcttgttgtgggtcaagcccagagggcagtgaccacagcctcctgggacccatatgctagactgggagccgccaaggagccagcagagcagggtctccccaggatgaggggcacaggtgcagggcgttgggttaaaggggctggaacctccccaagaatctggatgaacctgagcagggatttgagggggaacaggcttgacttctgcttggtcacaggggccttcctgttccaggaaacacaaatattaggcaagccttgaagactgctgggacctcacacctccctttcccagtcacatgtggctgcggtctggctaagtcaccaagaaacacatcagttggctgagggtgggaagccgcatcaaggggccgaggaacacagcggggctttggctttctgcgcctggggcatgcaaacctgcccccgggcctttgctccagggaggtcacatttgctcccagggagtgggaggacccgggctttctgaaggacagcctgagccccccttcccagcccgtccctggcaaacactgggcctctgcttcaggaagccagagtgtactgtccaaggaaggaagggaccagcaagtcaggaaccaccaccccaaccaccccgccccccaccgcccgcccgcaaaatctgttcaacatagactctgatttgctgaaggatgctttccagatgtgttctccagtaggctgctggagaagaagggctaaggtaattttctaaaaacagaaagaggaaaaatctgttttgtgtcactgttccagaaaattaccattgccctgctcttgctttgtcatcctaggcagcgtgtttttctaattgattttattcttcataagagatcagcagttttaacaaacttactgatgtttagtcatttaaaaaaatatgttttccccctaaactcctggggggaaaaaatagcttttcttaaaatgcttttgttctatgagctcatatggctgagaggtttttaagtaacacctcaggatccctagcttcagatttgttgagtttgttttggtcagtcttactgccctcagtgacacaggttaacctgttttaggcacagatgtgttaatacttcattttattgcactttgcagaagctgcgttttatacagattgaaagtttgcaacaaccctgcattgaacaagtctgtctgcaccgtttttccagtagcatttgcccactttgtgtctctgtgtcacattttggtaattcttatactagttaatacataagtgatttgttgtggtggtctgtgatcagtgacctttgatgttgttactgcatttgttttgacatttttaacagcagagtatttttaactaaggtatgtacattttttggacctaatggtatccctcactcaatgagaatgctatatagtaagagcaaaacttttacatccatgggaaagccagaatattcgtgtgacttgctttattgcagtatccgctttactacttgcaacatctctgaggtctgctcgtctgtcatataaagagcagcagaagatagattaagatacatatttctctgtgttgagcttgctgtatttttttgtggcaagtgcttcgcattagaatggaccagaaattggggagaaaagatgaaggggtgggaaataaaaatgtgttgcacaaagtgtattctaggctatgagcaaggtgcttcatataactgttagtaaggtatcacggcagccgtttaaggcaactctagttccattaccccattttcctggtgatgaaactgaggattagagatgtccaatacgtagctaagttatttaaccatggaaggtaggactgggattagaaacaggtgcatgccactcccagggtcgtcattttttggctcctccaactgacccactgagagtgctcttagaaactaaatgccttgagttgcgatccaagactttaaagccctaacactcatgttggattcagagcaaaacaccagtgatttctaacacaagcaaattaatgaatggattctattccaagcttctgagttgctagtgggtcagcaagtcttgcggtcctgtatgggcagtggtgttgccccaaagactccttaataaaaggtagcctcgtccatggacagactcaccaccttcttgggcagagtcctgcaccctctctgacccctcctgagccccagtgttgcctgcaaagtgctcactgaggctgtcagtggaatcttaggaagaggactgagaacactctggtcaaacagtgggaaggacagagcagttatacactgctgctgctgctgctaagtcgctccagtcgtgcctgactctgtgtgaccccatagatggagcccaccaggctcccctgtccctgggattctccaggcaagaacactagagtgggttgccattacactagatgactttaattcaattgcagggaaattaaagtccaacaccctccttacttcttccttcctgggctgttatagacagctagcaattcctcttccagtgaaagccctctgccaccactcaccatgtgacagtgagcaggaccttaacctctttggccctcagctttcttgcatgtgagatgtggggatcccgtagaaagatctggtctctagggacttcgctggtggcccagtggttaagccttcacctgccagtacaagagttctatcagcggggagctgagatcccgcatgccttggggccaaaaaccaaagcacaaatggaatcagtattgtaacaaattcaataaaagactttaaaaaatggtcttcatcaaaaacatcttaaaaaaaaaaaaagacgtgatctctatagtcaaaagaaaccaaatgccttgacactaaaatcgtgtgtaatggtggagtggaatcatggatgaaaataataataacgcatggaaaacaagtgggtttcttctccaagtagcttcaagtgtccaacagactgttctgaagtggaagggaaatccaaagttgtcaaacatcatatgatgatgttcaggaactctggttaattagtgtcagagcctggatcccattcaggatctcacagttctttgcccaaggctccattccatcaaccacacagcttgtgtccaagtatgggatttgccctggggagaagaaaaaaaaaacacaaacatttacaggattagaataatccaggatgagtgaaactgggcattactagtatgtttttgaactgttccccataactctattttcctaaaaatgttagcagaaaaagtaggtaaatgatttttcctccactgtcaaaagaccatctgctgtttgagaagactctcctagattttatcattataatcatttttctatttccagttagcagcatgcttcctttaatacgatattttctgcataaaccactgttttctcttcagcagatttcctaaggccaacttggcatttgcgggcatttttctcctctttaagggtgggaaatttctcctttcagagggagatacatttctacattagtgcctattcattttcttttcattttctcagagaaaaacaatcaggttcatcactgctgttgtcatggagatttcatgtctccagaagacatgcacttgtggaatttctttgttaatcccaccctggccgtgtaagctttcttgtttggatcactgcagcccaggcagaggaagtcattgaaaaaggttatcgtgtggttctttacccagggtgggcaggaggatatgtgtaccgggccgtcctgggatgcggggctggttggccactggacagatggaggtctgagaatcgtgcagcctgctagatgcttgactcccgtggtgccatgacctcagcacagtcacaagccacctctcttatcttccctgcaaactgtcctctcctgggtgccccactacactctgtgactccacagtctactgagctgattctgcccccttcttttttgcagtcttgattttcttttttaaaaatatttatttatttggctgtgtcgggtctttgttgtggcactgaggatcttcgatgcatcactcgggatctttggttgtggcacacaggctctggagcacgagggctcagtacttgtagcactagggcttagttgctccatagcatgtaggatcttggggcctttcaggtggtgctagtggtaaagaacatgcctgccaatgcaggagacataagggatgcgagtttgatccctcagtcaggaagatcccctggagggggacacggcaactcacttcagtattcttgcctgaagaaccccatggacagatgaatctggcgggctacagtccatagggtcacacagagtcagacacaactgaagcaacttagcacacaggcatgcgggatcttagttccccaatcaaggattgaacctgagttccgtgcattgtaagtcagattcttggccactggaccaccagggaaatccccagtcttgactttcattccatcaacacccaccatgagcttccctggtggatcagctggtaaataatctggctgcaatgagggagacttgggtttgatccctggattgggaagatcccctggagaagggaatggctacccactccagtattatggcctggagaattgcatggactctgtagtccatggggtcacaggttggactggactgagttgactttgactcactcactcaacacctaccattcagggcatcctccttcgctccttctcctctagtcctcagttcttgcctctgggtactcactggctcagcctccttccttcctttggccagtttcctgctcttctcatctctgctattgcagggagggcacagaagaagcttcaagcaaatctttgcttaagaacagaaacacccaagagtccttctcttctgcagacctgatcgtgttatttctctgtttaaaatcctttggtggttcccactgctttcaggataaaatccagcctcttaggtcattcaaggcccttgctgatcttgccagcaccatctcctgtcactcacctgtggccccgccatcccaagtgaacaattgacaattttctggatgaggcatgtgttagctgctgctgctgtttttgtttagttgccaagtcatgtctgagtctgtttgactccgtgtgttaggtgtagtggttgagaatgtcctatggctctggagtcccatagaatgtggtcacattacagctccagcacagagttctacaacctctgaggagttacttaatctctctttgcctcagtgtctccatctgctaaatgggactgggagtatcaactattacagttaaaattaaatatgtgaatagatgccagacacttcgatcagtgcctggcaggtacaaatactctatgattgttctcctgggatttttatgaatgtggttactttatcacttactgcttccttctgcagagggaacaccctcttccacctttacctgaaggattctactgatcctccaagcctcagtctcaatattacaacttctctgaaactagttcctgctgtgttaaaagcttaacttgtagagtaagaatttgttgttgttcagttgttaagaagtgttcaactctttgcaaccccatggactacaacatgccagacttccctgtccctcactaactcctggagtttgcccaagttcatgtccattgacatgtccattgatgctatgcaacagtctcatcctcttccaccttcttcttcttctgccttcagtctttcccagcataatggtcttttccaatgattcagctcttcacatcaggtggccaaagtattagagcttcagcttcagcatcagtccttccaatgagtattcaggattgatttcctttagaattgactgctttgatctccttgttgtccacgggactctcaagagttttctccagcaccacagttccaaagcatcagttctttggcactcagccttctttatggtccaactctcattgagtacatgactactggaaagaccatagctttgactgtacagacctttgtcagcaaagtgatgtctttgctttttaatatgctgtctaggtttgtcatagtttttaaatcctggttctgctattcactatttcactctagtcaaaactcaataagatgttgtttcctcctctgtgaaattaagataacagagtaattgtccttaccttattagagtcgccagggaaaaatataaagtatatgaagacgtacctaacatattctaaacacttgattcatgttgtgttacttttattagcaacctttcctgtcttttattgagaactagccatagtggtattatcatctttttacttgtttctctcggcttctagatggcaggttatttgggttcagggacagggaactcattcatccttattaacacatgcatttgacccgtcattcagtcaatacaaaggacaagaagtcagctgctttgtcaatacctgtaatcatttgcacacgttgctttgagagatttcagtggctgtgttgttgggatgggagcatctttagagagaactgttttctttctttggaagatactggagacgggaggttaatttatgacaccatttcacatgtcatttgacacaaaggtgattagggatgggagaggtagctcatccaggaagctctgaaattcaactcccaagaactttgagttcccaagaactcgaaggaagtaaagatacttcctttatctgtgggattgactacaccatgctggtattttgagagcattgtgctagtagaggaaaacaacagaatgggaaagactagggatctcttcaagaaaatcagagataccaaaggaacatttcatgcaaagatgagcttgataaaggacagaaatggtatggacctaacagaagcagaagatattaagaagagatggcaagaatacaaagaagaactgtacaaaaaagatcttcacaacccagataatcacgatggtgtggtcactgacctagagccagacatcctggaatgtgaagtcaagcggaccttagaaagcatcactacgaacaaagctagtggaggtgatagaattccagttgagctgttccaaatcctgaaagatgatgccgtgaaagtgctgcactcaatatgccagcaaatttggaaaactcagcagtggccacaggactggaaatggtcagttttcattccaatcccaaagaaggacaatgccaaagaatgctcaaactaccgcataattgcactcatctcacacgctagtaaagtaatgctcaaaattctccaagccaggcttcagcaatatgtgaaccgtgaacttcctgatgttcaagctggttttagaaaaggcagaggaaccagagatcaaattgccaacatctgctggatcatggaaaaagcaagagagttccagaaaaacatctatttctgctttattgactatgccaaagcctttgactgtgtggatcacaataaactgtggaaaattatgaaagagatgggaataccagaccacctgatctgcctcttgagaaatttgtatgcaggtcaggaagcaagagttagaactggacatggaacaacagactggttccaaataggaaaaggagttcgtcaaggctgtatattgtcaccctgtttatttaacttacatgcagaatacatcatgagaaacgctggactggaagaaacacaagctggaatcaagatttccgggagaaatatcaataacctcagatatgcagatgacaccacccttatggcagaaagtgaagaggaactcaaaagcctcttgatgaaagtgaaagaggagagtgaaaaagttgacttaaagctcaacattcagaaaatgaagatcatggcatctggtcccatcacttcatgggaaatagatggggaaacagtagatacagtgtcagactttatttttctgggctccaaaatcactacagatggtgactgcagccatgaaattcaaagacgcttactccttggaaggaaagttatgaccaacctagatagcatattcaaaagcagagacattactttgccaacaaaggtccatctagtcaaggctatggtttttccagtggtcatgtatggatgtgagagttggactgtgaagaaggctgagcgccgaagaattgatgcttttgaactgtagtgttggagaagactcttgagagtcccttggactgcaaggagatccaaccagtccattctgaaggagatcagccctgggatttctttggaaggaatgatgctaaagctgaaactccagtactttggccacctcatgcgaagagttgactcattggaaaagagtctgatgctgggagggattgggggcaggaggagaaggggacgacagaggatgagatggctggatggcatcactgactcgatggacgtgagtttgagtgaactccgggagttggtgatggacagggaggcctggtgtgctgcgattcatggggtcgcaaagagtcggacacgactgagtgactgatctgatctgatctctaagaacagcatagggttgtgatttgtagagctggcagattaccatgatgaaaattctcctacctagtccactttcaagctaccactttgaagttaaccaacttgcagaattcatgaaaatgtaccagtccacctgcttgtgcaactaccgtataaacttcctctgccttcagtttttgtatctgttaaatgtgaatcccataaagtgatcatcatgtctccaaacatgaatggcagagggaggtggatcagaggtgaaggaaccagcgaaatgttcatgtttcagcaatgcctcgtgggctcactgttatttttgaaactcttgccatccgctggctggttttgtcttagtactgagatctgggctgacaatcttctctccaaaaccagcctgttcagggcgatgctcagagcagtatatctagttttatcctcccaatcagggttatgttctctgcagcagactaactcaattagcctttcattccagagtccaattgtgagtcaattctgaggttgttaagcaaaatgagtctggtggcattagcctcccgctggaggatagtaatccgagtcacggtgctgttcagttaatagtctgctcgagagactacgagaagcaggaggcttggagacagtgcgacctttcatccctctctggggggcagctgggagcacggggctgccaggtcagggagctgatcagcttgtgtgcagagctggagtcccagctgcctgaaccacacagttggcctctgcaaagttagaagcccagtgccatgagatcattaactttcatgaccaggaaaactcttctgaaattgacactcgatccaagatggatgccattggtgccagttgagtaagtctgagtcacctttagggatttcttctgaaagataaatcctttaaccaggctacagaaaatggccaggttttacgcccttcggtgtgttcaattagagtcagaaaacaccagctaggctcagactgtgggagggatgcgggcacacacacacacacacacacacagacgcatgcaccagcctcatttggaggatttttgccaagccacttaactgcaccacatggccccgcttgggaagcattctgaggagtgtcaggtgctgtgcaaagaaaataaaacagaactccagcacctgccccggggagtcacagttatggaaccaggcacacgacatacggagtgtacagatcggtgcagataggaaagctcagacacgccttgtttacatgggttcaaggagagaccaatcccataggaacactgtgcagcctttccaatgtgctgtgcacacagatcatttcatcaggccttttaatcttcttccagttcaaattgtaggaaaagggcccagaatatgcttcctacagaccctttacaaacccctgtgatctccacacattctaccttgctcagccccacccaggcaaccctgtttataccattagagatgctaagtaaatacaatttcttgggtggaaagaggtaaaaacagccctgggtatgttggctgccaactcgagtgcgctaaagagaagaagctaagacagccatccattgtctcctgtaatcgccagataatttagcttctcttgaagtctgttgtgttcctcacatagaaacagagctgttgtctgctcggatgacctcagagatggcccagttctcatttagaatttctccaacaatatcctcttcagactgaaaaacagccggttggccacaaagagcccctttcctctgcagaccagtgatgtctgcagcccctttgtacttcggcaacttcaccaccacccagggcttctgggtggactgcgctggcccgtggaggcttcccgaactgcagacagatgctgcacttcgaccaggggaccccctacagccagtcgcatcctgagccagtgtcacccggttccttttgcctttccctgcagtgctggaaaggcgaacctccccctcagcatcccttggctggaatatactactgccctccctggggtggtagatgagagatgacttagaagaccagatacgcatatttttaaagccactaggaattacgggagcagatgggctgtaggatcctatttggatgccagggacatgtttctgtataaagtaaatattttcccactgtggatctccggtgagtgtgtgcagaagcatatttcctaaagatgcaaataagtctagtcagaaataaatattgaagggaaacttcaaagcgtcaagatttctccagttgcaggccctgaagacttctctgcaaaaaaggtgggcgaggggatggccgggggcttgtatataagagaagatgctaaccttttaagagtctacactctctgtatttggggatgttgaaaaaggcctatcgagaaaggaataaggaaaaattatgtctgtgcagaagcttgggctattttgaaacacatcctcccccctcccccaactttcttctgttgcagatctcgcctggccgattttttcaccaactgccagccagagtcaaggtctgtcagcagctgtctaaaggaaaattacgctgactgcctcctcgcctactcggggcttattggtaagatggggagacatgggatgggagaaaatgctggttcacaaatacccagagtccttccattgtagcctgtagcctctgtggttgtaaaaatctccccggaaccctctgtgctggcgttttgatgttcagttctttaggacttgtctctaaaaaagtggggtagaagaataatgaatgcacaaaggcagggattattaaagagcagggaggcagcaagtgtgaagaccggggacacatctcagttgaagaactgactgcacagaacagaagtaagcagggaaagcatggttatggaggaggagagcaagtcaaaccaagtgaagtacataactttggagagggggagcttggaagtcatgcagtctggggaagtcagaaaggctgtttccctgaaatggccctctggaaggaaaatccctaaagacttggtgttctgcttgccattaataatctgggtaattcagctcctggtggcggggggaggtttccctaatattttttccctgagaacaaacatagcagaagtagtcttgttctgtcttttaaaacctgaggttcttggcattcaggagagcagtgcaattaggcaaggcactggtgagttgggagaggacaggagtgaggcaggttcgtgcccagaaataaacagggcagcaggagaaaaggatgaggctcctggtaccggggctgtgggaagccttggaggcagggctccctcatgactgtcgtgtgagaaggaacacgtagtgaaagctggggaaggatgagggaaatcacagttgtcagaagcctgatggctcagccagggcgccagctcctgccactatcacactagtccctgggcagaagaatgtcctatcaagagtgacagcacaaaccaaagtctttgtcgccagcatgtcttcagcaagagtcaccgggaaaatcactggccatactgaatgctgggatatgcaggtgctgtacactagcccaattttatttgcaaggatacctgtgctgtcttgggtaatacctttcaccagcgactgaccattcacactgataggtcccatgaacttgtcttccttaagtcagccctccatttttatttatttttttatttggccatgctgtgagactggcaggactggcatgcggcttcttagttctctgaccagggatcaaacccatgaccaccctgcagtggaagcctggagtcttaaccactgggcggccagggaactcactcctccaaatttgcattcagtttttccttctcttaaataaagtagtccctagtaaggattatttatcaatgatagaattacatgtttaagaaataacaatgaattaagtcgtgttcattgtaaagagttttttaaaatgcaaaaaaacccacagtgttaaaagatctctagctccactcgcagatataatcactgttaataccttagtctgcacattcgaggtgcacatgctcattgaaaactccatgtgggttcaagctcaacaaactattacatacctgaggataatgaaatcacttaagggaagaactagttgaaacagtgcattctgtgagccaaaatttatttctctcttgggtccagatctttccctccactctggtcattgggttctaagatctctgaagacaaagctagttcctaccttttccaaatgtcagggatccattctggagccagacatacagtgggtgcttagcagacatactctaacctcttccttcagtgttgggggtgcctgttctttctcacctaaaggtacaagcttctctttgctgtgggtcgaagttttcctcccaaggtagaatgggacattgtttagttcaagtcagtaaggaatgtacagcaaagaagatatttccttgatccataagagcttaaaaagaattctttttttttttttctgtttctgaaccaaatatatcctgtggctcactggactgcctattacttcttacttccaataaatatataaaaacagaaggcttcttggcatgagtaggcattgcctggtgtgagccacctgggagaagacatttgggttgtgttaagtgtaagtgcattgtttatttgcaccaatacagaactcatcagaagaacaatggatacacaggaagatgtgggcaagagaccacccagcatttctttgaggatttttgaggactattaagacaatactggaggagccactggcttaggaaatttccactgtttgtttccaattccatcacaacttggaagaccttgtttaaatctgttgagagtctagcgagcagaggcgtccttgaatacatctagaacatggggtaacccataaggaaagcttctaagcttccctgggttactctgaaggagccatttgcttccgaaggaagtagatggcaaagtcctaaaaggccttttaagatatgtaaaaccagctctatctatttgtgaagattaatgatgttttcaagaaaatgcaagggatttggcaattcatgcattctaagttaattctctcctgctccatgacatccataccatttgtgcagagaaaaataccctaagcgcagctcaaggagttctgttagaattttttaagtaaatgtgtaaattattttatgtgctcaagataaattgccgagaaaatgcactgcttaacataatttcccactcagcaaacacagtgtttgtaattatccacggtgttatccacggcgcatccgtagtcagaggaacatttgtttcttttcaggctataggaaagtttatatcatcacacaagaataaaacgccttgtttttactcttttaggtacagtcatgacccccaactacatagactccagtagcctcagcgtggccccctggtgtgactgcaacaacagcggaaacgacctggaggaatgcttgaaatttctgaatttcttcaaggacaacacatgtcttagtgagtttgaaaaaatagacaacagccacacaaacgttttcccctaagttcaaactgtacttttgtgtttcctatgtccgcctcgtctgtgtgtgtgtgtatctctcttttttttttccctgatcgctcatggtgaatccagatccttttagccacactgcatgttgcagcaggaccagagcaagtgatgaacggggtgctgccaaggtggcgagtccagaggccccaggaagcgaaagactcaatagccaagggcacagcgtgtggggaggaacacttccacccacagatgaaacgttgatcttggcaaccagccccacatcagttccagaggtttttaaaacctgggcagcatacacaatgtgaaagactcgggcaagttagatgagggtaggcctccaagggctctgtgagaaaaattagaatgttaacatcaatagccccctggatgttcagggttggctcagaggagaagcagcagttcttgagtggaatgggctggtgatgcagccaggagcttcgaatacctccggagatgagatgagctgggggtgggagtcggggggaggtttctgcttggaaacagtaccattattattgcagggtcctttaggcaatttggaaacacgtttcccaggggaagagggatagagagaaatgtacgagtgtgaacgatgagcctcatcaaatgaacttcgaagcactgtgccaggtctccctcctatccaccgagatgtgtgcgccccaggcaaagatccagccgaatacctcattcgtgtcttgcagcagccaccattcatttatcggctgcctcctctgtgtgcaaactcccttttaggaggagtgtaacttatatccggttggccaaaaagcttgttcgggttttcacgtaagatgttagggaaaacttgatcaaactttttagccagcccaatacattgtctcattgactataacctgcacaagaatcctaggagatgcacttaatagtatctccatgtcacagtggaataagcggctggtattcacctagaccagccacgccagagataggatccaaagccccacccagttccttcctctgcagcccctgcaccccatgggcttctgcatcaggcctgagattgaggggctggtggataaaaacacaagcctgaacatcaaggaccaggtagaacttcccagtgggaagcgtgggccaaggttcacctacctctgagcactgtgcccttaggtgacaatttctccatggtactctgactgatttttaaagaattagcaaactttttactgcttgtacacaggaaagttattaagtgatacttcaaaaacctcctcattgtttcttttgttgaaaagagaaatctaagagattcactcagtgggtacttggctataaccaaatgctttgatcatcttaaaatgaaaacaagcttttggatgctgaagtgaaatttcaaaccagtctaaagtgtggagacatttgccagaataacattgggcactatatcatggaactataccttaagcctcttctttggtcttgactgaaataaatcatgtgtgtgtgtgtttgtgtgtgtgtgtgtgtaccctctttttcacgtcctgcacactcttctggcagaatccaggttttaatgaagataaagcaatgacaacagaggtcagtgtgagaaaaaaaagattatactcacttgggctggaaaaccactgcctcagagagaaattaaagtggtccctgcttaataatattttggtgaagggaagaatggcatcttaatgctaatgattttttaataaacataaacacatttggtcttcaatgaggaccaaagccagccagaatttttcatagtcgttccatttatactttgattagatggagcacagcctaccaaagacaaagactaaattcagcctcttgcaacaggccctagggtagaaggaattggaatttgtaggtttcaacccagatgtaataacccagttccccatcccttcaaattaaccagacctctctaattcccctagcacaagcatgacgccaatctattgcttccctggtggctcagagggtaaaagaacctgtctgcagtgcaggagacccgagttcagtccctgggaagggaaggtcccctgaagaagggaatggcaacccactccagtagtcttacctggagaaccgcatagacagagaagcctggcaggctacagttcatagggtcgcaaggagtcagacatgacttagcaactgaacagcaacagtgtattgtatacttcctctgtttgcctcctcaaccagaatatagcctccaggaggacagggacttctctgtcgtgttggtgtcgttcgtcccctgctgattctcagcacagaacggtgcctggcacttagtaggtattccattgacacctgaatgactgagtgagcaagtggacagtcttcccaaatgacaaggcaaacatgctggaaatcacagttccaacaaatgacgtgggtctcctgaatctacgtgccttaacgggacaaaggaaggttttgttttattgttgtagtccggggtcattgaaggaaactggttgcccaagtaccaccttctttaccctccacccacccgttaagctctgtaacacagccgggagctcaagaagcattgatattaatttgaagctgtttcctacagactaaaaaatgtaaagttttcaaaaacaagcttccaatttgctgaacttcaaggataacaattttagaacaaaatgtcacttctgagaggaagattaggcttaaattattctgtctcgtatatcgaaaaatgccagctaagagtcatatattaacaatttgcacattcaggtcccaaatggaagagtttgatttcttatacaatgaatatgaaataaaaaaaaaattcctaaatccaggccaaatccctgaggacctgattaaaacctgttttttattcatctcggggaggggaacaacacacaaccaatttactcttgccataaaaaacaatctaatcccatgaaattgtaataagcttttaaagttttacattttaaacttttcatttaagcaggactgcaccttcctaattatgcagattttgttcagaagactgggagcagaaaggaaaaaaataatcttgcttacattttcatgatatattattgatgaggtttgggatatatttttcttttaaagaaatcggttgtgtttatactttgagcaatgccgagctcatagtaggtgctctttggtagcagatcactttttttttttttttgctgtttgatttcactgcctgctgaattcgtatgaatgctactgagtcaccccagtctcataattagggatgtggtacttactaattagcttgcctttagtataaaattttgacacaagcatttttttgaaagatggaacagtgattatgatcccaaatagcagatctgaggctattttgagtcacgtttcaaaagacttgactgatggatgtttcagggaaaggatcttattcagtacttggtacctgaagagggacatggtaaatatttgttgcacttacagagcatagctaagtgtccagttcagggtttaatgagtgaattgattttagagcacctgacccatggctggctctcagtgtgtagggggtggatggatggatgggttttgcttagagaagtccagtgctctcatcagaatttctagtgtgcctctctggcttcacgtatcctgtctgttacatttgccgcttgactttgaaattggatttcctcctgttttactgcagaaaatgcaattcaagcctttggcaatggctcggatgtgactgtgtggcagccagccctcccagttcagaccaccactgccaccaccaccacagccttccgggtcaagaacaagcccctggggccagcaggctctgagaatgaaatccccactcatgttttaccaccttgtgcaaacttacaggtaagagaagaggcccgaactctccgtcggctcggtcttctgggtggggacgcagactcgttttctgaaatgcgttgagagatggttggaaagagatagaatgctgctcaggtttaaatcttaaaagtgcattttaaccacaagaggtgaagatcatgatgtactgtagagtggagagacagatggcctctgggaccaactctgccatttacgaggcgtgggcctttggtccaagcagtaaaatgaggggcttgggcagatgaatccttaagctttctgtttgcgaagtggtagatttgagctggctatttgtttatttagtatgcttttcagtgttctgtccaaagcagcagcatacagaaaaggaagaaggaactcttggctttctaaaaagaattttttttaattttatacaactgttaaaggttactctccatttacagctattacagaatagtgtctatattccctgtgttgtataatacatccttgaacctatcttctacccagcagtttgtgcctcccactcctgtattgcccccccccaacccttctctctacttataacctctagtttattctttctgtccatgagtctgctttgtttttgttatattcacaaatttattgtatcttttagattccacatataagtggtatacagtatttgtcttcttctgtctgacttatttcacttagtataatgccttccagggtcatccgtgttgctgcaaatggcaaagttccattctttttaatggctgagtagttgtccgttgtgtgtgggtgtatcttctttatccattcatctgttgatggacacttagtctgctttcgtgtcttggctactgtaaacaatgcttctgtgaacattggggtgcaggtatcttttcaaattagcattttgttttccttgtatatatacccaggagtggaactgcagactcatgtgatagctctgtctttagttttttttgagaaacctcccatactgttttccacagtggccatacggacttacattcccagcagcagtactcaagaatccccttttcccacatcctccacatgttgtccgtgtccttgttgatgatagccatgctgacaggtgtgagatgatatctccttgtggtttttatctgcatttcctctttggaaaaatgtgtattcagttcttctgcccatttgttaattgggttgtttatttctttgatgttatgttgtatgaactgtttatatgtgctggatattaatcccttatcagtcatatcacttggcaaatattttctcccattcagaggattgtcttgggagcccttggctctgtctccatcactagcatttagactcaatgtggagtatttaactgtgtctaaagctataccagagacagtgttattagtgaagtattaaggcaaatgtagcatcctggacagcttcattcttcaggaagcacaagaagatactctttcgtttgagttgctgtgaaacactgcactgaaagcccctcgcgtgaacagaatccagtgaaaatggcccagaggcttctttggggttgaggcagatctctaagggccaggaccacagtgacctacgaatggtcatttcctgactttagtcaaagcagagtgcctattctgcctttttatttttttcatcataatgagtattcagctatttgggaacctcttcatatcctcgccaaggatttccacacacctcacctcctcggatactaccctcctgggggctgggggagggatggtatgattgtgctcttactgcagcgttgtccaccctgttctttcatcagaggatgtggggctcagtgaggggacacttcggctcgagcccacagagctggcaagtcatggaaccgggcctggaatccagaggcttgtgttctttccacggttcctcctggatgtgagaggcagtgttgaccacatggcgactgctgcctcctgccatgctaggggaagggacatgtgccaagcacagtgctgtcccttcagccttgtcacagaaacatgtcctcccacctctccaccacccccgcccggggccccaccgagatccccctcaccagaacacttaccagaaacttctctgcgaataccttctcgactcaccgcctcgtctgttccctaggaagtatgggggcagtttttctttttttctgttttacactatttattcacttatttggctatgctgggtcttagttgtggcacatgggatccttcattgtggcgctcaggctccttgctagttcttctctctagtgcagtcctcaggctgagttgccctgctgcatgtgggattttcatttcctgaaatgggtcagggatcaaacctgcgtcccctgcattggaaggcagattcataatcactggaccaccagtgaggtcccgggggcagtttttctccttctgctttgcttgctatcttctcgccccaacactgccctcttcttcttagatatcccacctgccattcagctgagctatgtttctgttctcgtcctcctgggtccctaccaacatcacactctcaaacgcactcacacagaagactaaggagaaggtgggtgggaaggcatccagggtcaaccagatgttcttcatacttctaaagcatttcctgagctgtccccatcttcaaagcaccatgcataaaatgccagaagtaggaggaggcttggggaatactgcggcatctaaccaccctgcagcagagccctgcagttcagggaggggaagccgcttgcaccaagtcaccagctggttagagatgtgtctagaagcagatgtagcaccacagagcagtctcttctttctctgtggccaagcctcagtgtcctgcttcctcctaggtcagccttcagttcttatcgggtccgctcagactccagcatcaataaagactggtggccagatgaggacaggaccctgttataatgaggtgataacagtggccagaggtggttcctgaccacaggaagaggcaaagctctggctgcagcagaaacaggttcttctctgctcgtgtctaagtacatggagcttgacggcagaggcagtgtccagcctgcttattaacctcctttggtgttctggaaacttcatggaggtgggcattcaaaactgaaattctgtcctctgtgaatgtacaaatagtaggcaagatgcagctggtcagtgtctttggaagttcagactctgtgaataggtggcatctcagaagaatagtcattttggaagctccttgaaatcagtttttccccatgggagagcagtctgccagccatgaggaggtgttcttctctgcatctccctgcccgcccccaccccaccaggtcatctgtcctttgccttttggggtgtggtgcttggtctggccaccagtatttacctccatctagatctgctcgaccatctcccctgtaaggggaaccctataccaaggcagggtttccctgacaacgtcaggggatccatgagatctattcccaagaaatagcctttccttctcctatattagtggtagggactctgcctcataaacagagggacaaagtacaattaaagcgtgatgtttctctcaggattgccatttaacttctcagcagagtggggcagtggagacgggggaatacagaggaagcggggggacaggtatacctctcttcctgatgacacacgcacaatccacccacctgggagctaaacagcattttacttctagggagctcttaagggttccacgaaacctcaccttacctcctaagatgactccctatcagagctcatttttgcacatgtgaggcaaggggtgagggcttgggttttgcagtcaagcatgtgtgcagacccacactctcatcctcaccaacgctgtgacctctccaagcctccacttcctcatctacaaaatgctaggacttctttggagaaggaaatggtaacccactccagtattcttgcctgagaaatcccatgcacagaggaacctggccagctgcagtccacgaggttgcaaagagtcagacttcctagctgctagattgagaggattaaatggtggtaatttttcattgcctggggtagagtaaatgttccagaatgtcacatgtgatgcttaatcccaagtcagctggccatttagtccctttgcaagagatggatggtggcattgtgctttgcttggggtgttacactggggaacggtggcgtgagttagcactaacagttgtagagtgagtttcattttgtgaaacatttcacatgcattgtgctttttagtcctcacagcatcccacagtcaggtatggtagtcatccttctcttttacaaatgaggcaactgaggcagggacatgctaggaacttaactgagatcccacagtaattctttggtggggctaagatgtctgactccgaggccagtgcacgttcaccttgctccatgacctgggctctttctttgaattaggactggatagagaggaaaaagtaaaaaatagaaaaagaggtcatgcagtatttgtctgtgtctggcttatttcacttagcataatgtcctctaggttcatgatctcatttatatgtggaatctaaaaaagttgaactgtttaaagcagagactagaatggtggttaccaggggctaagagtggggaaaatgggaagatgctggttatagggtacaaactttcagacatgcaagatgaacaagatctggatatatatagcatggcgacagtacttagcaatgccatattacatgcttgaaattgcttagagaatacatgttagttgttcttaccccgccccacgcccaaaactgctaactatgtcaggtgtgtaataatatgttactcagcttgactgctgtaagcctttcacagtgtatgaaagtgaaagtgttagttgctcatttgtgtctgactctttgggaccctatggactgtagcccaccaaacttctctgcccatgtgattctccaggcaagaatactggagtgggtagccattcccttctccaggagatcttccaggcccaactatcatacctaggtcttttgcattgcaggcagattctttatcatctgagccaccaaggaagcccccaatatacacatatattgaaacatcaagttgtatgcctgaagtcaatacaatttttatttgtcagttatacctccataaagctatagcagggtagggaggatagaaaaaaaggaaaggagaagctggatttgacagacccatgtgtcccagtgcttcctgaaggcagcagttcgaatgatggaaaaaacagaaagctcatgctttaggcagggatgtttcaagactaaatcccaggagagagagtataaaggaaagacaaagtttgccagtgaaacttcttgcagcttgatgatctctttaaaatgacctacattaagaatggttgaaagaatcagtggaagccagaggatcgggagggatgagtgactgatgagaaatgggacttagcccatgagaacgtggtccctggcgttgatttaggactcctgcttcaacatagggctcccaggagactcattcttcccatctgtccttcctttgcctgcctggctgtcttcagacaaattaccatttgccagggggtcctagaaatatcttagcatggcgtttgtcccagtgcatgtgtcattactcagaatagaactgacttaaaccagcttgggatccattggtaacagatttggagaaagtcaactatctttggcagctttaaagttcacttcccttgagaagcaccctcttgtaatatttttaaatatacaataatatgagaaaactcggagggacttgggtttctgtcttaacatttgagatagatttcccccttggaagggaagaaataagaataaacattttcaaaagtcggtttcaaagtgtaattatcaacaaaccaaacacaattctgtttgaggaaaagagtagctttgggtgtggtgtgcatagggctcatctgcagttcctatgaaaggctcagctttaaaaaaagcaaaattgattgattagtataaaatgtcatagaaacatccaagagctaaccccctgaggtgataaaataccaggttttgggtaaccagccaagctcccatgtcaggattaattggcaggcatattagtaacaagcgtgacacaggctgtgctattgttggcatatccctttgacttgtacaaagtggtcacatgataaagaaacattaaaaatcctaccaattatttcatcttctgtgcatttgagtggcctgggagctggaaaaagctcagcatatttaaatcaaatggactgtacttgtcacttatttaaagtaaatatcttttggaagtgtaggtgttagaaaaaaatctctactttgcattgttgaacatcacttaactctggtggcttaaagtgacaacctcttaaagtagcctttttttcggccctagcttacaacgatcaaataagactgtacagattgggggatatagccttgggactagtgctgaggatccttgagaaatcttcactgcctctgtcccttttcttggaaggggatgtgatgtgtgattgagagaagaaaatctgataggtggggacacaccctcagagccaaagctgcaggatggaggtcaggtggttgtgggcacttggtggggactcttgggaacacggctagtgacgtccaaagtcagtttgtttttatgaaccagaaaaacatatctgtgagtgagacatggagggacagcagttgtgcatcttgggtttaagtcccctcccctcatcctagtgggacttttaaggcagcattctaggggaggattcataaaagcaactttttcaatcagaagggagatgtgagcagggtctaaatgaataatttttgcaagaatgcttattcctaaagtcagcaatgcaaagcctggctgtcccttccccacttcaacaatcgtgtcattttctttggcagtcagactgtgctagccctgagaaggaccgtgctggccctctgtgggccacgagggcaaagactttgaggtgggcattttttttctggagcagaccacctggattcagttgatttcaggctaggtctgcacctgcctggccgctcagcacagtcccttctcttggccgagtacagtttggagtttatagaaatatgctgggcgcttgaatgttgttgtttagtcattaagttgtgttcaactctttgcaaacccatggactgcagcccaggttcctctgtccatgggattttcaggcaagaaccctggaggggttgccatttccttcccccgggaatcttcccaacccagggatctaacccacgtctcctgcatgggcaggcggattctttaccactgagccaccagggaagccccttgtttgctgtagcacaacatttacagattggtagtaggggcaggaaagtgaaaagtgaaaatgaaagtcgctcagtcatgtccaactctttgtgaccccatggactatacagtccatggaattctctaggccaaaatactggagcaggtagccattcccttctccaggggatcttcccaacccagggatcaaacccaggtctcccacattgcaggtggattctttaccagctgagccaccaggaaagcccagtagggggcagggggtgatgcaatttggctgtctttaacctctgcatataacctctttaactcatacatattagaatgtttgaatattgaatattagaatattgcaaaaatctctatctgaaagaagatcttccatcttggaaaaacagcatgccttacccttccattgccccagtacttcatcagaccttaaacaaagtccatctgcccggatagggccagaccagaccagaaatgattttgaagctcttcccagacaggccaaaccccaaaagtctcaatatggcaagaaggcaattatccaaccctgctaaatacccaggactctgctggctgtgaccagtgaaaattttaagtgttgagttttaaaaggaaagctgccagtgggtgttctgtaaatcatttgtgccagcagactgaccccattatgtcagctaatcctggaaatacaggaggtaaatttaaatcttctagcatgaagcacttaatcaatattctaaatgttttccttgcaatttatgctagcagagcaatggggtgctttggcgtcccgttaatcttctggagaagaggcgctggagagcaaggccagggtggtggggttttgcacaggctgactgctgggagagatgcttattctacactcggtcccttgcatggcggacttgtcccctggactcatgccatgcttgccagtggttagagctgccttgcaggtgcctccttttcagcctgtggttggcagcctaagagttgctttgctgggaggagcaccagctttggagttggttgaacttgagtgagattcctagtcagttcccattgctctctgccctggggcatatttcttactgtctctaaatgtcttctgtaagttatcacccacctctggggtttgttgtgaggattagagaaccagtgccactaagcagagggctcagcgtgactagccagccctctgcacttggaggtggccgctgttgccatgattgatgagctttagctctggatcttggagccatacaggaaggttgttctctggtttgggctgggggtgagggggacatggatgaggaaagatggagccctttaggttttgcctccttaacttgtaagaagggtttgaacctcatgataccctgctgctttctctatgtggtcattgtaggatgtcccaaggaaggaggggaagggaaggaatattctatggacagccggccaagaagaaaaggaaacccaaggcaataaagaaaggacctgggattagaactgtttgaagctccatccctccatgcccaggagtctcatgagagaactgcctaaaagcagattctcggggctgtgaacttggagctatgacagtagtcaggtcttggtccagcctgggtgtcagagtaggactgtctgccctaggccgtcgccacatctccttaactagtgctgacactgggaagaggtgacacgctggtaaaaaggaagcactcttatttgctaaaaagatagtgtttgagagctaaccctgcctcttagcctcaaagaccaagagtttgcagtctttcctgtttctgcattccaccagcactcttatttgcctaatatttatctttaaactcaatgtctattaataggtataccgttaataacatttgtgaaaccagagtcaatgcaatagctctctatatttaaaaaaatcattatatatattaaagtaataaaaatgtttttatgctactaaataatgtcccatatgcaagtggttcccaaaccccactttaggaaacattaaaatctattcatatgacagatcctagagctattttctttggtaacttgcatcacctctgctttttatggttttgtattaataaagcaaatatgcaaatggttctctcggcgggtcaagaactattctaagcacattgcataaatatatttatatgctatatgatgctggaaaagattgggggcaggaggagaagagggtgactgaggatgacatagttggatggaattattgattcaatggacatgagtttcagcaaactccaggagatggtaaaggactaggaagcctgacatgctgcattctgtggggtcacaaatggtcggacatgactgagcagctgaacaacaatatgtatgtgtgcatatatatatatatgtatgtatataaactcattcaattttgatagtaacttcatgaggttactactgttatgattcccgtttttcagatgatagacctgagacacagagaggcttgatcaggagaagagaaattgagcagcagcctatttctcctctgtgcctttttaactggctatactgtacaatacgtaaatcagaacattttacctttttgtttctggataacatttttcattcagtagagactaaatcagtccctaaattcttgttgaacatctggcagattgaggtgccagactggttcataaaccagcataaagaaaggtaatgatgcttattctgatgccaactggtgtcctgcggtacaactcaattccaacgctagccccctgagttagcacagatgctacaggttaagcgcacggtcccaacaagactgcccacttcagatgccagccaaacttcaggggtctccaggccacctgcttttctgaccaactggctaaaaattcagtcgtggaaacttccgtaaaggctgttcagtgacctcttggctgtgaacggaagagttctgtaggtcaggcttactcagttccagccctattaacctgttggcctggtaatgctctgttgtgggggttgccctatgcctggtaggttgttgagcaacaccccgggcctctgccttctagatgccgggagcccctttccccaactgtgacaaccaaaagtggacccacacacctagcaaatgtccccagcaggacatactcacccctggtgggggaccactcaggcggctcagtggtaaagaatccacctgccagtgtgggagacacagaagactcaggttcgatccctgggtcaggaagaccccctggaggatcttcagtattcctgtattcctggaatacagtattcctgcctgaagaatcccatagacagaggagcctggcgatctgcagtccacagggtcgcaaagaatcagacacaaatgagcatacacactcgcccaggaaatgagggctgcttcaaaagccctctttctgctaatatgggagatggcggtatgtgagagtcttttcgggtttcttgtgtgtggtgaggaacatcaggagatggaagagcagacgggtgcgttggactaaatctttctaagttgtgccagcaagagcaacgaaaaggggctcttgtgaccaccacatgcccaatttgtgacgcttaactcccccaaggccacactcgcctcagagtgaggcccgtgatagagtcctaagcggtgttaacagaagtatttgacctgcttctcatcattcagggacccgcaggtgactgcggccttcggacctcctctgaggcctcagcccctctgagtgataatttcacccagccagcatcccagataagtgacacatcaaaagacttttactctgcaaccagattggatagaagtgtttagaccaaactgcagttgggtttctcctgcgtggactttgaggctgggggagttttaggaagggctgggacccactgggatcatgaggccagtaccagacggatcagcactgagccctcagcacatgtgctgaatctagataaatgtgacttctgggtgccatttcccgggccttctgtttaagaaaacagtcttggtgcggtatgttatttgtggtgcttggtttatggtttctgtgaggctgccgtcatttctgtgtaatacacagtgttttctagcacccactgttgtttcatgtgtcataaaaaaagaaaaaatccaaatttttgctgcacctggagtttcctttgcagaatgctctcctgctcagacggagtttatgtcctgggagaaatgagctgcggggagttggaaaggtgggggtggtcttcccttagcagaagcaggaaacctcagggccccccacccactgcggcccctgggttcccccagcagcctggtgacagagctcgggatcagactgccaagtggccccagggcctgtgtgatgggcccagctactcacagcgtggagactgacagtgacctcctgggacttaatcttcccggcttcaggtgccagaatccacccacggtagccccgggaggaaataggcttttgttgtaaaaacgcttttggaaataactgcttgtagagcgttttcaaagcccttcatccaggagagtgcttgaattcagaattcagaattggggaggactctgttaaggctgccatacaatccagcatgccagaacgcattttcaactgtgtagtcaaagcctattggaaatggaagctttcagggtaaaacaggtaccaaaacacaggaatttcatctcggagcctaggcttcctagtaggtgcgtatgcatatcaccctgtcgcccagagtgcagcgtcatgcctttgttcatttgctggggcagccttaacaaagtaccgtagactcagcgggtaagacaacagaaatgtattgagtagggaacctgaacaaactttttggccaacgcagtccttcccgcagtgttgaaggctggaagtccaatgtcaaggtgtcaacaggtttggtttctctggaggcgtctctccttggcttgtaaatacctttccttcttgctgtgtccttactctgtgtgtgcacaccctggagtctgtgtgtccaaatttccgttttgtataaggatgccagtcagactgggtgaggggccaccctaatggcctcattttaacttagtatctctttaaagaccctgtctccatccactgtcacctatgtgagcaccccaccgaaagtcatatcctgagatcactgaaggttaggaattcaatgtatgaatttttgagcctgtaacataacacagatggcaggtactcagcaacaaaacatttggcattttaaaaacctcacctatggagtgactttaaggggagaagtttgctgttaccataagcagttcttcaatccccagggctctgctcagtgtcccctgtgtagcctttgaacatgagaggatctgactgttggtggttctgtggttgaaggaatatggggacaggaaaggcacctccctaggggaccagctctcctgagaagcctacaaacatcaggtgttgtctgccgtgctgaatcagatgctatcagatttcaggcttctgcaaacttaattcaagtaatgatttctttgtgattggcatatttgcatggtagctgacttcagttcaacatatgttggaatccacaatactgtaatttaagattattttatcagcagtaaagggttttataattcatttaaaaagttgtaatgggcttgagcatcttcagtatagtttaataatatagcattttatagcttgctgtaaaatttcagattacgattttagcaattgcgcaattctattttatagtttaataattgttcaaatgaaggagagtgtaaagttcctaaaaaccaatttaatgactgagaaaaatagcctttatccaaatctattgtacacctttaatgaacccagatgtcaccttctcaacaaaggcagatcgcgacatctatttttctcagccgcaaagccctgcaaattaagcgcacagagtacagtatctctgagttataattttcccctttttccctcttaaatccctaatctcctagagctggaataagcctgtggcccccacggggtgaatggcagccaggccagtaaagtttcagtaggaacagggagtgaattggctttctcttaagaagattctgcattttacaaatagccccttgatctcaccacttcaaccccaactcttaactctaagaagttcataactctaagaactctatgaagtcttgtgtaaatgcctccagataaccccctactttaccattctgtggtgtgagtggcaacatggggagccctacattattacattgctctgctgtgcttggttgctcagtcatgtccaactctttgtgaccccgtggactgtagcctgccatgccctcctccaggggatcttcccaaaccagggatcgaacccacgtctcccacattgcaggcagattctttaccatctgagccaccagggaagcccacatgatcatacaattgtacactattgttgttgttcagttgctcagtctgtccgactctttgcaaccccatggactttagcacaccaggcttccctgtccttcactatctcccggagttcgctcagactcatgtctattgagttgatgatgccatccaaccatctcatcctctgccgcctccttctcctcctgtcctcaatctttcctgacatcatggtcatttctagtgcatcggctcttcatatcaggtggccaaagtattggagcttcagcttcagcatcagtctttccaatgaatattcaggattgatttcctcattacacataataaaagtgttgaaatatgatgtcagggagaaggcattgtgtcatgttatcattggaattatagttcgtgttattgtatttctggggttttccagggtatatcgtcccaccactaaatcagtagataagaggtcctgggccaggtacccaaacccggtaaccatggagcatgcagagcataccttgggaatacagtcccagaatccttgggtgcagtgtgtttctctggggttgtgaggggggtggagctctggctcagagccctgtttctgttacagagaaatggcatcaatcagaggaagccctgtgtggctggaatcctttctctctgagatgcaatctgaggacttgggccccacagcctgtttctactctgttataagcagccttgtctgtgaacagtggggaggctgactctgaaccgcccacctgagaagtaaaaagggagtggtcgtccacagtgactgcagccgtctcctgcatcccctggagatgatgcagccttagctccttttctgtctcacactcagggctctagttgactgttgttgtttagtccccgagtcatgtccaactcttttgcaaccccatggactatagcccaccaggcttctctgtccatgggatttcccaggcatgaatactggagtggggttgccatttccttctccagggaatcttcctgacccagggatcaaacccgcatctcctgcgttggcccacagagtctttaccaatgagccaccagggaagccctgctctagatgacacacccttcttagtcagctccagagaatttctatttgaaatttgatgttgcttaggagcaacataaggctgtccattatccaatggcatttggatatcttttgccgggtcagggtcatctcagcattgatgccacagctgaagggcattcttgctgcccagggacaggcatgtgctccaggcttcagctgctatctgcacttattccccaccccagcacttgacaacccctatgttatatcaggggagggtatcacttagtaattctggattcctttggagagtcaaggcaatgccatcatttgtttttctccaaaaccaagttcactttcaggtaatgtacctcaagccttgaccgtgccaatgctggacaagggatcatgcagacagtatatcaagagtggcatggttgtttctcaaagttgcttttttaggaaaaagaggccatgtgcttcgaagtagaaaatactgaaggaccagacttcttgctacaataaaaccgggccttcctgtggactgcggcagacagtagtttcagctcctcagtccttggattcattgtctgtaatatgggggaaatgtgcagaccacctgggagggttgtgataattaagcaaattaaaattcttaagcatagtaactgagcctccagcacagtaaccgtttatatcattattttatcctcttggttcgtctttgctgaccatgagacagaaggagcctctatggagggaccattcagtgacttttatcccagaataatctgggaaatgaacgtgggcattgtacagggtgtggaccctgtgggagtaggtgtcaagttgaaataatttcatatggtcactcatttatgatgtgttctcctaaactttttttttttttttttttgctacttttctcaaaaagggagaataaccacttaaggatggggaggggaagagtaggccccctcagtgcattctgggaaaagctatcaggccaccagcccctgtggtccagagggcagtgaggccggcccatcagagggatagtaattcagcagttggacaattgcttaacccaattgctgggtaataaacatgcttaataaaaaggaatagcacgtctgcaccatcttggctataaatatgttctcttgttctggggaacctacaagaatacctctgagcacacttagatcgcatactataaataaatccaatttcttgtatcccctactgaccttttatcctttggacttctaaccaacggtgcttccctgaataatagactgtgccttgtgtttgaaccgtggagctcacttgtgggaggaaactggctctagttctcttggggggaggttcaatgaagggctcccctcccctgcatccctcctgtttgaggcccagtcacatcatggataaggctagagtgtgagctcaggatggtgggtactgagaagacatcacaggattttcccccacggcagctgtcccctgcctgccccttggctccagggaccttaagtgtgatctgacagatgctcacttctgggggctcttgggggagcaaaacccaccaggcctggcttgacctctgaccctttgggttgattctaggctccctcagctggctctgtccatggtggctgtgtgaccccagacatcttactcttattattctaagcgttgatctaaggattatgggtgataataggtaatgtgtgccccggtggctcagacagaaagaagctgcctgcaatgcagagacccaggttcgatccctgggtggggaagatctgctagaggagaaaatggcaaccctctccagtattcttgcccaggatatcctgtggacagaggagcctggcaggctacagtccttggggttgcaaagagtgagcgacttaaaacttcagtttgccctgtctggcatggagttatatgcatgagttttacattcgaagagactgaaatcaaagagattcaaatctcaggacagccactcagagctgtggaaacttgggcagatcccaacctctctgaaacccctgtgttctgatctgtaccaaaggggaatgccccctcagaaagtcatttgagttcttggagagcattcagtaccatgtggatggaatcattatcaatggaggctgtactcaagagtagcagtcatgatcatcacagtcattatcactatcagtccacttggttttcctctattttctagaatatttttctctggctactaaaagcacagttttttaaaaaagtttttcagttttttgcctttacttgtttcttgctgcaccacgagtgctctgtcttgccatccagcaagtgaacaaaagcccagggtccgttctgtctccggacctgggtgttttatatcgaaccttagttgccaccataagtcacagaaccattacagggacactctcagctgcacataaatgcacaggagaatgagaggcttaataaatattatctgatggtgatgaggacacgagtctgatgagatcggctttatgcacttctcatcagggaccacgatgaatcttttctgctgctgtctgcacacttgcatttctgaatgcacatttgagaaaacagatttttccttttagggcttcccccccagtcactgttacagcatgatcactgaatattggctccacaaagctcctccccgacccccaaaccaaaacgtctgtttcaagcaacgtttggcataatcttcctgttaattgggcctaattttctaaaatgcagctcataattgcttcttgtctgaatattttactaatcaagcaagttaagcatgaatgaaaacacacggttctctgatgtagttggcaacctgtctttgccctaacttgggggaaagcgaaatagatgtaagatgatccggtgaagttagggaaagaagaaaggccaggccgaatgccacgttgagctccctgctgtagacagtgtctgcacacagtgctacttgccatcctgccattagtgttaaattacaagcgccaggctgcagacatccttttatggtgtgcaggagactgacaggccagcccgagggactgcttagctaatggaggactgttgatatatcttacaggactgtatctctttcatcacattcatttttaaatccgaggaaacacagaagatcacgagccagttctattcagtgtcacgttcacaaggcaaattagctgaagcttttgcatttttcccacccctgcctgaatccgagaacactcaacgtagggaccacagaagagctcagagatcatcaacagcaacttggattttaaaagcacttccaacacgagtatcacgtttgattctcacaactctgtgaagtgggcagcagggtgcggttattataattatagatgatggttgaaaagccaggacatgatcttcctaacctgtgttctttatagcataccatatcaatcaagctggtaccaggagtgagttctaaatcattaaatttagagttctcagtcaggtctagaagttctaactgacccacatttaaccaattcaccacataagccaacctctccaccattcccctgtaaatatctgatgcccaaggcaggctgaatgcctcacactaggggcctctctcctgctcacctgggccccttgctttctccaatggagatgcaagcttgaaagattcaggatcacttgttctcaaacctttaggtgccgttgtacttggtattttgcttacctattttagttaaataagctggctacaacataaattccccttccattgaaaaatggctttggattaggtgtaggacaaccaacagtaaaagactgaaaaaaaaatttaagtatatggaaattgtgcactgagattccctggtgggtatcaaacaaaagtcattcctgttttaaagaaatgaaaactggatgttgcagattgtaaatgataggtgtagcttaggcagaacgtaagccagggaattccagtcagcaaactctttctttaaaaaacttttcaaagtggccttgattacgtatccaaaaatcagtcaatgcatgacattcaatttttaaattaaaatgcttgatacaatacagacaatggaatattcagttcagttcagttcagttgctcagtcatgtccagctctttacaaccctatgaatcacagcacgccagaccttcctatccatcaccatctcctggagttcactcaaactcacgtccatcgagtcagtgatgccatccagccatctcatcctctgttgtccccttttcctcctgcccctaatccctcccaacatcagagtcttttccaatgagtcaagtcttcacatgaggtggccaaagtactggagtttcagctttaacatcattgcttccaaagaacacccaggtctggtcccatcacttcatgggaaatagatggggaaacagtggaaacagtgtcagactttacttttgggggctccaaaatcactgcagatggtgactgcagccatgaaattaaaagacgcttactccttggaaggaaagttatgaccaacctagatagcatattcaaaagcagagacattactttgccaacaaaggtccatctagtcaaggctatggtttttccagtggtcatgtatggatgtgagagttagactgtgaagaaagctgagtgccgaagaattgatgcttttgaactgtggtgttggagaagactcttgagagtcccttggactgcaaggaaatccaaccagtccattctaaagatcagccctgggtgttctttggaaggaatggaatattacttggccttttttaaaaaaggataagattctgcaatacgcaaggatgtgaacctcatgataagtgaaacatccgcatcacagggacaaatactgcatgattccactctcaggtggtatttaaaatagtcaaactcatggaagcaaagaacagaatttgtgtttcaggggctgagggaggggagcatgagatgttcagcagtgatgcggggtcacttatgcaagatgagtaagttctagagatctgctgtataacctcgtgcctgcccataacagtgctgtgttgtacttaaaaatttaggagggtaagtctcatgtgaagtgttcttatcacagtgatttaaaaaaaggagtttctaaattttttttaaaaatggatacaaatgtgtaattttaaaatgatttagtgattttgctcttaacttaatttctcaaatgaccaagggcccagcccccagcgctgtcaggttaggaagtttctactctaggcttggccgtgtgtgctaagtcaattcagtcgtgtccgactcttggcaaccctgtagactgtagcctgccaggctcctccgtccatgggattctccaggcaagaacactggagtgggttgctatgctctcctccacgtggttttcccgacccagggattgaacccacatctcttatgtctcctgcattggcaggcgtgttctttaccactaatgccaacagggattgagcctgcaccccctgcatcgcaaggcaaagtcttaaccactggaccacgagggaagttcctcaggcttggccgtaaactcaacctaaattgccatgttacacttggaacaacccacaggtaccaagaatccaagaccttctagactctagaacatgaatccgcatcaccttcaaggctaccacacacacacacgcacgcgcgcacacacacacacacacgcaatctagcctcatttgagttcactgcagtctggttgttggaggccttgtctaaacagcccaactgggatctgagcctgcgctttcttggggtgaggaagtatgccttcaggcctccagtcctctccccacccacttcttatgaccggcctcaacctgcccagagcctgccagactagctaaacttggcacccagtgagccttccgaaacctgtactgagtgaaacatctttataaggtcttcttagagcttaatataatgaaagccttttggctgaggttcagggaatatgcagacacagtgtcttggggggagtaggtagacgaaacgtggtaggaaagctgcccaagaattatttcactcctatctcatttgtaccctcctcacagacccttctccgccccttcccaacccgtggggctggcagaaatcggcgtggaggaaacagccgaacataggctctgaaagacaaacacccgaacccaggggcttagagcagaccttatttatgtgtccacttgttttacacgctgacttgactacacattattcagaccagtggttgcaagcggttgttaccaagttcgtagcaaaagcaaatcacctggaaggtgagtttcaaaacagacagggacagggattttagaaagcctagccaactgcttgtgacctgaccagaagtaaaacatctcctgcctctcccaaagtcaacaggcaaaatccgaaaagctgaaaagtgtgccactgggcacacgaccgttactttgctcagatccgctaagcaggcaacttcttttctctgtcttctgtgtgtgtagaaattaacttgggcaaatatgtgtcaagtgcacatacagaattgttatatagtaagatgtacccataaatgtctataatcgggtgaatccattttgttaaaatgcagtgtgaaaaatgagaaagtaataagtgattgctctgttatccttcaaattaggtacacagccttcttaggctttgtcttatgagaaaatataaaatgtgcagatttaaattgagaagtggctttgagttctggactatttcagaaagaactcatgcgatggttttccctagcaacggtccaatttgtgctgcagaagaggctcgtatgctgataaactggtattgacaagtgtgatagatacagcttggtacttagtgagtgctggagagcaaagttgattgcattttacagtctgtgtatttctctgtttattgcttgtattctgtcattaagcaaacaggttcttaatatagcaaatctctctaatcagtaaagcaagggagaaaagaaccagaagcatcattaattatgaccatttggctgcagcctgggcaatctgttgctattggattgagcaacttcctccagttttattttctggaggtagagcagaaactggcttttcttatacatggaggagagtagagaaatctccagaaaataaaactggttaaacactggcttctctacagaaatggagtcctggggcagcaaaagccaagctgcaagtgggtatggtattaaacctccagttctataaacagagcaagtgtgcaactccaaagcctgatcccgcccgaggctccgggagagcccactgtggccagaacgcgtggggcacgcggtgcctgatgagttagcttgaccaagagttctggactctatgtgtgtctgagcattctgtacaggaaagcagtcggcaccaggcaggctttccaaatgttaatcacagggttaaaaagcaggagagtcattgtctgactccaggtaacagagattaatagcatctaatgcatttattaaccctctagtgccagggactggcggagtccggcagcaattacagtgacatgaaaattctattatgtgacgtgcctggacttggggattactgccagtgacagtcttataagactgttggagggacaagggttgtgaagagcctaaagattaagttccagaaagagagaaaagctactgggttgtggatggatttctccagcagcctgttcactgtctcaagataaaaccttgtagtcccatgttttggtggtgttttccccaagttggaggcttctcccataaccacataactatcagttaggtgataggaaaccctctttcacaaaagagattaaataattcatttttcaatatgctggaactgtggttaaaattgacttttagaaaaactgaaatccattctcttcctttgtatcccccccttttttttaaagagaaagatagcatattttaaagccacagacggtatggtttggtgtctagatagatctagctagtcaagctctatgttgggccccttcctatctttctttctttctttcttccttttgactgcttcacatggcatatgggatcctagttcccagaccagggatagaatggcgacccctgcagtggaaggcggactctcaaccactagaccaccagggaagtcccctgtcttcttaataagcagggcttggcagatgggatgcattccattattacactcctgcttttatcaatttgggacttggaaatggtgctcttccagtttcttctaccttaccttatactttgcaaagtctcagaaatttttgttggtttgttttgttttcagttttcctgggactgttgcaattttcatcaattgaggccatgttcaaaatatgtaaataatctttgtaatagctaaaagtgggagatgccctgtaattgtatctgtttgcatttatcagacctgactagtaaggcagtgatagctgatgttttaaaaagcactcaggattgttgccattgttgttcagtcgctaagtcatgtccaactctgcggccccgtggactgcagcaggccaggctcctctgtcctccattgtctccctgagtttgctcagactcctgtttattgagttgctgatgctatctagtaatctcatcctctgccacccccttattcttttgccttcaatctttcccagcattggagtcttttccaatgagttggctctttgcttcaggtagccaaagtattggagcttcagcatcagttcttccaatgaatattcagggtcaattgcctttaagattgactggtttgatttcctcaaagtccaagggactttcaagagtcttctccagcaccaaatttgaaagtatcaatttttcagtgctcagccttctttatggcccaactatcacattcatacatgactactggaaaaagctttgactgtatggacctttgttggcaaagtgatgtctctgctttttaatatgctatctagatttgtcatagcgttccttccaaggagcaagtgtcttttaatttcatggtggaagtaacagattgcttaggcttctaaaagggacttcccaagagctggattttcttaagtgggctctggatacgcatagggggatttctgacccttactggtcacaggagggattcagaatgaatacaggacttataagcatgcacactgctagtgcatgcacaggtataaattcccaggctctgcccctccacctgcagctcatgaacccttagaggcactggacacccagctgacaccattgagtccaagcagcagaccactggccctccagcccaggtttggtgtgggattttaaaagatctcatgtctgtgtgcaatctgattcgtttatgcaggtgttatcttccctccacctcctaatcacatcacagatcttactgcagttctcatgaggttctggtggaactcagggtctgctctgccctgcctctgaggttgtgagggtttctgggctcccgcaacctctctatgggaagcagtgtcaactcagtatataagcagagtatttagaagccagcgtacatggactgctaagcttctggggtgctgagtgcttctgtggattggtcatgggtttgataggagagaaagcattttctcagagactctgcagaagcctggtggcacatggttactttccttcagcagttcatccagttctgcccactgaatagttcttgaagccagatgagcttgaagtttctgcctctagctttaatgaagccatcgggctgcagggagacaaggccagaagttgactatttgctttagacagaggccaacaagaggcccgccttgcagggaagaggagctcagagccttcacaggcttggagttgatctccatgtattttcctgtgtgatttgaccagtcatcccaaccattctcagacagaaagcaagagcctaggaatttagaccagggcgtgtctacccagttgtacaactgtaatcctgtacacagttgaactctcagtgactagttgaggaccagaatttacctgtgtcaatgcagaatccatttaagaaaattagttaaagtagcgacagggttgcaaagagttggacacgactgagcgactaagcatagcacagcacaaagaccttttaaactccgttggtggtagcccctttacaatcaggtaatatagacgggcatttttttctaatacatcagcaggttcctaaagtgacttagaagtgtagccagtgagtctctcagatggtgcaaacaaagagaaaaacactgtagtctgttgctaatcatagagaacaaaataggagggaaacccaaccagccattggatctcttggtgggcactgcagtctaggacacggaggtgtagcttaactatgacaatagcacagaagcaaatggaaggattaatgcatcccagacaagtcaggctgccttctctgcacgttttagtgaacttctgagttatgcaaaggcgcttggcaaggctttatgtctcttacagattgggggcttcccaggtggcactagtggtaaagaacctgcctgccagtgcagatagatactggagacattgattcctgtgttgggaagattccctggagtagaaattgacaacccactccagtagtcttgcctggagaaccccatggatggagaagcctcgccagcaggctacagttcatagtgttgcaaagagttggacgtcttagtgaacttcttacttatgcaaaggtacttagcaaggttttatgtcttttatagattaccacctggaaaagaaaagacaaaatgtgatgttttagggccattttggtgtgtggcacaattccagttttgagcagaaataaaatgtgcaaagttctacatattattctctataggttacaatgcagtggaccagatttgctattaatatagattaaaagtaggcttttgaggaaggctccatccctcttctgatttgccctctgatcttcttatacgtaacatttagtgacctttgaacagcccaggggtgcttcacagtgtctttttcttgatgcctgtttaaaggtttcaatttcatattcacagctggcatatgcaaatataggtaaagattatgatgcctcccaagtagcattatggtcaataaattactccttgtgtgactataccatgatctcagcctagcattcttcaacaagttgcaaaagttgtcctgcagcaggcagcctgccaatgcatccccatagagctggttgcccagatgctcctagattcaggtgtgatgcttaaccagggccatcctgttgacaaactaactctctgtcttttgctaccaaagggctcctagtttcagggggagggggtataggagggtttctctgagtggatttggggggcaagtagcgcatgaagagtcaagcatgctagtatctccgggagaaacactggaaagcaggaaagagcacgattccattcagaagtttcagagacctgtattctaaatgttgactacaacttagatcactctgtgaccctgaataagtctcttaacgtctctgctctctaatttgtcttcctttataaaacaagagagctgtattgggtcagcagtttttattataattgagcaatggagtgtttcctctaacttgatttctcaggtagaaacccagtggaaacttgttccatttgtgagaaaacagggagcttagagccctcacagctcaggaacttataacttcccaggaagcagttttaagatggatagagctggggtctgtgatacccttcatgattcttggaagggacctgtcaaagggtccctgccctgtgcctgagccccccagcgctgagtagtttacccaccagaaggcaggcccaggagcccaggaccacagcctttttgtcatatcactgtatgcccaaccctggaaaattgttcaataagtatttgtggactggaacaactaataaacaaatgaacaaatacacaagattcaggaagacctatctctatattactgatgaatgaggatttttattacaacatttttacctagaaggcacttcataaacattaatattcagaacttataaatgagtacagatcgcaaatgactttaaaacttttttcttttgtaacctgtgcagtggctttgagagggggcgggtacaaactcaggtgtattttgtccaacacaccaaatttttttttcacaccaaattttgaagacttagtatgaaagtttaacaaatgtagaaaagtatctcattaatcatcttttatactggttacatgttgaaatggtaatatattgagttaaagaaaatgtcattaaaattttctttacctgttatctctttatttatgggattagtagaaaaatttaaattatgtatgggctcacagttgtgactcacatatttctaacaatgacacagtcttatactgttgatcatcaataactactcttccataatggtttcgtcacttagtcgtgtccatctctttgcgaccccacggactgtagcctgccaggctcctctgtccatgggattcccaggcaagaacgctgcagtgggctgccatttccttctccaactcttacatagtatgtacaaataattgtgtgtgtttgtcttttcccaggcacagaagctgaaatccaacatatcaggcagtactcacctctgtctttctgatgtaagttgtttctttctgagtctccctacatctagaaacagagcctttgggtccctggaacagggtgtgatgggaaagggctcaaagacaacaaggtttcaggcccagaaaaaaaatctaagtctcccttggtgtccctctgccctcggaaccaggtgcagatgctggtgtacaaagcagcgccaagtgtgggaccctcatctcagaactggaccagcacagataccattgtcctggtaccatgagaacctctgtgatcacagtcacataagttcaactggcacagagaagatacataacacctcttgctgcttaccaaatatcccctagactgctcctttgactttcacagcgacccagaagtagccaatgcaggccacgttaccctccccaatttacagttatagctcatcatggcaggatgtgattttctggagttgagcaggtcctagggatgggggtgaaggaatccatcctggggaagaacagctgtgctgactgagagttcaggagcctgggtccttgctgggactctgacaagcagggcaagatcctccatctataaagtccagggttgaaccagccggtggagggaaaggctactgccaggctccaatgcgatttgtgacccatctctatgtgagcccgtacttgcctgggcaggcttcctgagatgattcttcctgccctccttcatgacagagagagagggcaagcatcccttctcagtcagtgatacccaaagggaggatggttccatgttgcacagctgatgggtgaccttaggagcaggggtaaaaatcttgggctttggtaaaccatatagctagttgggggcagggatcgaagagggaaagagtgcagctaaaatatgccgtaatagaacaaatacatgcagtgggtgataatgaaaacgttctttcttacagcgtgattatgaaaaggatggtctcgctggggcttccagccacataaccacaaaatcaatggctgctcctccaagctgcggtctgagcccactgctggccctggtagtaactgccctgtccaccctattgtctttatctttggccgaagcatcgtagctgcatttacagaaccatatggacgtgtgaaaagacaaaaaccaagttatctgtttcctgtcttcttgtatatccaaaattttaggagctcagttgagacactgctaaaacagtttcatccaactggaacttttttctttctttttttttttttctaagaaagcttcttgtgatccttaaggcttttatgggatcccccgatacagtgctacattccacactcagaaggctttggggcatgctggattttaaagagatggtttgttgttgatgtggctgtgaagcggactggagccttggctttcaagatcctgataaggatgatgatttttaacagtttcactctggtcttgactagctagagaaggagtcagtaatcctaaagaatcttctatatctcatggaataggtttgctttccgatgacatgaccataccatggcagtggtacgtggaagccaaactttttttgtcacaaagcgaagattctcatgacaatcttgatggactttgtgcaaacaagcttgtaccagtgttctttctatacgtactagcgtttgccacgctaatgtttatgtacagtaaacagctctgcagtctcttatgaaaacaaaaaacacacctgccacattcaatgtagtgtctgtctcctagtcaaaatagagaatgagagtgggtgtgagacttcgcagaaccttgagcattgagacagtctccaaagccttacttgagtgagaagcccttacactaacaagagtcaaatatagctgaagcatcattctaataatctttacatgtatgaggttatatgtagaagaaaaattttactactaaatgatttcaactattgactttctatattttgaaagtaatgatattgtattctttttttttttttttttttttttttaccggtggtttaatacaaaatacatggagcttgttttcctctcataagtagcgttagcttcgatgttgacttcttgaagacaggtcttcaacagcagactctaaggagcctcatgctttagcagaaagctctgcatcacgttactgtggacaggcagcaggaaatagagcagccaagcattgtcttttatcatttctggaaacacaagcgtgcatacctgtgggaaaactggtggccactcagtgttctacaaacactacaaatggcaccgttgccagctgacacaaaccagggccttggttttcatgttacctgacaatggcagctttgactgggacaggagacaggacgtgctgccctgtcttctgaagcagatccatgcccaggatacacagctcttatcctaaggattgtcttgactttcacctgtgatggcttggccgctctgtggatcggtttggtctgaagtcatcagatatctgctcgcactgtgttctgtgtcctctgtgatagactgtgactgactctgcgaaaaagaaaaaagtttgggggatcaagagagtcacggtcaccattaggtccatggagataagttggcccagagacacagaacagggactctggatataaggatctgacttgttaactgcacgtcccttccatcattctcttccttcagtgaatagccccgaaagtgcacttctgaagtctggttgaaatgctgagatttgaaatctacaaggatcgagtgctagtagccatgtttctttgcaactgaacagaggcgaggaaatttcacccaatagagtatccgaaaagttggctttcttctgacatcagtagagtggatggaggagaaaacccacagtgcccatttcatcctgattccctttgcccacggatgtttgccttgtaagaattaatccacttactagggcgggaaatctgttaaacccaagatgaaggcatagtcttttagctctgttggtgtttgaagtacatttatatccaaatgttaataaacataaatgtataatactaa

SEQ ID NO: 3 - protein, 456 aa, Bos taurus
MFLATLYFVLPLLDVLLSAEVSGGDRLDCVKASDQCLKEQSCSTKYRTLRQCVAGKETNFSLTSGLEAKDECRSAMEALKQKSLYNCRCKRGMKKEKNCLRIYWSMYQSLQGNDLLEDSPYEPVNSRLSDIFRVVPFIPDVFQQVEHISKGNNCLDAAKACNLDDTCKKYRSAYITPCTTSMSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCNNSGNDLEECLKFLNFFKDNTCLKNAIQAFGNGSDVTVWQPALPVQTTTATTTTAFRVKNKPLGPAGSENEIPTHVLPPCANLQAQKLKSNISGSTHLCLSDRDYEKDGLAGASSHITTKSMAAPPSCGLSPLLALLVFLS

SEQ ID NO: 4 - DNA, 24 bp, Artificial Sequence
  Primer
gatcacctaacatcttgcctaatc

SEQ ID NO: 5 - DNA, 22 bp, Artificial Sequence
  Primer
ctattgctatccagtcatgtgc

SEQ ID NO: 6 - DNA, 26 bp, Artificial Sequence
  Primer
tgtgtctctctatctatccctgtctc

SEQ ID NO: 7 - DNA, 20 bp, Artificial Sequence
  Primer
cgttcctgcctctgtctctc

SEQ ID NO: 8 - DNA, 25 bp, Artificial Sequence
  Primer
aaatagcagaaatttagaagcagca

SEQ ID NO: 9 - DNA, 21 bp, Artificial Sequence
  Primer
tgtgagcacgtgtgtgtatga

SEQ ID NO: 10 - DNA, 22 bp, Artificial Sequence
  Primer
caagacaacgcttatcccacag

SEQ ID NO: 11 - DNA, 26 bp, Artificial Sequence
  Primer
cagtcctaagaaactaacatagcacc

SEQ ID NO: 12 - DNA, 28 bp, Artificial Sequence
  Primer
atgctcctcacggtacctctgtcctaaa

SEQ ID NO: 13 - DNA, 21 bp, Artificial Sequence
  Primer
gttcccttccagagctcaagc